(19) United States
(12) Patent Application Publication
(10) Pub. No.: US 2024/0131072 A1
(43) Pub. Date: Apr. 25, 2024

(54) ACTIVITY-INDUCIBLE FUSION PROTEINS HAVING A HEAT SHOCK PROTEIN 90 BINDING DOMAIN

(71) Applicant: Seattle Children's Hospital d/b/a Seattle Children's Research Institute, Seattle, WA (US)

(72) Inventors: Ryan Koning, Seattle, WA (US); Adam Johnson, Seattle, WA (US); Chris Saxby, Seattle, WA (US); Michael C. Jensen, Bainbridge Island, WA (US); Ian Blumenthal, Seattle, WA (US); Aquene Reid, Seattle, WA (US)

(73) Assignee: Seattle Children's Hospital d/b/a Seattle Children's Research Institute, Seattle, WA (US)

(21) Appl. No.: 18/546,199

(22) PCT Filed: Feb. 11, 2022
(86) PCT No.: PCT/US22/16119

§ 371 Date: Aug. 10, 2023

Related U.S. Application Data
(60) Provisional application No. 63/226,554, filed on Jul. 28, 2021.
(60) Provisional application No. 63/149,131, filed on Feb. 12, 2021.

(51) Int. Cl. A61K 35/17; A61K 39/00; C07K 14/72; C07K 16/18

(52) U.S. Cl. CPC A61K 35/17; A61K 39/4611; C07K 14/721; C07K 16/18; A61K 2239/15; C07K 2319/02; C07K 2319/03; C07K 2319/30

(57) ABSTRACT

Activity-inducible fusion proteins whose activity is post-translationally regulated utilizing a hsp90 binding domain and a drug molecule are described. In the absence of the drug molecule, the activity-inducible fusion proteins are inactivated but can be activated by a relevant physiological parameter in the presence of the drug molecule. Examples of the activity-inducible fusion proteins include chimeric antigen receptors (CAR) wherein the relevant physiological parameter is antigen binding.

### CROSS-REFERENCE TO RELATED APPLICATIONS

This application is the U.S. National Phase Application of International Patent Application No. PCT/US2022/016119, filed on Feb. 11, 2022, which claims priority to U.S. Provisional Patent Application No. 63/149,131 filed Feb. 12, 2021 and U.S. Provisional Patent Application No. 63/226,554 filed Jul. 28, 2021, the contents of both of which are incorporated by reference herein in their entirety.

### REFERENCE TO SEQUENCE LISTING

The Sequence Listing associated with this application is provided in text format in lieu of a paper copy, and is hereby incorporated by reference into the specification. The name of the text file containing the Sequence Listing is 2XS0676_txt. The text is 564 KB, was created on Aug. 2, 2023, and is being submitted electronically via Patent Center.

### FIELD OF THE DISCLOSURE

The current disclosure provides activity-inducible fusion proteins having a heat-shock protein 90 (hsp90) binding domain. The activity of the fusion protein is post-translationally regulated utilizing a drug molecule that can bind the hsp90 binding domain with a higher affinity than hsp90. In the absence of the drug molecule, the fusion protein is in an inactivated state but can be activated in the presence of the drug molecule.

### BACKGROUND OF THE DISCLOSURE

Using genetic engineering, significant progress has been made in activating and directing cells of the immune system to kill cancer cells and infected cells. For example, T cells have been genetically engineered to express molecules having extracellular components that bind particular target antigens and intracellular components that direct actions of the T cell when the extracellular component has bound the target antigen. As an example, the extracellular component can be designed to bind target antigens found on cancer cells or infected cells and, when bound, the intracellular component activates the T cell to destroy the bound cell. Examples of such molecules include chimeric antigen receptors (CAR).

Although CAR-expressing T cells can demonstrate potent anti-tumor activity, significant toxicities can also arise, for example, by engraftment-induced cytokine storm (cytokine release syndrome), tumor lysis syndromes (TLS) and ongoing B cell cytopenias, each of which are attributable to unregulated functional outputs of constitutively expressed and active CAR. Such toxicities can limit the applicability of CAR-based therapies.

Methods to eliminate CAR-T cells such as suicide gene-mediated elimination of these cells, for example, can ameliorate such toxicities; however, this approach risks premature attenuation of anti-tumor activity and can significantly impact curative potential. As such, there is a need to identify methods for controlling CAR activity following expression by gene-modified immune cells.

Beyond CAR, there are numerous proteins for which the ability to control their activation state would be beneficial. Additional examples include transmembrane receptors, such as those transmitting co-stimulatory or inhibitory immune cell signaling.

### SUMMARY OF THE DISCLOSURE

The current disclosure provides fusion proteins whose activation state can be controlled through the administration of drug molecules. The fusion proteins include a heat shock protein 90 (hsp90) binding domain that binds the drug molecule.

One exemplary fusion protein of the current disclosure is a CAR whose ability to be activated by antigen binding in vivo is controlled with the administration of drug molecules. The ability to control post-expression activation in vivo provides an important safety improvement to CAR-based cellular immunotherapies.

The current disclosure achieves these advances by incorporating a hsp90 binding domain within the intracellular component of the CAR. In the absence of the drug molecule, the hsp90 binding domain is bound by hsp90 preventing the CAR from interacting with other key intracellular molecules required for CAR activation following antigen binding.

When the drug molecule is present, the drug molecule displaces bound hsp90 from the hsp90 binding domain site and/or otherwise results in a conformational change, such that intracellular signaling can occur following antigen binding.

Certain embodiments disclosed herein utilize a hormone binding domain as the hsp90 binding domain. The hormone binding domain can be an estrogen receptor binding domain (EBD). The EBD can be derived from the natural estrogen receptor but include at least one mutation such that the EBD no longer binds estrogen, but instead binds a drug molecule with a higher affinity than hsp90. Exemplary drug molecules include tamoxifen or derivatives or metabolites thereof with fewer side effects such as 4-hydroxytamoxifen (4-OHT), CMP8 or ES8.

In the absence of tamoxifen or a derivative or metabolite thereof, hsp90 binds the EBD and the CAR is in the “OFF” state. Nanomolar concentrations of cytosolic tamoxifen, however, can actively out compete hsp90 for EBD binding and/or otherwise result in a conformational change, allowing CAR-based activation signals following antigen binding.

This strategy can be used to control the activity of other proteins that similarly interact with hsp90 with a lower affinity than the drug molecule. Additional examples include transmembrane receptors, such as those transmitting co-stimulatory or inhibitory immune cell signaling.

One benefit of the current disclosure is the ability to control the activity of a single chain protein without reliance on dimerization or multimerization with another protein, and without reliance on protein stabilization/destabilization, for example through the incorporation of a degron sequence.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIGS. 1A-1C. A visual representation of the role EBD (e.g., ERT2, EBD(CMP8), or EBD(ES8)) plays in the signaling of CAR. (1A) The EBD binds heat shock protein 90 (hsp90) in the absence of a drug molecule, sterically inhibiting the signaling regions of CD3zeta and 4-1 BB from interacting with the SRC family kinase lymphocyte-specific protein tyrosine kinase (LCK), and the LCK-mediated phosphorylation of CD3ζ and ζ-chain of TCR-associated protein kinase (ZAP70). (1B, 1C) However when the drug molecule (e.g., 4-hydroxytamoxifen (4-OHT), CMP8, or ES8) is added, the hsp90 dissociates and the two signaling domains can signal as normal when the CAR binding domain (e.g., scFv) interacts with its associated antigen.

FIGS. 2A, 2B. CAR Schematics. (2A) Generalized schematic of a CAR disclosed herein. The CAR includes a ligand binding domain, a spacer, a transmembrane domain, a signaling domain, an estrogen binding domain (EBD) and selection/transduction markers. (2B) General schematic of a 2nd generation 806-ERT2 CAR. This CAR incorporates the IgG4 hinge between the scFv and the transmembrane region. There is a Gly3 linker consisting of three glycines that connect the signaling domain to the ERT2 EBD. This ERT2 EBD has the sequence as set forth in SEQ ID NO: 13 and can be referred to herein as ERT2, EBD(4-OHT), or an EBD or ERT2 with G400V, M543A, and L544A mutations. The ERT2 domain is then followed by P2A-DHFRdm-T2A-CD19t as markers for selection and transduction respectively. The 806-ERT2 CAR differs from the 806EGFR CAR, in that the 806EGFR CAR does not contain and an EBD or DHFRdm and uses EGFRt as a transduction marker

FIG. 3. Jurkat-Dual NF-kB-Luc Reporter T cells. Jurkat-Dual cells feature the Lucia luciferase gene, a secreted luciferase reporter gene, driven by a promoter fused to five copies of the NF-κB consensus transcriptional response element. Secreted Lucia luciferase is measured as a proxy for NF-kB response via CAR activation.

FIGS. 4A, 4B. Jurkat-Dual NF-kB reporter cells were transduced with either the 806EGFR CAR or the 806EGFR-ERT2 CAR and selected on MTX if possible. 24 hours prior to assays, the transduced cells were co-cultured with K562+EGFRvIII, and various concentrations (0-16,000 nM) 4-OHT. At the 24 hr timepoint, 10 μl of supernatant was collected and combined with 50 ul QUANTI-Luc, and immediately read on the SpectraMax. Transduced Jurkat-DUAL cells were accessed via flow cytometry prior to assays. Flow cytometry of (4A) CD19t (CD19 APC) and (4B) EGFRt (Centuximab APC) confirming expression of 806EGFR CAR or 806EGFR-ERT2 CAR respectively in Jurkat-DUAL cells.

FIGS. 5A, 5B. Jurkat Dual NF-kB-Luc Reporter Assay. Endpoint Luminescence assay measuring secreted Lucia luciferase. Assays were run at either a 2:1 (5A) or 1:1 (5B) effector:target ratios. These cells were stimulated with K562+EGFRvIII and a set concentration of 4-OHT for 24 hours prior to running the assay. Both cell lines were 80% positive for CAR. 4-OHT was added 24 hours before the assay. Off MTX selection was conducted for 1 day prior to assay. Jurkat-DUAL cells transduced with 806EGFR-ER2T CAR cells demonstrate dose dependent activation of CAR T cells by detection of increasing levels of secreted Lucia luciferase corresponding with increasing levels of 4-OHT. As stated previously, ERT2 can also be referred to herein as EBD(4-OHT) and corresponds to SEQ ID NO: 13.

FIGS. 6A, 6B. S1R1D6. Post stimulation culture (S1) Mock, 806EGFR CAR (6B), and 806EGFR-ERT2 CAR (6A) T cells were expanded in a Rapid Expansion Protocol (REP) by stimulation with irradiated feeder cells (20E6 TM-LCL and 100E6 PBMCs) in the presence of rhIL-2, rhIL-15, and exogenous OKT3 antibody. On Day 6 of REP CAR positivity and percentage of CD4 and CD8 T cells was measured via flow cytometry. Mock (40% CD4/60% CD8); 806 CAR (30% CD4/70% CD8); 806-ERT2 (46% CD4/53% CD8).

FIGS. 7A, 7B. Intracellular Cytokine Staining (ICCS). IL-2, IFN-gamma, TNFa. 4-hr ICCS assay using primary T cells expressing the EGFR806 CAR (7A) or EGFR806-ERT2 CAR (7B). Cells were gated on: Lymphocytes/Single Cells/Live/CD4/CD19t to evaluate CD4 CAR containing T cells. 4-OHT was added 24 hrs prior to assay and continued to be present during 4 hr incubation. The EGFR806-ERT2 CAR was able to elicit a cytokine response against EGFRvIII+ target cells only in the presence of 500 nm of 4-OHT or (Z)-endoxifen (ON state).

FIG. 7C. Intracellular Cytokine Staining (ICCS). CD107a Degranulation. 4-hr ICCS assay using primary T cells expressing the EGFR806 CAR or EGFR806-ERT2 CAR. Cells were gated on: Lymphocytes/Single Cells/Live/CD8/CD19t to evaluate CD8 CAR containing T cells. 4-OHT was added 24 hrs prior to assay at each dose level (20 nM, 100 nM and 500 nM), and continued to be present during 4 hr incubation. The EGFR806-ERT2 CAR demonstrates increased degranulation by CD107a marker expression corresponding to increasing levels of 4-OHT concentration.

FIG. 7D. Intracellular Cytokine Staining (ICCS). Early activation marker Nur77. 4-hr ICCS assay using primary T cells expressing the EGFR806 CAR or EGFR806-ERT2 CAR. Cells were gated on: Lymphocytes/Single Cells/Live/CD8/CD19t to evaluate CD8 CAR containing T cells. 4-OHT was added 24 hrs prior to assay and continued to be present during 4 hr incubation. The EGFR806-ERT2 expresses early activation marker Nur77 against EGFRvIII+ target cells only in the presence of 500 nm of 4-OHT or (Z)-endoxifen (ON state).

FIGS. 8A-8D. Chromium Release Assay. 4-hr Chromium Release Assay using primary T cells expressing the EGFR806 CAR or EGFR806-ERT2 CAR. The EGFR806-ERT2 cells were dosed to 78% CAR positivity to match the 806EGFR cells. 4-OHT was added 18 hrs prior to assay, and continued to be present during 4 hr incubation. The EGFR806-ERT2 CAR was able to lyse EGFRvIII+ target cells only in the presence of either 4-OHT or (Z)-endoxifen. 4-OHT or (Z)-endoxifen was added 24 hours before assay, and during incubation. S1R1D10 (8A) K562 Parental; (8B) K562+OKT3 (positive control); (8C) K562+EGFRvIII; (8D) Chromium Release Assay. FIG. 8D is broken down into each dose of 4-OHT or (Z)-endoxifen.

FIG. 9. Depiction of the Jurkat iSynPro:GFP-ffluc model (top) and associated results (bottom).

FIG. 10. Activation curves for huCD19-EBD (ERT2, also referred to herein as EBD(4-OHT) (SEQ ID NO: 13)) with different Gly linkers.

FIG. 11. Design of B7H3 CAR EBD mutant study. The objective of the study was to examine EBD in the context of the B7H3CAR and to investigate the activation efficacy and differences between two EBD mutants (EBD(4-OHT) and EBD(CMP8)) interacting with their respective estrogen analogs (4-OHT vs CMP8). Jurkat iSynPro lines included cJ10792: Jurkat iSynPro-GFP:ffluc; cJ13093: Jurkat iSynPro-GFP:ffluc+B7H3 CAR; cJ13094: Jurkat iSynPro-GFP:ffluc+B7H3 CAR EBD (4-OHT); and cJ13095: Jurkat iSynPro-GFP:ffluc+B7H3 CAR EBD (CMP8) with a K562 parental target. 4-OHT and CMP8 concentrations of 0 nM, 500 nM and 1000 nM were tested for each cell line. EBD (4-OHT) is ERT2 (SEQ ID NO: 13) and EBD (CMP8) is SEQ ID NO: 11.

FIG. 12. GFP induction indicating activation regulation in B7H3 CAR utilizing EBD(4-OHT (SEQ ID NO: 13)) and EBD(CMP8) (having L384M, M421G, G521R mutations (SEQ ID NO: 11)). In this FIG., EBD(CMP8) is referred to as B7H3 ERT2-(L384M|M421G|G521R (CMP8)).

FIG. 13. Activation curves for B7H3 CAR ERT2 (SEQ ID NO: 13 (mid-shaded gray and diamonds)) and EBD(CMP8) mutants (SEQ ID NO: 11 (light gray, dark gray, squares and circles)).

FIG. 14. Design of activation curve study. The objective of the study was to examine activation curves of CAR EBD Jurkat iSynPro lines in an increasing presence of 4-OHT and (Z)-endoxifen. Jurkat iSynPro lines included cJ10792: Jurkat iSynPro-GFP:ffluc; cJ13227: Jurkat iSynPro-GFP:ffluc+huCD19 CAR; cJ13097: Jurkat iSynPro-GFP:ffluc+huCD19 CAR 1GLY EBD; cJ13098: Jurkat iSynPro-GFP:ffluc+huCD19 CAR 2GLY EBD; and cJ13096: Jurkat iSynPro-GFP:ffluc+huCD19 CAR 3GLY EBD with K562 and CD19 targets. The study was run as a 1:2.5 drug dilution series. The highest concentration was 2500 nanomolar for all drugs tested. The EBD in these studies is ERT2 (also referred to herein as EBD (4-OHT); SEQ ID NO: 13).

FIG. 15. Dose response and comparisons of huCD19 CAR-EBD linker variants.

FIG. 16. Design of activation curve study. The objective of the study was to examine activation curves of CAR EBD Jurkat iSynPro lines in an increasing presence of 4-OHT, (Z)-endoxifen, or CMP8. Jurkat iSynPro lines included cJ10792: Jurkat iSynPro-GFP:ffluc; cJ13093: Jurkat iSynPro-GFP:ffluc+B7H3 CAR; cJ13094: Jurkat iSynPro-GFP:ffluc+B7H3 CAR ERT2(4-OHT) (SEQ ID NO: 13); and cJ13095: Jurkat iSynPro-GFP:ffluc+B7H3 CAR EBD(CMP8) (SEQ ID NO: 11) with a K562 target. A drug dilution series (1:2.5 curve) with a 1000 nM start for 4-OHT and CMP8 and a 6250 nM start for (Z)-endoxifen was used.

FIG. 17. Dose response and comparisons of B7H3 CAR-EBD linker variants (EBD(4-OHT) (SEQ ID NO: 13)) and EBD(CMP8) (SEQ ID NO: 11)).

FIG. 18. Drug dependent specific cell lysis with different B7H3CAR-3Gly-EBD mutants in the presence of estrogen analogs 4-OHT, CMP8 and (Z)-endoxifen. Specific cell lysis was evaluated using a chromium release assay. First the B7H3CAR-3Gly-EBD(4OHT) and B7H3CAR-3Gly-EBD(CMP8) mutants were cultured for 24 hr in differing drug concentration (0 nM, 1 nM, 50 nM or 500 nM) of three different estrogen analogs (4-OHT, CMP8 and (Z)-endoxifen), they were then co-incubated for 4 hrs with an antigen expressing tumor line that had previously been labeled with radioactive Cr-51. Following the 4 hr incubation the Perkin Elmer TopCount was used to quantify the amount of Cr51 released into each well as tumor lysis occurred. Results show that no specific cell lysis was observed for either CAR-EBD mutants when no estrogen analog was added. B7H3CAR-3Gly-EBD(4OHT) activity is regulated by 4OHT and (Z)-endoxifen but is not regulated by CMP8. This is indicated by the 0% specific lysis in the presence of 500 nM CMP8. B7H3CAR-3Gly-EBD(CMP8) activity is regulated by all three estrogen analogs. Generally, 1 nM of estrogen analog is not enough to induce maximum activity. Both 50 nM and 500 nM of estrogen analog conferred the highest degree of antigen-specific cell lysis regardless of the EBD mutant. Specific cell lysis resulting from B7H3CAR-EBD activity is dose dependent when cultured in increasing concentrations of estrogen analogs.

FIGS. 19A, 19B. (19A) Drug dependent specific cytokine release with different B7H3CAR-3Gly-EBD mutants in the presence of estrogen analogs 4-OHT, CMP8 and (Z)-endoxifen. (19B) Drug dependent specific cytokine release with different B7H3CAR-3Gly-EBD mutants in the presence of estrogen analogs 4-OHT, CMP8 and (Z)-endoxifen. (20A and 20B) Cytokine release was evaluated using a Meso Scale Diagnostic assay. First the B7H3CAR-3Gly-EBD(4OHT) and B7H3CAR-3Gly-EBD(CMP8) mutants were cultured for 24 hr in differing drug concentration (0 nM, 1 nM, 50 nM or 500 nM) of three different estrogen analogs (4-OHT, CMP8 and (Z)-endoxifen), they were then co-incubated for 24 hrs with various tumor lines that either expressed B7H3 (antigen of interest) or had had B7H3 Knocked Out (KO). Following the 24 hr incubation the supernatant from each co-incubation was collected and run on the MESO QuickPlex SQ 120 instrument to ascertain the amount of cytokine released. Results show that no cytokine was released by either B7H3CAR-EBD mutant, regardless of drug condition, when co-incubated with the non-antigen expressing tumor line (K562 B7H3 KO). B7H3CAR-3Gly-EBD(4OHT) activity is regulated by 4OHT and (Z)-endoxifen but is not regulated by CMP8. B7H3CAR-3Gly-EBD(CMP8) activity is regulated by all three estrogen analogs. Generally, 1 nM of estrogen analog is not enough to induce maximum activity. 50 nM-500 nM of estrogen analog confer the highest degree of antigen-specific cytokine release, regardless of the CAR-EBD mutant. Cytokine release by both CAR-EBD mutants is dose dependent when cultured in increasing concentrations of estrogen analogs.

FIGS. 20A, 20B. (20A) Glycine linker dependent differences in target cell lysis when used in combination with huCD19CAR-EBD(4-OHT) against K562+CD19 tumor cells. (20B) Glycine linker dependent differences in target cell lysis when used in combination with huCD19CAR-EBD(4-OHT) against Raji tumor cells (20A and 20B) Specific cell lysis was evaluated using a chromium release assay. First the huCD19CAR-EBD(4OHT) effector lines with either a 1Glycline, 2Glycine or 3Glycine between the CAR and the EBD linker were cultured for 24 hr in differing drug concentration (0 nM, 1 nM, 50 nM or 500 nM) of two different estrogen analogs (4-OHT or (Z)-endoxifen), they were then co-incubated for 4 hrs with an antigen expressing tumor line (K562+CD19 or Raji Parental) that had previously been labeled with radioactive Cr-51. Following the 4 hr incubation the Perkin Elmer TopCount was used to quantify the amount of Cr51 released into each well as tumor lysis occurred. Minimal cell lysis was observed for all CAR-1/2/3Glylinker-EBD(4OHT) lines when no estrogen analog was added. The greatest activity in the absence of inducer drug was observed for the CAR-2Gly-EBD(4OHT) construct. In the context of the huCD19CAR-EBD(4OHT), the 1 Glycine linker has the least specific cell lysis in the absence of estrogen analog and comparable max specific cell lysis to the 2 and 3 glycine linker in the presence of 50 & 500 nM estrogen analog conditions. Specific cell lysis resulting from CAR-EBD activity continues to be dose dependent when cultured in increasing concentrations of estrogen analogs.

FIG. 21. Drug dependent specific cell lysis remains reproducible in multiple T cell donors. Specific cell lysis was evaluated using a chromium release assay using the huCD19CAR-EBD(4OHT) effector lines with either a 1Glycline, 2Glycine or 3Glycine linker in two different T cell donors. Both donors were cultured for 4 hr in differing drug concentration (0 nM, 1 nM, 50 nM or 500 nM—only low and high drug conditions shown) of two different estrogen analogs (4-OHT or (Z)-endoxifen), they were then co-incubated for 4 hrs with an antigen expressing tumor line (K562 CD19) that had previously been labeled with radioactive Cr-51. Following the 4 hr incubation the Perkin Elmer TopCount was used to quantify the amount of Cr51 released into each well as tumor lysis occurred. Results show that drug dependent specific cell lysis by CAR-EBD is reproducible across different T cell donors.

FIG. 22. Glycine linker dependent differences in cytokine release when used in combination with huCD19CAR-EBD(4-OHT) against K562+CD19 tumor cells. Cytokine release was evaluated using a Meso Scale Diagnostic assay. First the huCD19CAR-EBD(4OHT) effector lines with either a 1Glycline, 2Glycine or 3Glycine linker between the CAR and the EBD were cultured for 24 hr in differing drug concentration (0 nM, 1 nM, 50 nM or 500 nM) of two different estrogen analogs (4-OHT or (Z)-endoxifen), they were then co-incubated for 24 hrs with an antigen expressing tumor line (K562 CD19). Following the 24 hr incubation the supernatant from each co-incubation was collected and run on the MESO QuickPlex SQ 120 instrument to ascertain the amount of cytokine released. Minimal cytokine was released in the absence of estrogen analog. Cytokine release by all CAR-1/2/3Glylinker-EBD lines was dose dependent when cultured in increasing concentrations of estrogen analogs. Generally, 1 nM of estrogen analog is not enough to induce maximum activation. 50 nM-500 nM of estrogen analog confer the highest degree of antigen-specific cytokine release regardless of the CAR-1/2/3Gly linker-EBD variety.

FIG. 23. Glycine linker dependent differences in cytokine release when used in combination with huCD19CAR-EBD(4-OHT) against Raji tumor cells. Cytokine release was evaluated using a Meso Scale Diagnostic assay. First the huCD19CAR-EBD(4OHT) effector lines with either a 1Glycline, 2Glycine or 3Glycine linker between the CAR and the EBD were cultured for 24 hr in differing drug concentration (0 nM, 1 nM, 50 nM or 500 nM) of two different estrogen analogs (4-OHT or (Z)-endoxifen), they were then co-incubated for 24 hrs with an antigen expressing tumor line (Raji Parental). Following the 24 hr incubation the supernatant from each co-incubation was collected and run on the MESO QuickPlex SQ 120 instrument to ascertain the amount of cytokine released. Minimal cytokine was released in the absence of estrogen analog. Cytokine release by all CAR-1/2/3Glylinker-EBD lines was dose dependent when cultured in increasing concentrations of estrogen analogs. Generally, 1 nM of estrogen analog is not enough to induce maximum activity. 50 nM-500 nM of estrogen analog confer the highest degree of antigen-specific cytokine release regardless of the CAR-1/2/3Gly linker-EBD variety.

FIG. 24. Drug dependent cytokine release remains reproducible in multiple T cell donors. Cytokine release was evaluated using a Meso Scale Diagnostic assay on the huCD19CAR-EBD(4OHT) effector lines with either a 1Glycline, 2Glycine or 3Glycine linker between the CAR and the EBD in two different T cell donors. Both donors were then co-incubated for 24 hrs with various tumor lines that expressed CD19 (antigen of interest). Following the 24 hr incubation the supernatant from each co-incubation was collected and run on the MESO QuickPlex SQ 120 instrument to ascertain the amount of cytokine released. For Donor 1, huCD19CAR was tested in all drug conditions whereas for Donor 2, huCD19CAR was only evaluated in the no drug and 500 nM conditions. Drug dependent cytokine release by CAR-EBD is reproducible across different T cell donors as indicated by the trend in IFNg, IL2 and TNFa release as estrogen analog concentrations increase in both donors.

FIG. 25. Drug dependent specific cell lysis huCD19CAR-EBD(4OHT) with different linkers (No Gly, 1Gly) between the CAR and the EBD(4OHT) in the presence of estrogen analogs 4-OHT and (Z)-endoxifen. Donor 3. Specific cell lysis was evaluated using a chromium release assay. First the huCD19CAR-EBD(4OHT) linker variants (NoGly and 1Gly) were cultured for 24 hr in differing drug concentration (0 nM, 1 nM, 50 nM or 500 nM) of two estrogen analogs (4-OHT and (Z)-endoxifen). They were then co-incubated for 4 hrs with an antigen expressing tumor line that had previously been labeled with radioactive Cr-51. Following the 4 hr incubation the Perkin Elmer TopCount was used to quantify the amount of Cr51 released into each well as tumor lysis occurred. Results show that the huCD19CAR-EBD(4OHT) system has greater stringency in the absence of inducer drug than initially observed in donors 1 or 2 (FIG. 22. 1Gly for direct comparison). Still, a small amount of killing still occurred for both the no gly and 1gly variants. Both NoGly and 1Gly variants were successfully regulated by by 4OHT and Z-endoxifen, with full killing (equivalent to the constitutive huCD19CAR) achieved by the 50 nM and 500 nM conditions of each drug. Neither drug appeared to outperform the other in this assay. The NoGly and 1Gly linker variants appear to confer similar regulatory capacity to the CAR-EBD system with regard to antigen-specific lysis as measured by a chromium release assay.

FIG. 26. Anti-zeta Western Blot evaluating huCD19CAR-EBD(4OHT) with differing Glycine linkers in 0 nM and 500 nM of 4OHT OR (Z)-endoxifen. Protein processing and expression of huCD19CAR-EBD with different linkers between the CAR and the EBD was evaluated using an anti-CD3zeta western blot analysis. Whole cell lysates were made for the huCD19CAR-EBD(4OHT) T cell lines with either a 1Glycline, 2Glycine or 3Glycine linker between the CAR and EBD domains in 0 nM and 500 nM 4OHT/(Z)-endoxifen drug conditions following a 24 hr incubation. Whole cell lysates were also made for the Mock T cell line and the CAR alone T cell line under the same conditions. The western blot was imaged and analyzed on the LiCOR Odyssey. Additionally, protein band quantification was performed using the the LiCOR Odyssey. CAR zeta chain expression is observed for all CAR-1/2/3Glylinker-EBD lines in both the no drug and maximum drug conditions. CAR-EBD zeta chain band quantification revealed about a 3-fold increase in CAR-EBD protein expression with the addition of 500 nM 4OHT or 500 nM (Z)-endoxifen. No such change was observed for the constitutive CAR zeta chain band.

FIG. 27. Cell surface huCD19CAR-1Gly-EBD(4OHT) expression changes with induction/removal of drug. CD4 and CD8 T cells (mixed at a 1:1 ratio) expressing the huCD19CAR-1Gly-EBD(4OHT) construct were cultured in media containing 1 micromolar 4-OHT for various periods of time or were removed from culture conditions containing 1 micromolar 4-OHT for various periods of time. Each culture was then stained with an fluorophore-conjugated antibody specific for the scFv component of the huCD19CAR-1Gly-EBD(4OHT) construct. The MFI (median fluorescence intensity) was then measured for each population of cells at each time point listed. The cell surface expression of CAR-EBD constructs changes upon drug addition and withdrawal. The kinetics of this change are very slow in both directions (on the order of days), which is dissimilar to known degrons, and demonstrates an alternative mechanism of regulation.

FIGS. 28A, 28B. (28A) CD107a activation assay: huCD19CAR-EBD(4OHT) linker variants vs K562+CD19t. (28B) CD107a activation assay: B7H3CAR-3Gly-EBD variants vs K562+CD19t. (29A and 29B) CD4 and CD8 T cells (mixed at a 1:1 ratio) expressing a range of huCD19CAR-EBD and B7H3CAR-EBD constructs containing different linkers separating the CAR and the EBD domains (1Gly, 2Gly, 3Gly) as well as different EBD domains (EBD(4OHT), EBD(CMP8), EBD(ES8)) were assayed to determine a) their regulatibility (e.g., On/Off with multiple drugs) and b) the dose response of their activity to drug induction. To achieve this, T cells (including mock and constitutively active CAR controls) were preconditioned with target drug concentration(s) for 24 hrs, then exposed to a tumor line expressing their target antigen (CD19 or B7H3). After 4 hours of co-culture with target tumors at various drug concentrations, the T cells were assessed for a) activation and degranulation by staining for CD107a and b) cell surface CAR-EBD by staining for human scFv (which works for both B7H3CAR and huCD19CAR), then running on flow cytometry. MFI (median fluorescence intensity) was determined for each stain on each sample under each condition and plotted on a log scale of drug concentration. EC10 and EC90 (effective concentration 10% and 90%) were calculated in Graphpad Prism for CD107a expression and function as estimates for the drug concentration required for each construct to turn “on” or “off”. Positive and negative controls for each CAR are presented in the bar graph. Small shifts in CD107a MFA can be observed between mock T cells and un-induced CAR-EBD T cells, suggesting a small degree of unregulated degranulation for all EBD constructs, though noticeably more for the huCD19CAR-EBD constructs than the B7H3CAR-EBD constructs. EBD(4OHT) responds to 4-OHT and (Z)-endoxifen, but not to CMP8 or ES8. The responsiveness of degranulation to target antigen in the presence of inducer molecule is largely similar between the Gly linker variants. EBD(4OHT) is very sensitive to its intended inducer molecules, beginning to activate (EC10) at single nanomolar concentrations and becoming fully activated (EC90) at double digit nanomolar concentrations. EBD(4OHT) may be used to regulate both the huCD19CAR and the B7H3CAR, and has similar dose response characteristics between the two (similar EC10/EC90), even if the huCD19CAR construct exhibits higher background. EBD(CMP8) is less sensitive to drug induction than EBD(4OHT). EBD(ES8) is very sensitive to induction with ES8, however, the maximum magnitude of its induction with ES8 is less than its induction with 4OHT. Both EBD(CMP8) and EBD(ES8) respond to 4OHT. EBD(CMP8) does not respond to ES8 and EBD(ES8) does not respond to CMP8, demonstrating capability to regulate multiple EBD domains independently.

FIGS. 29A, 29B. (29A and 29B) CAR expression: huCD19CAR-EBD linker variants vs K562+CD19t. CD4 and CD8 T cells (mixed at a 1:1 ratio) expressing a range of huCD19CAR-EBD and B7H3CAR-EBD constructs containing different linkers separating the CAR and the EBD domains (1Gly, 2Gly, 3Gly) as well as different EBD domains (EBD(4OHT), EBD(CMP8), EBD(ES8)) were assayed to determine a) their regulatibility (e.g., On/Off with multiple drugs) and b) the dose response of their activity to drug induction. To achieve this, T cells (including mock and constitutively active CAR controls) were preconditioned with target drug concentration(s) for 24 hrs, then exposed to a tumor line expressing their target antigen (CD19 or B7H3). After 4 hours of co-culture with tumors at various drug concentrations, the T cells were assessed for a) activation and degranulation by staining for CD107a and b) cell surface CAR-EBD by staining for human scFv (which works for both B7H3CAR and huCD19CAR), then running on flow cytometry. MFI (median fluorescence intensity) was determined for each stain on each sample under each condition and plotted on a log scale of drug concentration. The change in cell surface CAR-EBD that was previously observed (FIG. 25) appears not only time dependent, but concentration dependent, as evident in 30A. The change in cell surface CAR-EBD correlates directly with EBD inducibility (i.e., it increases when cells are able to be activated, but do not have to be activated for it to increase), rather than just presence of non-specific estrogen analog (e.g., CMP8). (29B) The relative activation and cell surface CAR-EBD in the exact same cells (stacked points represent two different fluorophores on the same cell population). The drug-induced changes in cell surface CAR-EBD expression are decoupled from drug-induced activation (higher concentrations of drug are required to increase CAR expression compared to increases in activate-ability). This suggests that while correlated, the mechanism of activation is distinct from mere cell surface expression.

FIGS. 30A-30C. Incucyte Incucyte Cytotoxicity Assay: huCD19CAR-1Gly-EBD(4OHT). CD4 and CD8 T cells (mixed at a 1:1 ratio) expressing a huCD19CAR-EBD-1Gly-EBD(4OHT) were either preincubated for 24 hrs with 500 nM inducer drug or not preincubated. These T cells were subsequently plated with CAR-target expressing tumor lines ((30A) K562+CD19, (30B) Raji, and/or (30C) Be2+CD19) constitutively expressing mCherry (for detection and quantification). Tumors were plated at 10,000 per well, and co-culture was performed at 8:1 or 4:1ratio (T cell:tumor cell-ratio annotated where necessary) with or without 500 nM 4-OHT. Samples were placed in an Incucyte and imaged every 3-4 hours for several days. mCherry signal was then quantified and plotted as a function of time. Positive and negative controls are the appropriate constitutively active CAR and mock T cells, respectively. There appears to be no difference in the functionality of CAR-EBD T cells preincubated with drug (“24 hr pretreatment”) vs without preincubation (“0 hr treatment”). This further indicates that CAR-EBD are mechanistically distinct from degrons. Degron technologies take time (typically 3-6 hours) to accumulate in sufficient quantity to exhibit activity. In contrast, the on-kinetics of CAR-EBD killing in an incucyte cytotoxicity assay appear to be nearly instantaneous.

FIGS. 31A-31B. (31A) Incucyte Cytotoxicity Assay: B7H3CAR-3Gly-EBD variants. CD4 and CD8 T cells (mixed at a 1:1 ratio) expressing a either B7H3CAR-3Gly-EBD(4OHT), B7H3CAR-3Gly-EBD(CMP8), or B7H3CAR-3Gly-EBD(ES8) were either preincubated for 24 hrs with 500 nM inducer drug or not preincubated. These T cells were subsequently plated with CAR-target expressing tumor lines (K562, and/or Be2) constitutively expressing mCherry (for detection and quantification). Tumors were plated at 10,000 per well, and co-culture was performed at 4:1 ratio (T cell:tumor cell) with or without drug at 500 nM (B7H3CAR-3Gly-EBD(4OHT) received 500 nM 4OHT, B7H3CAR-3Gly-EBD(CMP8) received 500 nM CMP8, B7H3CAR-3Gly-EBD(ES8) received 500 nM ES8). Plates were placed inside of an Incucyte and imaged every 3-4 hours for several days. mCherry signal was then quantified and plotted as a function of time. Positive and negative controls are the appropriate constitutively active CAR and mock T cells, respectively. Profound ability to regulate the activity of the B7H3CAR-3Gly-EBDvariants was observed with all three EBD variants, with potent anti-tumor on-states and well controlled off-states. (31A and 31B) Show the B7H3CAR-EBD(CMP8) construct vs K562+mCherry. (31A) No drug vs drug, while 31B compares no drug vs drug added at 24 hrs (black vertical line). While the amount of tumor that had grown by 24 hrs was insurmountable for this donor and ratio of B7H3CAR-3Gly-EBD(CMP8) T cells, once activated with CMP8, the change in tumor growth kinetics was immediate.

FIGS. 32A-32C. (32A-32C) CD4 and CD8 T cells (mixed at a 1:1 ratio) expressing either B7H3CAR-3Gly-EBD(4OHT), B7H3CAR-3Gly-EBD(CMP8), or B7H3CAR-3Gly-EBD(ES8) (were either preincubated for 24 hrs with 500 nM inducer drug or not preincubated (B7H3CAR-3Gly-EBD(4OHT) received 500 nM 4OHT, B7H3CAR-3Gly-EBD(CMP8) received 500 nM CMP8, B7H3CAR-3Gly-EBD(ES8) received 500 nM ES8). These T cells were subsequently plated with CAR-target expressing tumor lines (K562, and/or Be2) constitutively expressing mCherry (for detection and quantification). Tumors were plated at 10,000 per well, and co-culture was performed at a 4:1 ratio (T cell:tumor cell) with or without the appropriate drug at 500 nM. Samples were placed inside of an Incucyte and imaged every 3-4 hours for several days. mCherry signal was then quantified and plotted as a function of time. Positive and negative controls are the appropriate constitutively active CAR and mock T cells, respectively. In one instance, cells were repeatedly chaIlenged with tumor in order to assess differential impacts on functional longevity between constructs. (32A) Incucyte Cytotoxicity Assay: Influence of estrogen analogs on B7H3CAR. Control assay demonstrating that the addition of an estrogen analog (e.g., 4-OHT) does not impair tumor growth (note mock condition) or CAR T cell functionality (note B7H3CAR condition). (32B) Incucyte Cytotoxicity Assay: B7H3CAR-3Gly-EBD variants. This demonstrates no difference in the killing kinetics of CAR-EBD T cells with a 24 hr pretreatment of inducer drug vs drug addition at the time of experiment start. (32C) Incucyte Cytotoxicity Assay B7H3CAR-3Gly-EBD variants. Demonstrates highly regulated killing of Be2+mCherry tumor cells by CAR-EBD(4OHT) and CAR-EBD(ES8) cells. Be2 cells were allowed 24 hrs to adhere to the plate before the addition of T cells.

FIG. 33. Exemplary sequences supporting the disclosure as follows: Estrogen Receptor (SEQ ID NO: 1), Wild-type Estrogen Binding Domain (EBD) with optional N- and C-terminal truncation positions underlined (SEQ ID NO: 2); Estrogen Receptor with G521R mutation (SEQ ID NO: 3), EBD with G521R mutation (SEQ ID NO: 4), Estrogen Receptor with E353A mutation (SEQ ID NO: 5), EBD (E353A) (SEQ ID NO: 6), EBD (E353A) Coding Sequence (SEQ ID NO: 7), Estrogen Receptor with L384M and M421G mutations (SEQ ID NO: 8), EBD with L384M and M421G mutations (SEQ ID NO: 9), Estrogen Receptor with L384M, M421G, and G521R mutations (SEQ ID NO: 10), EBD (L384M, M421G, G521R) Pairs with estrogen analog CMP8 (SEQ ID NO: 11, also referred to herein as EBD(CMP8)), EBD with G400V, M543A, and L544A mutations (SEQ ID NO: 12), EBD with G400V, M543A, and L544A mutations (referred to herein as ERT2 or EBD(4-OHT)) (SEQ ID NO: 13), ERT2 coding sequence (SEQ ID NO: 14), EBD(CMP8) (L384M, M421G, G521R) Coding sequence (SEQ ID NO: 15), EGFRVIII (806) scFv (SEQ ID NO: 16), EGFRVIII (806) scFv Coding Sequence (SEQ ID NO: 17), EGFR scFv (SEQ ID NO: 18), huCD19 (G01S) scFv (SEQ ID NO: 19), muCD19 (FMC63) scFv (SEQ ID NO: 20), CD19 scFv (SEQ ID NO: 21), CD19 scFv coding sequence (SEQ ID NO: 22), CD20 (Leu 16) scFv (SEQ ID NO: 23, CD20 scFv encoding sequence (SEQ ID NO: 24), CD22 (m971) scFv (SEQ ID NO: 25), B7H3 (hBRCA84D) scFv (SEQ ID NO: 26), L1CAM (CE7) scFv (SEQ ID NO: 27), EphA2 (2A4) scFv (SEQ ID NO: 28), EpHA2 (4H5) scFv (SEQ ID NO: 29), FITC (E2) scFv (SEQ ID NO: 30), GD2 (hu3F8) scFv (SEQ ID NO: 31), Her2 (Herceptin) scFv (SEQ ID NO: 32), IL13Ra2 (hu08) VIVh scFv (SEQ ID NO: 33), IL13Ra2 hu08 VhV1 scFv (SEQ ID NO: 34), IL13Ra2 (hu07) VhV1 scFv (SEQ ID NO: 35), IL13Ra2 (hu07) VhVI scFv (SEQ ID NO: 36), oaGD2 (8B6) VIVh scFv (SEQ ID NO: 37), ROR1 (R12) scFv (SEQ ID NO: 38), CD33 (h2H12) VhVI scFv (SEQ ID NO: 39), CD33 (h2H12) VIVh scFv (SEQ ID NO: 40), Mesothelin (P4) scFv (SEQ ID NO: 41), VAR2CSA (ID1-DBL2Xb) scFv (SEQ ID NO: 42), Anti-IL13Ra2 (IL13 zetakine) amino acid (SEQ ID NO: 43), FITCE2 scFv (SEQ ID NO: 44), FITCE2 TyrH133Ala scFv (SEQ ID NO: 45), FITCE2 HisH131Ala scFv (SEQ ID NO: 46), FL (4M5.3) scFv (SEQ ID NO: 47), FL (4D5FIu) scFv (SEQ ID NO: 48), FL (4420) scFv (SEQ ID NO: 49), DNP scFv (SEQ ID NO: 50), DNP scFv (SEQ ID NO: 51), DNP scFv (SEQ ID NO: 52), DNP scFv (SEQ ID NO: 53), VH of DNP scFv (Ab-1; 1BAF) (SEQ ID NO: 54), VL of DNP scFv (Ab-1; 1BAF) (SEQ ID NO: 55), VH of DNP scFv (Ab-2; XC) (SEQ ID NO: 56), VL of DNP scFv (Ab-2; XC) (SEQ ID NO: 57), VH of DNP scFv (Ab-3) (SEQ ID NO: 58), VL of DNP scFv (Ab-3) (SEQ ID NO: 59), VL-linker-VH of DNP scFv (Ab-3) (SEQ ID NO: 60), VH-linker-VL of DNP scFv (Ab-3) (SEQ ID NO: 61), VH-linker-VL of DNP scFv (1BAF) (SEQ ID NO: 62), VL-linker-VH of DNP scFv (1BAF) (SEQ ID NO: 63), VH-linker-VL of DNP scFv (XC) (SEQ ID NO: 64), VL-linker-VH of DNP scFv (XC) (SEQ ID NO: 65), VH-linker-VL of DNP scFv (1BAF) (SEQ ID NO: 66), VL-linker-VH of DNP scFv (1BAF) (SEQ ID NO: 67), VH-linker-VL of DNP scFv (XC) (SEQ ID NO: 68), VL-linker-VH of DNP scFv (XC) (SEQ ID NO: 69), VH-linker-VL of DNP scFv (Ab-3) (SEQ ID NO: 70), DNP scFv VH-linker-VL of DNP scFv (Ab-3) (SEQ ID NO: 71), Glycine-serine linker (SEQ ID NO: 72), Glycine-serine linker (SEQ ID NO: 73), Glycine-serine linker (SEQ ID NO: 74), Glycine-serine linker (SEQ ID NO: 75), Coding sequence of (G₄S)₃ linker (SEQ ID NO: 76), Coding sequence of (G₄S)₄ linker (SEQ ID NO: 77), Short Spacer (IgG4 (short hinge)) (SEQ ID NO: 78), Short Spacer (IgG4 (short hinge)) coding sequence (SEQ ID NO: 79), Short Spacer 2, Alternative IgG4 linker (SEQ ID NO: 80), Medium Spacer (SEQ ID NO: 81), Long spacer with single (L235D) mutation (SEQ ID NO: 82), Long spacer with double (L235D, N297Q) mutations (SEQ ID NO: 83), Glycine Linker, Glycine Linker coding sequence (SEQ ID NO: 84), P2A (SEQ ID NO: 85), P2A coding sequence (SEQ ID NO: 86), T2A (SEQ ID NO: 87), T2A coding sequence (SEQ ID NO: 88), E2A (SEQ ID NO: 89), F2A (SEQ ID NO: 90), DHFRdm (SEQ ID NO: 91), DHFRdm coding sequence (SEQ ID NO: 92), CD19t (SEQ ID NO: 93), CD19t coding sequence (SEQ ID NO: 94), truncated Her2 (Her2) (SEQ ID NO: 95), Her2tG (SEQ ID NO: 96), EGFRt (SEQ ID NO: 97), GM-CSF Signal Peptide (SEQ ID NO: 98), CD8 signal peptide (SEQ ID NO: 99), His tag (SEQ ID NO: 100), Flag tag (SEQ ID NO: 101), Flag tag (SEQ ID NO: 102), Flag tag (SEQ ID NO: 103), Xpress tag (SEQ ID NO: 104), Avi tag (SEQ ID NO: 105), Calmodulin binding peptide (CBP) tag (SEQ ID NO: 106), Polyglutamate tag (SEQ ID NO: 107), HA tag (SEQ ID NO: 108), HA tag (SEQ ID NO: 109), HA tag (SEQ ID NO: 110), Myc tag (SEQ ID NO: 111), Strep tag (SEQ ID NO: 112), STREP® tag II (SEQ ID NO: 113), Softag 1 (SEQ ID NO: 114), Softag 3 (SEQ ID NO: 115), V5 tag (SEQ ID NO: 116), MND promoter (SEQ ID NO: 117), EGFR806CAR-ERT2 (SEQ ID NO: 118), EGFR806CAR-ERT2-P2A-DHFRdm-T2A-CD19t protein sequence in table form (coIlectively creating SEQ ID NO: 1306) and EGFR806CAR-ERT2-P2A-DHFRdm-T2A-CD19t coding sequence in table form (coIlectively creating SEQ ID NO: 125), CD8a Hinge/Transmembrane domain (SEQ ID NO: 126), Alternative CD8 hinge/Transmembrane domain (SEQ ID NO: 127), Alternative CD8 hinge/Transmembrane domain (SEQ ID NO: 128), Alternative Human CD8 alpha chain hinge (SEQ ID NO: 129), 4-1BB/CD3z intracellular signaling domain (SEQ ID NO: 130), Coding sequence of 4-1BB/CD3z intracellular signaling domains (SEQ ID NO: 131), Alternative Coding sequence of 4-1BB/CD3z intracellular signaling domains (SEQ ID NO: 132), CD3ζ ICD (SEQ ID NO: 121), CD3ζ ICD coding sequence (SEQ ID NO:124), T-cell surface glycoprotein CD3 epsilon chain (CD3E) (SEQ ID NO: 133), CD3E ECD (SEQ ID NO: 134), CD3E ICD (SEQ ID NO: 135), CD3E TM (SEQ ID NO: 136), T-cell surface glycoprotein CD3 delta chain (CD35) (SEQ ID NO: 137), CD35 ECD (SEQ ID NO: 138), CD35 ICD (SEQ ID NO: 139), CD35 TM (SEQ ID NO: 140), T-cell surface glycoprotein CD3 zeta chain (CD3ζ) (SEQ ID NO: 141), CD3ζ ECD (SEQ ID NO: 142), CD3ζ ICD (SEQ ID NO: 143), CD3ζ TM (SEQ ID NO: 144), CD70 [Homo sapiens] (SEQ ID NO: 145), TL1A or Tumor necrosis factor (ligand) superfamily, member 15 (SEQ ID NO: 146), OX40 (SEQ ID NO: 147), OX40 ECD (SEQ ID NO: 148), OX40 ICD (SEQ ID NO: 149), OX40 TM (SEQ ID NO: 150), CD27 antigen precursor (SEQ ID NO: 151), CD27 ECD (SEQ ID NO: 152), CD27 ICD (SEQ ID NO: 153), CD27 TM (SEQ ID NO: 154), Cytokine receptor CD30 (SEQ ID NO: 155), CD30 ECD (SEQ ID NO: 156), CD30 ICD (SEQ ID NO: 157), CD30 TM (SEQ ID NO: 158), CD40 or Human tumor necrosis factor receptor superfamily member 5 (SEQ ID NO: 159), CD40 ECD (SEQ ID NO: 160), CD40 ICD (SEQ ID NO: 161), CD40 TM (SEQ ID NO: 162), HVEM (SEQ ID NO: 163), HVEM ECD (SEQ ID NO: 164), HVEM ICD (SEQ ID NO: 165), HVEM TM (SEQ ID NO: 166), DR3 (SEQ ID NO: 167), DR3 ECD (SEQ ID NO: 168), DR3 ICD (SEQ ID NO: 169), DR3TM (SEQ ID NO: 170), 4-1EE or Human tumor necrosis factor receptor superfamily member 9 precursor (SEQ ID NO: 171), 4-1BB ECD (SEQ ID NO: 172), 41BB ICD (SEQ ID NO: 120), 4-1EE ICD coding sequence (SEQ ID NO: 123), 4-1EE TM (SEQ ID NO: 173), Interleukin-2 receptor subunit alpha (CD25) (SEQ ID NO: 174), CD25 ECD (SEQ ID NO: 175), CD25 ICD (SEQ ID NO: 176), CD25 TM (SEQ ID NO: 177), T-cell-specific surface glycoprotein CD28 (SEQ ID NO: 178), CD28 ECD (SEQ ID NO: 179), CD28 ICD (SEQ ID NO: 180), CD28 ICD (SEQ ID NO: 181), Coding sequence of ICD of human CD28 (SEQ ID NO: 182), CD28 TM (SEQ ID NO: 183), CD28 transmembrane domain (SEQ ID NO: 119), CD28tm coding sequence (SEQ ID NO: 122), CD79a (SEQ ID NO: 184), CD79a ECD (SEQ ID NO: 185), CD79a ICD (SEQ ID NO: 186), CD79a TM (SEQ ID NO: 187), CD79b (SEQ ID NO: 188), CD79b ECD (SEQ ID NO: 189), CD79b ICD (SEQ ID NO: 190), CD79b TM (SEQ ID NO: 191), Signaling lymphocytic activation molecule family member 1 (SLAMF1, CD150) (SEQ ID NO: 192), SLAMF1 ECD (SEQ ID NO: 193), SLAMF1 ICD (SEQ ID NO: 194), SLAMF1 TM (SEQ ID NO: 195), Inducible T-cell co-stimulator (ICOS, CD278) (SEQ ID NO: 196), ICOS ECD (SEQ ID NO: 197), ICOS ICD (SEQ ID NO: 198), ICOS TM (SEQ ID NO: 199), TNFRSF18 protein (CD357, GITR) (SEQ ID NO: 200), GITR ECD (SEQ ID NO: 201), GITR ICD (SEQ ID NO: 202), GITR TM: LGWLTVVLLAVAACVLLLTSA (SEQ ID NO: 203), Caspase recruitment domain-containing protein 11 (CARD11) (SEQ ID NO: 204), DAP10 (SEQ ID NO: 205), DAP10 ECD (SEQ ID NO: 206), DAP10 ICD (SEQ ID NO: 207), DAP10 TM (SEQ ID NO: 208), DAP12 (SEQ ID NO: 209), DAP12 ECD (SEQ ID NO: 210), DAP12 ICD (SEQ ID NO: 211), DAP12 TM (SEQ ID NO: 212), High affinity immunoglobulin epsilon receptor subunit alpha (SEQ ID NO: 213), High affinity immunoglobulin epsilon receptor subunit beta (SEQ ID NO: 214), FcR-γ, high affinity immunoglobulin epsilon receptor subunit gamma precursor (SEQ ID NO: 215), Tyrosine-protein kinase Fyn (SEQ ID NO: 216), Tyrosine-protein kinase Lck (SEQ ID NO: 217), LAT (SEQ ID NO: 218), LRP (SEQ ID NO: 219), NKG2D (SEQ ID NO: 220), NOTCH1 (SEQ ID NO: 221), NOTCH2 (SEQ ID NO: 222), NOTCH3 (SEQ ID NO: 223), NOTCH4 (SEQ ID NO: 224), Tyrosine-protein kinase transmembrane receptor ROR2 (SEQ ID NO: 225), Tyrosine-protein kinase RYK (SEQ ID NO: 226), Lymphocyte cytosolic protein 2 (SIp76) (SEQ ID NO: 227), pre-T cell receptor alpha-type chain precursor (pTa) (SEQ ID NO: 228), T-cell receptor alpha chain (TCRα) (SEQ ID NO: 229), T-cell receptor beta chain (TCRβ) (SEQ ID NO: 230), T-cell receptor interacting molecule (TRIM) protein (SEQ ID NO: 231), ZAP70 protein (SEQ ID NO: 232), Patched 2 (PTCH2) (SEQ ID NO: 233), Programmed cell death protein 1 (PD1) (SEQ ID NO: 234), PD1 ECD (SEQ ID NO: 235), PD1 ICD (SEQ ID NO: 236), PD1 TM (SEQ ID NO: 237), Programmed cell death 1 ligand 1 (PD-L1) (SEQ ID NO: 238), PD-L1 ECD (SEQ ID NO: 239), PD-L1 ICD (SEQ ID NO: 240), PD-L1 TM (SEQ ID NO: 241), Programmed cell death 1 ligand 2 (PD-L2) (SEQ ID NO: 242), PD-L2 ECD (SEQ ID NO: 243), PD-L2 ICD (SEQ ID NO: 244), PD-L2 TM (SEQ ID NO: 245), CTLA4 (SEQ ID NO: 246), CTLA4 ECD (SEQ ID NO: 247), CTLA4 ICD (SEQ ID NO: 248), CTLA4 TM (SEQ ID NO: 249), T cell immunoglobulin mucin-3 (TIM3) (SEQ ID NO: 250), Carcinoembryonic antigen-related cell adhesion molecule 1 (CEACAM-1) (SEQ ID NO: 251), CEACAM-1 ECD (SEQ ID NO: 252), CEACAM-1 ICD (SEQ ID NO: 253), CEACAM-1 TM (SEQ ID NO: 254), Carcinoembryonic antigen-related cell adhesion molecule 3 (CEACAM-3) (SEQ ID NO: 255), CEACAM-3 ECD (SEQ ID NO: 256), CEACAM-3 ICD (SEQ ID NO: 257), CEACAM-3 TM (SEQ ID NO: 258), Carcinoembryonic antigen-related cell adhesion molecule 5 (CEACAM-5) (SEQ ID NO: 259), Lymphocyte activation gene 3 protein (LAG3) (SEQ ID NO: 260), LAG3 ECD (SEQ ID NO: 261), LAG3 ICD (SEQ ID NO: 262), LAG3 TM (SEQ ID NO: 263), V-type immunoglobulin domain-containing suppressor of T-cell activation (VISTA) (SEQ ID NO: 264), VISTA ECD (SEQ ID NO: 265), VISTA ICD (SEQ ID NO: 266), VISTA TM (SEQ ID NO: 267), B- and T-lymphocyte attenuator (BTLA) (SEQ ID NO: 268), BTLA ECD (SEQ ID NO: 269), BTLA ICD (SEQ ID NO: 270), BTLA TM (SEQ ID NO: 271), T-cell immunoreceptor with Ig and ITIM domains (TIGIT) (SEQ ID NO: 272), TIGIT ECD (SEQ ID NO: 273), TIGIT ICD (SEQ ID NO: 274), TIGIT TM (SEQ ID NO: 275), Leukocyte-associated immunoglobulin-like receptor 1 (LAIR1) (SEQ ID NO: 276), LAIR1 ECD (SEQ ID NO: 277), LAIR1 ICD (SEQ ID NO: 278), LAIR1 TM (SEQ ID NO: 279), T-lymphocyte activation antigen CD80 (SEQ ID NO: 280), CD80 ECD (SEQ ID NO: 281), CD80 ICD (SEQ ID NO: 282), CD80 TM (SEQ ID NO: 283), T-lymphocyte activation antigen CD86 (SEQ ID NO: 284), CD86 ECD (SEQ ID NO: 285), CD86 ICD (SEQ ID NO: 286), CD86 TM (SEQ ID NO: 287), CD160 antigen (SEQ ID NO: 288), Natural kiIler cell receptor 2B4 (SEQ ID NO: 289), 2B4 ECD (SEQ ID NO: 290), 2B4 ICD (SEQ ID NO: 291), 2B4 TM (SEQ ID NO: 292), CD276 antigen (B7-H3) (SEQ ID NO: 293), B7-H3 ECD (SEQ ID NO: 294), B7-H3 ICD (SEQ ID NO: 295), B7-H3 TM (SEQ ID NO: 296), V-set domain-containing T-cell activation inhibitor 1 (VTCN1) (SEQ ID NO: 297), VTCN1 ECD (SEQ ID NO: 298), VTCN1 ICD, VTCN1 TM (SEQ ID NO: 299), KiIler-cell Immunoglobulin-like Receptor (SEQ ID NO: 300), adenosine receptor A2a (SEQ ID NO: 301), Galectin-9 (GAL9) (SEQ ID NO: 302), transforming growth factor beta receptor I (TGFR β) (SEQ ID NO: 303), MHC class I (SEQ ID NO: 304), and MHC class II, partial (SEQ ID NO: 305)

### DETAILED DESCRIPTION

Using genetic engineering, significant progress has been made in activating and directing cells of the immune system to kill cancer cells and infected cells. For example, T cells have been genetically engineered to express molecules having extracellular components that bind particular target antigens and intracellular components that direct actions of the T cell when the extracellular component has bound the target antigen. As an example, the extracellular component can be designed to bind target antigens found on cancer cells or infected cells and, when bound, the intracellular component activates the T cell to destroy the bound cell. Examples of such molecules include chimeric antigen receptors (CAR).

As used herein, CAR include a synthetically designed protein including a ligand binding domain that binds to an antigen associated with a disease or disorder. The ligand binding domain is linked to one or more intracellular signaling domains of an immune cell. Ligand binding domains can be derived from, for example, antibodies, receptors (e.g., T cell receptors), and receptor ligands (e.g., cytokines or chemokines).

Although CAR-expressing T cells can demonstrate potent anti-tumor activity, significant toxicities can also arise, for example, by engraftment-induced cytokine storm (cytokine release syndrome), tumor lysis syndromes (TLS) and ongoing B cell cytopenias, each of which are attributable to unregulated functional outputs of constitutively expressed and active CAR. Such toxicities can limit the applicability of CAR-based therapies.

Methods to eliminate CAR-T cells such as suicide gene-mediated elimination of these cells, for example, can ameliorate such toxicities; however, this approach risks premature attenuation of anti-tumor activity and can significantly impact curative potential. Additional methods to control the activity of proteins is through the use of degron sequences that can lead to the controllable degradation or stability of the protein. However, these effects are delayed, relying on cellular degradation of proteins. As such, there is a need to identify more rapid and controllable methods for controlling CAR and other protein activity following expression in vivo.

The current disclosure provides CAR whose ability to be activated by antigen binding following expression in vivo is controlled with the administration of drug molecules. The ability to control activation, without relying on protein degradation provides an important safety improvement to CAR-based cellular immunotherapies.

The current disclosure achieves these advances by incorporating a heat shock protein 90 (hsp90) binding domain within the intracellular component of the CAR. In the absence of the drug molecule, the hsp90 binding domain is bound by hsp90, preventing the CAR from interacting with other key intracellular molecules required for CAR activation following antigen binding.

When the drug molecule is present, the drug molecule can displace hsp90 from the hsp90 binding domain site and/or otherwise result in a conformational change, such that intracellular signaling can occur following antigen binding.

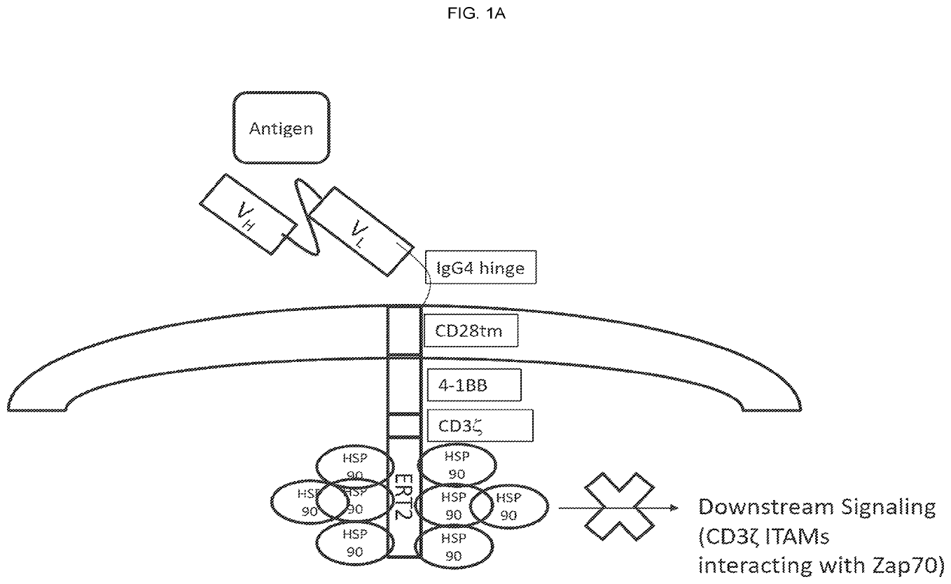

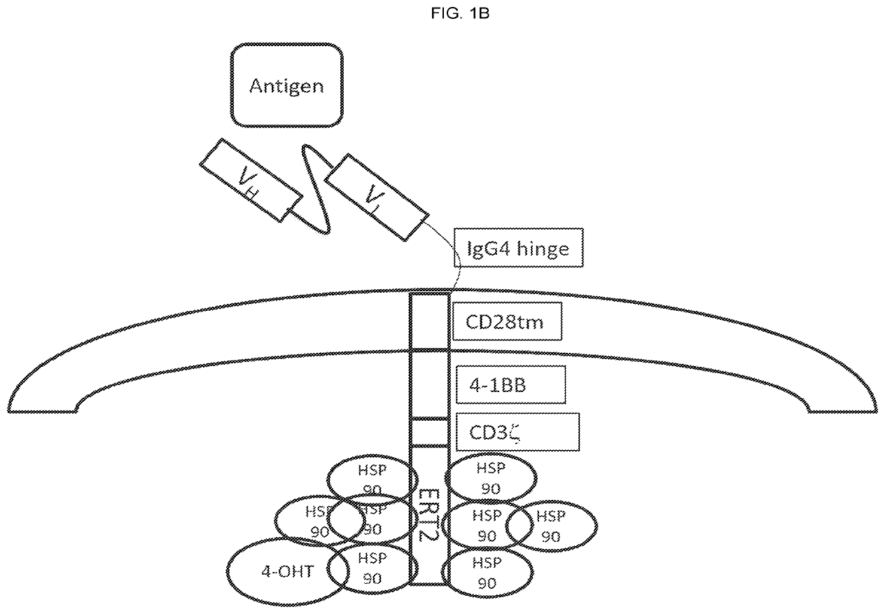

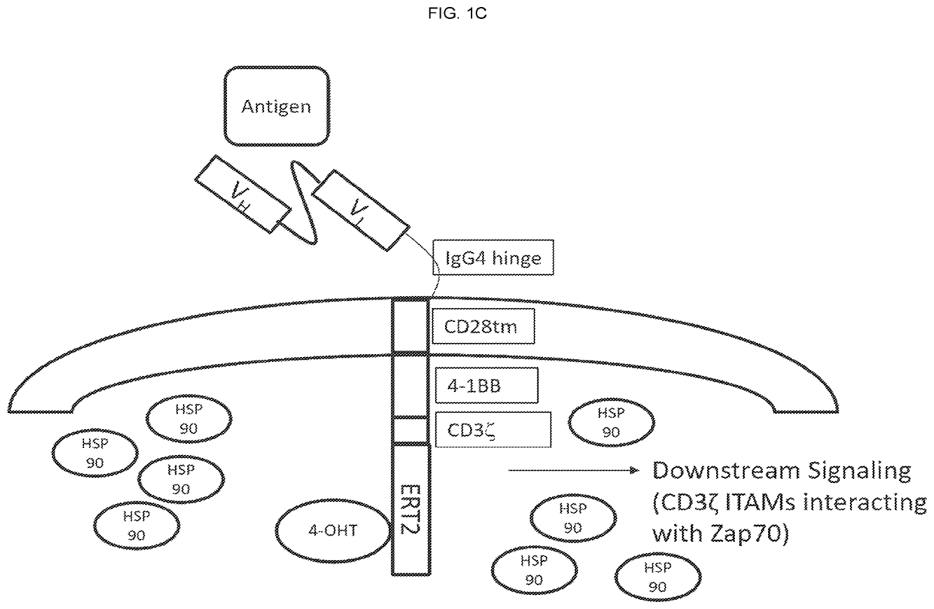

This mechanism is depicted in FIGS. 1A-1C depicting an estrogen binding domain (EBD) as the hsp90 binding domain. The EBD can be derived from the natural estrogen receptor but should include at least one mutation so that the EBD no longer binds estrogen but does bind a drug molecule. In the embodiment depicted in FIGS. 1A-1C, the drug molecule is 4-OHT. In the absence of the drug molecule, hsp90 binds the EBD and the CAR is in the “OFF” state (FIG. 1A). Intracellular drug molecule, however, can actively out compete hsp90 for EBD binding and/or otherwise result in a conformational change, allowing CAR-based activation signals following antigen binding (FIGS. 1B, 1C).

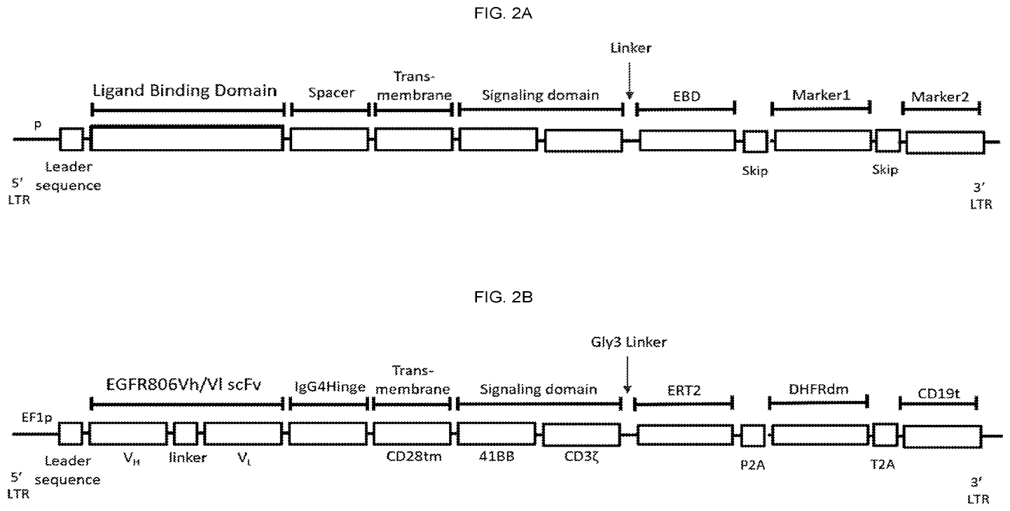

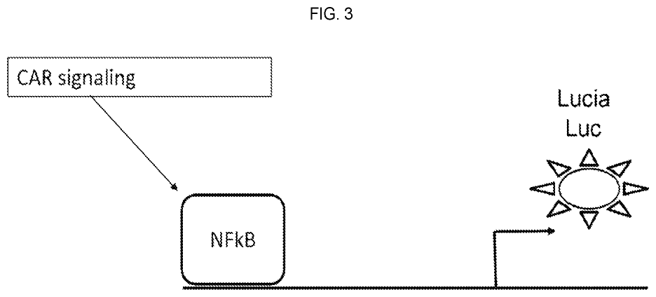

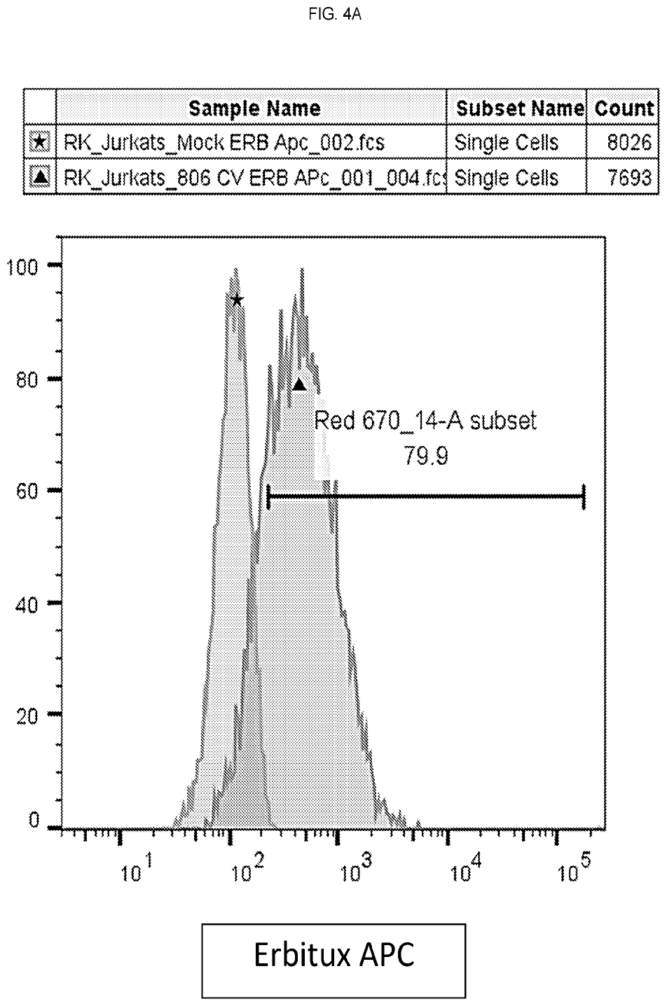

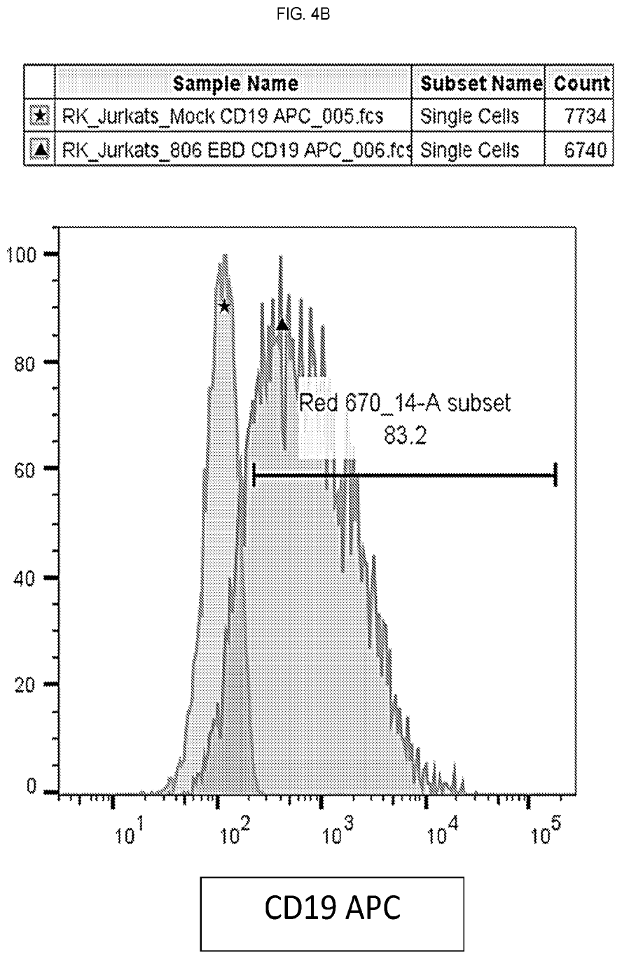

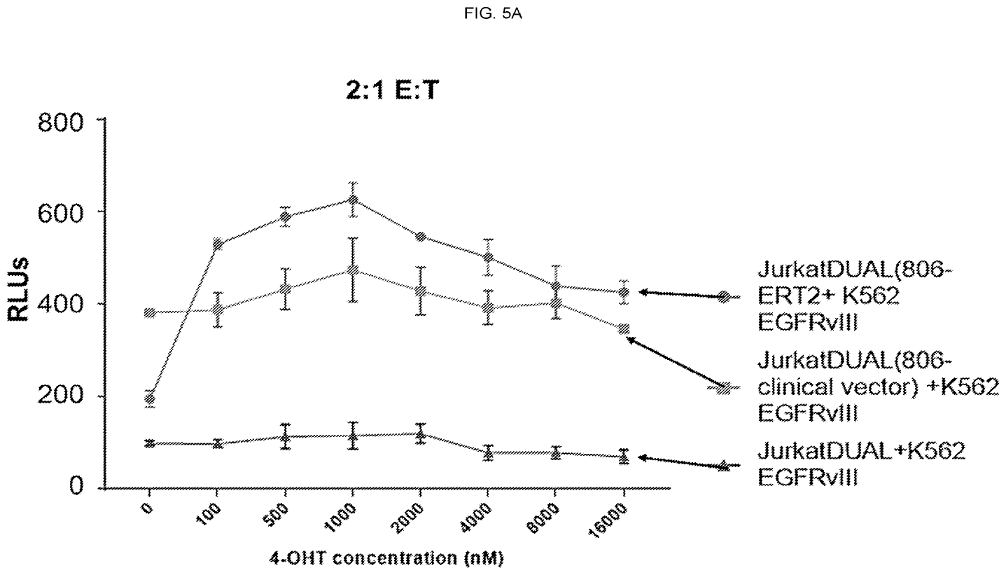

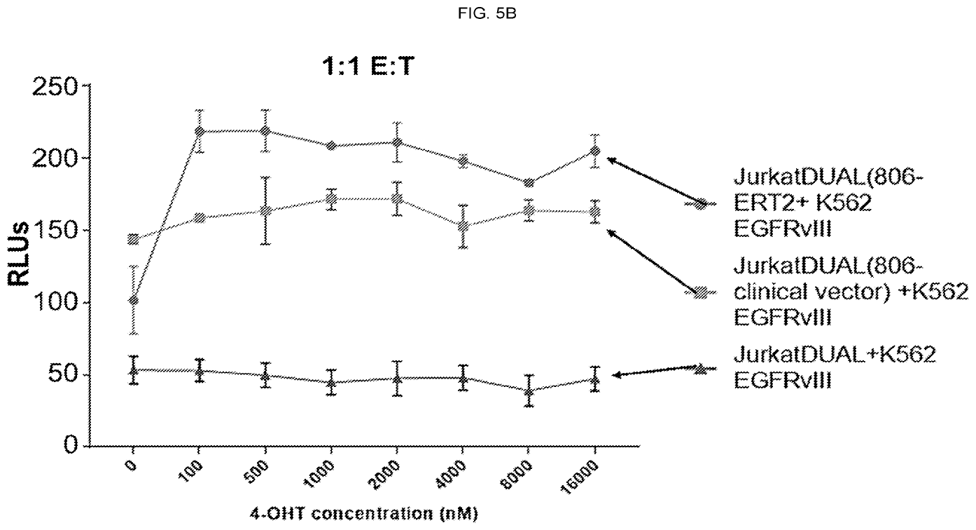

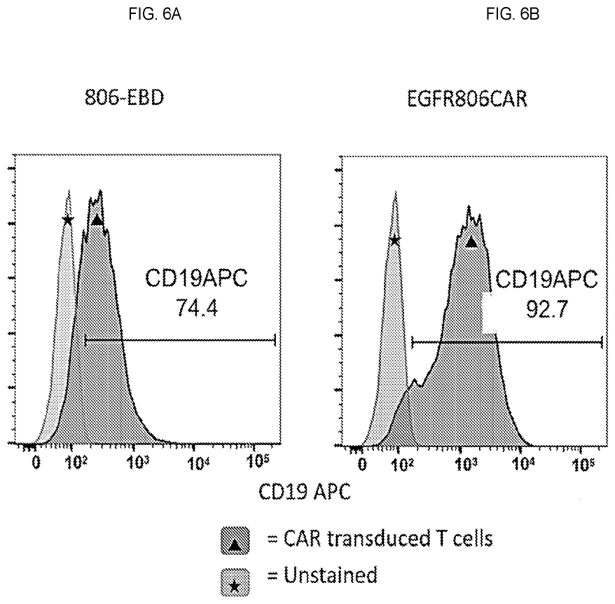

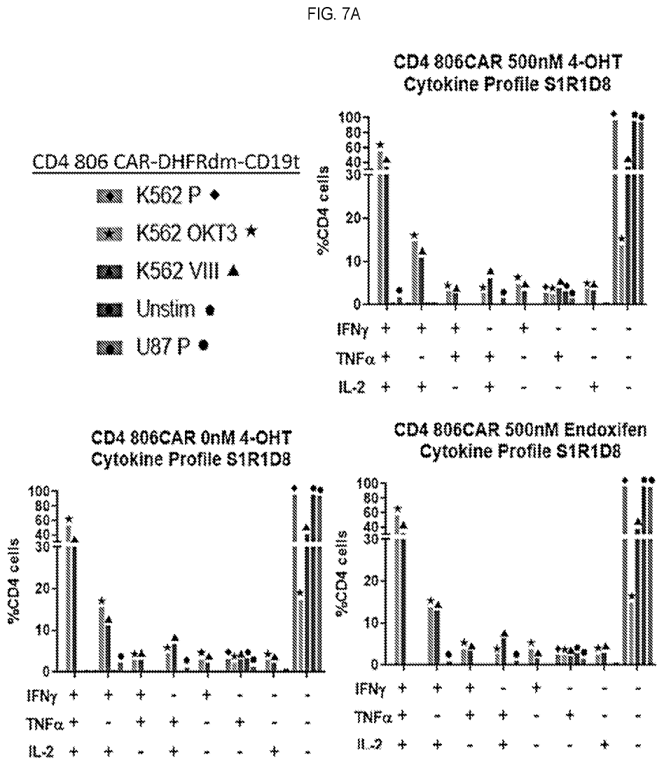

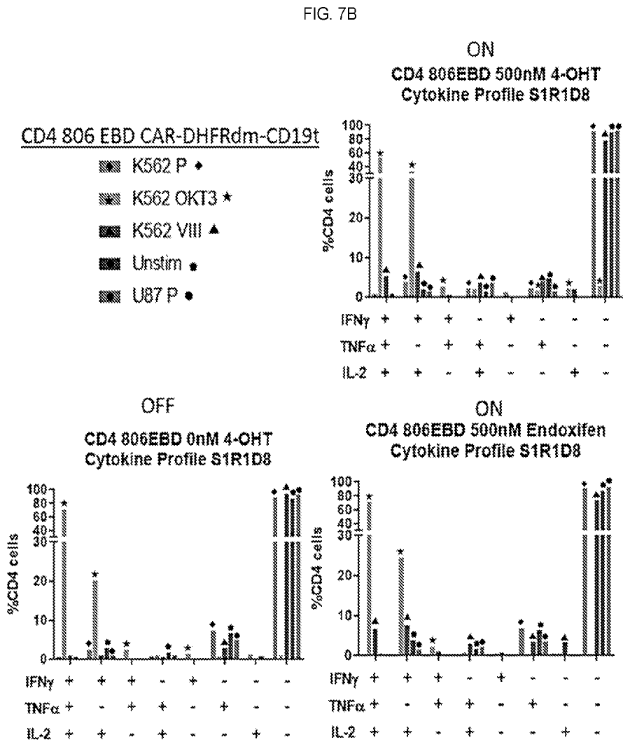

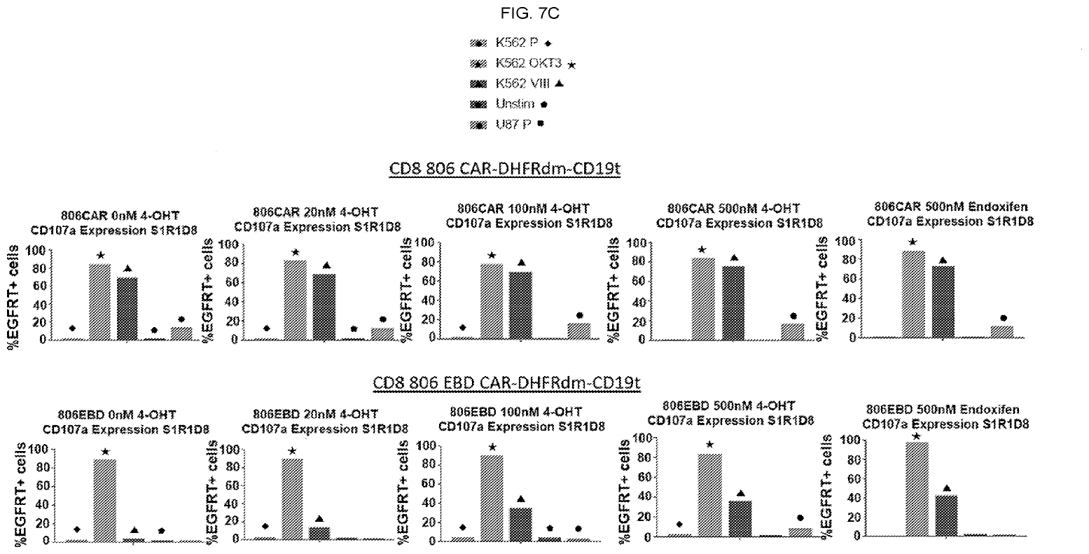

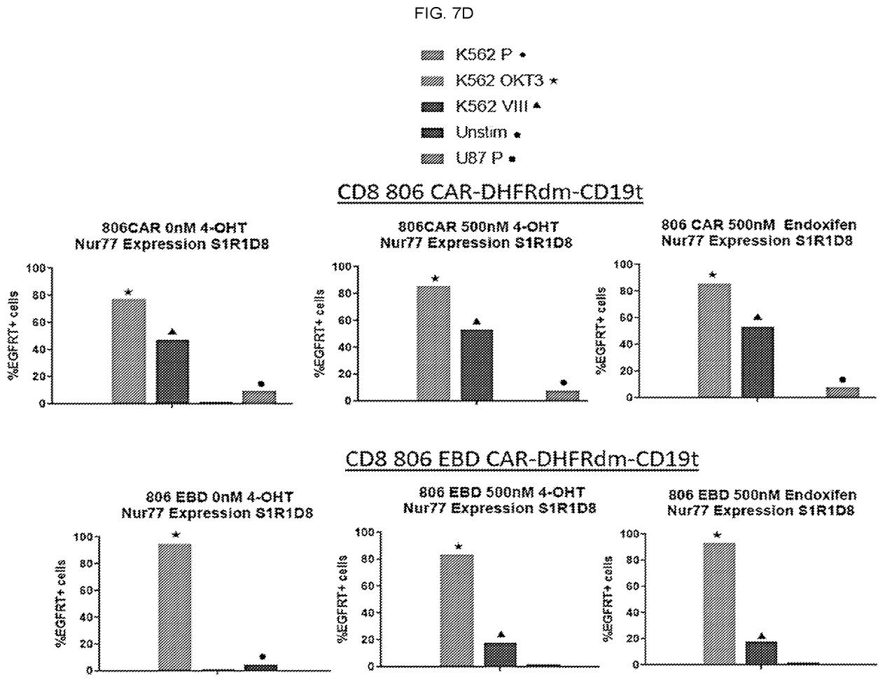

FIGS. 2A and 2B depict exemplary schematic of CAR that can be used to practice aspects of the disclosure. As shown in FIG. 2A, the CAR include a ligand binding domain, a spacer, a transmembrane domain, a signaling domain, and an estrogen binding domain (EBD). The CAR can also include selection/transduction markers. FIG. 2B depicts a particular CAR referred to as the 806-ERT2 CAR. This CAR incorporates an IgG4 hinge between the EGFR806 Vh/VI scFv and the transmembrane region. There is a Gly3 linker consisting of three glycines that connect the signaling domain to the ERT2 EBD. The ERT2 domain is then followed by P2A-DHFRdm-T2A-CD19t as markers for selection and transduction respectively. Additional CAR disclosed herein include Gly2 or Gly1 linkers (within this context, also referred to herein as junction amino acids) in place of the Gly3 linker or include no linker between the signaling domain and EBD. Additionally, the EBD can be within the signaling domain (e.g., between 4-1BB and CD3 zeta), 5′ of the signaling domain and 3′ of the transmembrane domain, or 5′ of the transmembrane domain, yet remaining within the intracellular space. In certain examples, the ligand binding domain is a B7H3 binding scFv.

Hsp90 binding domains can also be derived from the binding domains for cortisol, androgens, progesterone, and aldosterone. Further, hsp90 binding domains can be derived from numerous other proteins that bind hsp90, commonly referred to as hsp90 clients. Hsp90 clients typically include hormone receptors, transcription factors, and kinases, among other types of molecules.

One benefit of the current disclosure is the ability to control the activity of a single chain protein without reliance on dimerization or multimerization with another protein, and, and without reliance on protein stabilization/destabilization, for example through the incorporation of a degron sequence. As used herein, a degron sequence refers to an amino acid sequence recombinantly linked to a fusion protein for the purpose of controlling the stability/degradation of the protein. Degron sequences are typically linked to fusion proteins at the C-terminal end. Examples are described in, for example, US 2014/0255361, and include RRRG and RRRGN (SEQ ID NO: 306).

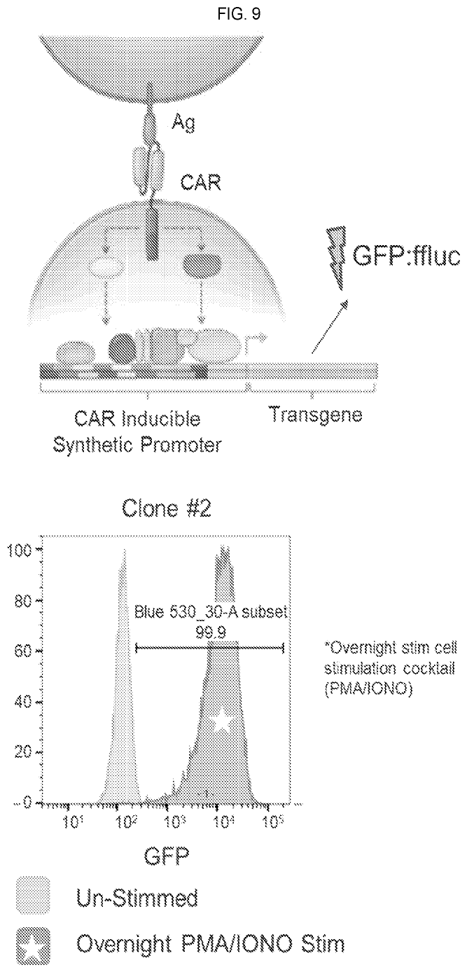

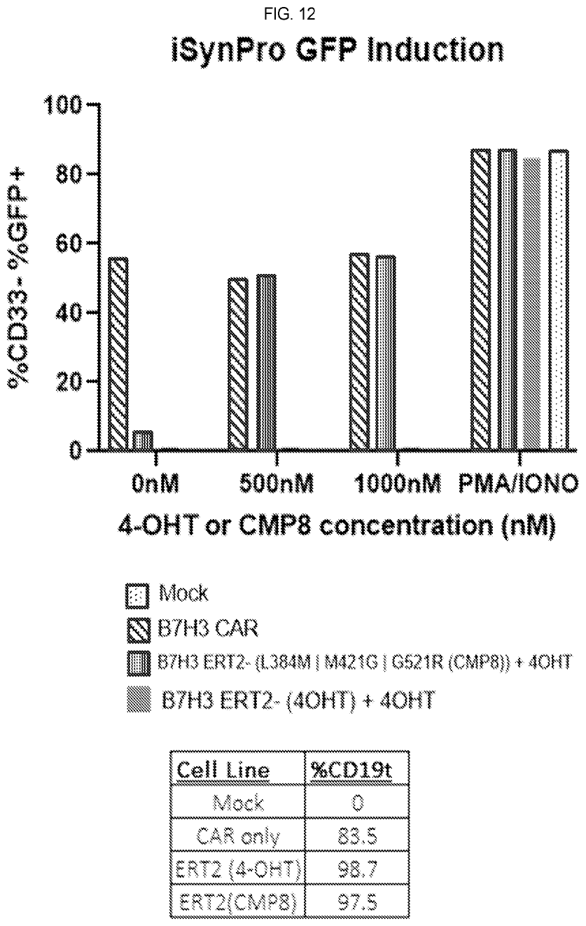

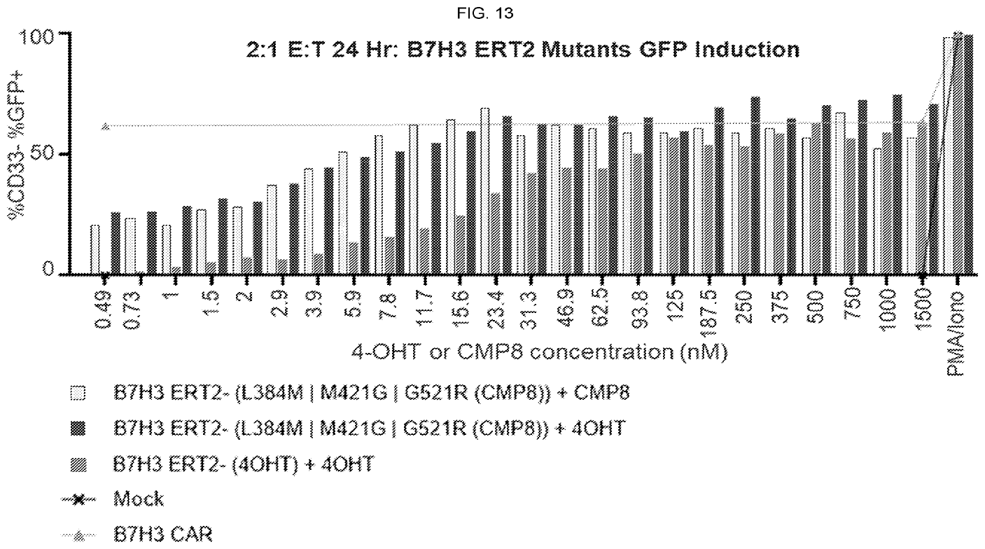

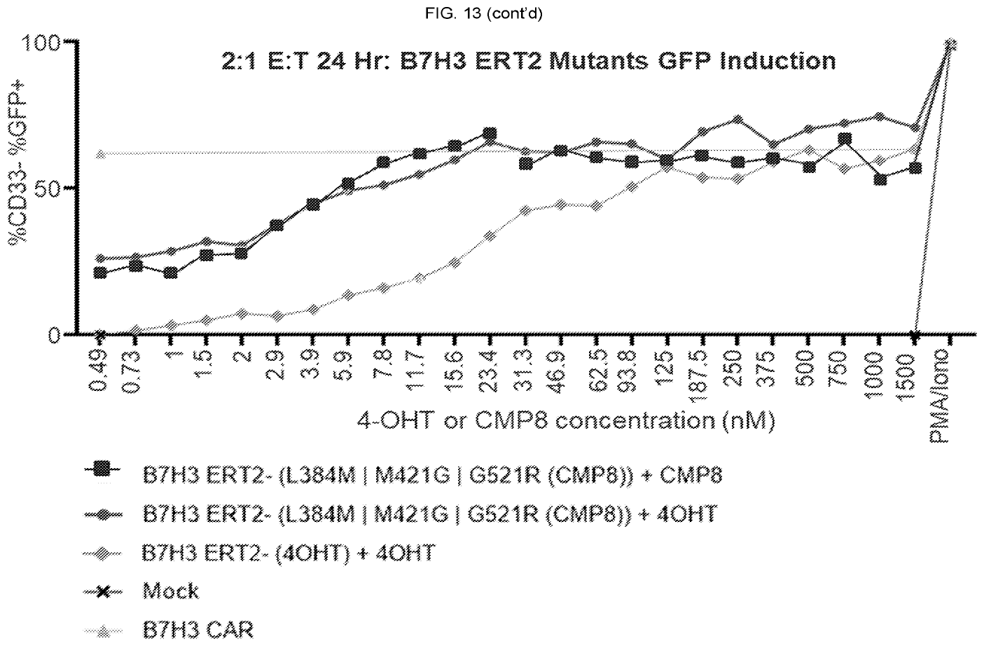

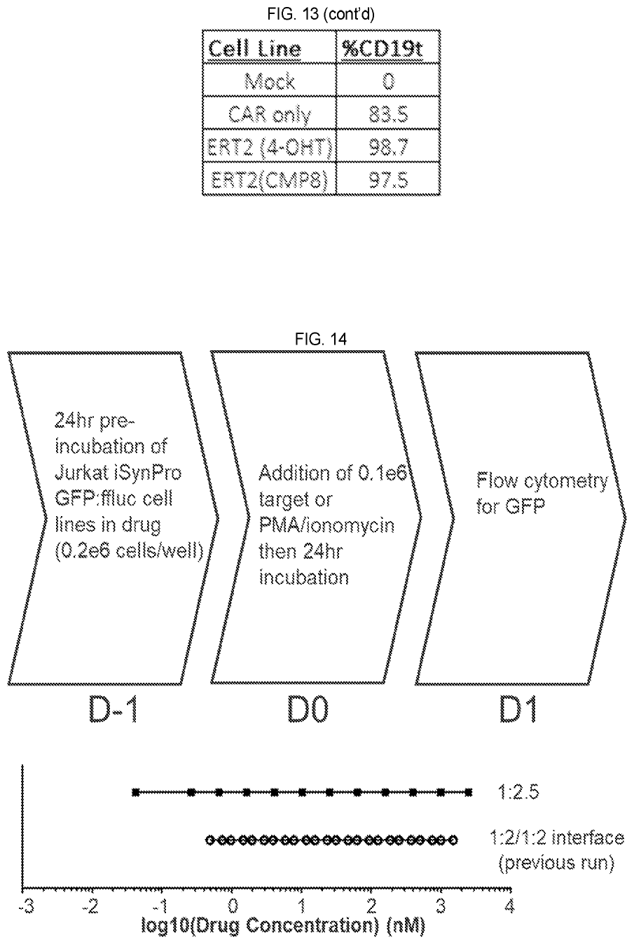

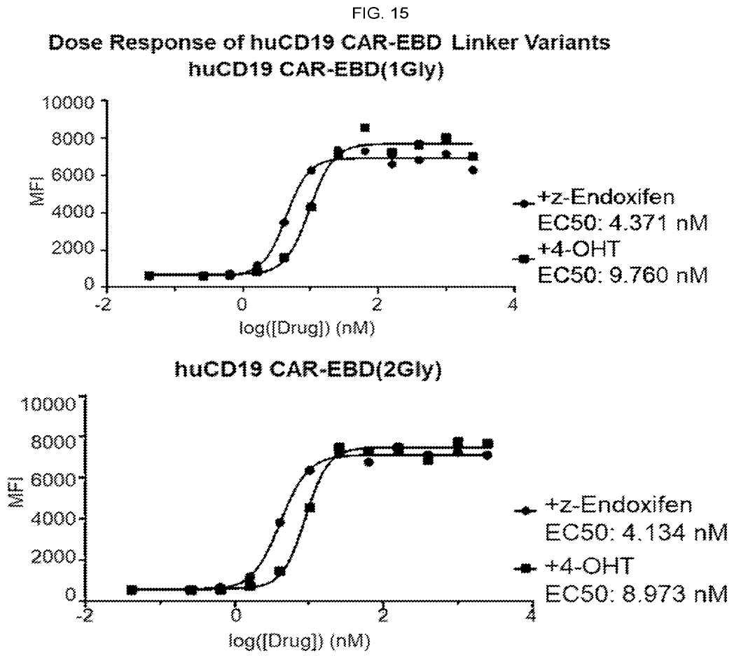

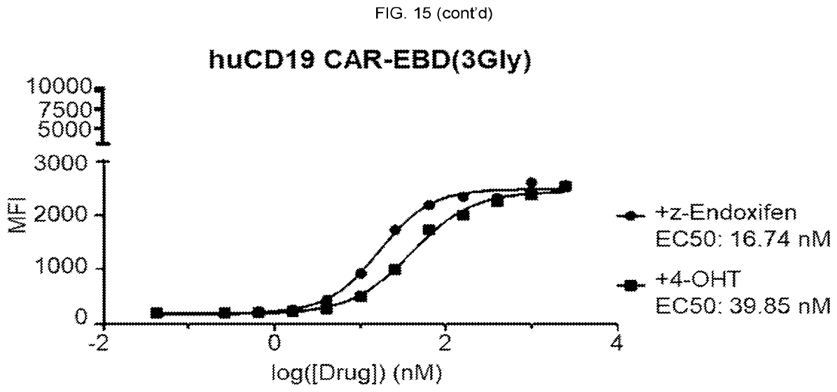

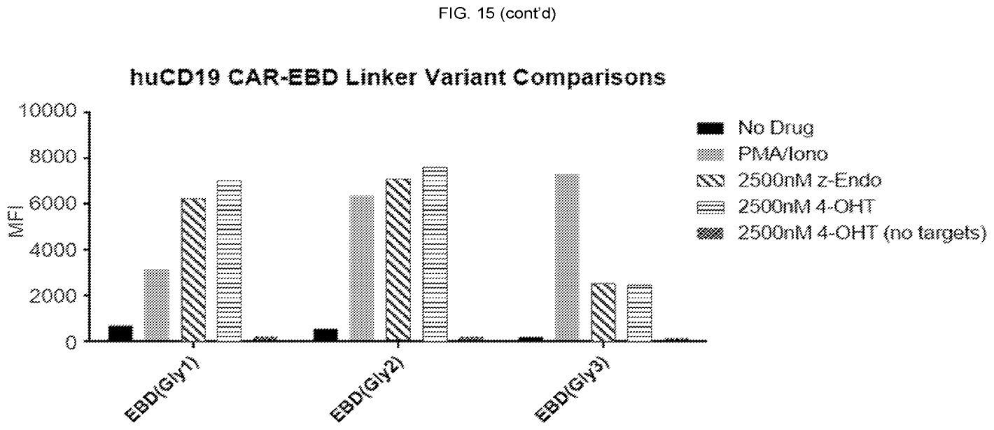

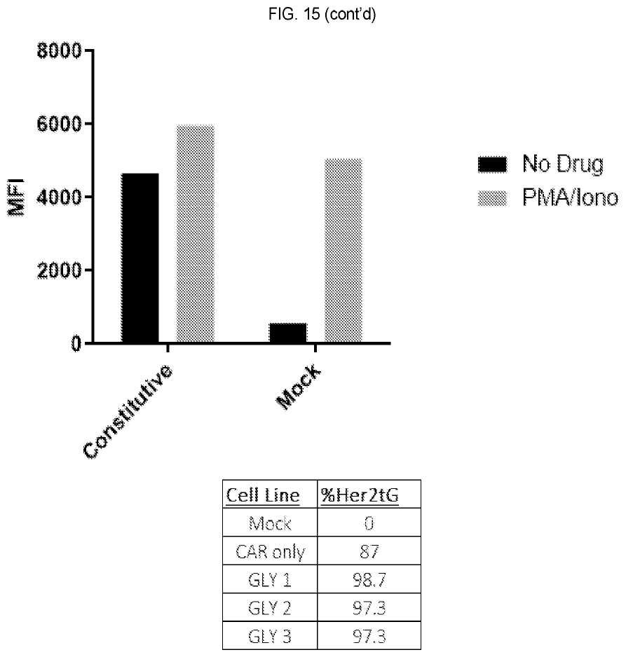

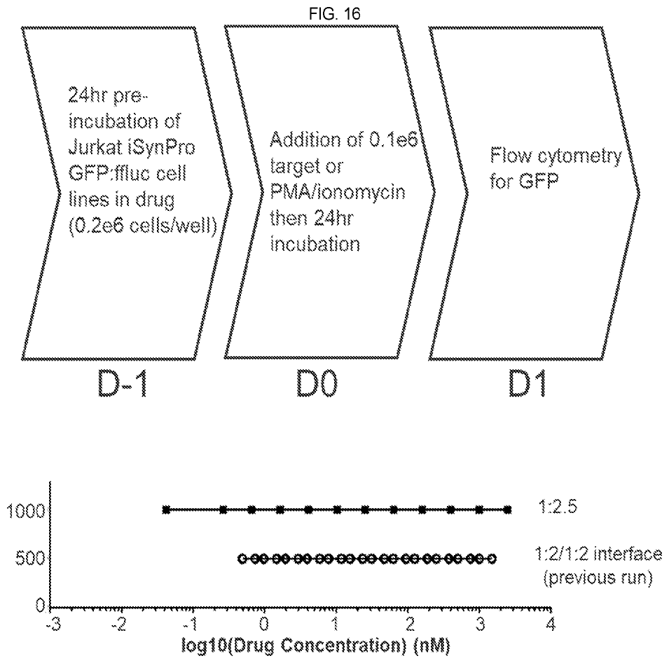

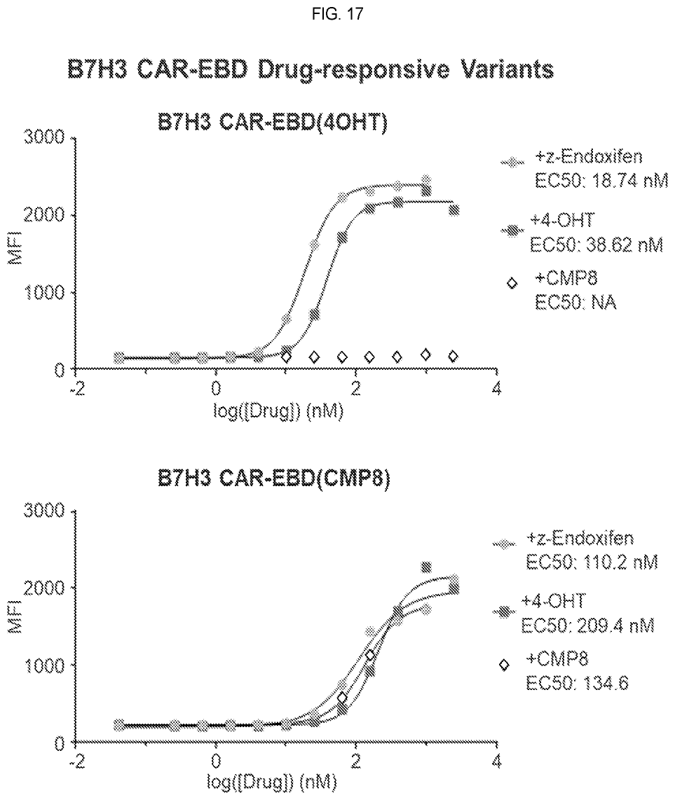

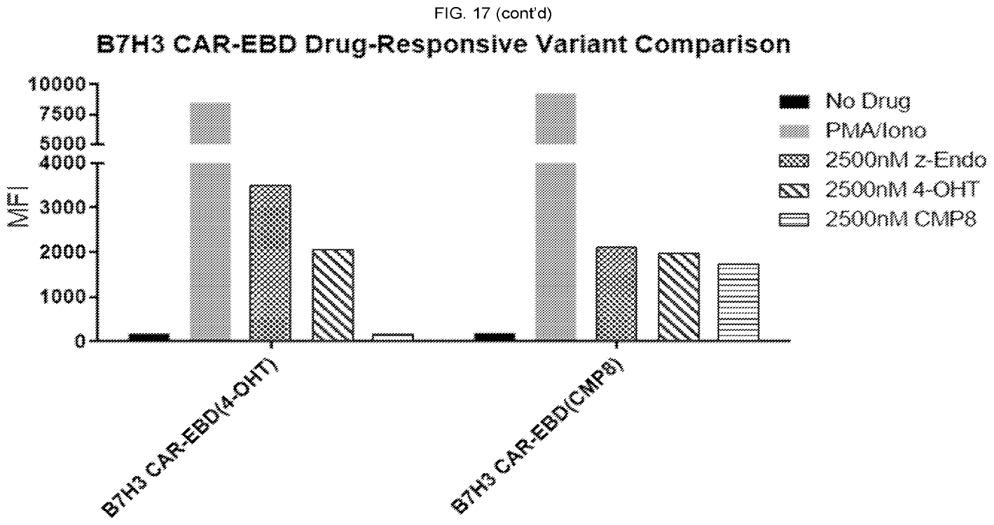

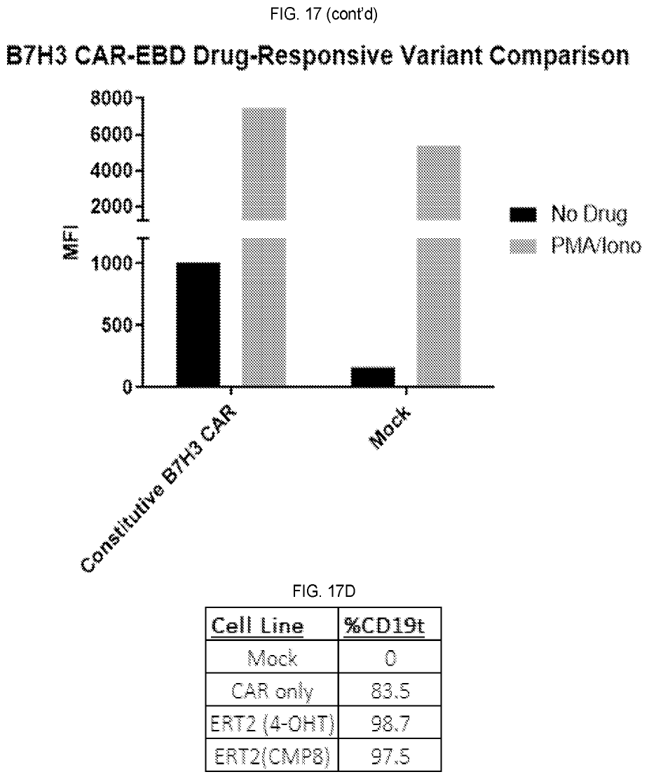

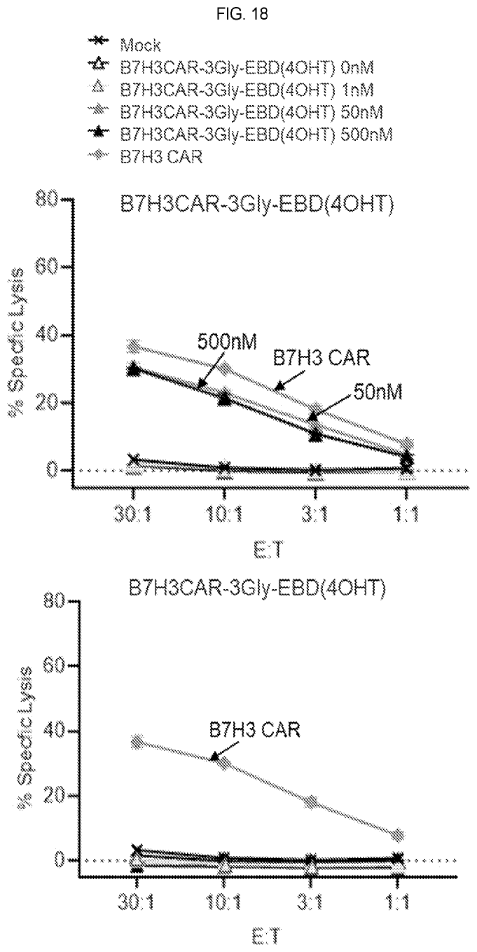

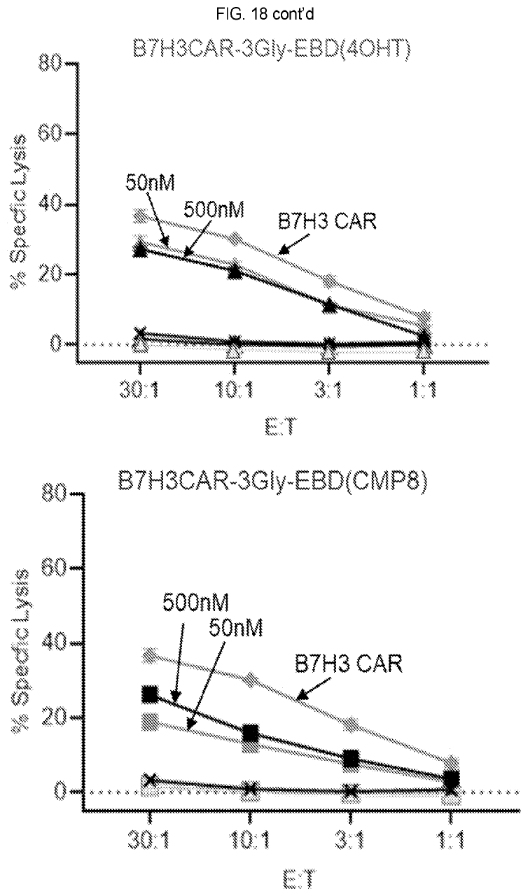

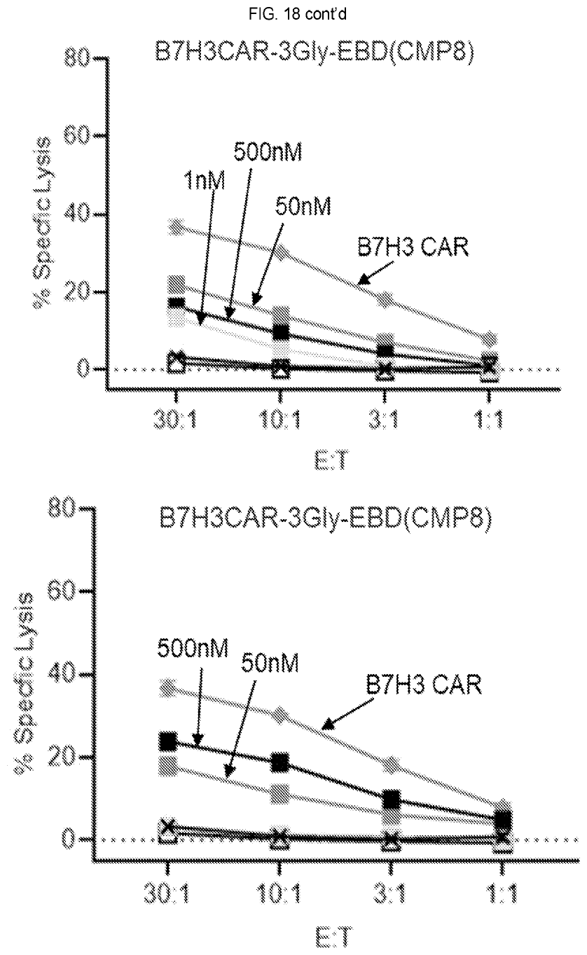

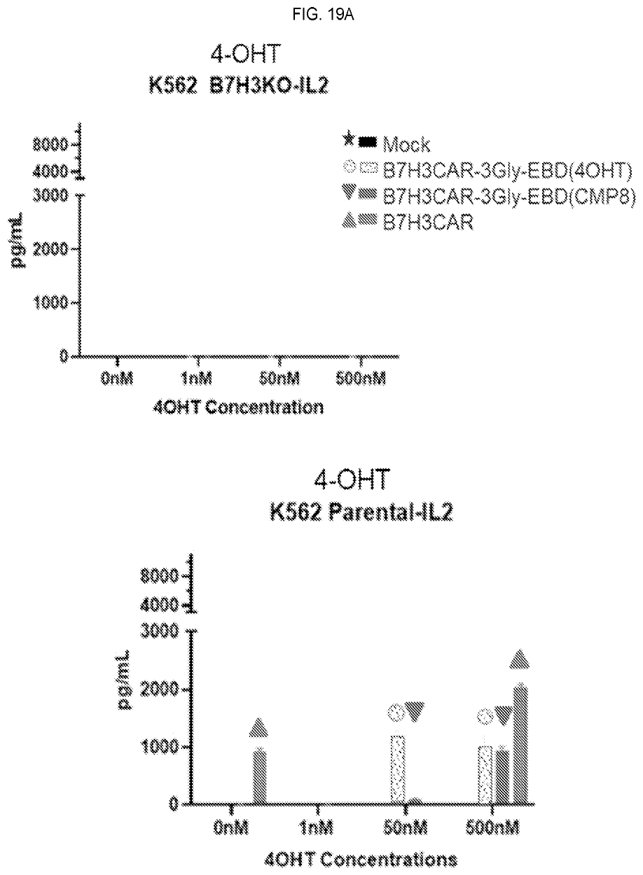

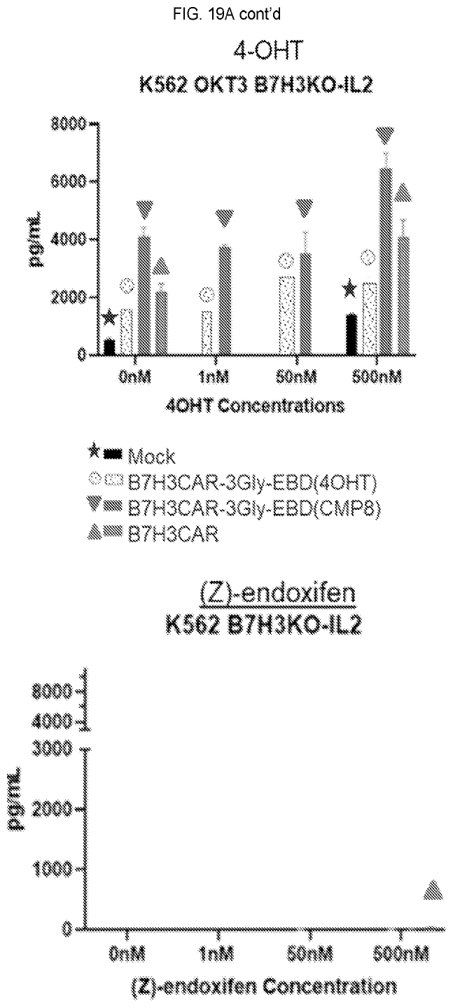

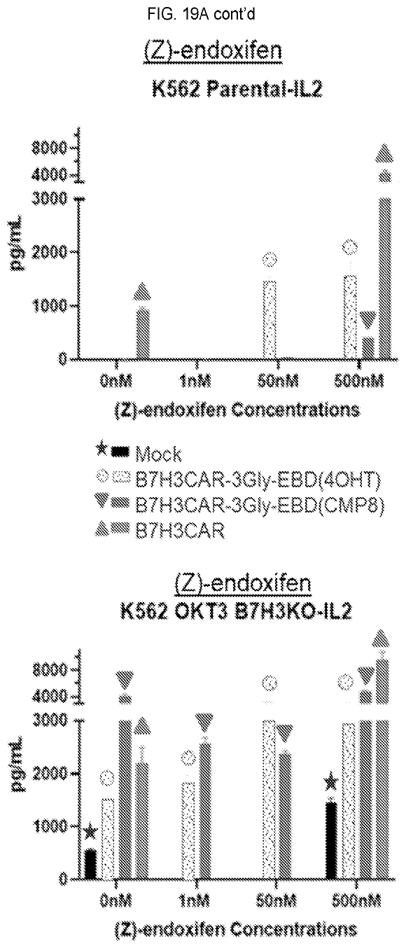

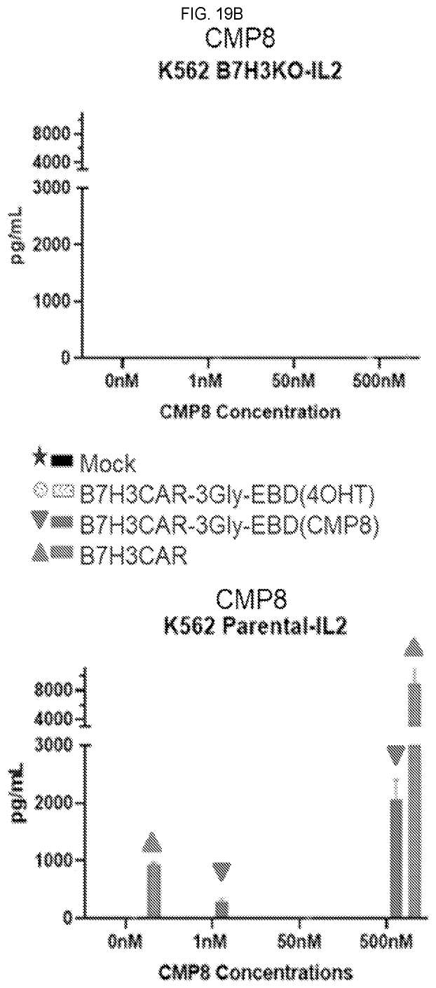

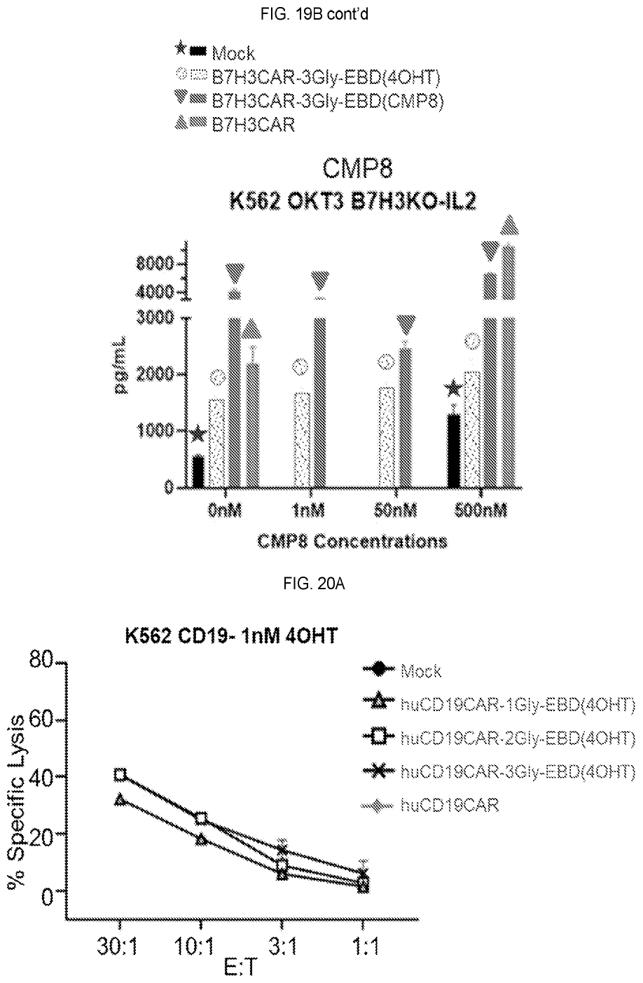

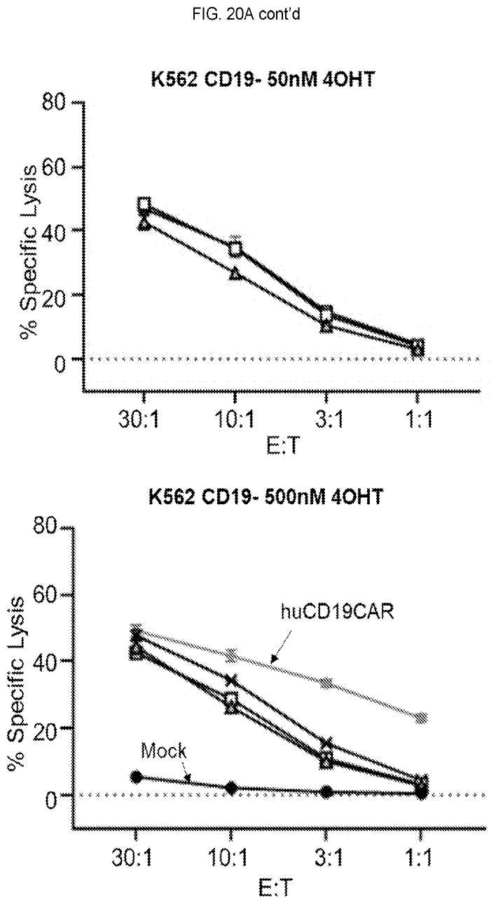

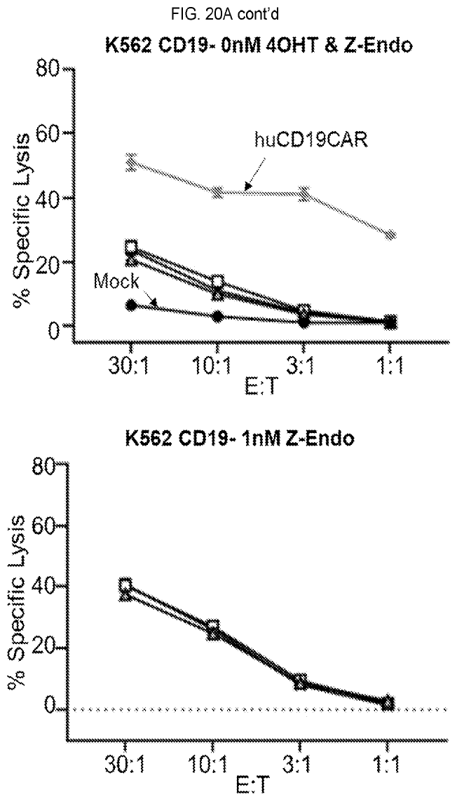

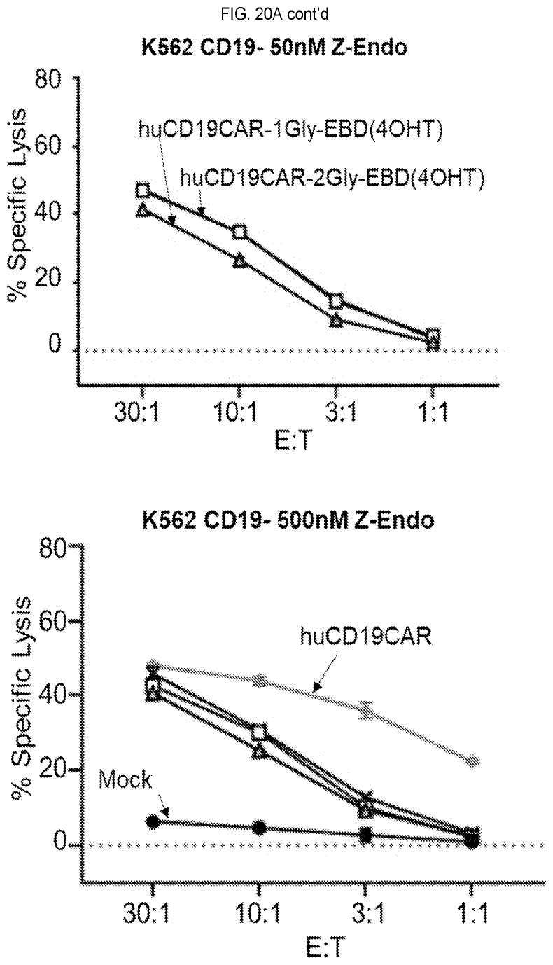

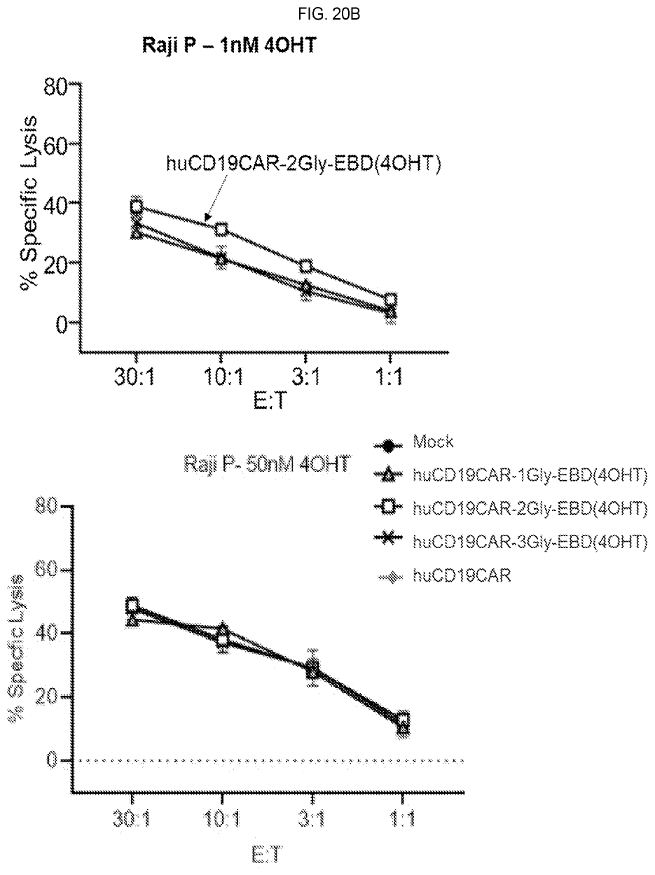

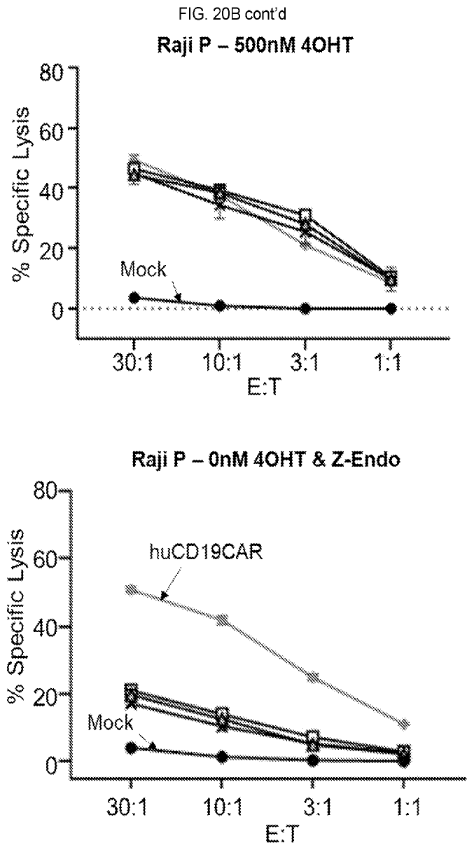

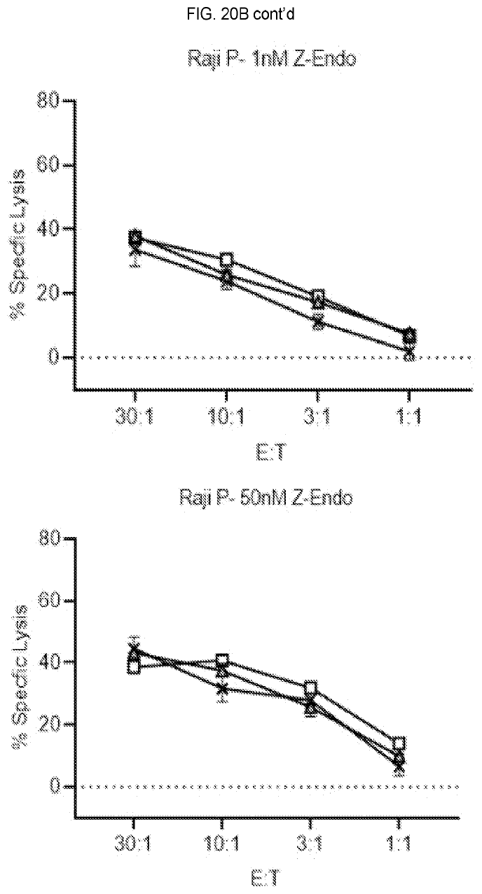

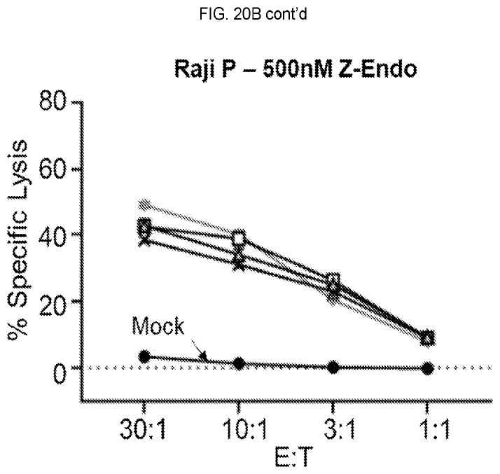

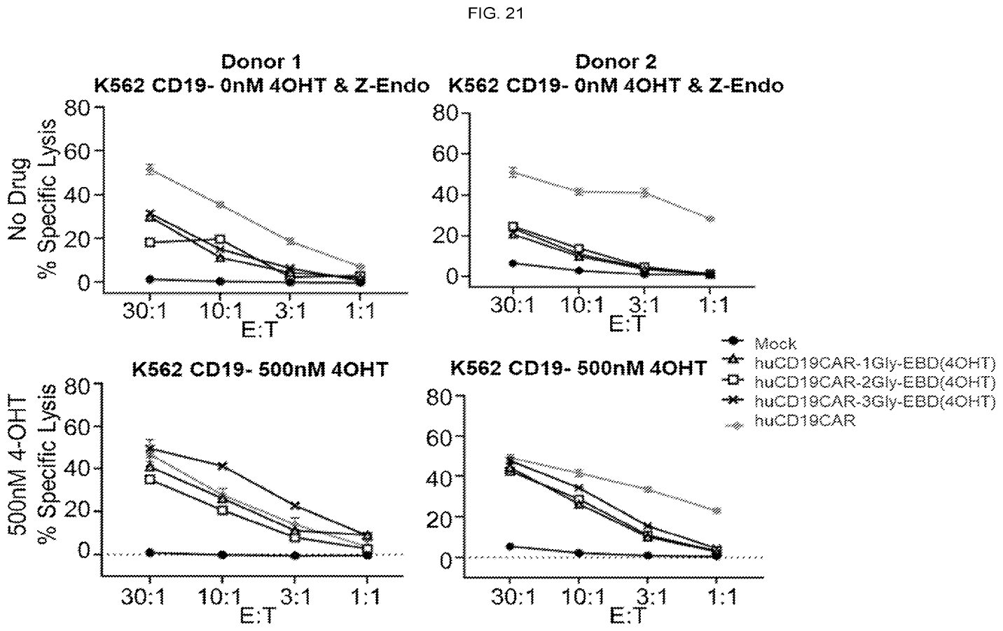

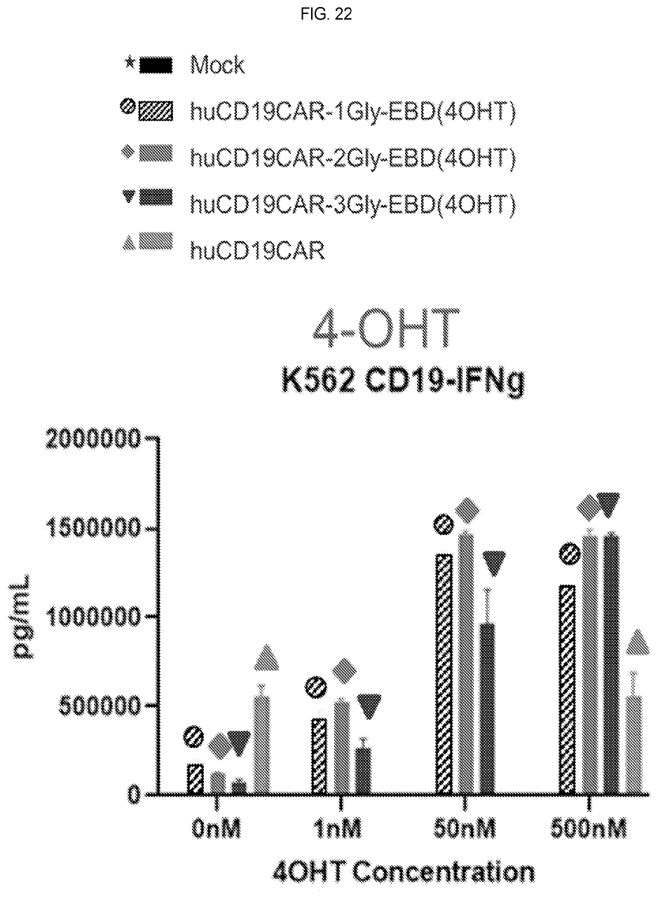

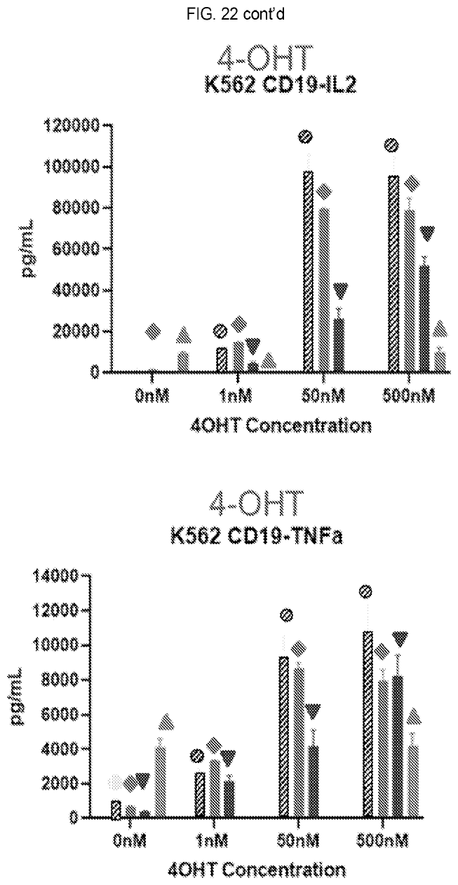

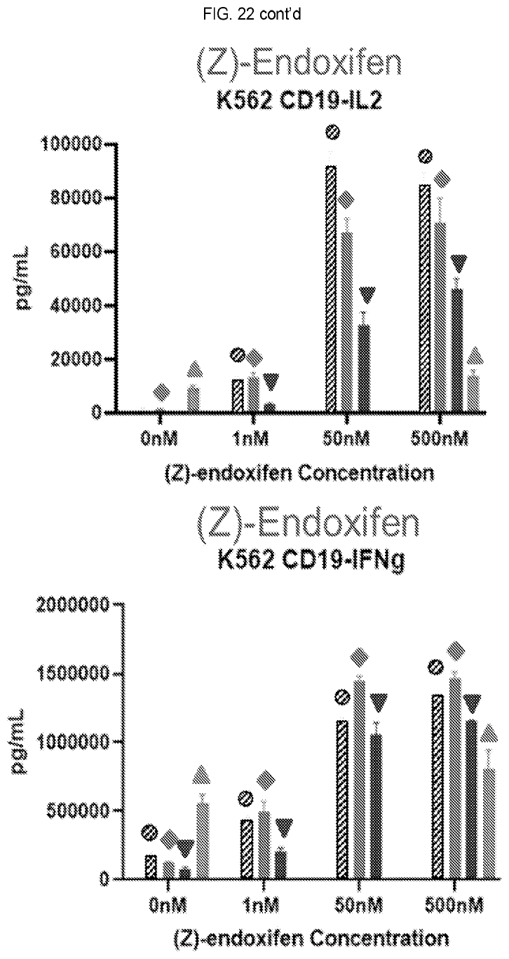

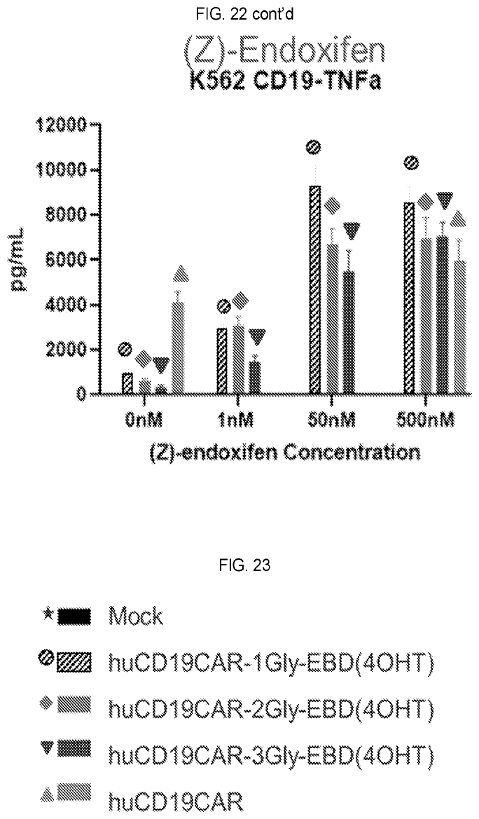

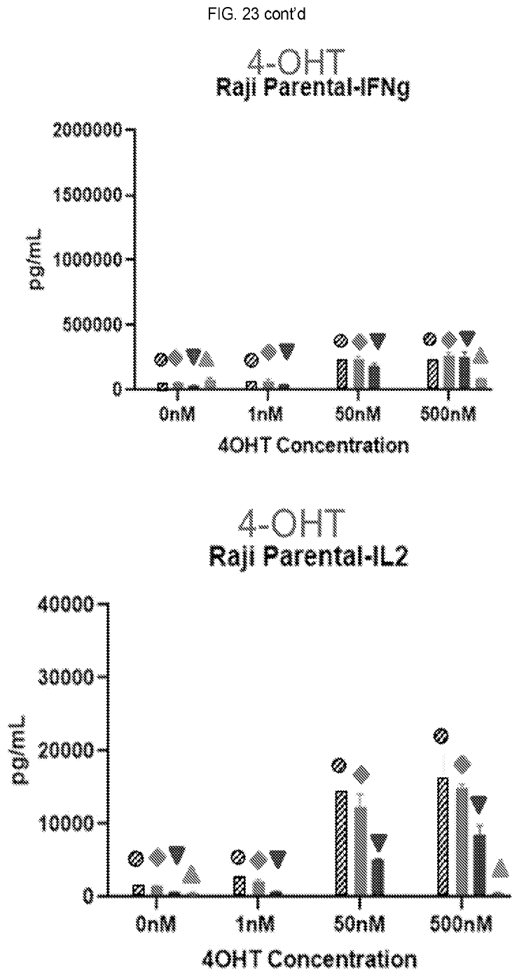

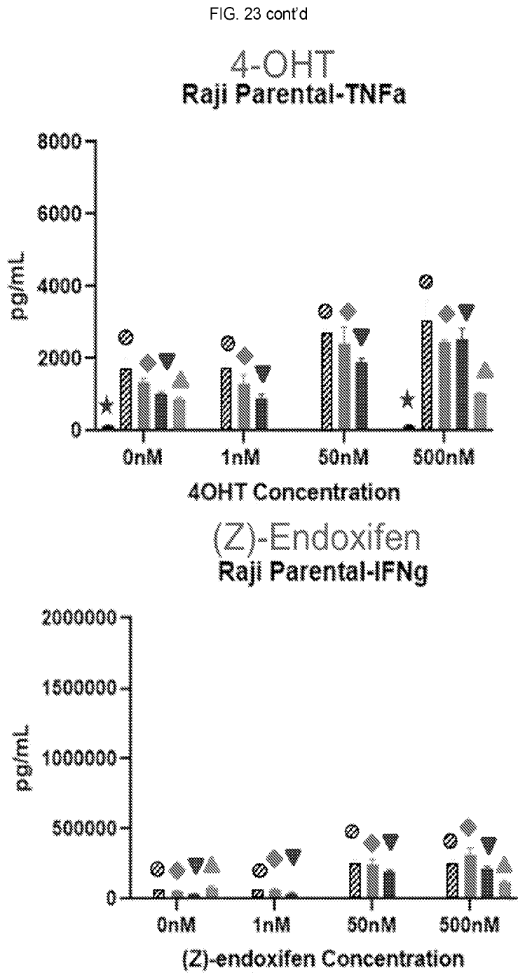

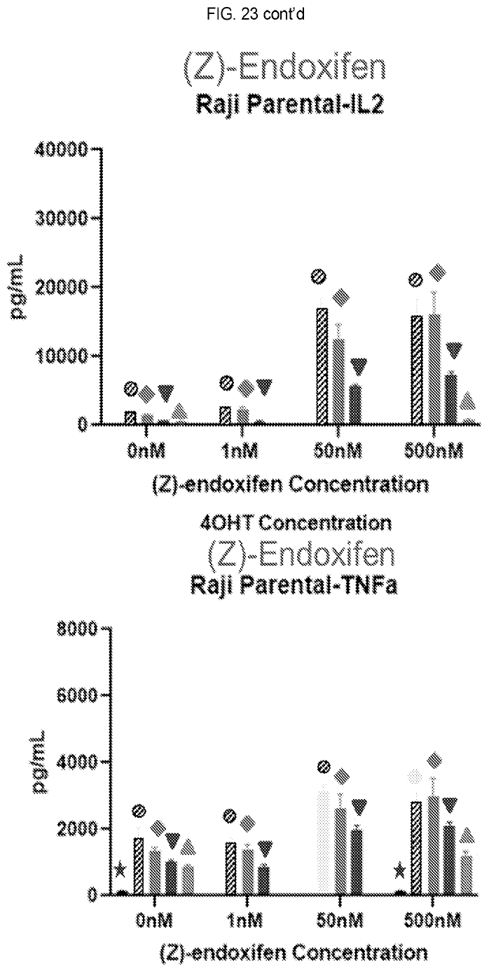

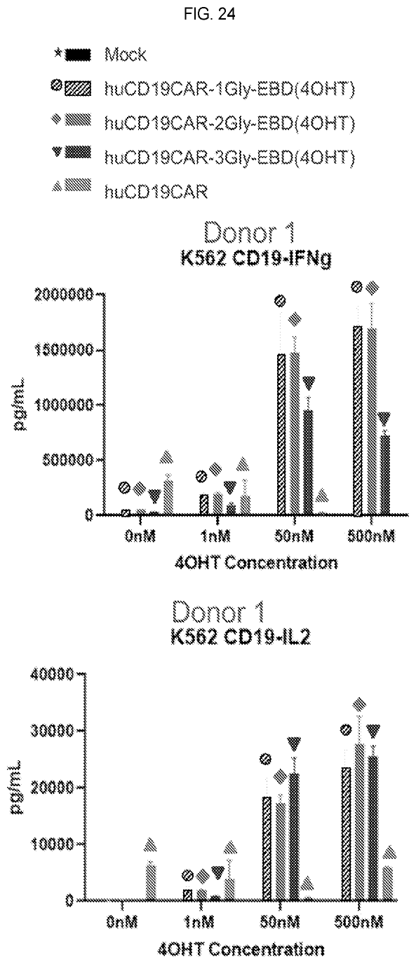

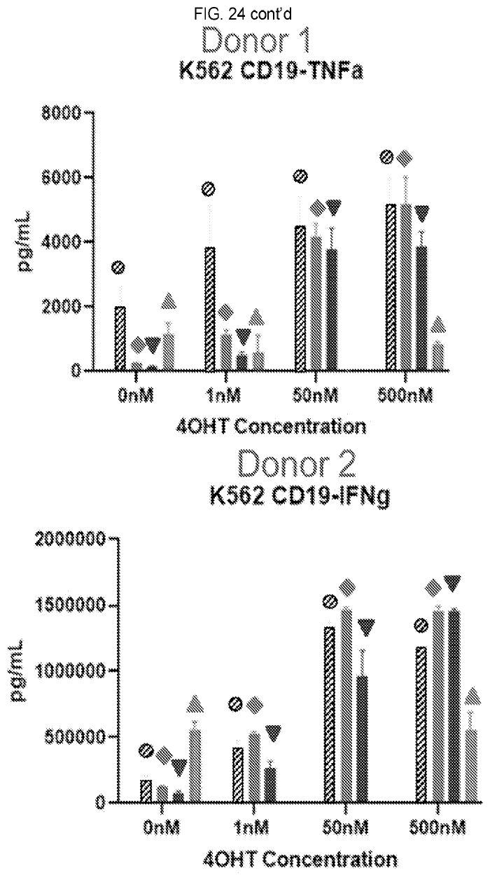

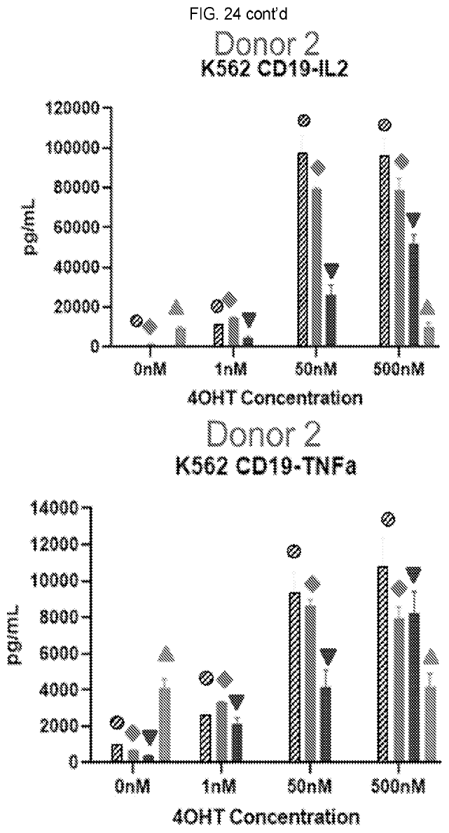

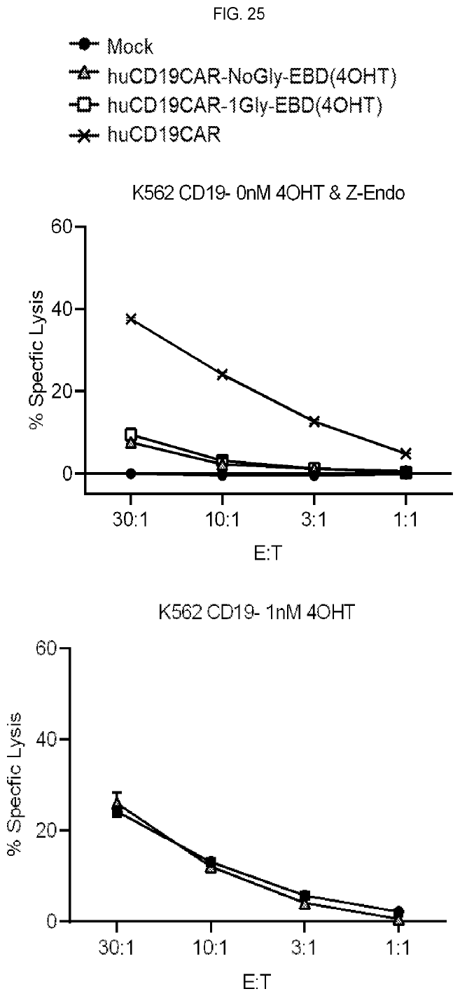

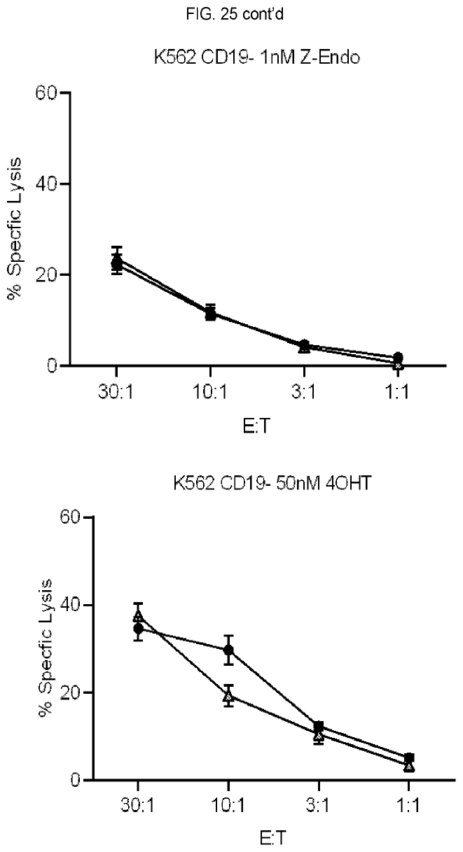

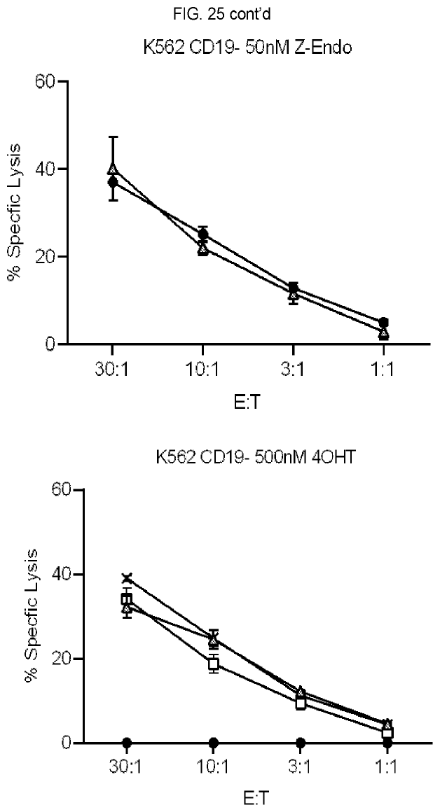

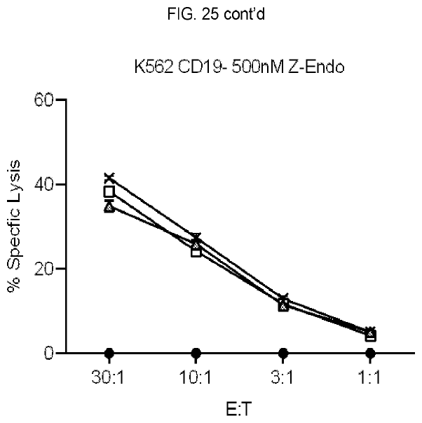

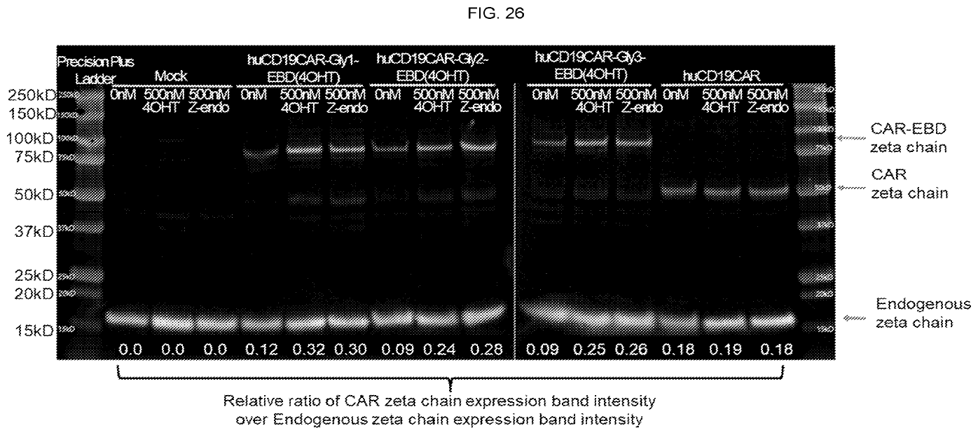

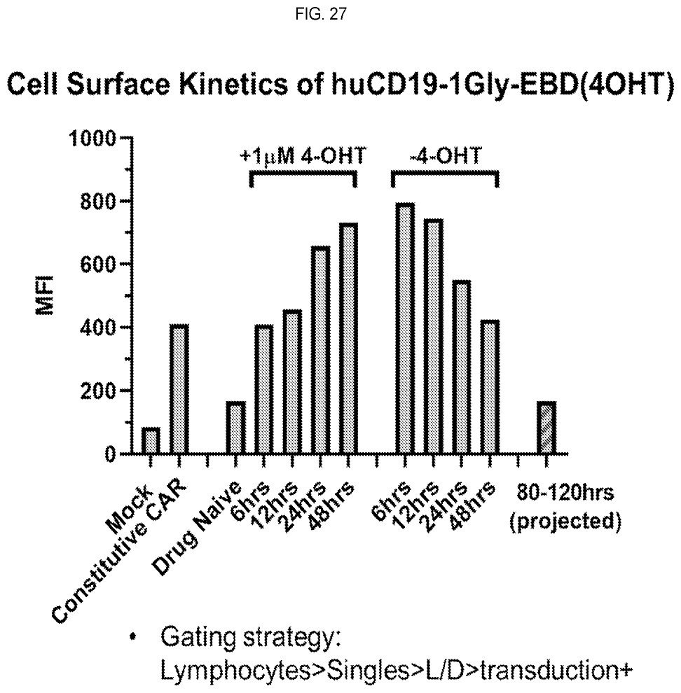

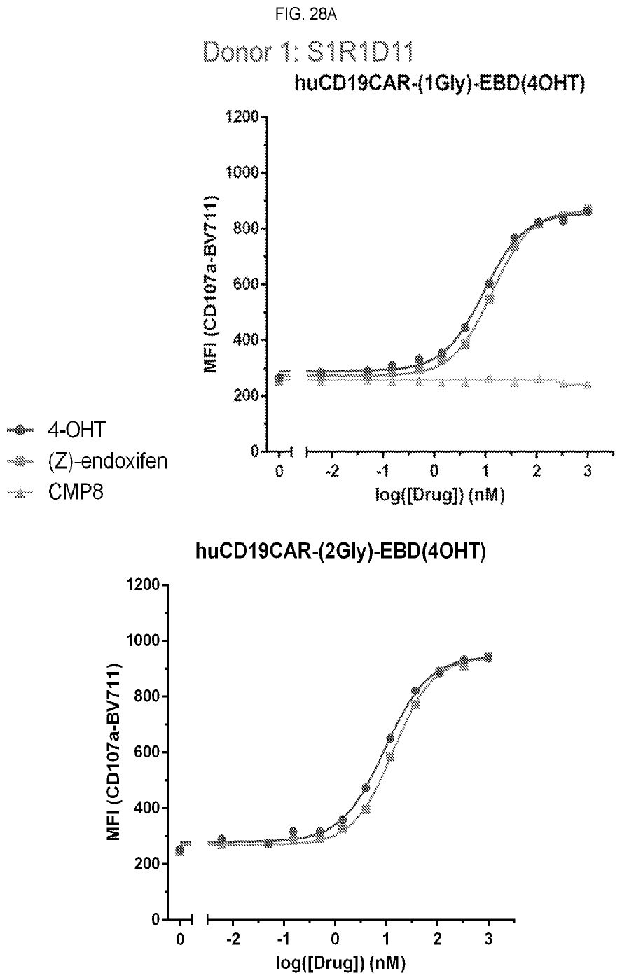

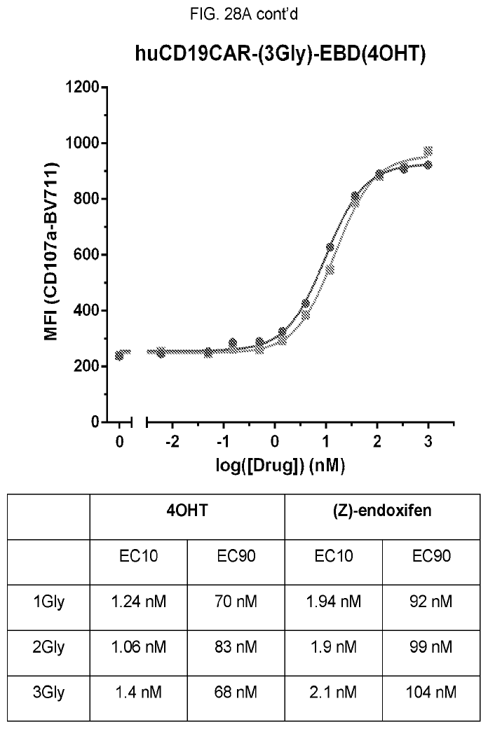

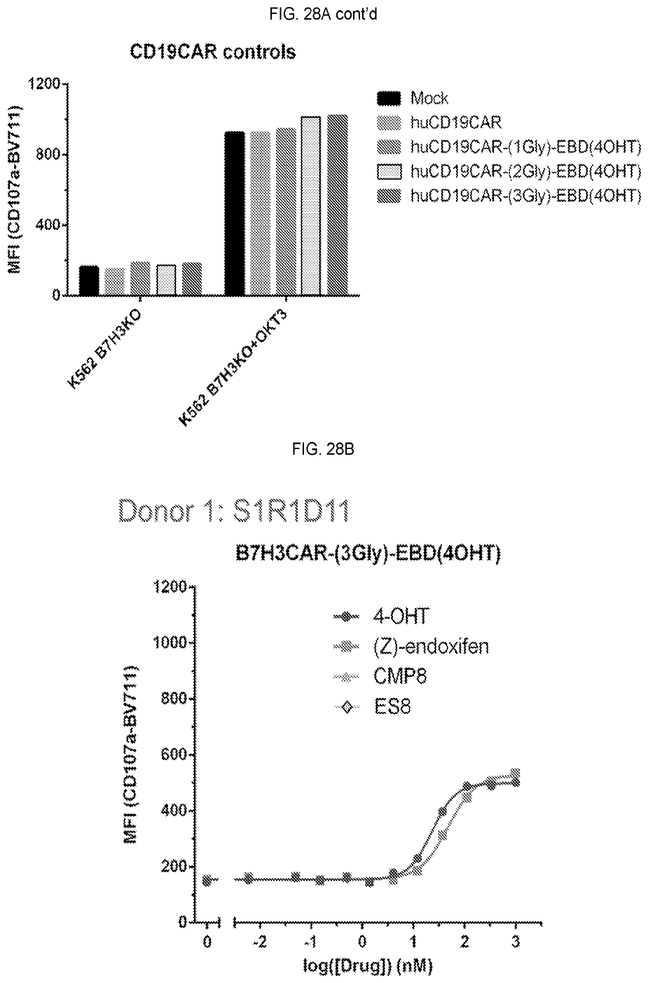

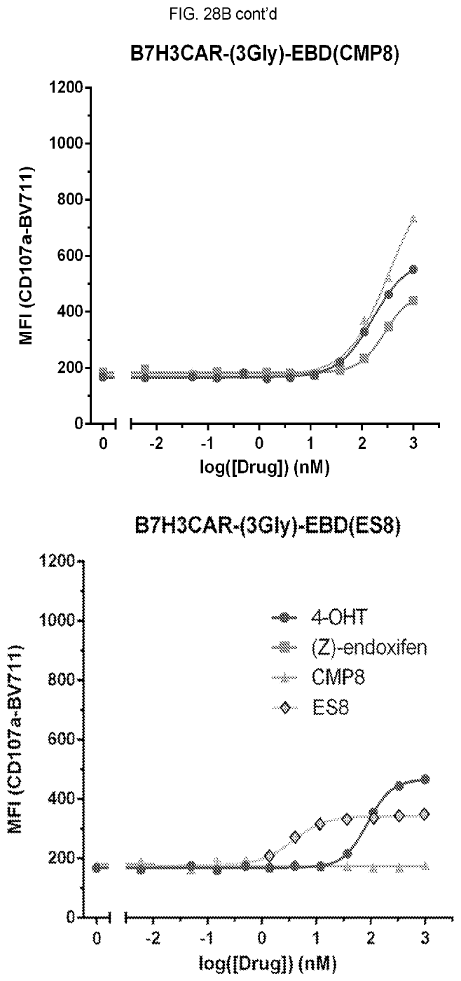

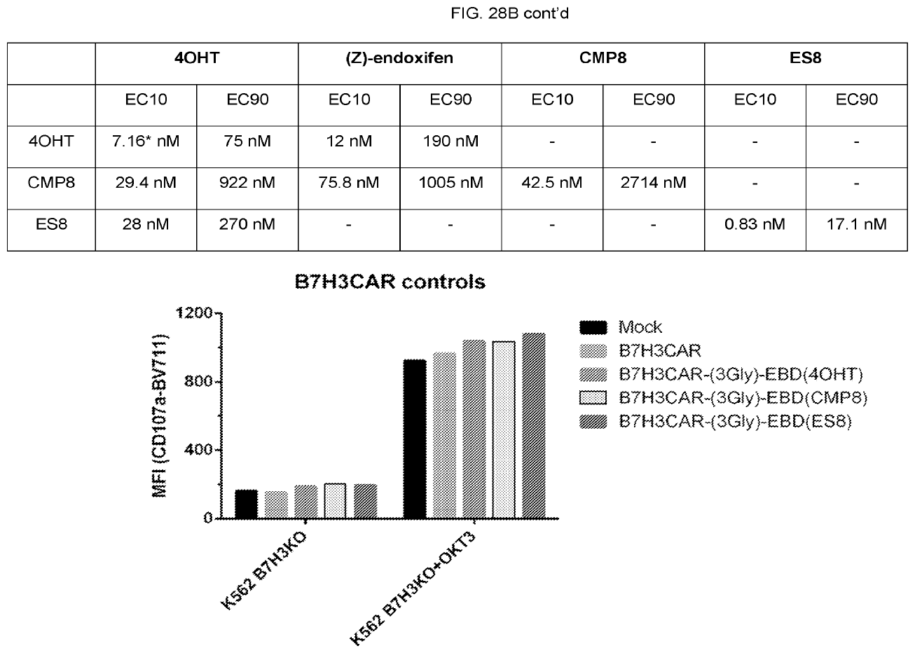

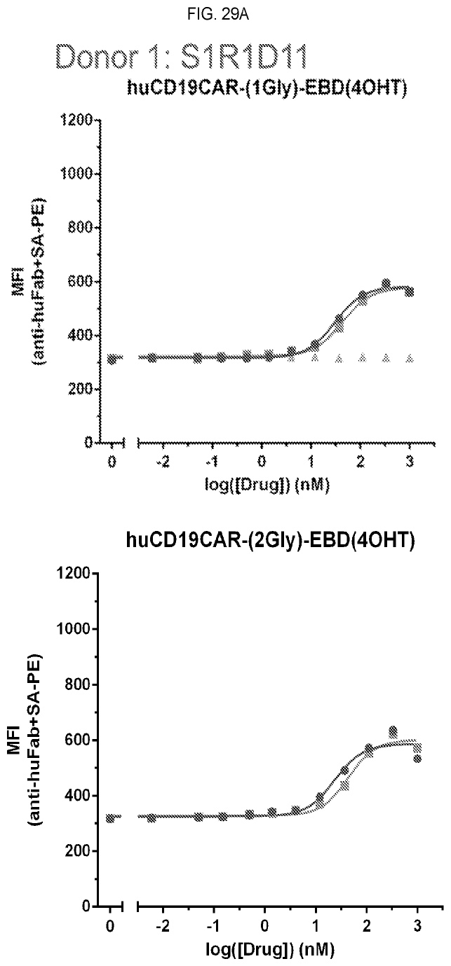

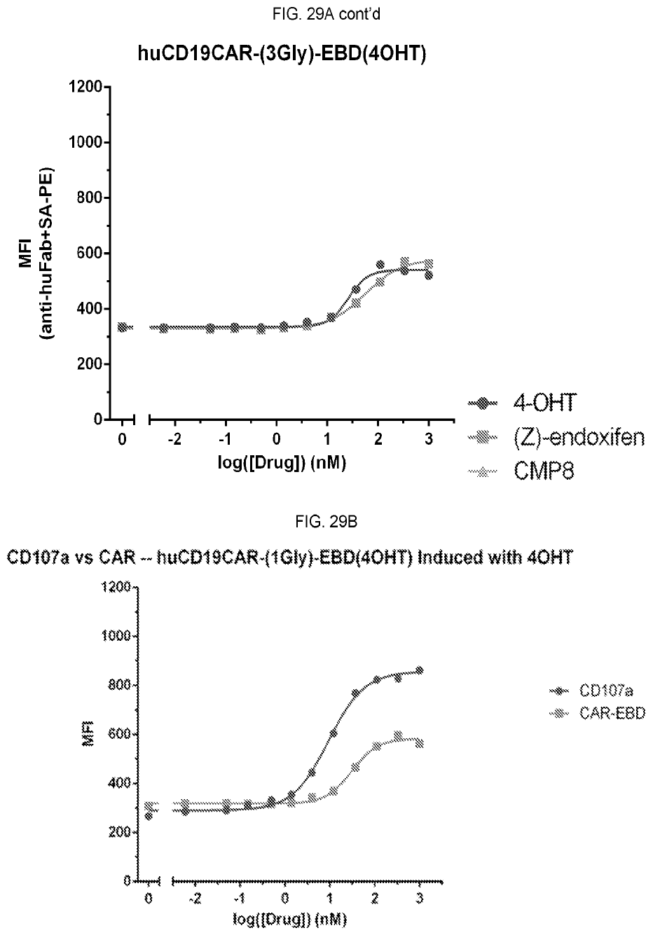

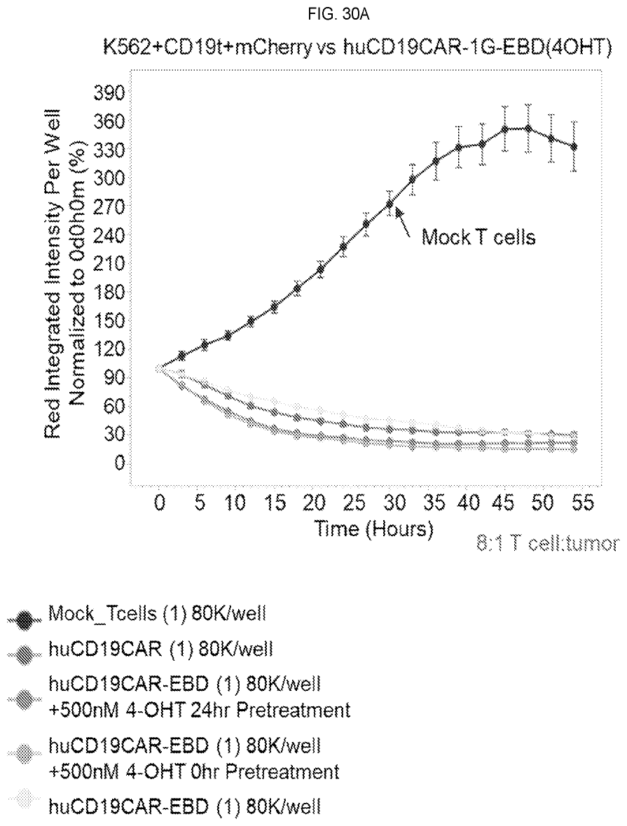

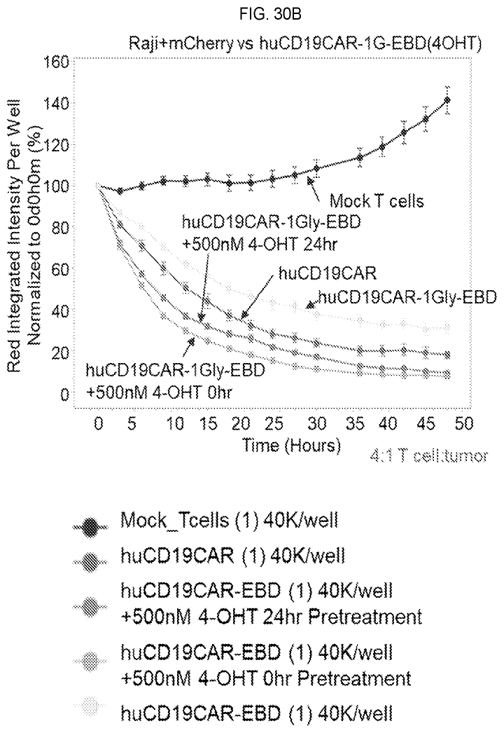

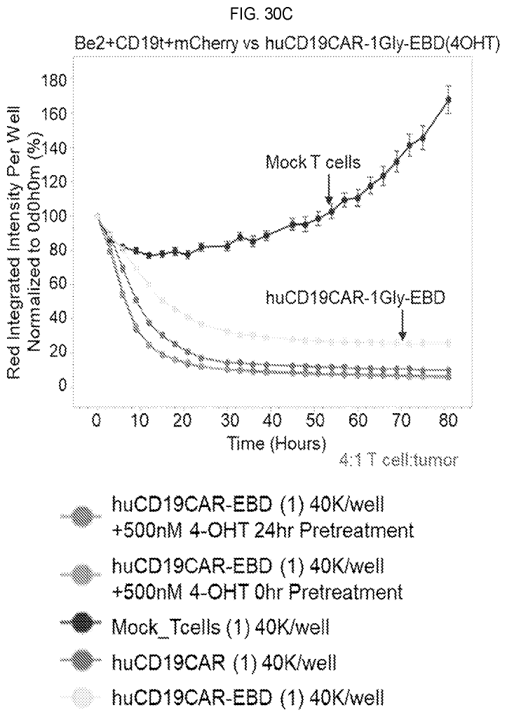

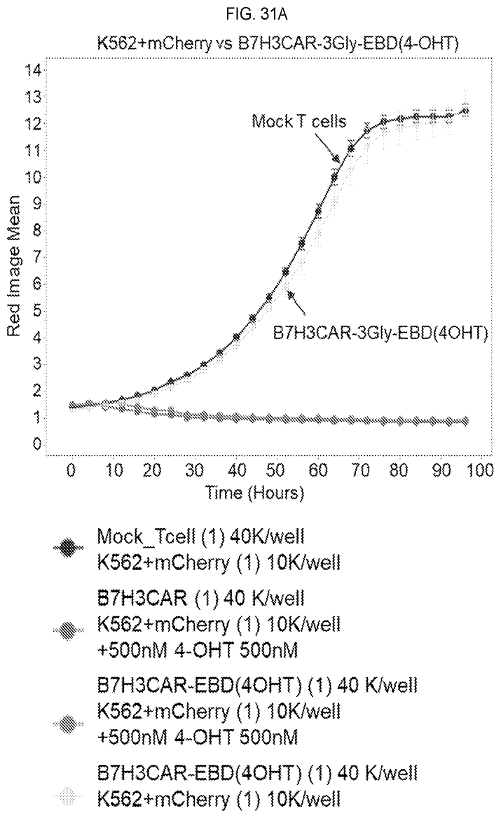

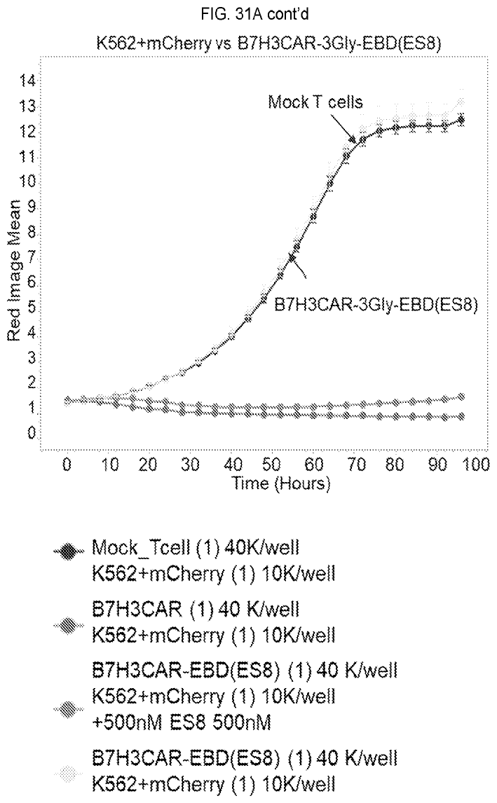

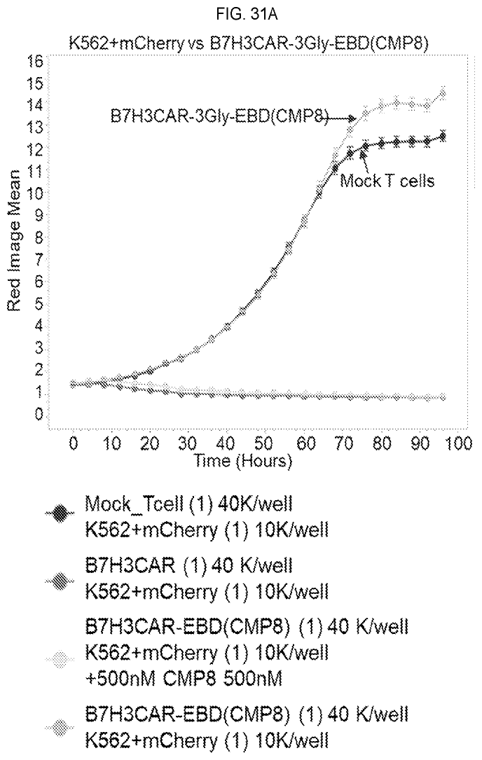

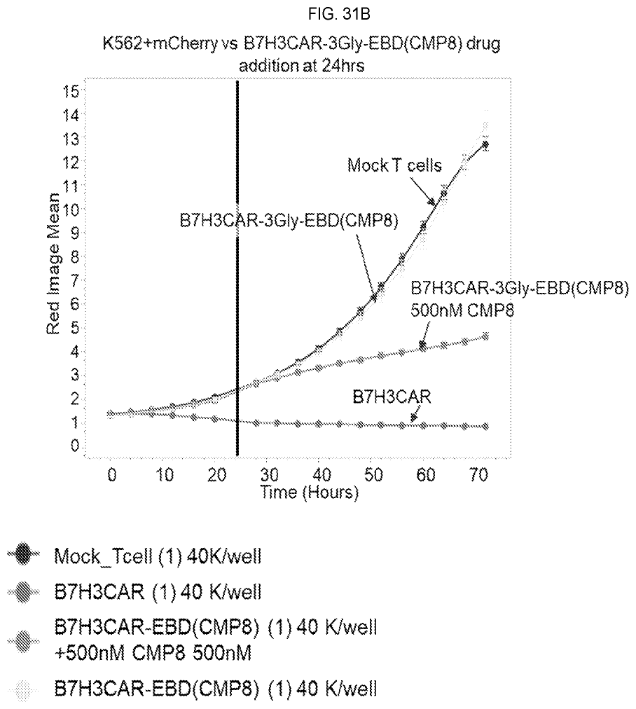

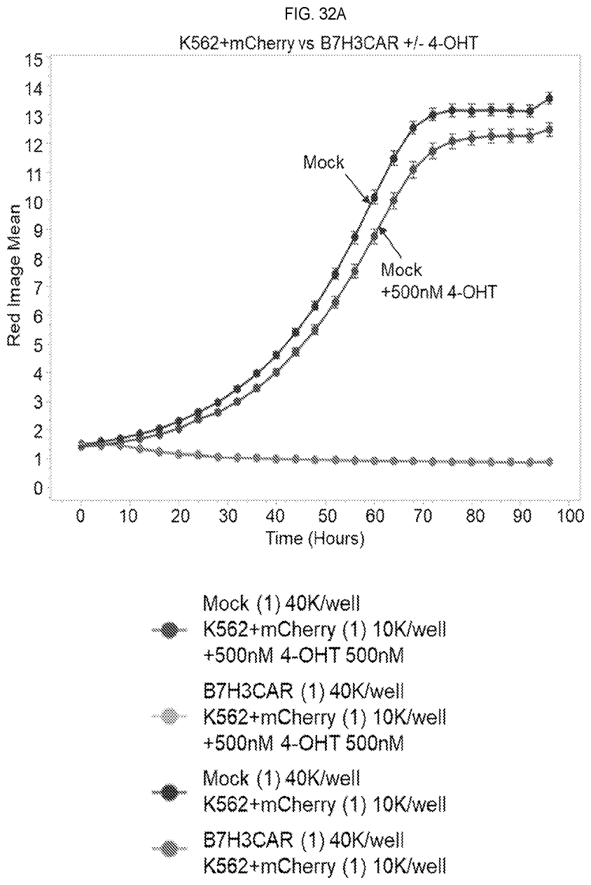

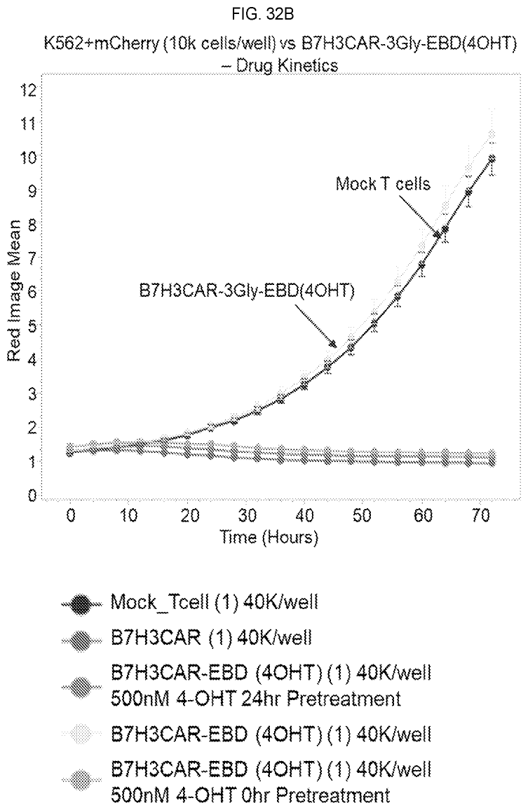

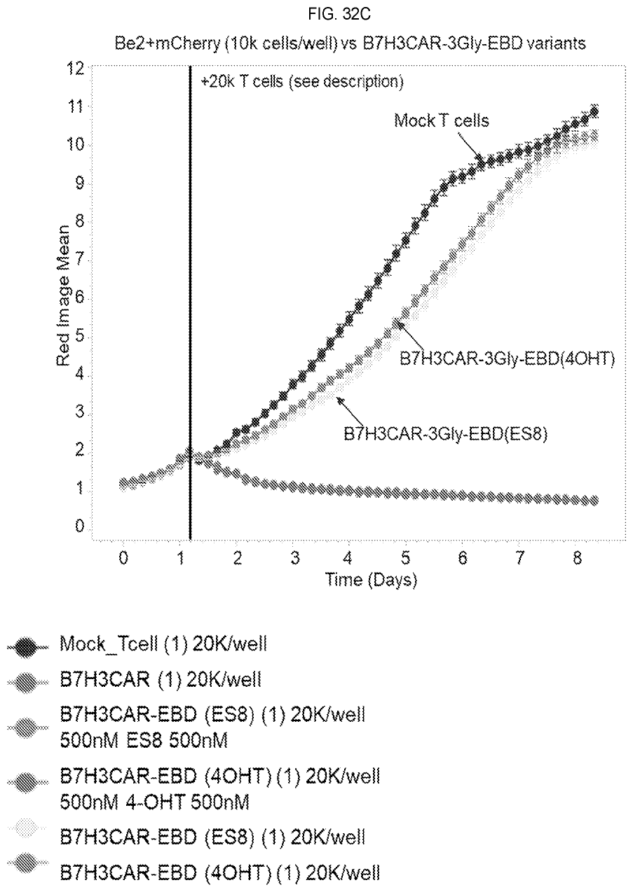

Aspects of the current disclosure are now described with additional detail and options as follows: (i) Drug Molecules and hsp90 Binding Domains; (ii) Ligand Binding Domains; (iii) Intracellular Signaling Domains; (iv) Transmembrane Domains; (v) Linkers; (vi) Tags and Selectable Markers; (vii) Additional Transmembrane Receptors; (viii) Cells Genetically Modified to Express Activity-Inducible Fusion Proteins; (ix) Methods to Modify Cells Ex Vivo and In Vivo; (x) Production of Activity-Inducible Fusion Proteins; (xi) Modified Formulations, Modifying Formulations, and Drug Compositions; (xii) Methods of Use; (xiii) Kits; (xiv) hsp90 Clients; (xv) Variants; (xvi) Exemplary Embodiments; (xvii) Experimental Example describing FIGS. 9-17; and (xviii) Closing Paragraphs. These headings are provided for organizational purposes only and do not limit the scope or interpretation of the disclosure.

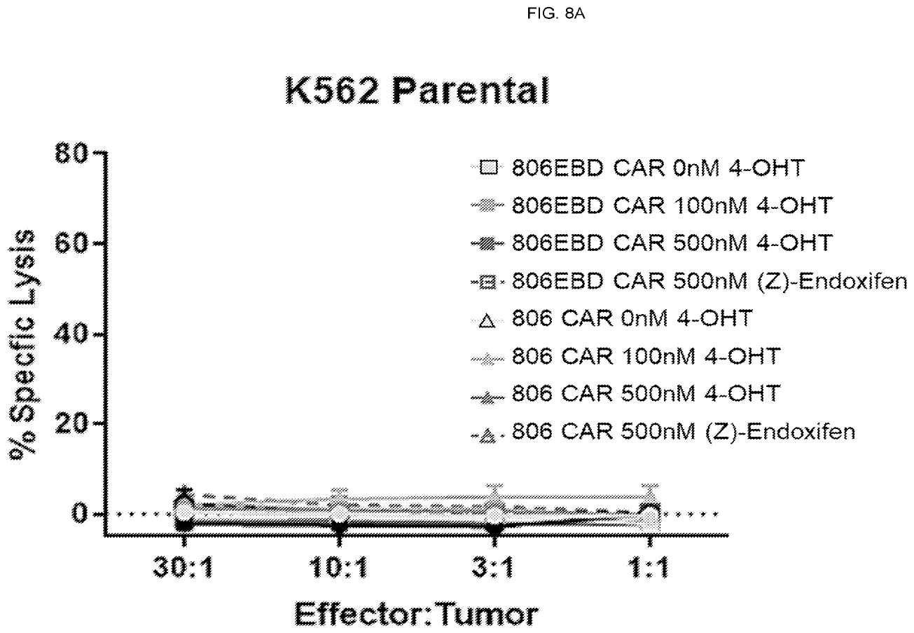

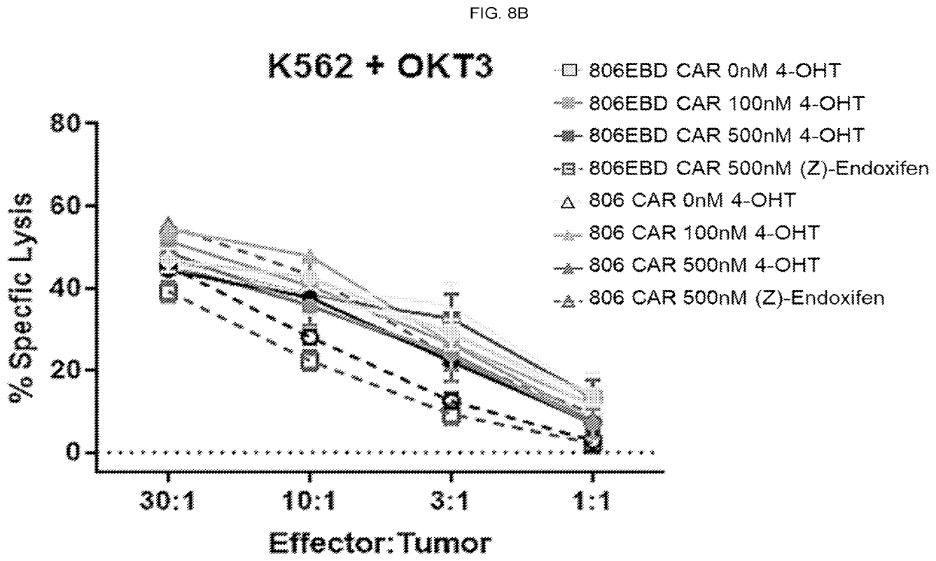

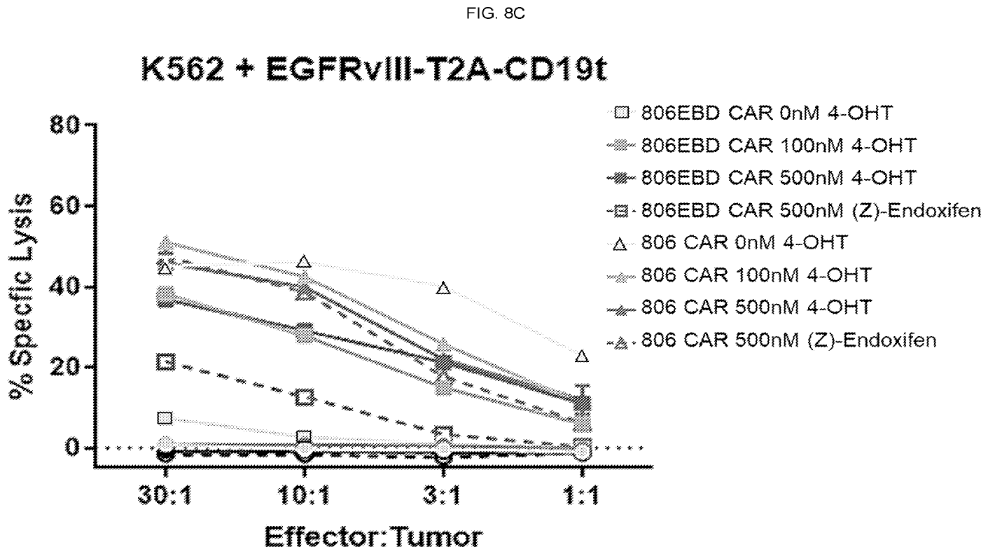

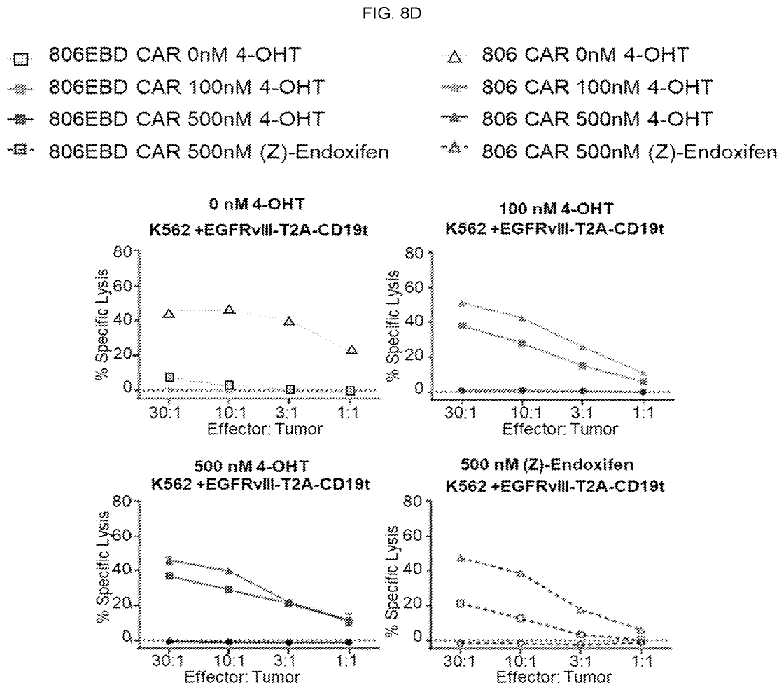

(i) Drug Molecules and hsp90 Binding Domains. Drug molecules used with activity-inducible fusion proteins disclosed herein can out-compete hsp90 for binding to a hsp90 binding domain present on the activity-inducible fusion protein and/or otherwise result in a conformation change that permits intracellular signaling. In certain examples, the binding domain present on the activity-inducible fusion protein is present on the intracellular segment of a CAR. This out-competition and/or conformational change allows activation of the CAR following antigen binding (see, e.g., FIGS. 8A-9C). In certain examples, the hsp90 binding domain is a hormone binding domain or modified form thereof.

In some alternatives, the drug molecule is a small molecule estrogen analog. Small molecule estrogen analogs include tamoxifen and salts and metabolites thereof, as well as compounds with structural similarity as described herein.

Tamoxifen is an estrogen antagonist/partial agonist that is an FDA-approved and commercially available drug. Tamoxifen has a proven safety record, favorable pharmacokinetic profile, exceIlent tissue distribution and a low partition coefficient between the extracellular space and cytosol. Tamoxifen is frequently administered orally as a pharmaceutically acceptable salt. For example, Tamoxifen citrate (RN 54965-24-1, M.W. 563.643) is indicated for treatment of metastatic breast cancer, and as an adjuvant for the treatment of breast cancer in women following mastectomy axillary dissection, and breast irradiation. Tamoxifen citrate is also indicated to reduce incidence of breast cancer in women at high risk for breast cancer.

Tamoxifen, CAS RN: 10540-29-1, is also known as 2-(4-((1Z)-1,2-diphenyl-1-butenyl)phenoxy)-N,N-dimethyl-ethanamine, or (Z)-2-(para-(1,2-Diphenyl-1-butenyl)phenoxy)-N,N-dimethylamine (IUPAC), and has a molecular formula of C26H29NO and a molecular weight (M.W.) of 371.52 g/mol.

Metabolites of tamoxifen that can be useful in some approaches described herein, include the major metabolites N-desmethyltamoxifen (RN 31750-48-8, M.W. 357.494), 4-hydroxytamoxifen (4-OHT) (RN 68392-35-8, M.W. 387.52, Afimoxifene), and Endoxifen. These compounds are well known and described in Robinson et al. (Metabolites, pharmacodynamics, and pharmacokinetics of tamoxifen in rats and mice compared to the breast cancer patient. Drug Metab Dispos January 1991 19:36-43). Additional metabolites, useful in some approaches described herein, include cis-4-hydroxytamoxifen (RN 174592, M.W. 387.52; Afimoxifene, E-isomer), and 4′-hydroxytamoxifen ((Z)-4-(1-(4-(2-(dimethylamino)ethoxy)phenyl)-1-phenylbut-1-en-2-yl)pheno-I) as described in Crewe et al. (Metabolism of Tamoxifen by recombinant human cytochrome P-450 enzymes: Formation of the 4-hydroxy, 4′-hydroxy and N-desmethyl metabolites and isomerization of trans-4-hydroxytamoxifen, Drug Metab Dispos, 30(8): 869-874, 2002, FIG. 1).

Compounds with structural similarity to tamoxifen, useful in some approaches described herein, include cis-tamoxifen (RN 13002-65-8, M.W. 371.521), 4-methyltamoxifen (RN 73717-95-5, M.W. 385.548), N-desmethyltamoxifen (RN 31750-48-8, M.W. 357.494), (Z)-desethyl methyl tamoxifen (RN 15917-50-7, M.W. 357.494), (E)-desethyl methyl tamoxifen (RN 31750-45-5, M.W. 357.494), trans-4-hydoxytamoxifen (RN 68047-06-3, M.W. 387.52), Afimoxifene (RN 68392-35-8, M.W. 387.52, 4-hydroxytamoxifen), Afimoxifene, E-isomer (RN 174592-47-3, M.W. 387.52), 4-chlorotamoxifen (RN 77588-46-6, M.W. 405.966), 4-fluorotamoxifen (RN 73617-96-6, M.W. 389.511), Toremifene (RN 89778-26-7, M.W. 405.966), desethyl tamoxifen (RN 19957-51-8, M.W. 343.47), (E)-desethyl tamoxifen (RN 97151-10-5, M.W. 343.47), (Z)-desethyl tamoxifen (RN 97151-11-6, M.W. 343.47), Miproxifene (RN 129612-87-9, M.W. 429.6), 2-(p-(beta-ethyl-alpha-phenyl styryl)phenoxy)triethylamine (RN 749-86-0, M.W. 399.575), Droloxifene (RN 82413-20-5, M.W. 387.52), 4-iodo-tamoxifen (RN 116057-68-2, M.W. 497.413), dihydrotamoxifen (RN 109640-20-2, M.W. 373.537), (E)-N,N-dimethyl-2-(4-(1-(2-methylphenyl)-2-phenyl-1-butenyl)phenoxy)ethanamine (RN 97150-96-4, M.W. 385.548), 4-hydroxytoremifene (RN 110503-62-3, M.W. 421.965); and pharmaceutically acceptable salts, hydrates or solvates thereof.

Citrate salts of tamoxifen, or citrate salts of compounds with structural similarity to tamoxifen, useful in some approaches described herein, include tamoxifen citrate (RN 54965-24-1, M.W. 563.64), 2-(p-(1,2-diphenyl-1-butenyl)phenoxy)-N,N-dimethylethylamine citrate (RN 7244-97-5, 563.64), (E)-tamoxifen citrate (RN 76487-65-5, M.W. 563.64), Toremifene citrate (RN 89778-27-8, M.W. 598.088), Droloxifene citrate (RN 97752-20-0, M.W. 579.64), 2-(p-(1,2-bis(p-methoxyphenyl)-1-butenyl)phenoxy)triethylamine citrate (RN 42920-39-8, M.W. 651.748), 2-(4-(1,2-diphenylethenyl)phenoxy)-N,N-diethyl-ethanamine 2-hydroxy-1,2,3-propanetricarboxylate (RN 40297-42-5, M.W. 563.643), 2-(p-(alpha-phenyl styryl)phenoxy)triethylamine citrate (RN 102433-95-4, M.W. 563.64), 2-(p-(2-(p-methoxyphenyl)-1-phenyl-1-butenyl)phenoxy)triethylamine citrate (1:1) (RN 42824-34-0, M.W. 637.72), 2-(p-(1-(p-methoxyphenyl)-2-phenylpropenyl)phenoxy)triethylamine citrate (RN 13554-24-0, M.W. 607.696), 2-(p-(alpha-(p-methoxyphenyl)styryl)phenoxy)triethylamine citrate monohydrate (RN 13542-71-7, M.W. 593.669), 2-(p-(p-methoxy-alpha-phenylphenethyl) phenoxy)triethylamine citrate (RN 16421-72-0, M.W. 595.685), alpha-(p-(2-(diethylamino)ethoxy)phenyl)-beta-ethyl-p-methoxy-alpha-pheny-Iphenethyl alcohol citrate (1:1) (RN 35263-93-5, M.W. 639.737), 1-(p-(2-(diethylamino)ethoxy)phenyl)-2-(p-methoxyphenyl)-1-phenylethanol citrate (M.W. 611.68), alpha-p-(2-(diethyl amino)ethoxy)phenyl)-beta-ethyl-alpha-(p-hydroxyphenyl)-p-methoxyphenethy-I alcohol citrate (RN 35263-96-8, M.W. 655.737), and 2-(p-(p-methoxy-alpha-methylphenethyl)phenoxy)-triethylamine citrate (RN 15624-34-7, M.W. 533.614).

Particular embodiments utilize tamoxifen, 4-OHT, ES8, or CMP8 as the drug molecule. Particular embodiments utilize fulvestrant or raloxifene as the drug molecule.

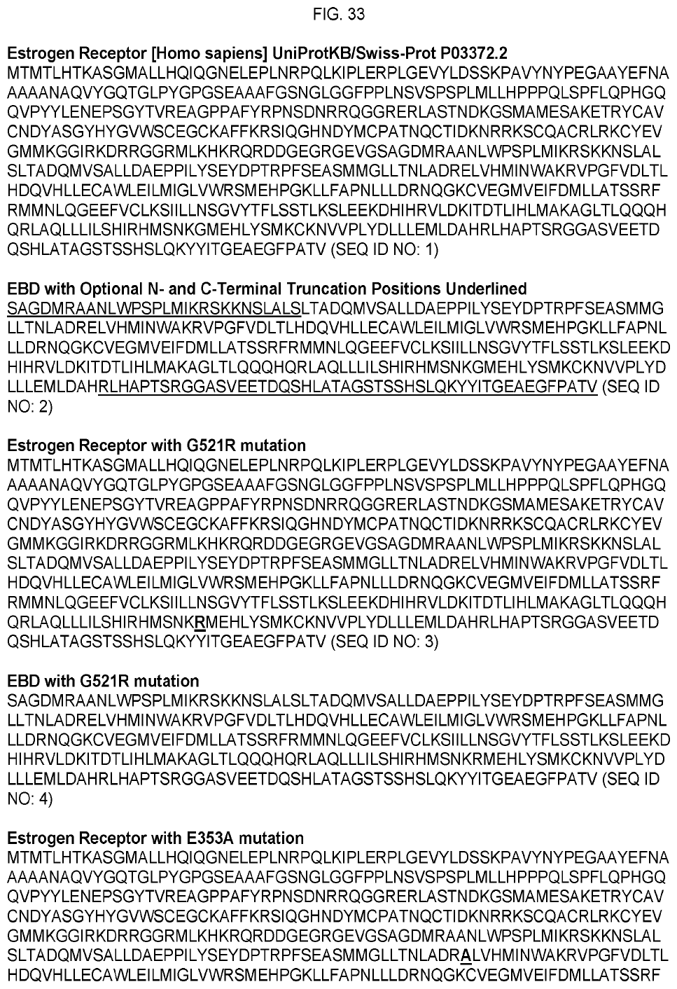

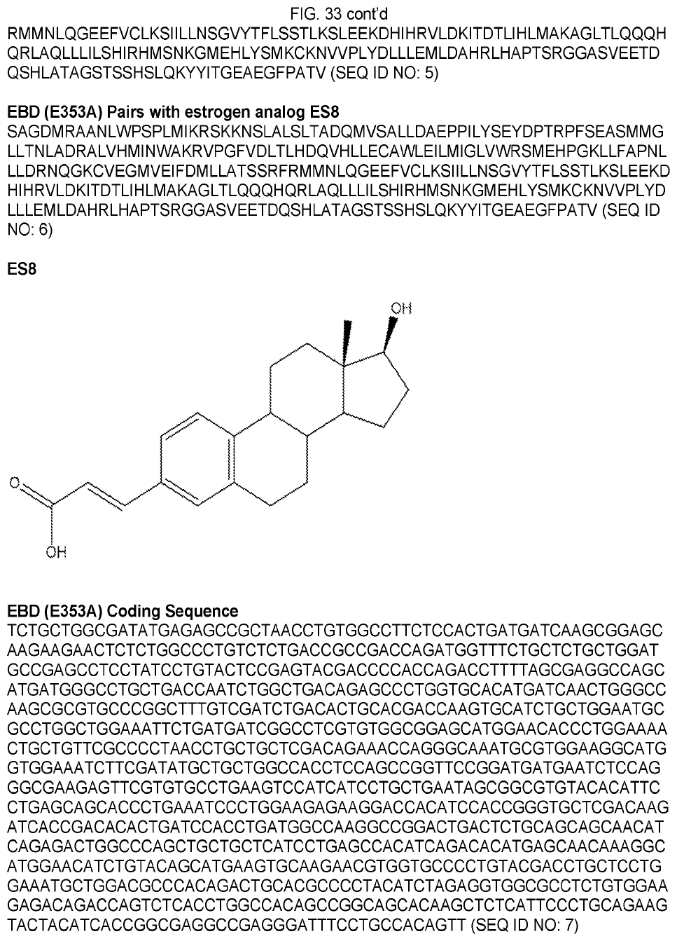

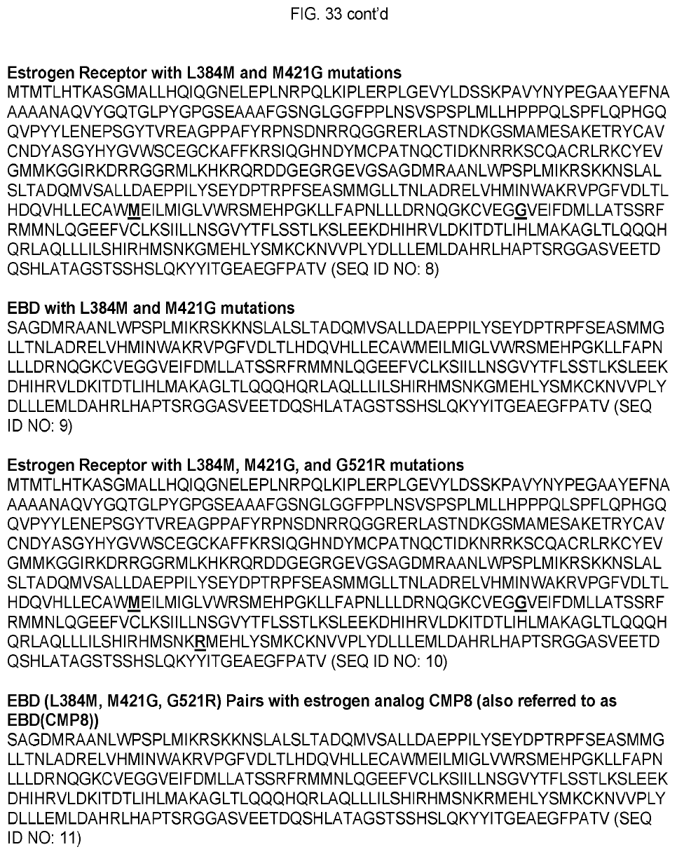

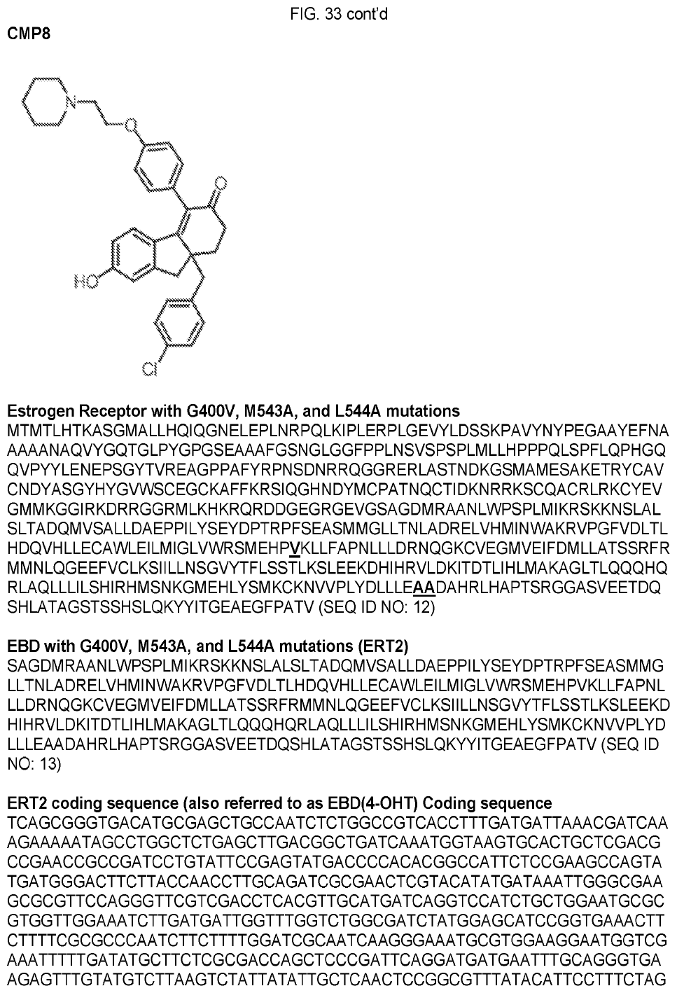

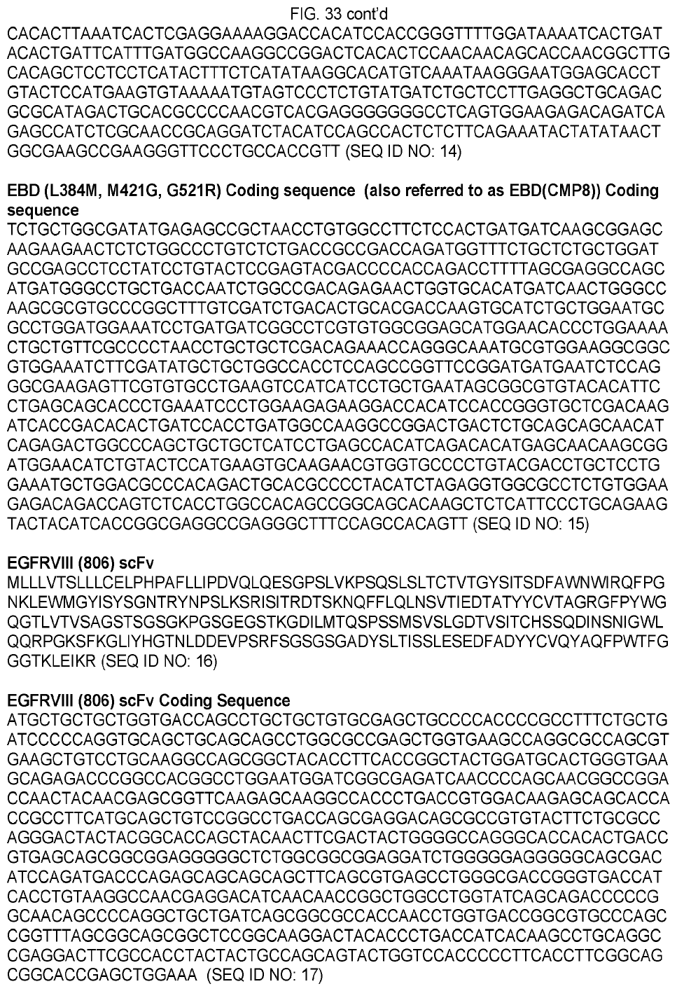

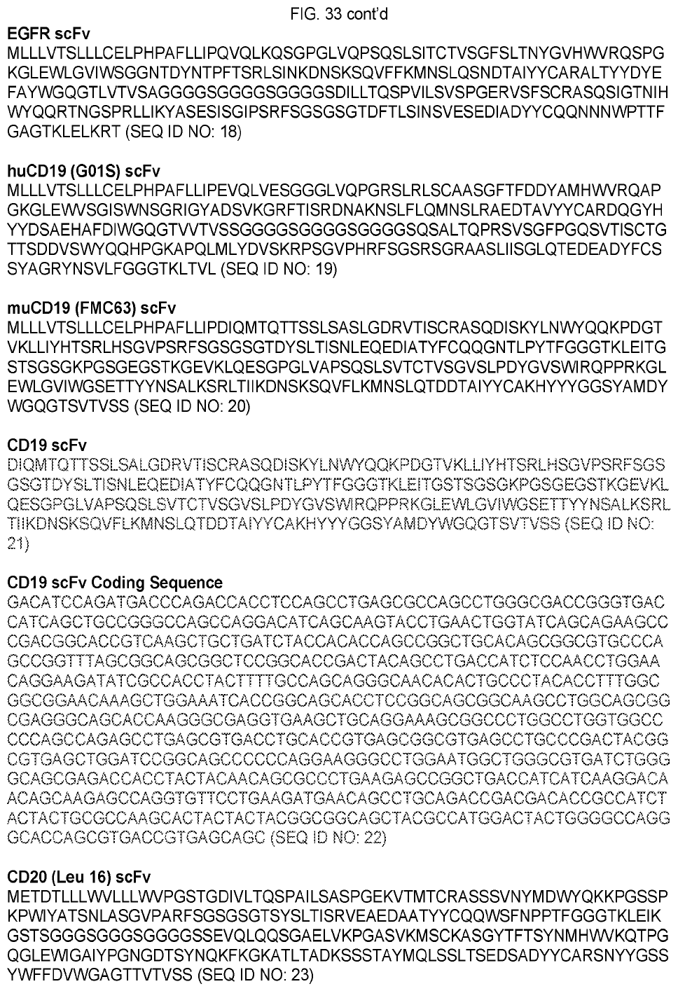

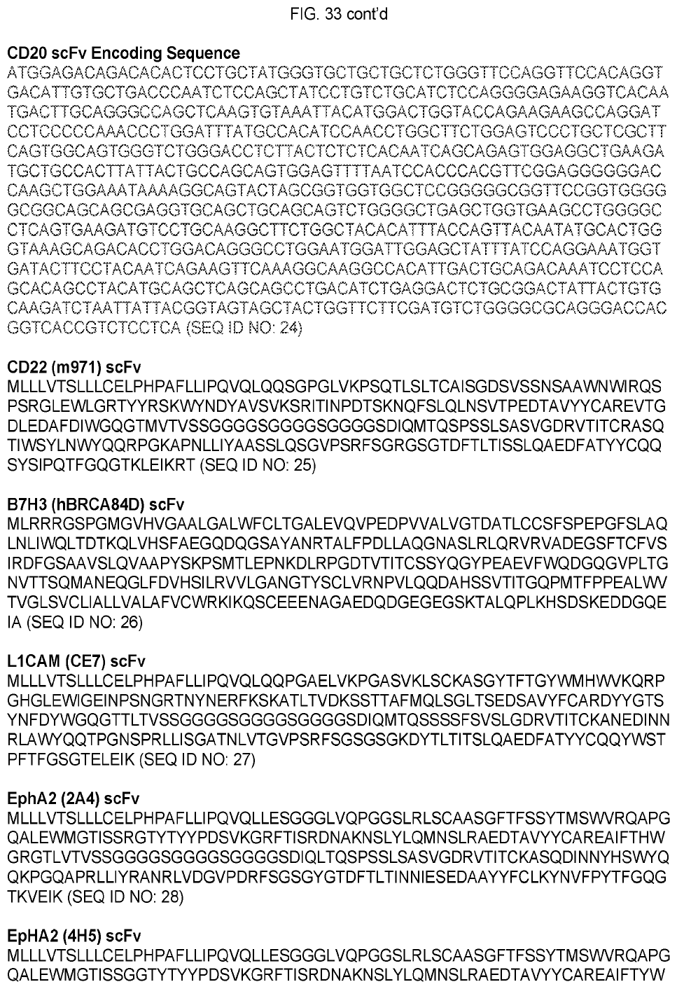

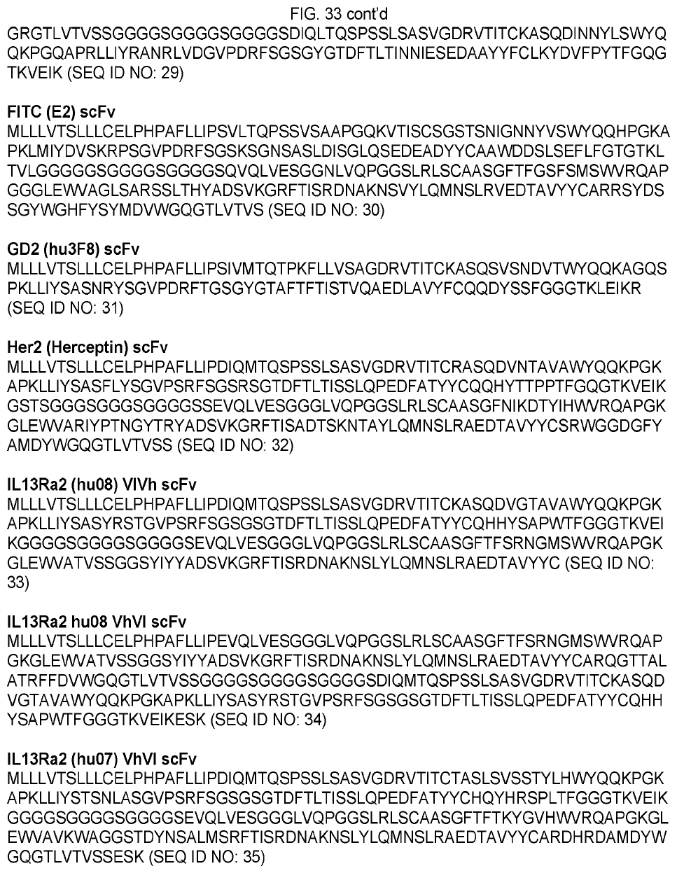

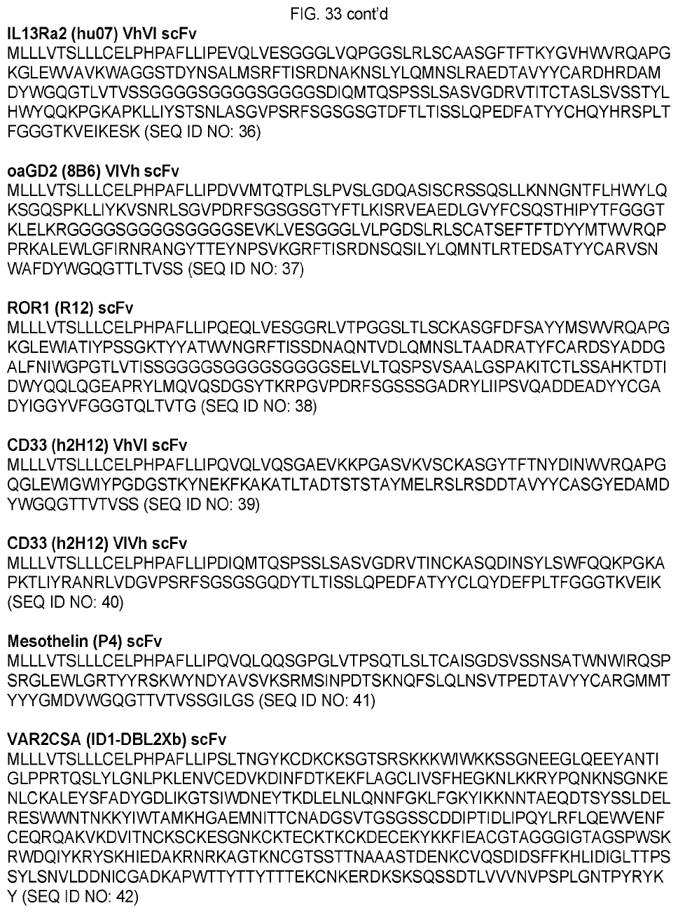

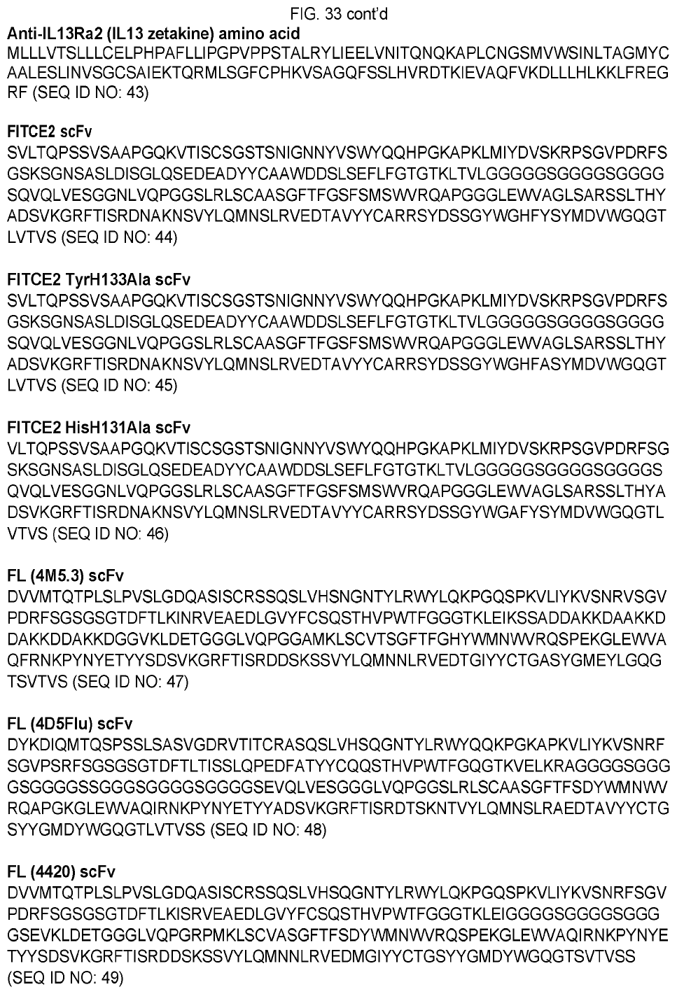

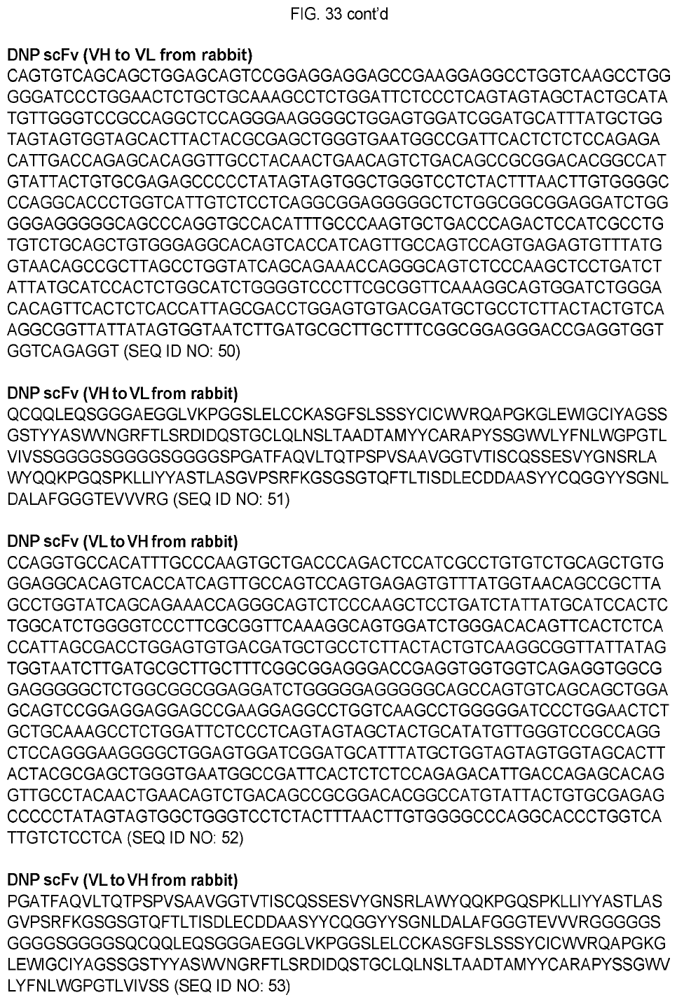

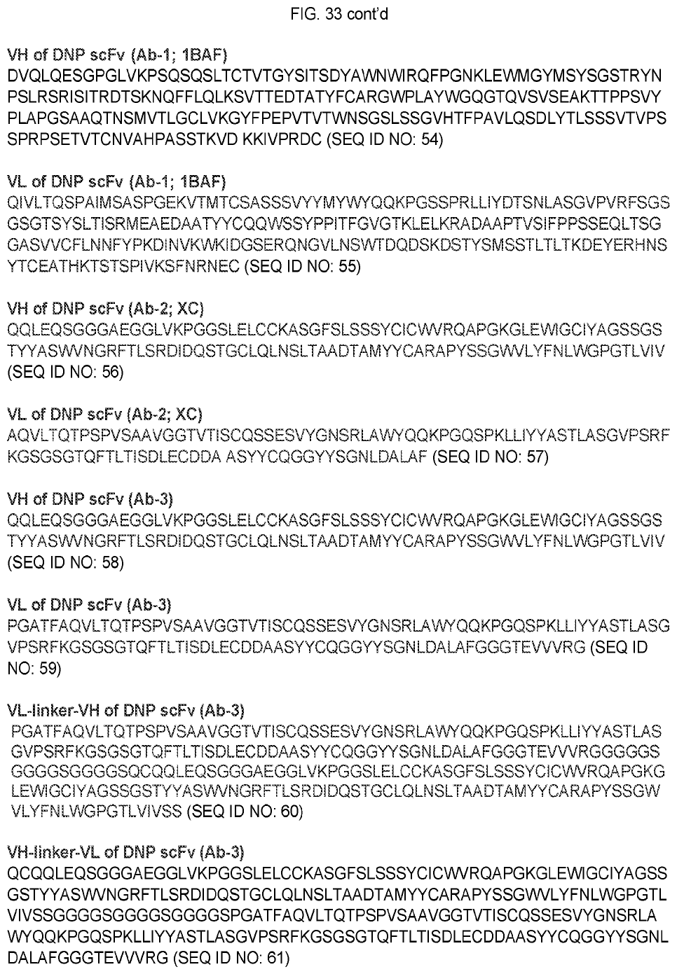

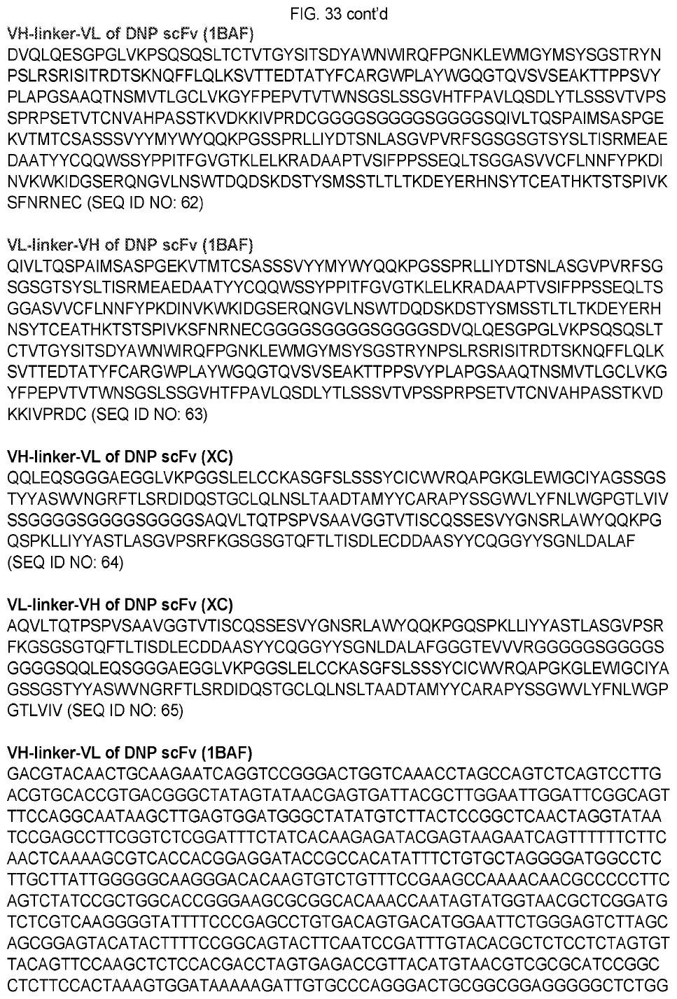

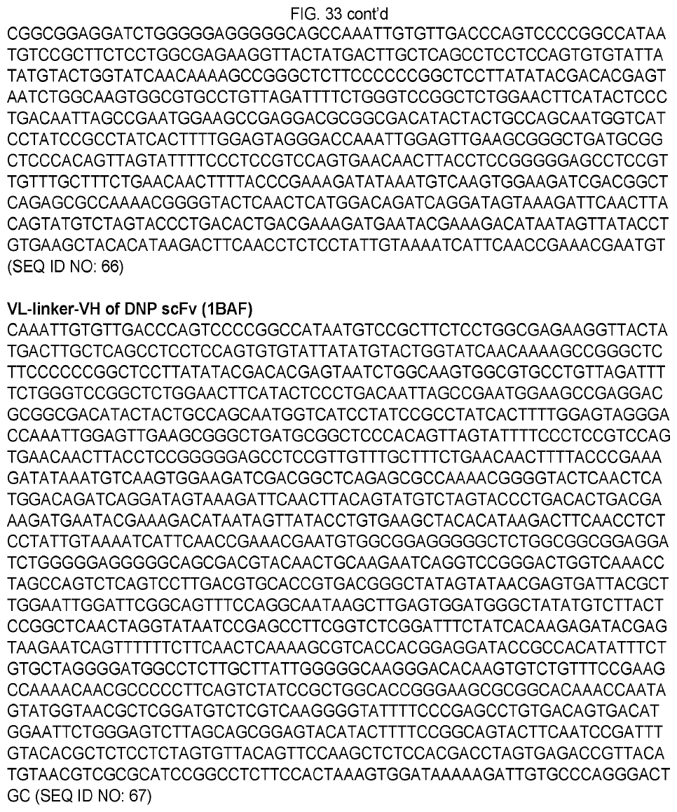

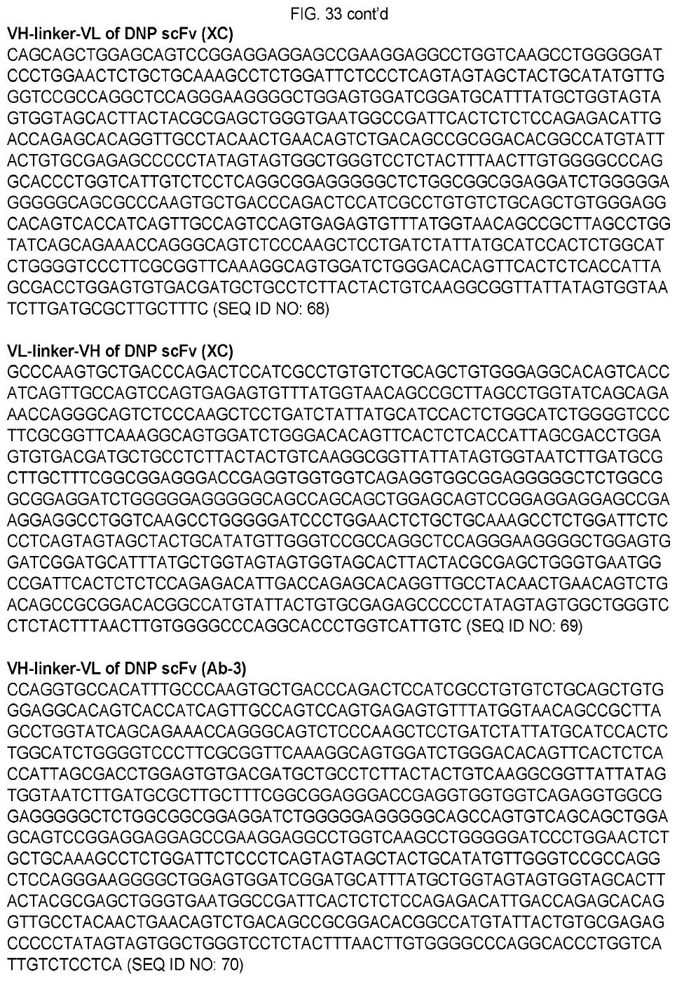

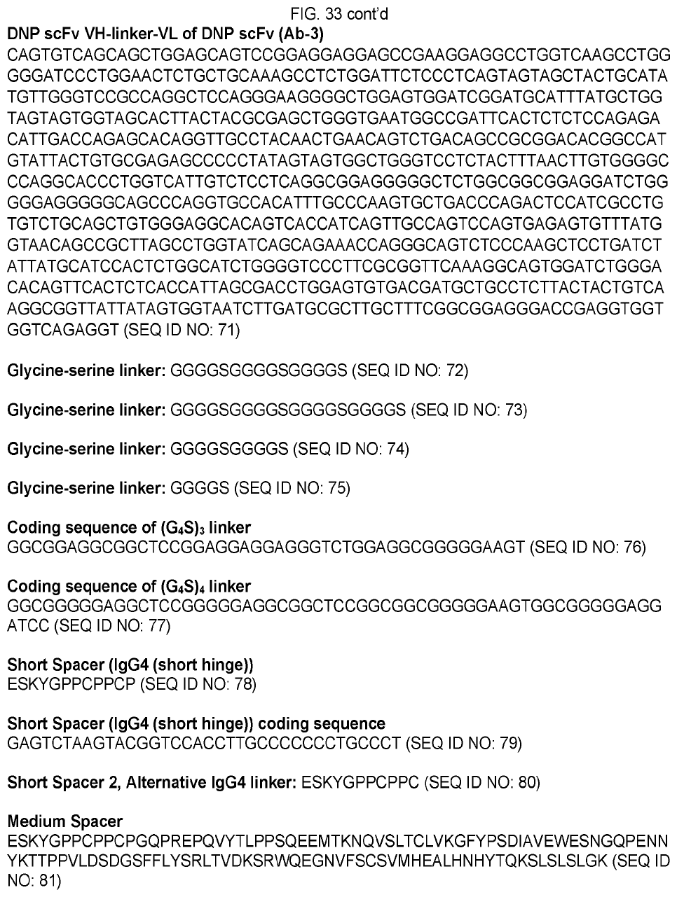

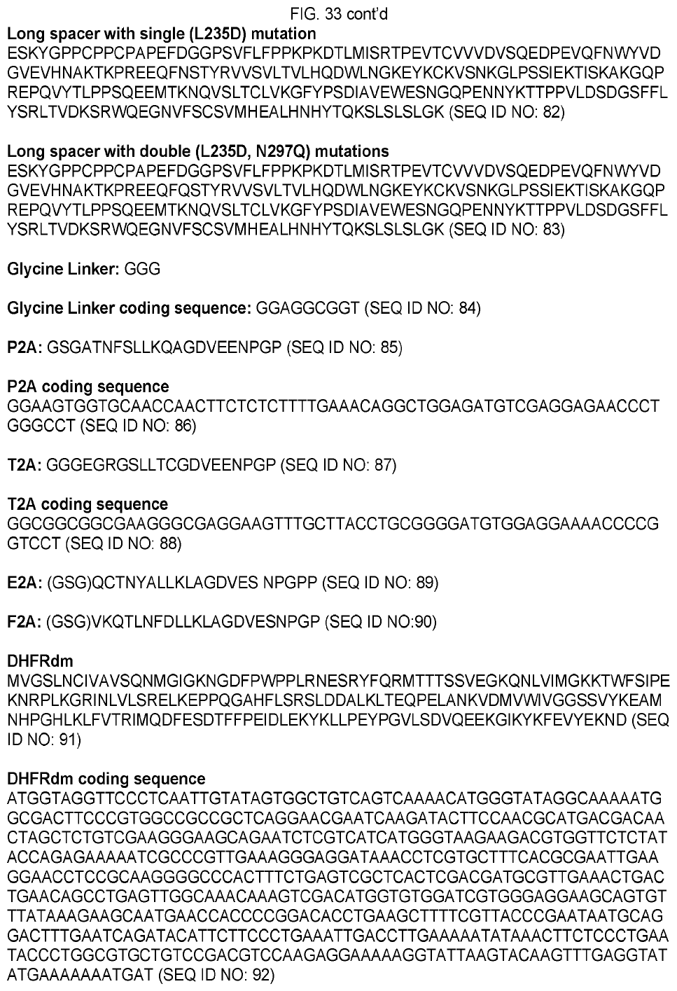

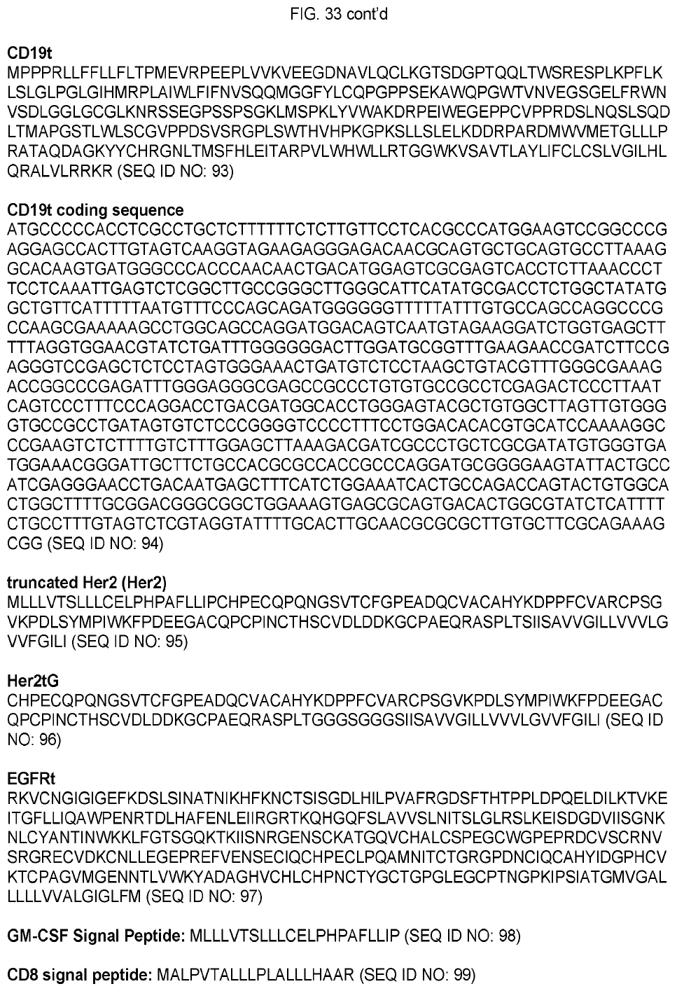

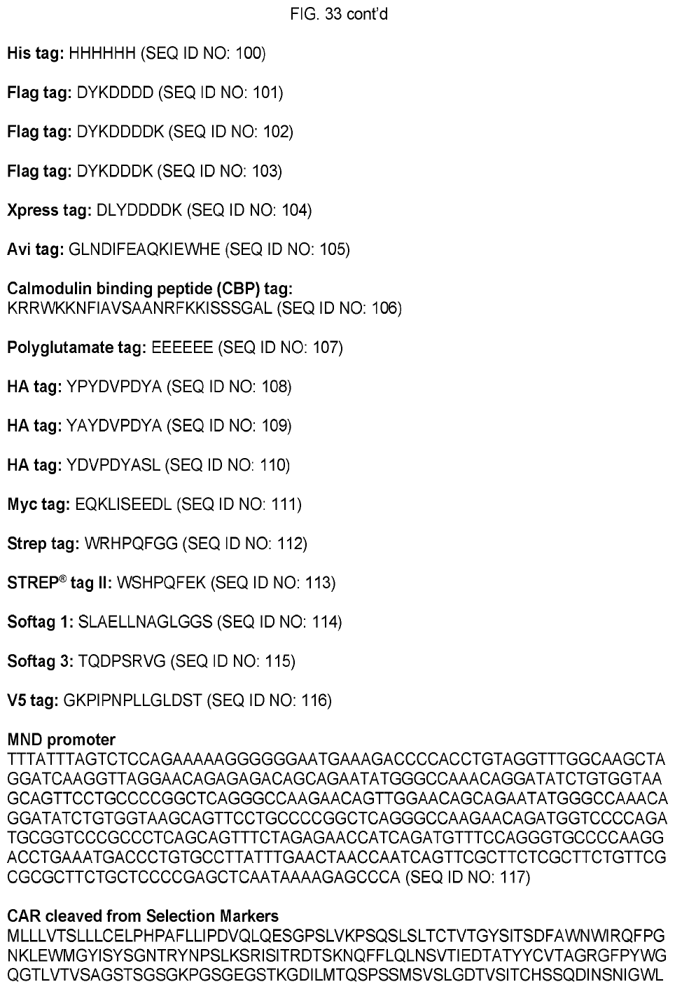

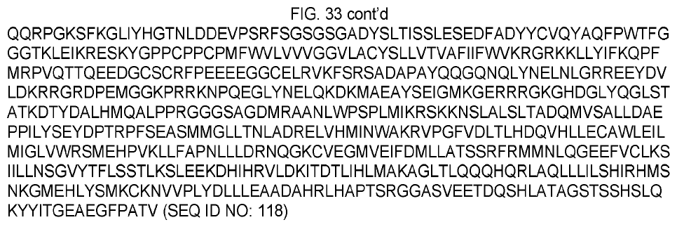

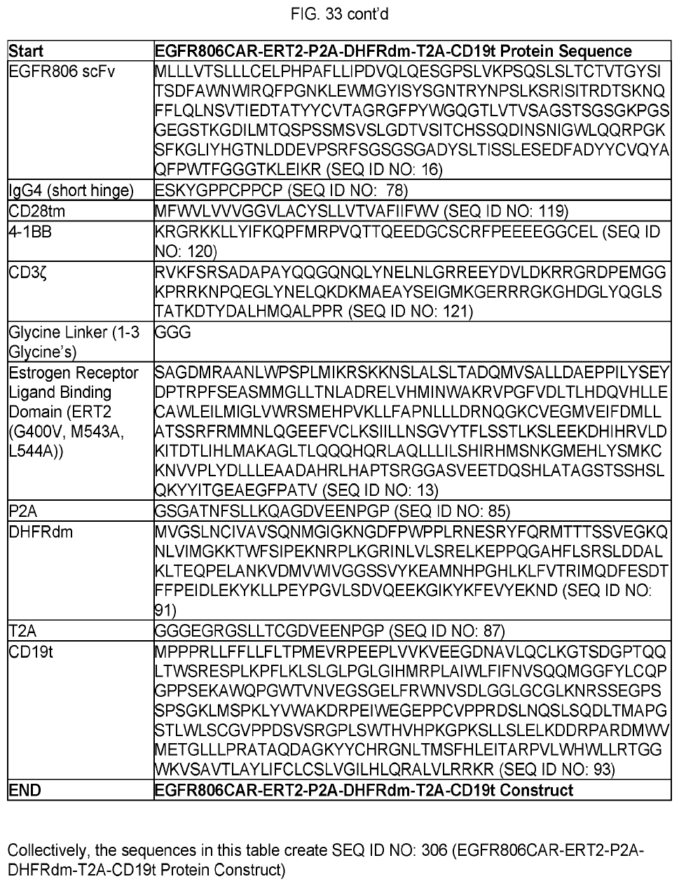

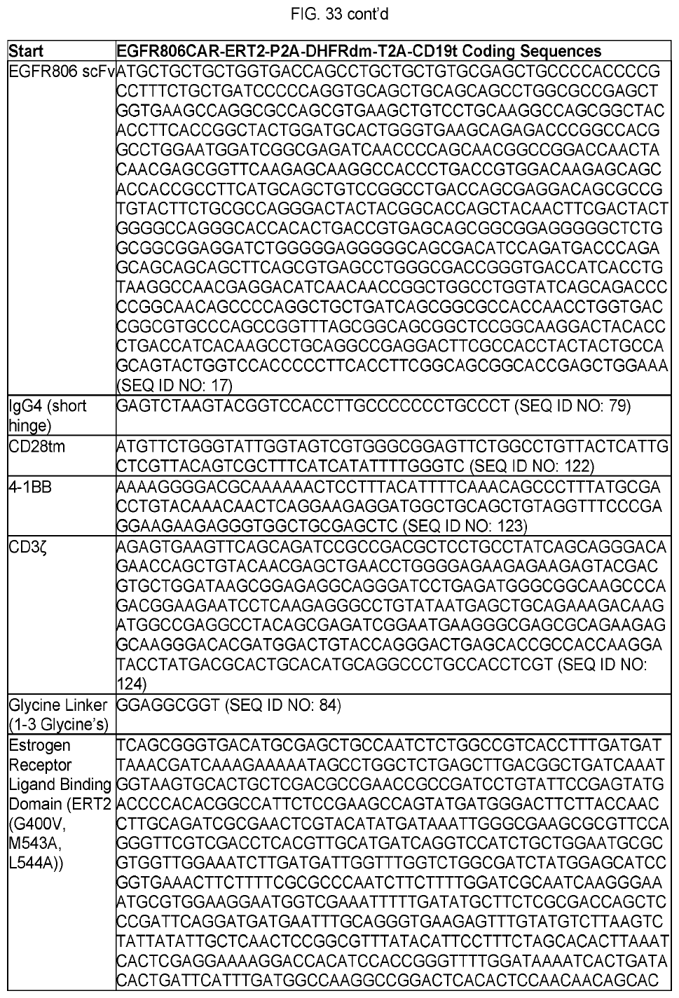

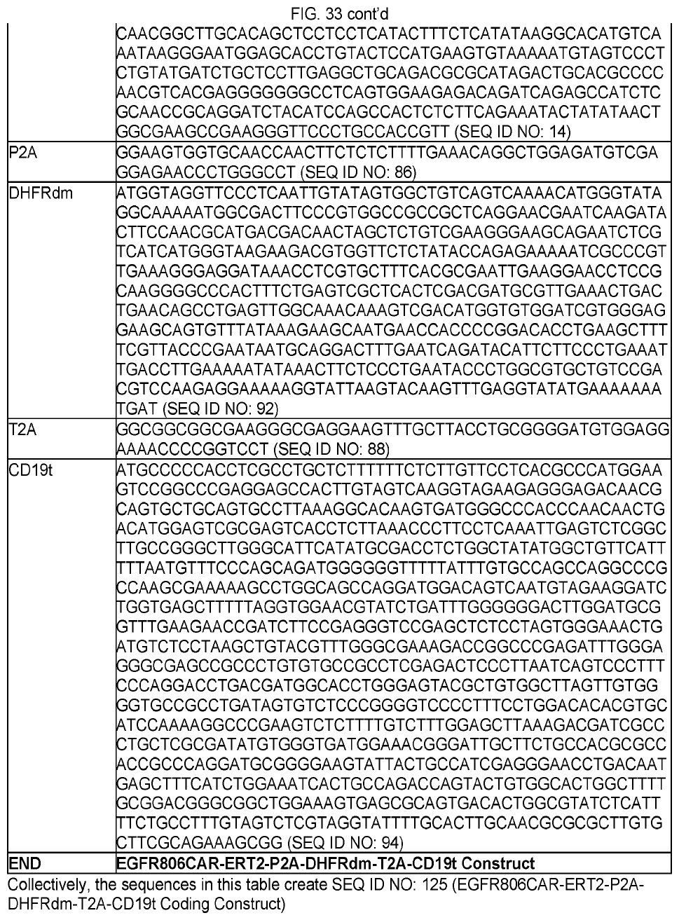

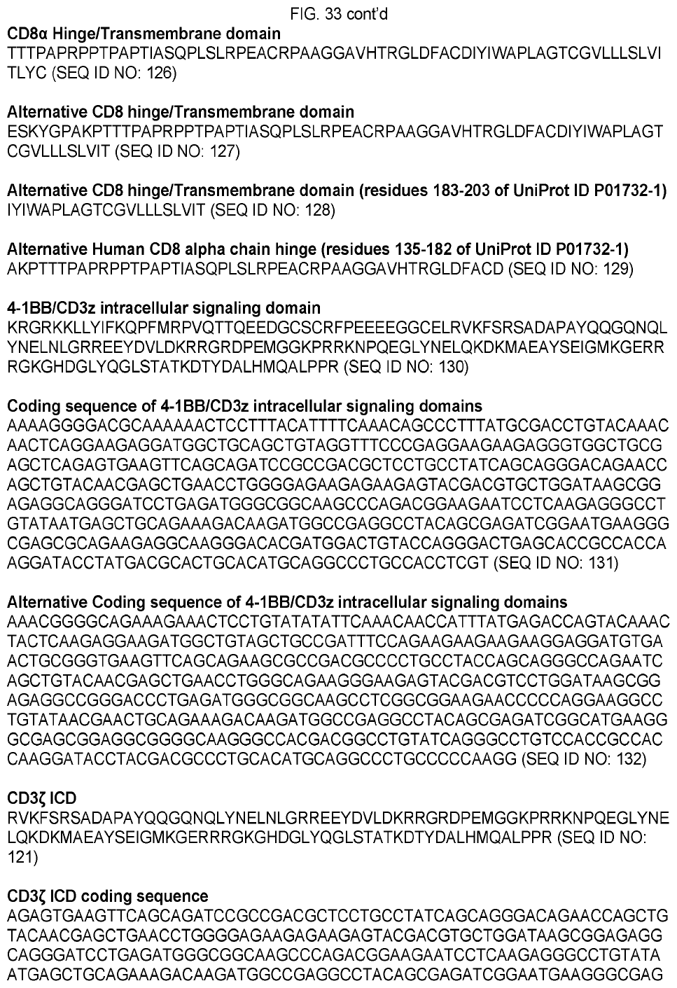

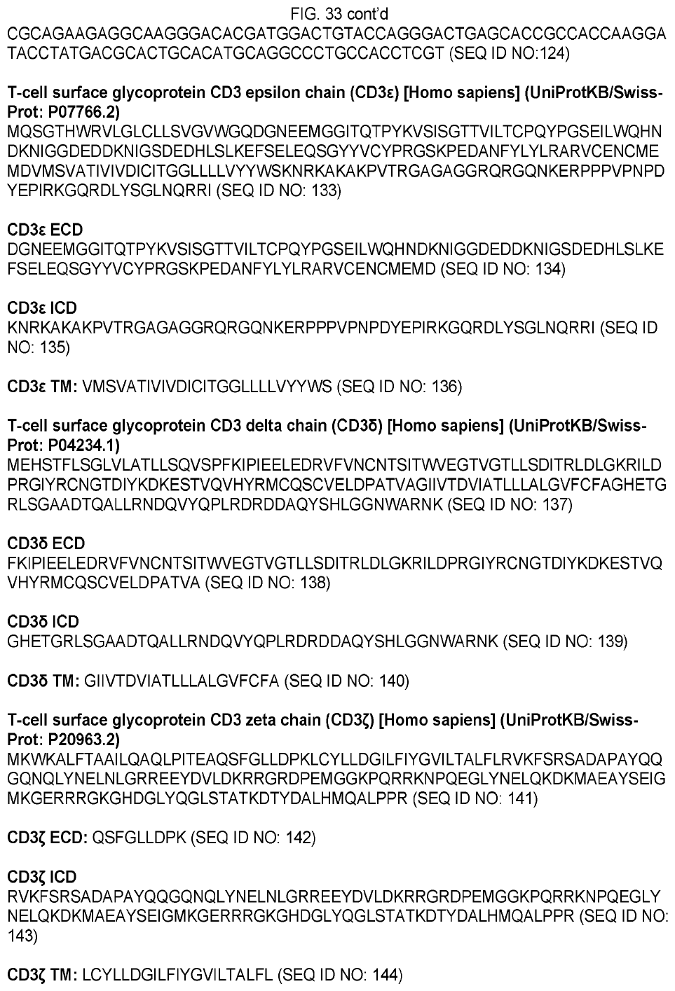

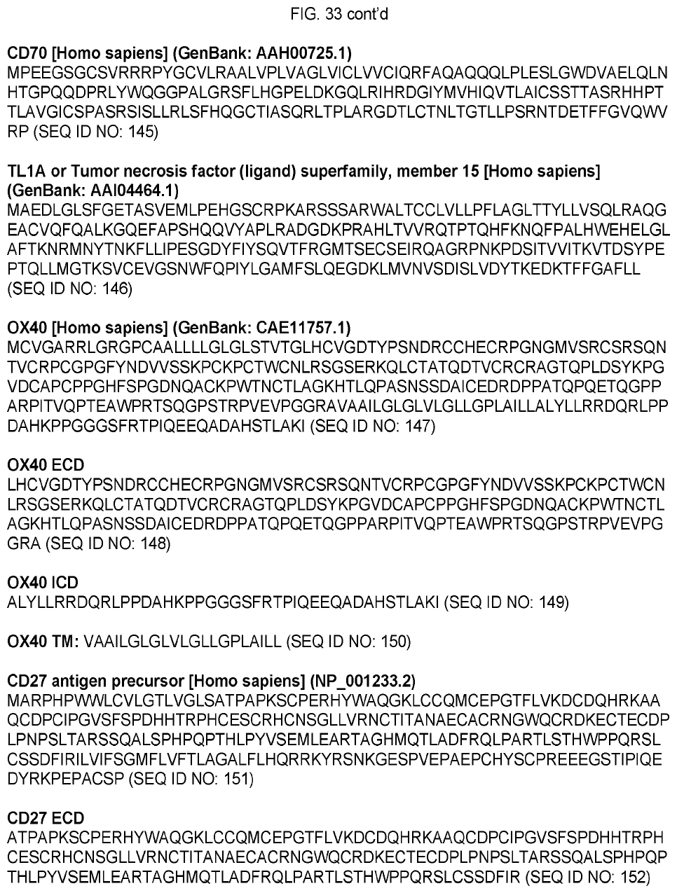

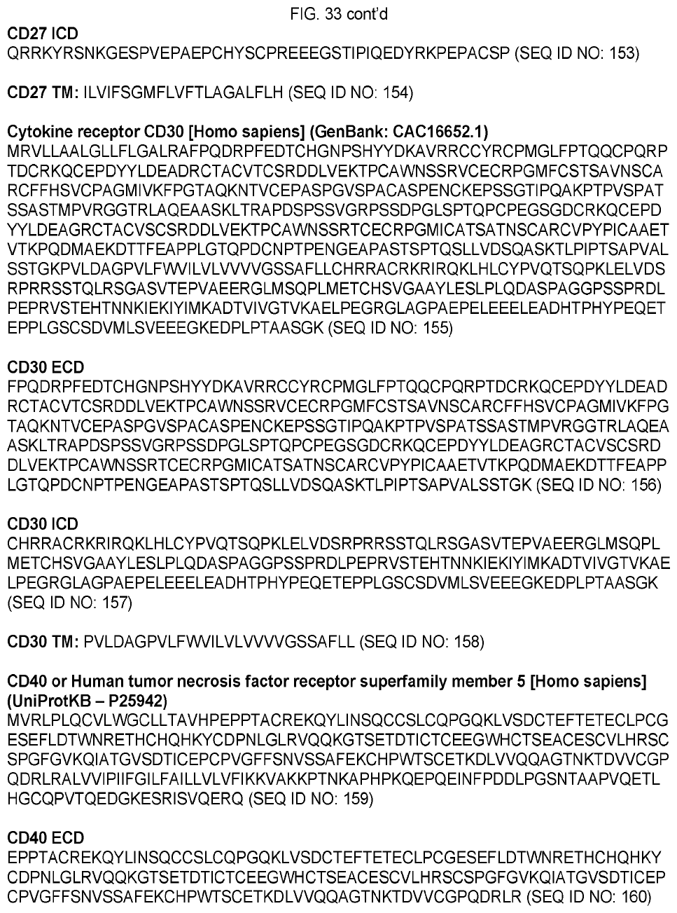

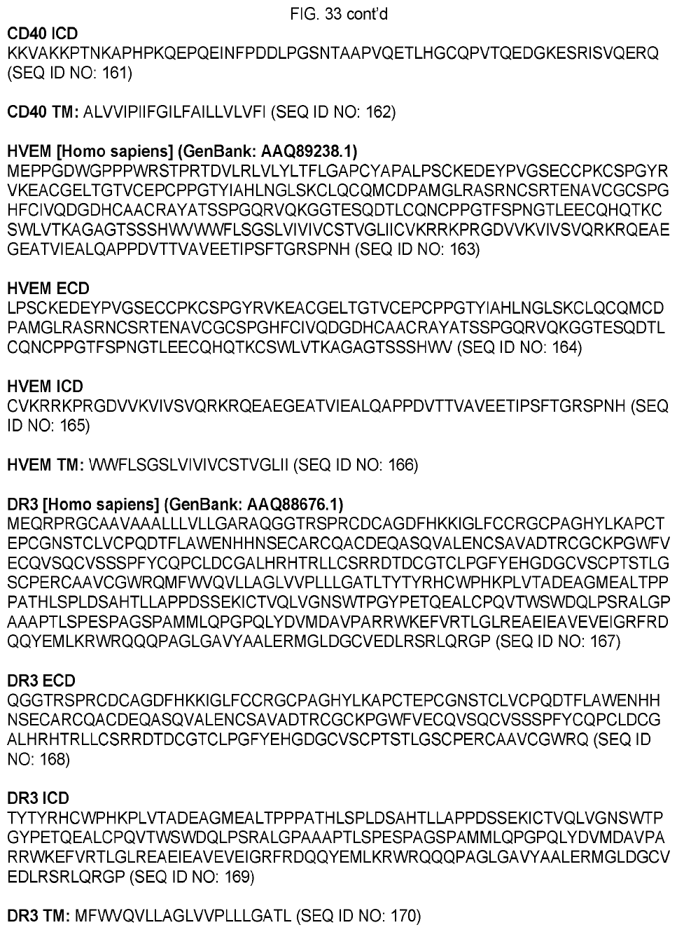

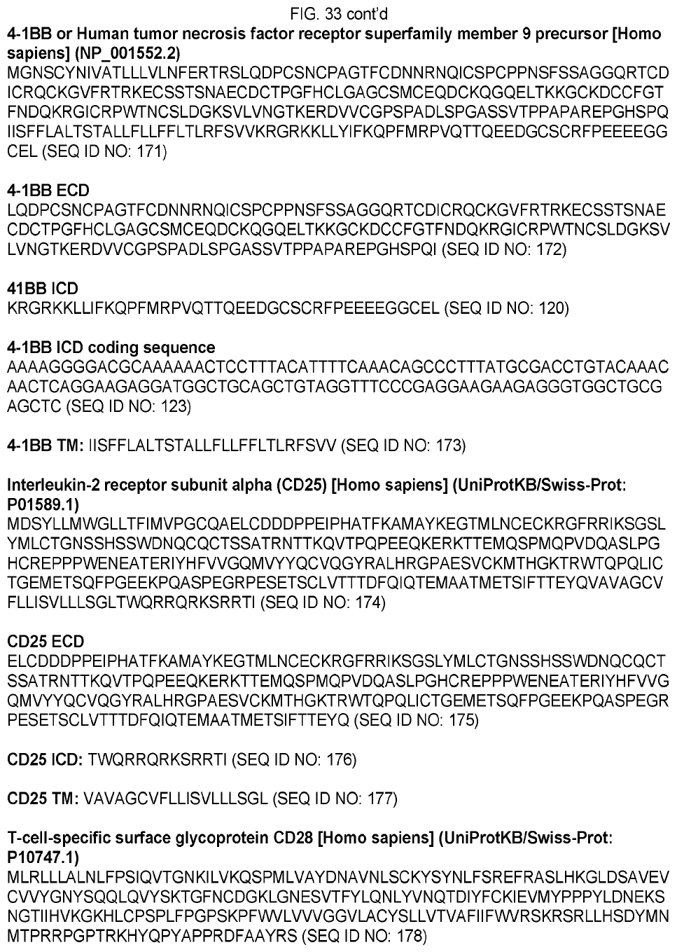

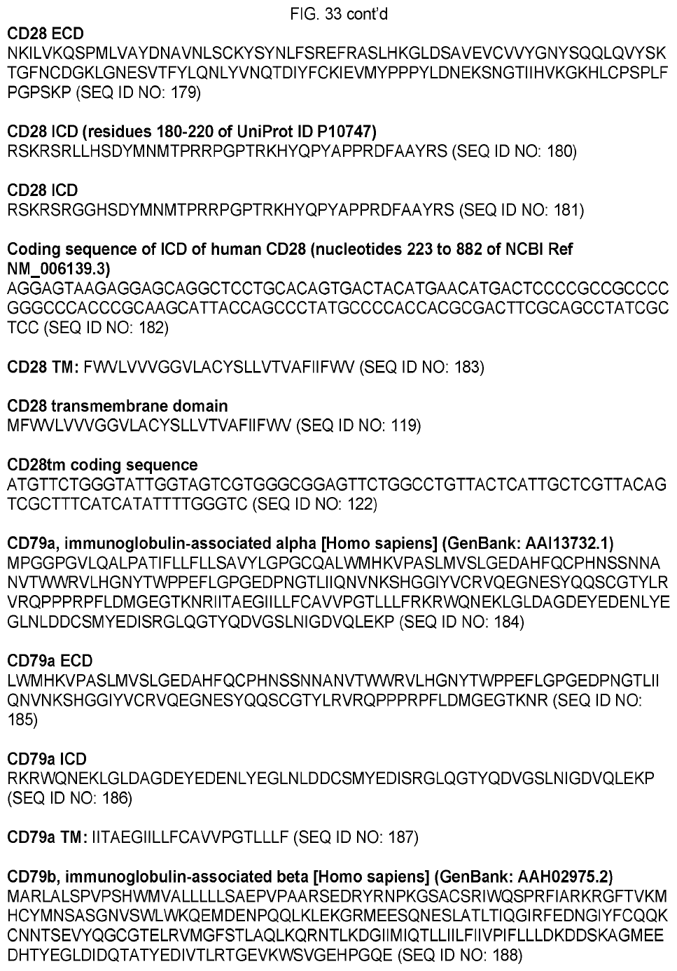

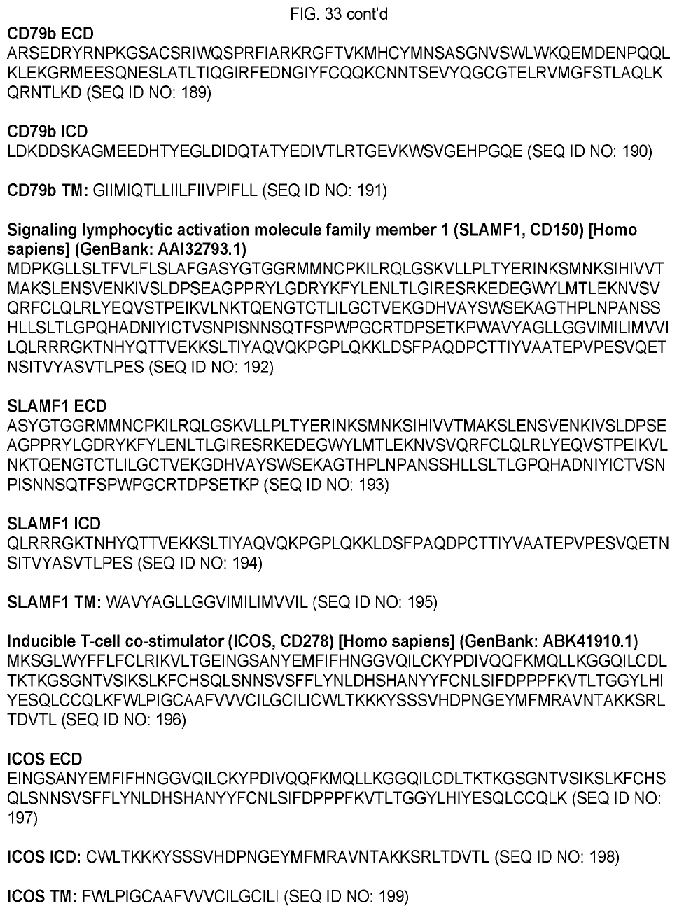

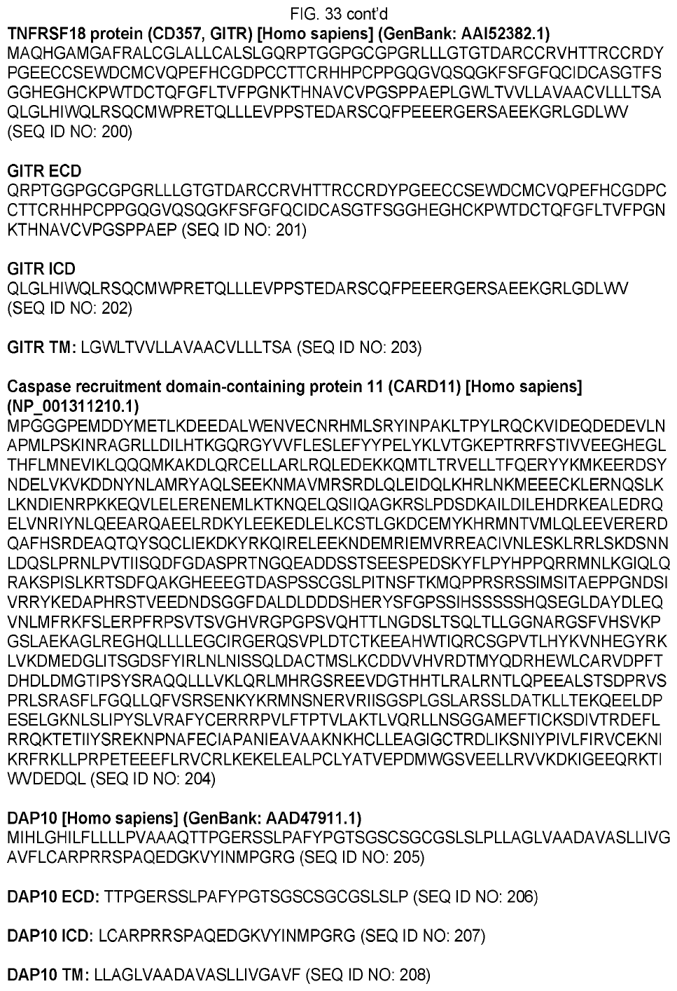

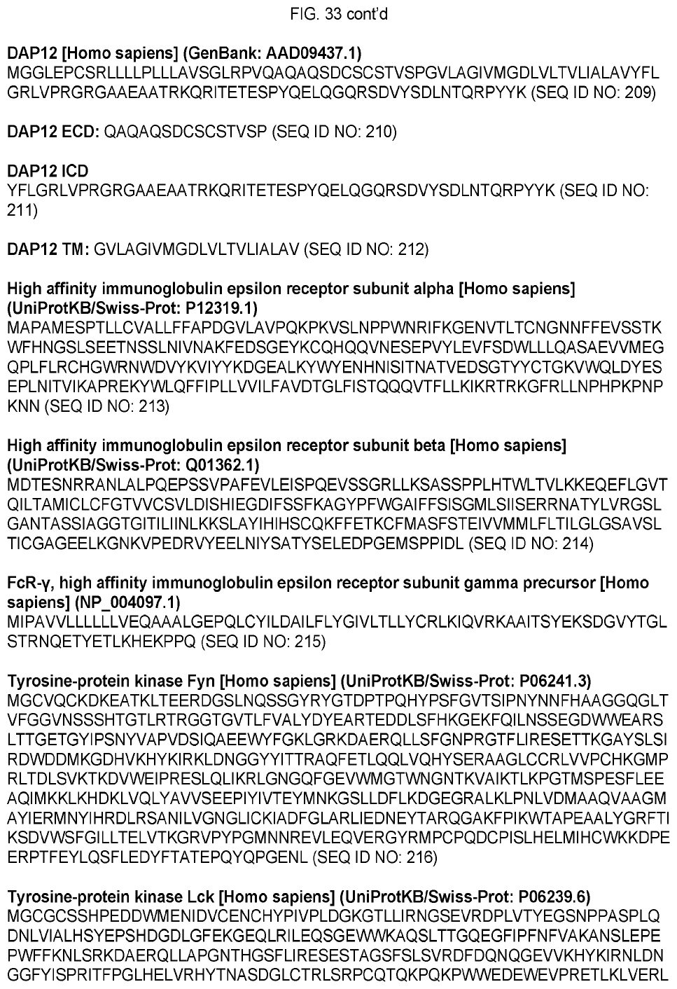

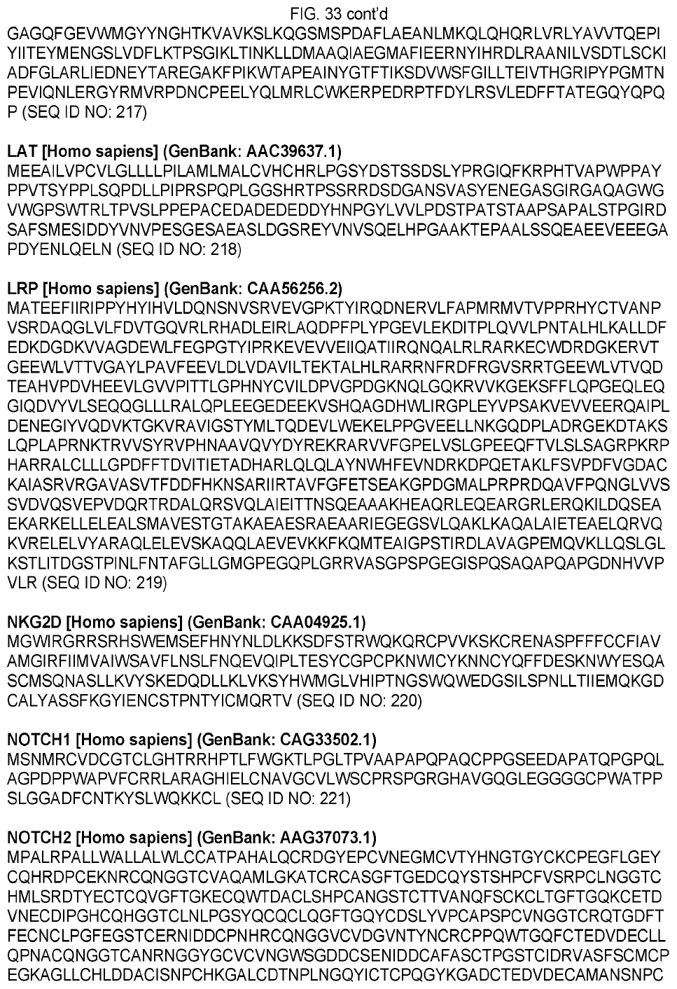

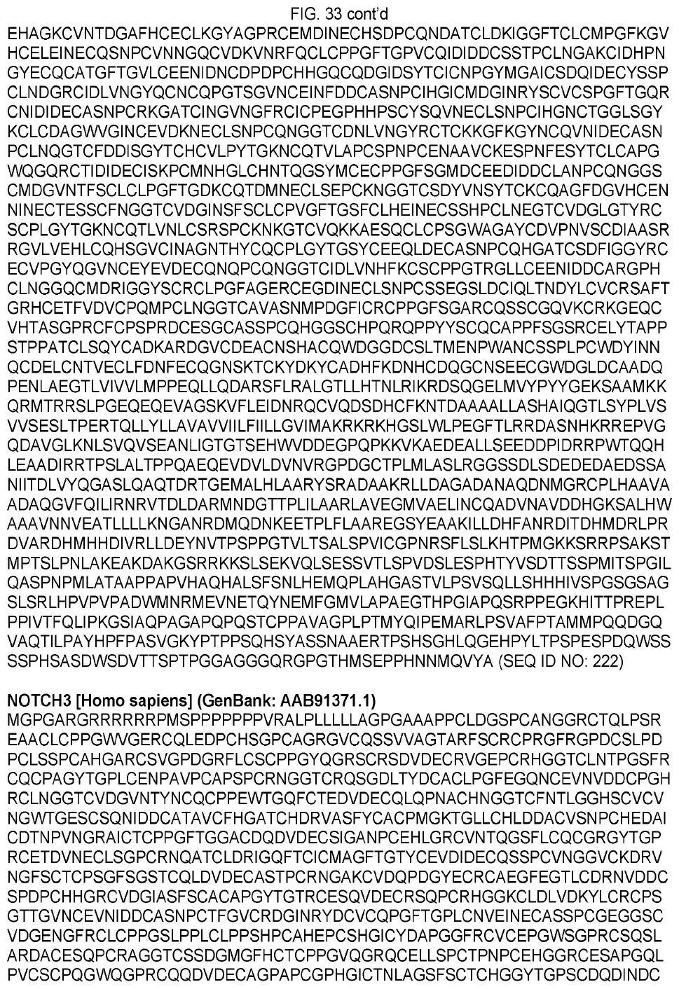

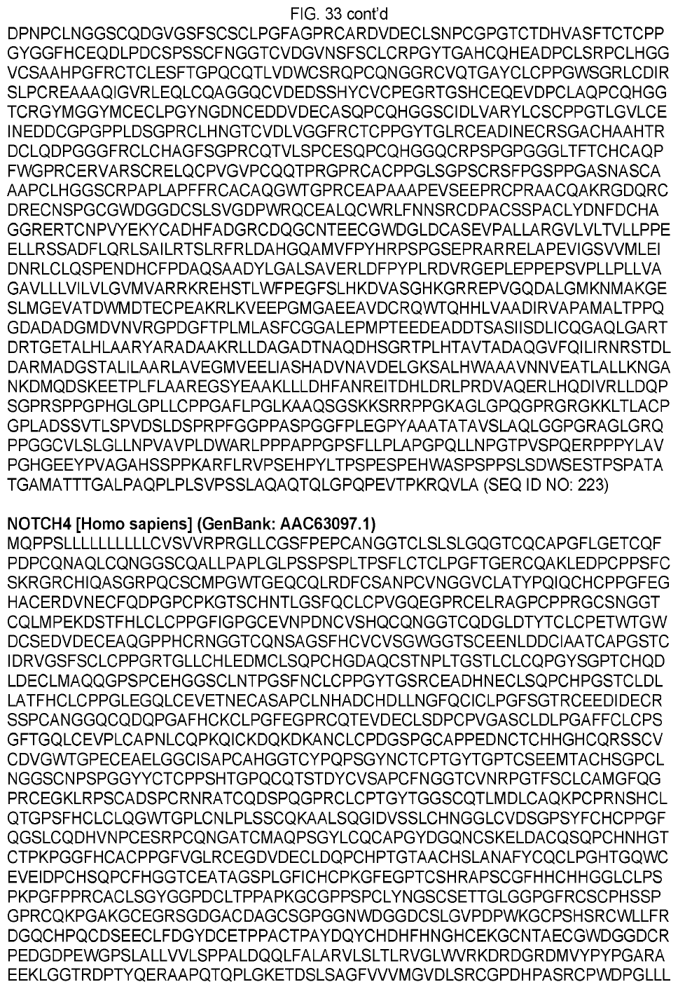

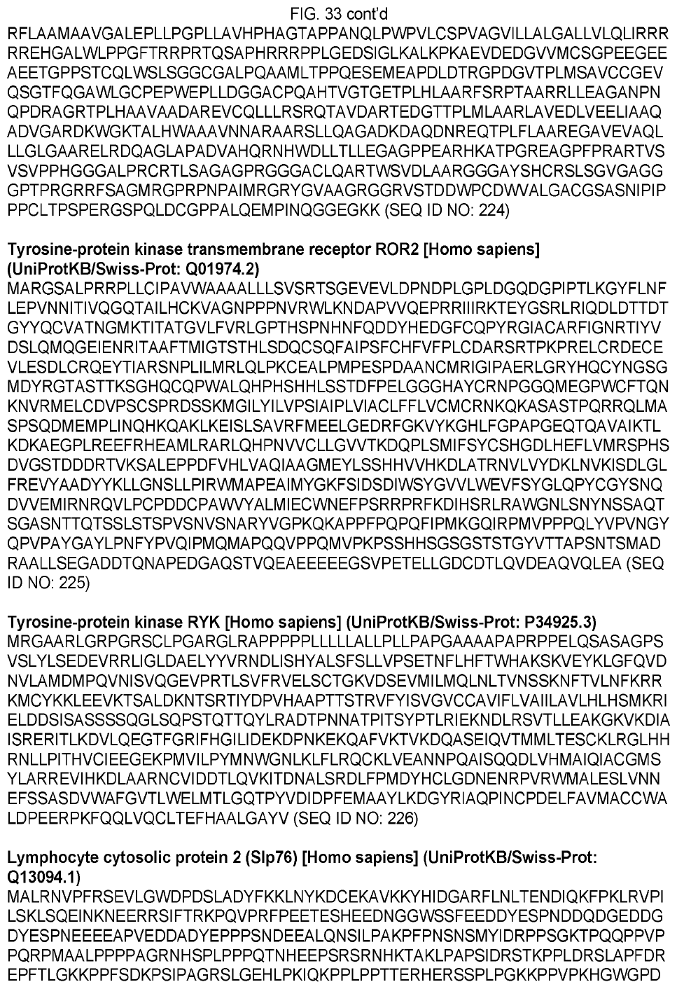

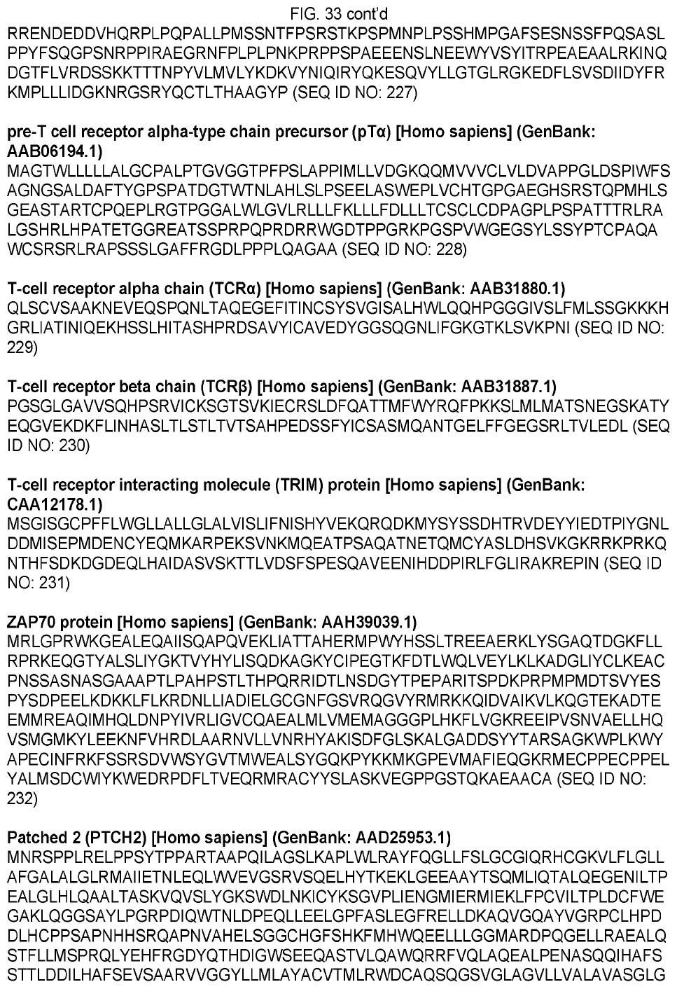

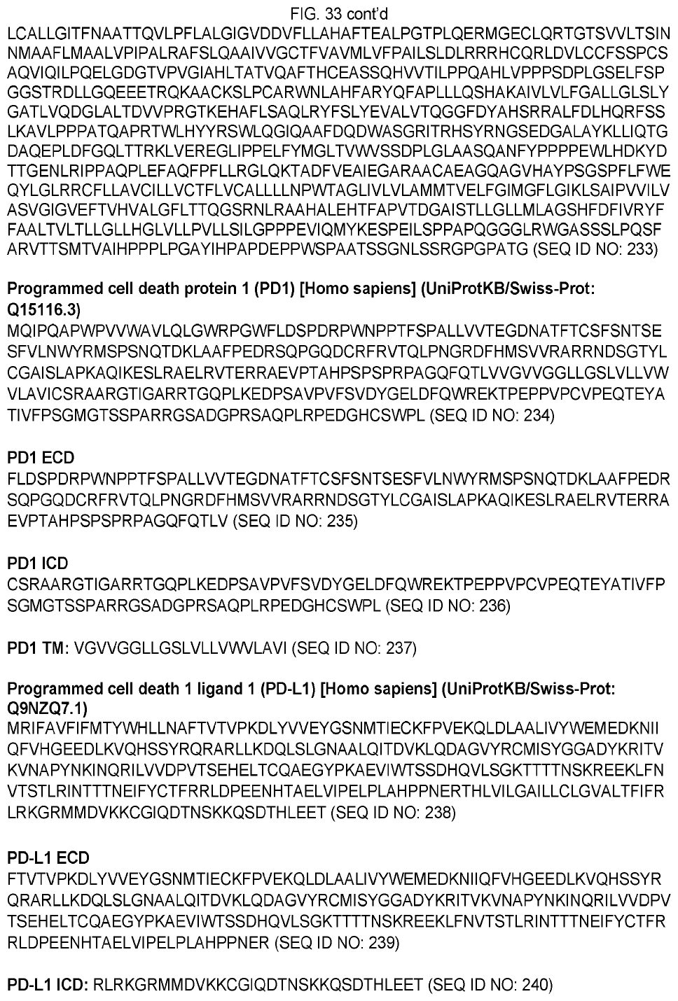

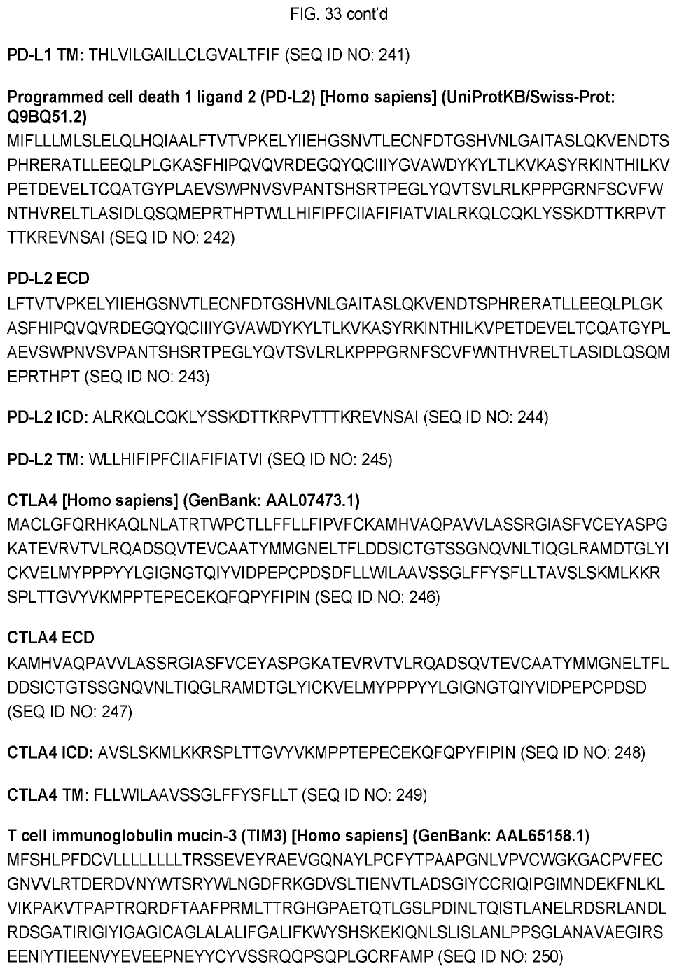

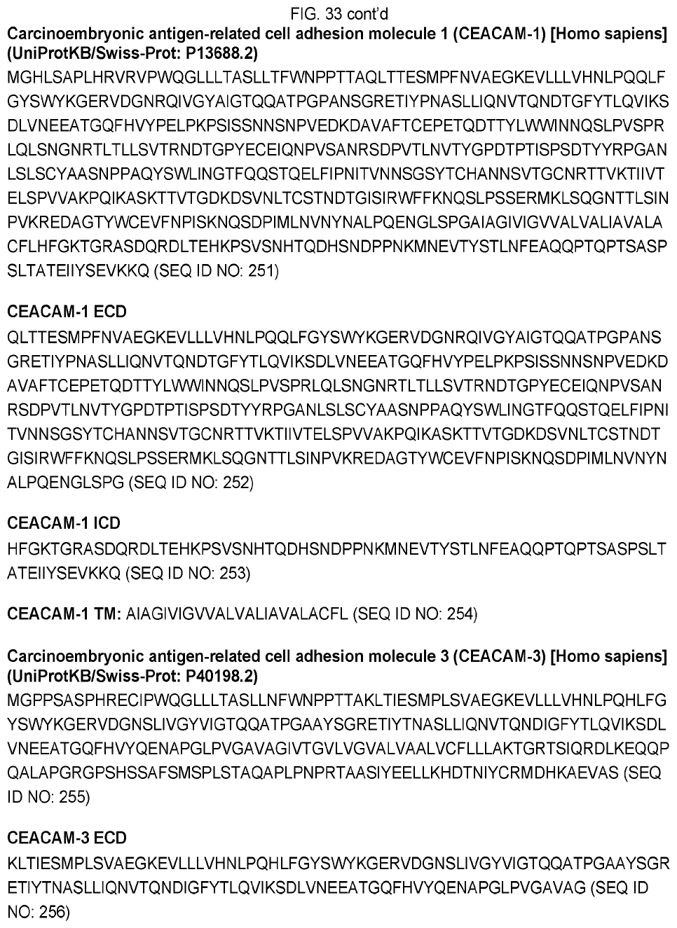

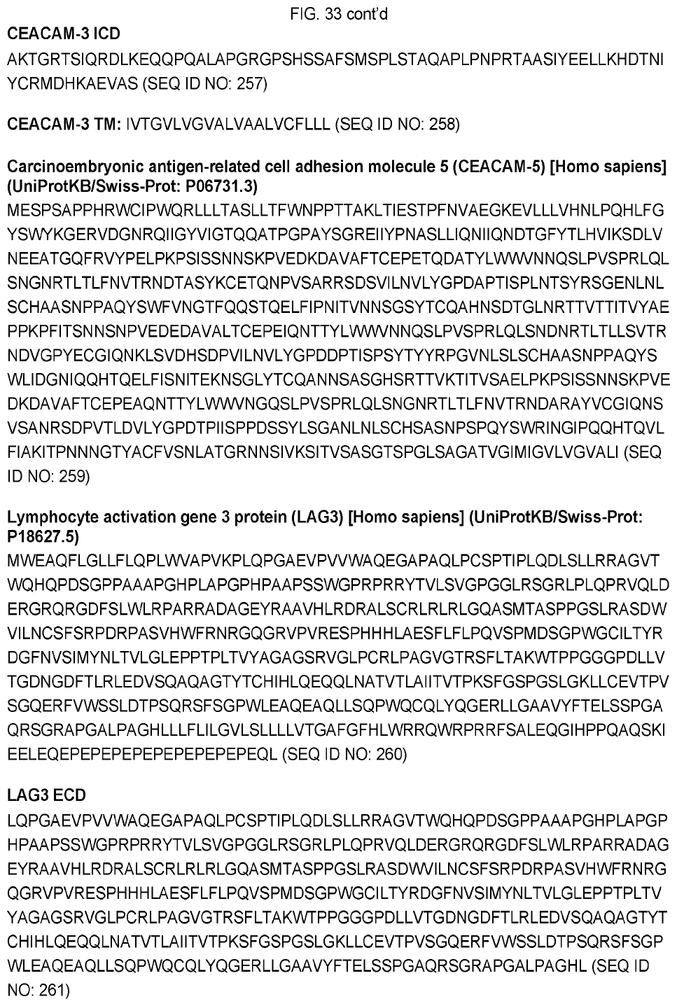

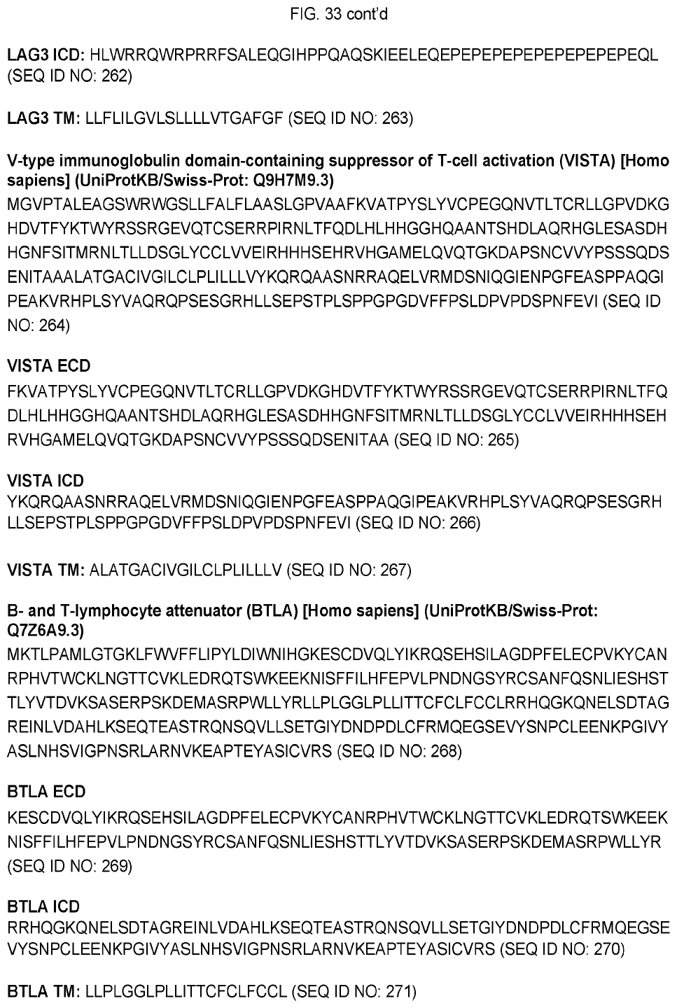

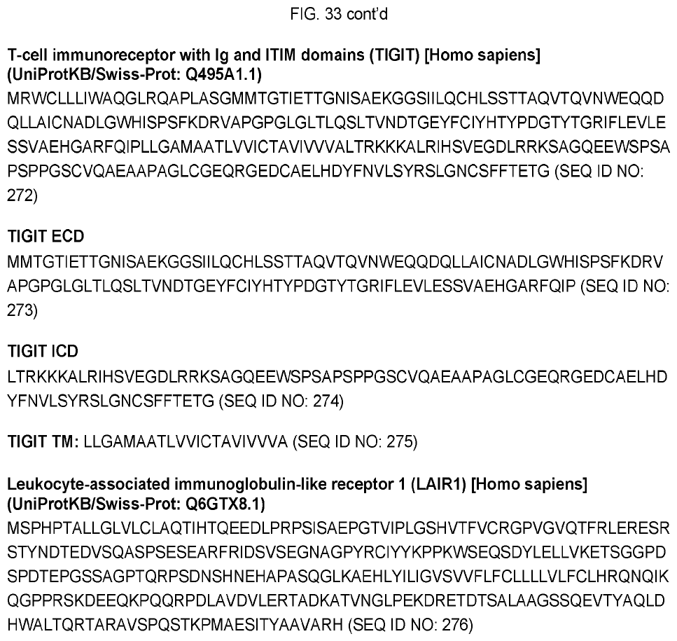

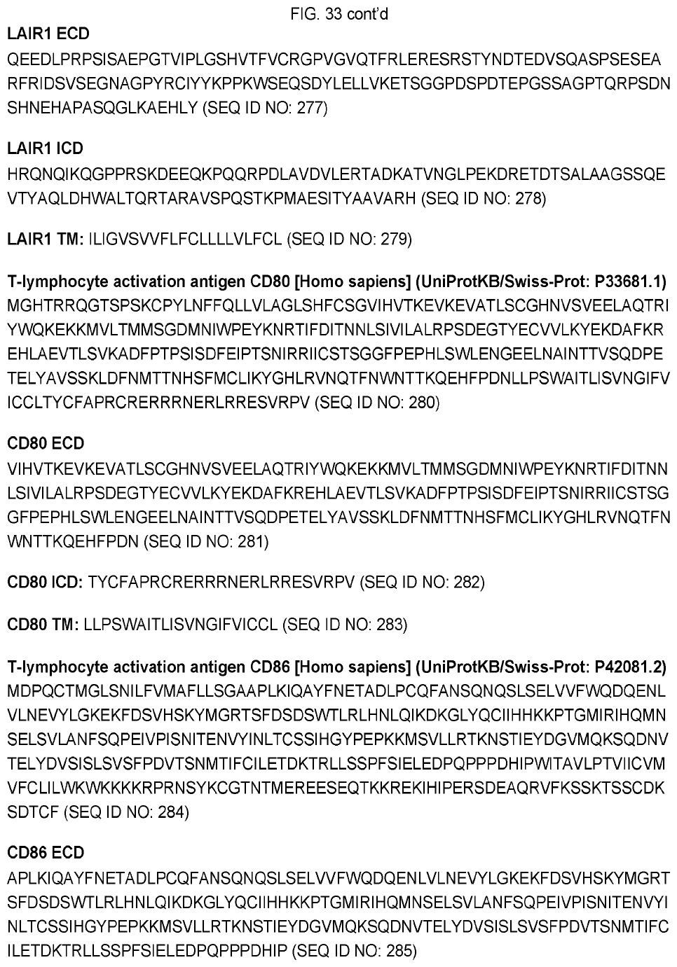

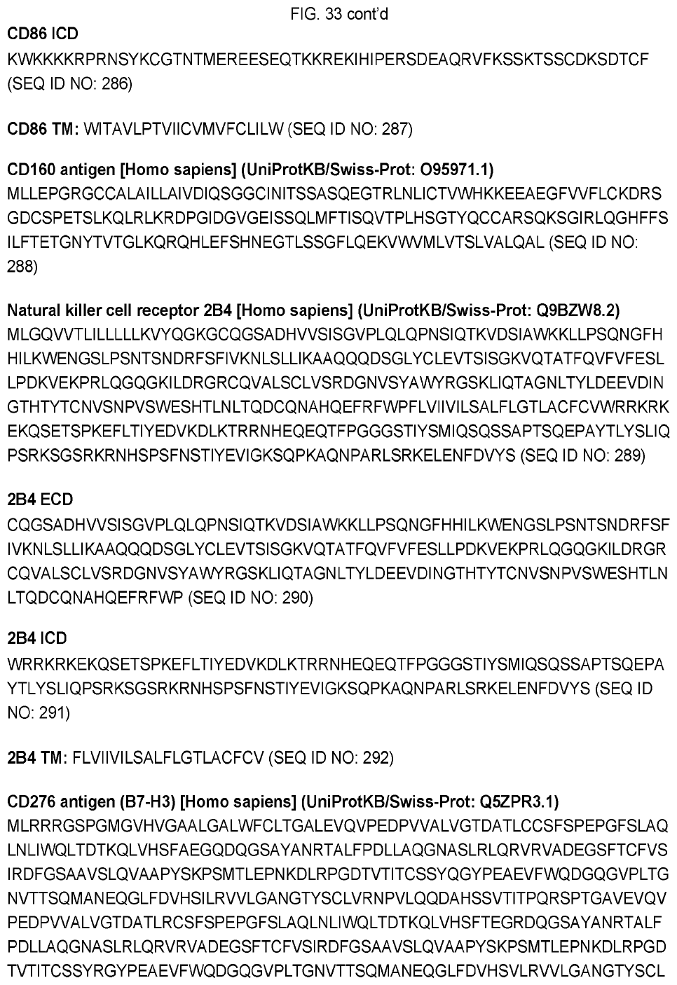

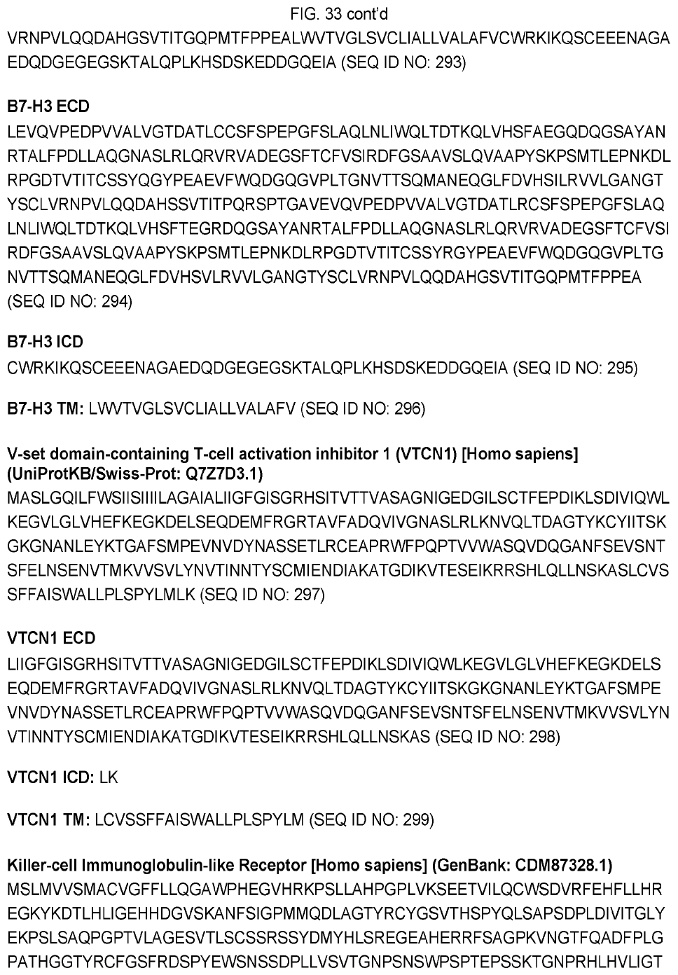

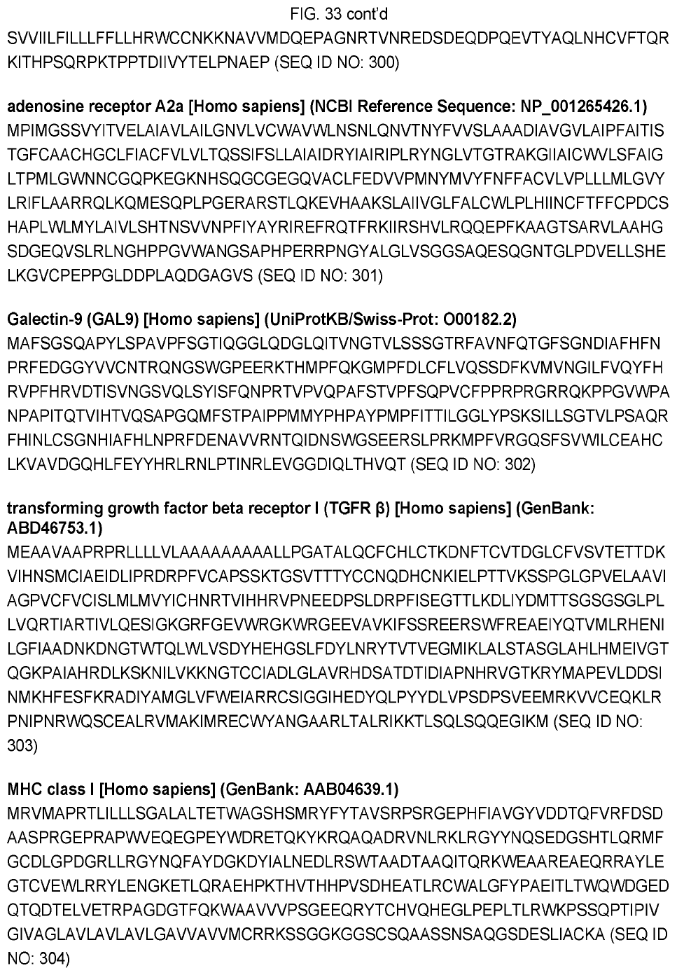

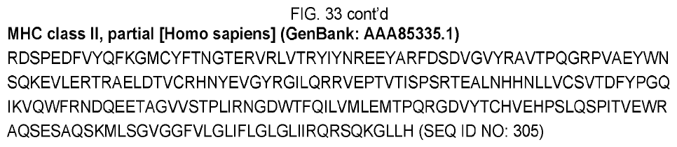

Exemplary hormone binding domains include the estrogen receptor having at least one mutation that reduces or eliminates binding to endogenous estrogen/estradiol. The protein sequence of the estrogen receptor is provided in FIG. 33 as SEQ ID NO: 1. The ER point mutation (G521R (SEQ ID NO: 3)) ablates binding to endogenous estrogen but confers nanomolar specificity to the tamoxifen metabolite 4-OHT, fulvestrant, and other estrogen analogs. Particular embodiments herein utilize a G521R estrogen receptor binding domain (EBD) as set forth in SEQ ID NO: 4. Certain embodiments utilize a E353A mutated EBD (SEQ ID NO: 6) with the drug molecule ES8 as described in Shi & Koh, Chemistry & Biology 8 (2001) 501-510. Other embodiments can utilize EBD with 2-point mutations (L384M and M421G (SEQ ID NO: 9)) or 3-point mutations (L384M, M421G, and G521R (SEQ ID NO: 11)) as described in Gallinari et al., Chemistry & Biology, Vol. 12, 883-893 (2005) with the drug molecule CMP8. Mutations (G400V, M543A, and L544A (SEQ ID NO: 7)) also abolish estradiol binding but permit binding to tamoxifen metabolites and other estrogen analogues. Accordingly, some embodiments utilize an EBD having the sequence as set forth in SEQ ID NO: 13. For additional information regarding ER-based drug systems, see Indra et al., Nucleic Acids Research, 1999, Vol. 27, No. 22 (4324-4327) and Giacomello et al., Int. J. Dev. Biol. 45: 833-838 (2001).

In some alternatives, an effective amount of the drug for allowing fusion protein activity in the presence of a relevant physiological event is an amount that provides for an increase in fusion protein activity over uninduced and/or basal activity. For example, in some alternatives, an effective amount of the drug for allowing CAR activity in the presence of antigen binding is an amount that provides for an increase in CAR activity over uninduced and/or basal activity. In other alternatives, an effective amount of the drug allows stimulatory, co-stimulatory or inhibitory immune signaling activity in the presence of ligand binding over uninduced and/or basal activity. These amounts can be determined using known dosages and pharmacokinetic profiles of the drug.

Further, in addition to the examples provided herein, other drugs can be selected based on safety record, favorable pharmacokinetic profile, tissue distribution, a low partition coefficient between the extracellular space and cytosol, and/or low toxicities.

(ii) Ligand Binding Domains. In particular embodiments, an extracellular ligand binding domain is any molecule capable of specifically binding a target antigen. Exemplary ligand binding domains include antibodies or binding fragments thereof, receptors (e.g., T cell receptors), and receptor ligands (e.g., a cytokine or chemokine).

As is understood by those of ordinary skill in the art, a complete antibody includes two heavy chains and two light chains. Each heavy chain consists of a variable region and a first, second, and third constant region, while each light chain consists of a variable region and a constant region. Mammalian heavy chains are classified as α, δ, ε, γ, and μ, and mammalian light chains are classified as λ or κ. Immunoglobulins including the α, δ, ε, γ, and β heavy chains are classified as immunoglobulin (Ig)A, IgD, IgE, IgG, and IgM. The complete antibody forms a “Y” shape. The stem of the Y consists of the second and third constant regions (and for IgE and IgM, the fourth constant region) of two heavy chains bound together and disulfide bonds (inter-chain) are formed in the hinge. Heavy chains γ, α and δ have a constant region composed of three tandem (in a line) Ig domains, and a hinge region for added flexibility; heavy chains p and E have a constant region composed of four immunoglobulin domains. The second and third constant regions are referred to as “CH2 domain” and “CH3 domain”, respectively. Each arm of the Y includes the variable region and first constant region of a single heavy chain bound to the variable and constant regions of a single light chain. The variable regions of the light and heavy chains are responsible for antigen binding.

Light and heavy chain variable regions contain a “framework” region interrupted by three hypervariable regions, also caIled “complementarity determining regions” or “CDRs”. CDR sets can be based on, for example, Kabat numbering (Kabat et al. (1991) “Sequences of Proteins of Immunological Interest,” 5th Ed. Public Health Service, National Institutes of Health, Bethesda, Md. (“Kabat” numbering scheme)); Chothia (AI-Lazikani et al. (1997) JMB 273:927-948 (“Chothia” numbering scheme)); Martin (Abinandan et al. (2008) Mol Immunol. 45:3832-3839 (“Martin” numbering scheme)); Gelfand (Gelfand and Kister (1995) Proc Natl Acad Sci USA. 92:10884-10888; Gelfand et al. (1998) Protein Eng. 11:1015-1025; Gelfand et al. (1996) Proc Natl Acad Sci USA. 93:3675-3678; Gelfand et al. (1998) J Comput Biol. 5:467-477 (“Gelfand” numbering scheme)); Contact (MacCallum et al. (1996) J. Mol. Biol. 262:732-745 (Contact numbering scheme)); IMGT (Lefranc et al. (2003) Dev Comp Immunol 27(1):55-77 (“IMGT” numbering scheme)); AHo (Honegger and Plückthun (2001) J Mol Biol 309(3):657-670 (“AHo” numbering scheme)); North (North et al. (2011) J Mol Biol. 406(2):228-256 (“North” numbering scheme)); or other numbering schemes.

Software programs and bioinformatical tools, such as ABodyBuilder (Leem et al. (2016) MAbs 8(7):1259-1268), PIGSPro (Lepore et al. (2017) Nucleic Acids Res 45(W1):W17-W23), Kotai Antibody Builder (Yamashita et al. (2014) Bioinformatics 30(22):3279-3280), Rosetta Antibody (Weitzner et al. (2017) Nature Protocols 12:401-416), Paratome (Kunik et al. (2012) Nucleic Acids Res 40:W521-W524), Antibody i-Patch (Krawczyk et al. (2013) Protein Eng Des Sel 26(10):621-629), and proABC-2 (Ambrosetti et al. (2020) Bioinformatics 36(20):5107-5108 can also be used to determine CDR sequences.

The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, the CDRs located in the variable domain of the heavy chain of the antibody are referred to as CDRH1, CDRH2, and CDRH3, whereas the CDRs located in the variable domain of the light chain of the antibody are referred to as CDRL1, CDRL2, and CDRL3. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are caIled specificity determining residues (SDRs).

References to “V_{H}” or “VH” refer to the variable region of an immunoglobulin heavy chain. References to “V_{L}” or “VL” refer to the variable region of an immunoglobulin light chain.

Antibodies that specifically bind a cell surface molecule can be prepared using methods of obtaining monoclonal antibodies, methods of phage display, methods to generate human or humanized antibodies, or methods using a transgenic animal or plant engineered to produce human antibodies. Phage display libraries of partially or fully synthetic antibodies are available and can be screened for an antibody or fragment thereof that can bind to the target molecule. Phage display libraries of human antibodies are also available. Once identified, the amino acid sequence or polynucleotide sequence coding for the antibody can be isolated and/or determined. Many relevant antibodies are also publicly known and commercially available.

In some alternatives, antibodies specifically bind to a cancer cell or virally-infected cell surface molecule and do not cross react with nonspecific components such as bovine serum albumin or other unrelated antigens.

The term “antibody fragment” refers to at least one portion of an antibody, that retains the ability to specifically binding an antigen. Examples of antibody fragments include Fab, Fab′, F(ab′)₂, Fv fragments, single chain variable (scFv) antibody fragments, disulfide-linked Fvs (sdFv), a Fd fragment including VH and constant CH1 domains, linear antibodies, single domain antibodies such as sdAb (either VL or VH), camelid variable heavy only (VHH) domains, multi-specific antibodies formed from antibody fragments such as a bivalent fragment including two Fab fragments linked by a disulfide bridge at the hinge region, and an isolated CDR or other epitope binding fragments of an antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). An antigen binding fragment can also be incorporated into single domain antibodies, maxibodies, minibodies, intrabodies, diabodies, triabodies, tetrabodies, v-NAR and bis-scFv (see, e.g., Hollinger and Hudson (2005) Nature Biotechnology 23:1126-1136).

In particular embodiments, a binding domain can include humanized forms of non-human (e.g., murine) antibodies or antigen binding fragments thereof. A humanized antibody includes an antibody in which the constant and variable framework region of one or more human immunoglobulins is fused with the binding region, e.g., the CDR, of an animal (non-human) immunoglobulin. Such humanized antibodies are designed to maintain the binding specificity of the non-human antibody from which the binding regions are derived but avoid an immune reaction against the non-human antibody. In particular embodiments, a binding domain can include a fully human antibody or antibody fragment thereof, where the whole molecule is of human origin or includes an amino acid sequence identical to a human form of the antibody or immunoglobulin.

The term “scFv” refers to an engineered fusion protein including the VH and VL of an antibody linked via a linker and capable of being expressed as a single chain polypeptide. The scFv retains the specificity of the intact antibody from which it is derived. In particular embodiments, a linker connecting the variable regions can include glycine-serine linkers, including, for example, those shown as SEQ ID NOs: 72-75 or described elsewhere herein. In particular embodiments, an scFv may have the VL and VH variable regions in either order, e.g., with respect to the N-terminal and C-terminal ends of the polypeptide, the scFv may include VL-linker-VH or may include VH-linker-VL.

There are also numerous ways to identify and select particular TCR for use within CAR. For example, the sequences of numerous TCR that bind particular antigen fragments are known and publicly available.

TCR can also be identified for use with a particular antigen by, for example, isolating T cells that bind a particular antigen/MHC complex and sequencing the TCR chains binding the complex. TCR genes encoding TCR can be readily cloned by, for example, the 5′ RACE procedure using primers corresponding to the sequences specific to the TCR α-chain gene and the TCR β-chain gene.

In particular embodiments, it may be necessary to pair TCR chains following sequencing (i.e., to perform paired chain analysis). Various methods can be utilized to pair chains, when necessary. For example, chain pairing may be assisted in silico by computer methods, such as immunology gene alignment software available from IMGT, JOINSOLVER, VDJSolver, SoDA, iHMMune-align, or other similar tools for annotating VDJ gene segments. Assays such as PairSEQ® (Adaptive Biotechnologies Corp., Seattle, WA) have also been developed.

In particular embodiments, an engineered TCR includes a single chain T cell receptor (scTCR) including Vα/β and Cα/β chains (e.g., Vα-Cα, Vβ-Cβ, Vα-Vβ) or including Vα-Cα, Vβ-Cβ, Vα-Vβ pair specific for a target of interest (e.g., peptide-MHC complex).

Cancer antigens are proteins that are produced by cancer cells and viral antigens are proteins produced by virally-infected cells. Ligand binding domains of CAR disclosed herein can be selected to bind cancer antigens or viral antigens. In some alternatives, cancer or viral antigens are selectively expressed or overexpressed on the cancerous or infected cells as compared to other cells of the same tissue type. In some alternatives, a cancer or viral antigen is a cell surface molecule that is found on cancer cells or virally-infected cells and is not substantially found on normal tissues, or restricted in its expression to non-vital normal tissues.

Exemplary cancer antigens include carcinoembryonic antigen (CEA), prostate specific antigen, Prostate Stem Cell antigen (PSCA), PSMA, Her2/neu, estrogen receptor, progesterone receptor, ephrinB2, CD19, CD20, CD22, CD23, CD123, CS-1, CE7, hB7H3, ROR1, mesothelin, c-Met, GD-2, MAGE A3 TCR, EGFR, EGFRvIII, EphA2, IL13Ra2, L1CAM, oaGD2, GD2, B7H3, CD33, FITC, VAR2CSA, MUC16, PD-L1, ERBB2, folate receptor (FOLR), CD56; glypican-2, disialoganglioside, EpCam, L1-CAM, Lewis Y, WT-1, Tyrosinase related protein 1 (TYRP1/gp75); GD2, B-cell maturation antigen (BCMA), CD24, SV40 T, carboxy-anhydrase-IX (CAIX); and CD133 Other examples are known to those of ordinary skill in the art.

Particular embodiments utilize ligand binding domains that specifically bind HER2, CE7, hB7H3, EGFR, EGFRvIII, CD19, CD20, CD22, EphA2, IL13Ra2, L1CAM, oaGD2, B7H3, CD33, Mesothelin, ROR1, FITC or VAR2CSA.

In particular embodiments, an scFv utilized with the teaching of this disclosure includes an huCD19 (G01S) scFv, a muCD19 (FMC63) scFv, a CD20 (Leu 16) scFv, a CD22 (m971) scFv, a B7H3 (hBRCA84D) scFv, an L1CAM (CE7) scFv, an EGFR scFv, an EGFRVIII (806) scFv, an EphA2 (2A4) scFv, an EpHA2 (4H5) scFv, an FITC (E2) scFv, a GD2 (hu3F8) scFv, a Her2 (Herceptin) scFv, an IL13Ra2 (hu08) VIVh scFv, an IL13Ra2 hu08 VhV1 scFv, an IL13Ra2 (hu07) VhV1 scFv, an IL13Ra2 (hu07) VhVI scFv, an oaGD2 (8B6) VIVh, a ROR1 (R12) scFv, a CD33 (h2H12) VhVI scFv, a CD33 (h2H12) VIVh scFv, a mesothelin (P4) scFv, a VAR2CSA (ID1-DBL2Xb) scFv, or an IL13Ra2 (IL13 zetakine) amino acid sequence. See FIG. 33 for sequences of these exemplary scFv.

Binding domains that bind the following exemplary viral antigens can also be used: coronaviral antigens: the spike (S) protein; cytomegaloviral antigens: envelope glycoprotein B and CMV pp65; Epstein-Barr antigens: EBV EBNAI, EBV P18, and EBV P23; hepatitis antigens: the S, M, and L proteins of hepatitis B virus, the pre-S antigen of hepatitis B virus, HBCAG DELTA, HBV HBE, hepatitis C viral RNA, HCV NS3 and HCV NS4; herpes simplex viral antigens: immediate early proteins and glycoprotein D; HIV antigens: gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; influenza antigens: hemagglutinin and neuraminidase; Japanese encephalitis viral antigens: proteins E, M-E, M-E-NS1, NS1, NS1-NS2A and 80% E; measles antigens: the measles virus fusion protein; rabies antigens: rabies glycoprotein and rabies nucleoprotein; respiratory syncytial viral antigens: the RSV fusion protein and the M2 protein; rotaviral antigens: VP7sc; rubella antigens: proteins E1 and E2; and varicella zoster viral antigens: gpI and gpII. See Fundamental Virology, Second Edition, eds. Fields, B. N. and Knipe, D. M. (Raven Press, New York, 1991) for additional examples of viral antigens.

In particular embodiments, the binding domain is specific for a B-cell ligand, wherein the binding domain is specific for CDId, CD5, CD19, CD20, CD21, CD22, CD23/Fc epsilon RII, CD24, CD25/IL-2 R alphaCD27/TNFRSF7, CD32, CD34, CD35, CD38, CD40 (TNFRSF5), CD44, CD45, CD45.1, CD45.2, CD54 (ICAM-1), CD69, CD72, CD79, CD80, CD84/SLAMF5, LFA-1, CALLA, BCMA, B-cell receptor (BCR), IgMs, IgD, B220/CD45R, Clq R1/CD93, CD84/SLAMF5, BAFF R/TNFRSF13C, B220/CD45R, B7-1/CD80, B7-2/CD86, TNFSF7, TNFRSF5, ENPP-1, HVEM/TNFRSF14, BLIMP 1/PRDMI, CXCR4, DEP-1/CD148 or EMMPRIN/CD 147.

CAR binding domains can also bind other immune cell antigens found on, e.g., natural kiIler T (NKT) cells, natural kiIler cells (also known as K cells and kiIler cells), tumor-infiltrating lymphocytes (TILs), marrow-infiltrating lymphocytes (MILs), MAIT cells, macrophages, monocytes, and/or dendritic cells. These cells and exemplary cell surface antigens are described elsewhere herein.

Binding domains described herein can also bind haptens. Haptens include any small molecule which, when combined with a larger carrier such as a protein, elicits the production of antibodies which bind specifically to it (in the free or combined state). Haptens can include peptides, other larger chemicals, and aptamers. In some embodiments, a hapten can be any hapten provided in the hapten database accessible on the World Wide Web under the URL crdd.osdd.net/raghava/haptendb/.

In some embodiments, the hapten is fluorescein, urushiol, quinone, biotin, or dinitrophenol, and/or derivatives thereof. In particular embodiments, the hapten is Alexa Fluor 405; Alexa Fluor 430; Alexa Fluor 500; Alexa Fluor 514; Alexa Fluor 532; Alexa Fluor 546; Alexa Fluor 555; Alexa Fluor 568; Alexa Fluor 594; Alexa Fluor 610; Alexa Fluor 633; Alexa Fluor 635; Alexa Fluor 647; Alexa Fluor 660; Alexa Fluor 680; Alexa Fluor 700; Alexa Fluor 750; Alexa Fluor 790; Cascade Blue; Alexa Fluor 488; BODIPY; Dansyl chloride; Oregon Green; Lucifer yellow; Rhodamine; Tetramethylrhodamine; Nitrotyrosine; digoxigenin; 2,4-Dichlorophenoxyacetic acid; Atrazine (2-Chloro-4-(ethylamino)-6-(isopropy lamino)-s-triazine); Nicotine (3-(I-Methyl-2-pyrrolidyl)pyridine; Black Leaf); Morphine (morph; Morphine Sulfate); 2,4-Dinitrochlorobenzene (1-Chloro-2,4-dinitrobenzene; DNCB; Dinitrochlorobenzene); 4-chloro-6-(ethylamino)-1,3,5-triazine-2-(6-aminohexanecarboxylic acid); Structurally related s-triazines (Modifications: H/CI/C6 R1=NH2-, R2=—Cl, R3=—NH—(CH2)5-COOH; iPr/CI/nBu R1=CH3)2-CH—NH—, R2=—Cl, R3=—NH—(CH2)3-(CH3)); Ametryn (2-Ethylamino-4-isopropylamino-6-methylthio-1,3,5-triazine); Deethylatrazine (DEA) (Structurally related s-triazines); Deisopropylatrazine (DIA) (Structurally related s-triazines); Deethyldeisopropylatrazine (DEDIA) (Structurally related s-triazines); Deethyldeisopropylatrazine (DEDIA) (Structurally related s-triazines); HydroxyAtrazine (HA) (Structurally related s-triazines); DeisopropylHydroxyAtrazine (DIHA) (Structurally related s-triazines); DeethylDeisopropylHydroxyAtrazine (DEDIHA) (Structurally related s-triazines); Simazine (Structurally related s-triazines); Desmetryne (Structurally related s-triazines); Prometryne (Structurally related s-triazines); 2-hydroxyatrazine (atrazine derivative); 2-hydroxypropazine (structurally related s-triazine); 2-hydroxysimazine; N-(4-Amine-6-hydroxy-[1,3,5]triazin-2-yl)-4-aminobutanoic Acid (Modification: R1=NH2, R2=NH(CH2)3COOH, R3=OH); SulcoFuron; 5-chloro-2-{4-chloro-2-[3-(3,4-dichlorophenyl)ureido]phenoxy} benzenesulfonic acid; FlucoFuron (1,3-bis(4-chloro-a,a,a-trifluoro-m-tolyl)urea); Agatharesinol; Sequirin C; Sugiresinol; Hydroxysugiresinol; Hinokiresinol; Coniferyl alcohol; Cinnamyl alcohol; p-Coumaric acid; Cinnamic acid; p-Coumaric acid; Cinnamic acid; Hinokinin; Guaiacylglycerol-beta-guaiacyl ether; Morphine-3-glucuronide(M3G); Codeine; Nor-Codeine; 6-Monoacetylmorphine; (+)Methamphetamine; Ceftazidime; Phenobarbital; p-hydroxyPhenobarbital; p-aminophenobarbital; Cyclobarbital; 3′-Ketocyclobarbital; 3′-Hydroxycyclobarbital; Secobarbital; Barbital; Metharbital; Barbituric acid; Thiopental; Thiobarbituric acid; Primidone; Glutethimide; Pentobarbital; Heroin; Diacetylmorphine; Levallorphan; L-11-Allyl-1,2,3,9,10,10a-hexahydro-4H-10,4a-iminoethanophenanthren-6-ol; Pethidine (Demerol; Dolantin; Meperidine; Ethyl 1-methyl-4-phenylpiperidine-4-carboxylate; Isonipecaine); Methamphetamine; d-Desoxyephedrine; Methedrine; Tolopropamine; Pratalgin; Pragman. Benzoylecgonine; 3-Carboxymethylmorphine; Cocaine; 5-benzimidazolecarboxy lie acid; ABA (4-acetyl benzoic acid); Dexamethasone; Flumethasone; 6alpha; 9 alpha-difluoro-11 beta,17,21-trihydroxy-16 alpha-methylpregna-1,4-diene-3,20-dione; 9 alpha-fluoro-11 beta,17,21-trihydroxy-16 beta-methylpregna-1,4-diene-3,20-dione; 9-alpha-fluroprednisolone; Desoxymethasone; Triamcinolone; 9 alpha-fluoro-11 beta,16 alpha; 17,21-tetrahydroxypregna-1,4-diene-3,20-dione; Fluocortolone; 6 alpha-fluoro-11 beta,21-dihydroxypregna-1,4-diene-3,20-dione; Cortisol; 11 beta,17,21-trihydroxypregna-4-ene-3,20-dione; Prednisone; 17,21-dihydroxypregn-4-ene-3, 11,20-trione; Methylprednisolone; 11 beta,17,21-trihydroxy-6 alpha-methylpregna-1,4-diene-3,20-dione; Triamcinolone hexacetonide; 21-(3,3-dimethyl-1-oxobutoxy)-9 alpha-fluoro-11-hydroxy-16,17-[(1-methylethylidene) bis(oxy)]pregna-1,4-diene-3,20-dione; Carbofuran; 2,3-dihydro-2,2-dimethyl-7-benzofuranyl methylcarbamate; BFNP (3-[[(2,3-dihydro-2,2-dimethyl-7-benzofuranyloxy)carbonyl]amino]propanoic acid); Carbofuran derivative; 2,3-dihydro-2,2-dimethyl-7-benzofuranol; Bendiocarb; Carbaryl; Methiocarb; Propoxur; Aldicarb; Methomyl; Benalaxyl; methyl N-(phenylacetyl)-N-(2,6-xylyl)-DL-alaninate; Bn-Ba (4-[2-(N-pheny lacetyl-N-2, 6-xylylamino)propionamido] butyric acid); Bn-COOH (4-[2-(N-phenylacetyl-N-2,6-xylyl-DL-alanine); Benalaxyl derivative; Furalaxyl; Metalaxyl; Acetochlor; Dimetachlor; Metolachlor; 2-chloro-6′-ethyl-N-(2-methoxy-1-methylethyl)acet-o-toluidide; Diethathyl-ethyl; Benzoylprop-ethyl; Benzoylprop-ethyl; 2,4,5-Trichlorophenoxyacetic acid; 2-chloro-6′-ethyl-N-(2-methoxy-1-methylethyl)acet-o-toluidide; Diethathyl-ethyl; Benzoylprop-ethyl; Propachlor; Propachlor; 2,4,5-Trichlorophenoxyacetic acid; 2,4,5, T; Weedone; 2,4-Dichlorophenoxybutyric acid (2,4-DB); 2,4-DB; Butanoic acid; 4-(2,4-dichlorophenoxy)-; Butoxone; Embutone; MCPA; 2-Methyl-4-chlorophenoxyacetic acid; Metaxon; Dichlorprop (2,4-DP); 1-[(2-chloro)phenylsulfonyl]monoamidosuccinic acid; Chlorsulfuron; chlorbromuron; amidosulfuron; chlortoluron; isoproturon; diuron; Linuron O-Methyl-O-(4-nitrophenyl)-N-(4-carboxybuty 1)-phosphoramidothioate Parathion-methyl; 0,0-dimethyl 0-4-nitrophenyl phosphorothioate; Methaphos; Wolfatox; Dimethylparathion; Metacide.,Parathion-ethyl; diethyl p-nitrophenyl thiophosphate; 0,0-diethyl0-(p-nitrophenyl) phosphorothioate; Fenitrothion; 0,0-dimetyl 0-4-nitro-m-tolyl phosphorothioate; Fenthion,0,0-dimethyl 0-4-methylthio-m-tolyl phosphorothioate; Bromophos, 0-4-bromo-2,5-dichlorophenyl 0,0-dimethyl phosphorothioate; chlorpyrifos-methyl,0,0-dimethyl 0-3,5,6-trichloro-2-pyridyl phosphorothioate; Oxidized parathion-methyl,Paraoxon; phosphoric acid; 0,0-diethyl0-(4-nitrophenyl)ester,Diazinon,0,0-diethyl0-2-isopropyl-6-methylpyrimidin-4-yl phosphorothioate; Azinphos-methyl; pirimiphos-methyl; 0-2-diethylamino-6-methyl pyrimidin-4-yl 0,0-dimethyl phosphorothioate; Methidathion; S-2,3-dihydro-5-methoxy-2-oxo-1,3,4-thiadiazol-3-ylmethyl 0,0-dimethyl phosphorodithioate; Dimethylchlororothiophosphate; 4-nitrophenol; p-nitrophenol; Phenolic derivative (Modification On benzene ring; RI=OH R2=N02 R3=H R4=CH2COOH R5=H R6=H); 2-Nitrophenol; o-Nitrophenol; 3-Nitrophenol; m-nitrophenol; 2,4-Dinitrophenol; 3,4-Dinitrophenol; 2,5-Dinitrophenol; 2,4-Dinitro-6-methylphenol; 2,3,6-trinitrophenol; 2-Chlorophenol; 4-Chloro-3-methylphenol, Fenitroxon; 3-Methyl-4-nitrophenol; Nonylphenol,HOM(3-[2-hydroxy-5nitro benzylthio] propionic acid; Phenol,Delor 103; Polyclorinated Biphenyls; Delor 104; Polyclorinated Biphenyls; Delor 105,Polyclorinated Biphenyls,Delor 106; 4,4′-Dichlorobiphenyl,PCB congeners; 2,4,4′-Trichlorobiphenyl; PCB congeners,2,4′-; PCB congeners; 2,2′-Dichlorobiphenyl,PCB congeners; 2,4,5-Trichlorobiphenyl,PCB congeners; 3,3′,4,4′-Tetrachlorobiphenyl,PCB congeners; PCB congeners; 2,2′,4,4′,5,5′-Hexachlorobiphenyl; 2-(5-Carboxypentanoylamino)4,4′-dichlorobiphenyl,Biphenyl derivative,4-chlorophenoxyacetic acid,2-Chlorophenoxyacetic acid; DDT, 1, 1, 1,-trichloro-2; 2-bis-(p-chlorophenyl)ethane,DDE, 1, 1-dichloro-2; 2-bis(p-chlorophenyl)ethylene,p-Chlorophenol; 4-Chlorophenol; m-Chlorophenol 3,4-Dichlorophenol; 3,5-Dichlorophenol; 2,3,4-Trichlorophenol; 2,3,5-Trichlorophenol; 3-methylindole; 3-methylindole Derivatives; 4-(3-methylindol-5-yloxy)butanoic acid; 4-(3-methylindol-5-yloxy)butanoic acid; 3-methylindole Derivatives; 6-[n-3-methylindol-5-yloxy carbonyl)amino]hexanoicacid; 6-[n-3-methylindol-5-yloxy carbonyl)amino]hexanoicacid; 3-methylindole Derivatives; 2-[4-(3-methylindol-6-yl)but-1-ylthro]acetic acid; 2-[4-(3-methylindol-6-yl)but-1-ylthro]acetic acid; 3-methylindole Derivatives; 4-(3-methylindol-6-yl-4-oxo)butanoic acid; 4-(3-methylindol-6-yl-4-oxo)butanoic acid; 3-methylindole Derivatives; 6-(3-methylindol-7-yloxy)hexanoic acid; 6-(3-methylindol-7-yloxy)hexanoic acid; Indole; Indole-3-Carboxylic acid; Indole Derivative-Indole-3-Acetic acid; Indole-3-Acetic acid; Indole Derivative-Indole-3-Propionic acid; Indole-3-Propionic acid; Indole Derivative-Indole-3-Carbinol,lndole-3-Carbinol; Tryptophan; Tryptamine; 5-Methoxyindole-3-carboxaldehyde,5-Methoxytryptamine; 5-Methoxyindole; 6-Methoxyindole; 7-Methoxyindole, EB1089(Seocalcitol); EB1089(Seocalcitol) Derivative; (22E,24E)-Des-A,B-24-homo-26,27-dimethyl-8-[(E)-N-(2-carboxyethyl)-carbamoylmethylidene]-cholesta-22,24-dien-25-ol; 1 alpha-25-dihydroxyvitamin D3; 25(OH)D3,25-hydroxyvitamin D3,24R,25(OH)2D3; 24R,25-dihydroxyvitamin D3; Vitamin D2,ergocalciferol; Vitamin D3; cholecalciferol; EB1446; EB1 436; EB1445; EB1470; DeethylHydroxyAtrazine (DEHA) (Structurally related s-triazines); Irgarol 1051; Flourescein Isothiocyanate; FITC,Metanephrine,N orMetanephrine; Propazine; Terbutylazine; Terbuthylazine; 6-chloro-N-(1,1-dimethylethyl)-N′-ethyl-1,3,5-triazine-2,4-diamine; (Structurally related s-triazines); Ametryn (2-Ethylamino-4-isopropylamino-6-methylthio-1,3,5-triazine (Modification iPr/SCH3/Et R1=(CH3)2-CH—NH—, R2=—SCH3, R3=—NH-CH2-CH3; Irgarol; Cyanazine (Modification R1=Cl, R2=NHCH2CH3, R3=NHCCN(CH3)2); OH-Terbutylazine; Terbutylazine-20H; Hydroxytriazine (EQ-0027); Deisopropylatrazine (Structurally related S-triazine); Desethylterbutylazine (Structurally related S-triazine); Desethyl-deisopropylatrazine (Structurally related S-triazine); Atraton; Terbutryn (Structurally related s-triazines); Atrazine derivative (Modification R1=—NHCH(CH3)2, R2=—S(CH2)2COOH, R3=—NHC2H5); Cyanuric chloride; Trifluralin; (Structurally related s-triazines) tBu/C4/SCH3 (Modification R1=—NH—C—(CH3)3, R2=—NH(CH2)3COOH, R3=—SCH3); Sulphamethazine; (Structurally related s-triazines) 6-[[[4-Chloro-6-(methylamino)]-1,3,5-triazin-2-yl]amino]hexanoic Acid (Modification Me/CI/C6 R1=—NHCH3, R2=—Cl, R3=—NH(CH2)5COOH); (Structurally related s-triazines) Procyazine (Modification R1=—Cl, R2=—NHcyclopropyl, R3=—NHCCN(CH3)2); (Structurally related s-triazines); Prometon (Modification R1=—OCH3, R2=—NHCH(CH3)2, R3=—NHCH(CH3)2); (Structurally related s-triazines) Atrazine Mercapturic Acid (AM) (Modification R1=—SCH2CH(NHAc)COOH, R2=—NHCH2CH3, R3=—NHCH(CH3)2); (Structurally related s-triazines),desethyl atrazine mercapturic acid (desethyl AM) (Modification R1=-NAcCys, R2=—NH2, R3=—NHCH(CH3)2); (Structurally related s-triazines); deisopropyl atrazine mercapturic acid (deisopropyl AM) (Modification R1=-NAcCys, R2=—NHCH2CH3, R3=—NH2); (Structurally related s-triazines); didealkylated atrazine mercapturic acid (didealkylated AM) (Modification R1=-NAcCys, R2=—NH2, R3=—NH2); (Structurally related s-triazines); simazine mercapturate (Modification R1=-NAcCys, R2=—NHCH2CH3, R3=—NHCH2CH3); (Structurally related s-triazines) (Modification R1=—S(CH2)2COOH, R2=—NHCH2CH3, R3=—NHCH2CH3); (Structurally related s-triazines) (Modification R1=—Cl, R2=—NHCH(CH3)2, R3=—NH(CH2)2COOH); (Structurally related s-triazines) (Modification R1=—Cl, R2=—NHCH2CH3, R3=—NH(CH2)2COOH); (Structurally related s-triazines); atrazine mercapturic acid methyl ester (AM methyl ester) (Modification R1=-NAcCysME, R2=—NHCH2CH3, R3=—NHCH(CH3)2); N-acetylcysteine; S-benzyl mercapturate; (Structurally related s-triazines); simetryn (Modification R1=—SCH3, R2=—NHCH2CH3, R3=—NHCH2CH3); Metribuzin; 4-amino-6-tert-butyl-4,5-dihydro-3-methylthio-1,2,4-triazin-5-one; Sulpha Drugs; N4-acetyl-sulphamethazine (Modification N4-acetyl-sulphamethazine); Sulpha Drugs; Sulphathiazole; Sulphathiazole; Sulphamerazine; Sulphamerazine; Sulphaquinoxaline; Sulphaquinoxaline; Sulphachlorpyridazine; Sulphachlorpyridazine; Sulphapyridine; Sulphadimethoxine; Sulphadimethoxine; Sulphamethoxazole; Sulphamethoxazole; Sulphisoxazole; Sulphisoxazole; Sulphamethizole; Sulphamethizole; Sulphanilamide; Sulphanilamide; Sulphaguanidine; Sulphaguanidine; Sulphadiazine; Sulphadiazine; Sulphamethoxypyridiazine; Sulphamethoxypyridiazine; Pentachlorophenoxipropionic acid; Pentachlorophenol; PCP; 2,3,5,6-Tetrachlorophenol; 1,2,4,5 Tetrachlorobenzene; 2,4,6 Trichlorophenol; 2-Methoxy-3,5,6-trichloropyridine; 1,3,5 Trichlorobenzene; 1,3 Dichlorobenzene; 2,4,5-Trichlorophenol; 2,6-Dichlorophenol; 3,5,6-Trichloro-2-pyridinoxiacetic acid; 3,5,6-Trichloro-2-Pyridinol; TCP; 2,4-Dichlorophenol; 2,5-Dichlorophenol; DNC; 4,4′-dinitrocarbanilide; (Structurally related s-triazines); Dichloroatrazine; (Structurally related s-triazines); Dichlorosimazine; 1-((6-chloropyridin-3-yl)methyl)imidazolidin-2-imin; Pyridine Derivative; 6-chloropyridine-3-carboxylic acid; Nicotinic acid; Pyridine Derivative; N-((6-chloropyridin-3-yl)methyl)-N-methylacetamide; (6-chloropyridin-3-yl)-N-methylmethanamine; (6-chloropyridin-3-yl)methanol; Imidacloprid; 1-(6-chloro-3-pyridylmethyl)-N-nitroimidazolidin-2-ylideneamine; Acetamiprid; (E)-N1-[(6-chloro-3-pyridyl)methyl]-N2-cyano-N1-methylacetamidine; Nitenpyram; Deltamethrin; 1(R)-cis-alpha(S)-3-(2,2-dibromoethenyl)-2,2-dimethylcyclopropane carboxylic acid cyano(3-phenoxyphenyl)methyl ester; DON; deoxynivalenol; DON derivative; 15-AcDON (15-acetyldeoxynivalenol); DON derivative; -AcDON (3-acetyldeoxynivalenol); DON derivative; 3,15-DiacDON (3,15-diacetyldeoxynivalenol); DON derivative; 3,7,15-TriacDON (3,7,15-Triacetyldeoxynivalenol); NIV (nivalenol); nivalenol; NIV Derivative; 4-AcNIV (fusarenon X); Flutolanil; alpha,alpha,alpha-trifluoro-3′-isopropoxy-o-toluanilide; Mepronil; Mebenil; Benodanil; 24,25(OH)2D3; (24R)-24,25-dihydroxyvitamin D3; 24S,25(OH)2D3; 24S,25-dihydroxyvitamin D3; 25R,26(OH)2D3; 25R,26-dihydroxyvitamin D3; 25S,26(OH)2D3; 25S,26-dihydroxyvitamin D3; I,24,25(OH)3D3; 1,24,25-trihydroxyvitamin D3; 1,25-lactone; (23S,25R)-1,25(OH)2 D3 26,23-lactone; 24,25(OH)2--7-DHC; 24,25(OH)2-7-dehydrocholesterol; 25(OH)D3 3S; 25(OH)D3 3-sulfate; 24,25(OH)2D3-Hemiglutarate Derivative; 11 alpha-hemiglutaryloxy-(24R)-24,25-dihydroxyvitamin D3; 24,25(OH)2D3-Hemiglutarate Derivative; (24R)-24,25-dihydroxyvitaminD3-3-hemiglutarate; 24R,25(OH)2D2; 24S,25(OH)2D2; 25(OH)D2; 1,24(OH)2D3; 2,3,6-Trichlorophenol; Tetrachlorohydroquinone; Pentachloroaniline; Pentachlorobenzene; 2,3-Dinitrotoluene; 4-Dinitrotoluene; 2,4,5-Trichloronitrobenzene; 3-(3-Hydroxy-2,4,6-trichlorophenyl)-propanoic acid; 2,3,4,6-Tetrachlorophenol; 2,4,6-Trichloroanisol; 2,4,6-TCA; Pentabromophenol; PBP; 2,4,6-Tribromophenol; 2,4,6-TBP; 2-Bromo-4-Chlorophenol; 2-B-4-CP 2,4-Dibromophenol; 2,4-DBP; 2,6-Dibromophenol; 2,6-DBP; 4-Bromophenol; 4-BP; Furosemide; Ampicillin; Amoxicillin; 6-amino-penicillanic acid (6-APA); Azlocillin; Bacampicillin; Carbenicillin; Epicillin; Cloxacillin; Dicloxacillin; Metampicillin; Methicillin; Moxalactam; Oxacillin; Penicillin G; benzyl penicillin; Penicillin V; phenoxy methyl penicillin; Pheneticillin; Piperacillin; Ticarcillin; Ampicillin hydrolyzed; Penicillin G hydrolyzed; 3-phenoxybenzoic acid (3-PBAc) Chlorpyrifos; Chlorpyrifos derivatives; HClol; Synthesized directly from chlorpyrifos technical grade by substitution of the chlorine in position 6 by a 3-mercaptopropanoic acid spacer arm; Chlorpyrifos derivatives; HTCP (Modification HTCP of TCP metabolite was prepared from HClol by hydrolysis of the thiphosphate ester); Zeatin Riboside (trans isomer); Zeatin (trans isomer); N6-(2-isopentenyl)-adenosine; IPA; N6-(2-isopentenyl)-adenine; 2-iP; Benzyladenine; Kinetin; monuron; monolinuron; fenuron; neburon; propanil; propham; chloropropham; 4-chloroaniline; Methyl Urea Derivative; 1-(3-Carboxypropyl)-3-(4-chlorophenyl)-1-methylurea; Methyl Urea Derivative; 1-(5-Carboxypenty 1)-3-(4-chlorophenyl)-1-methylurea; metabromuron; Sennoside B; SB; Sennoside B possessed a erythro configuration between C-10 and C-10′; Sennoside A (Modification Sennoside A possessed a threo configuration between C-10 and C-10); Rhein; Emodin; Aloe-emodin; Barbaloin; 1,4 Dihydroxyanthraquinone; Rhaponticin; Galic acid; Vanillic acid; Caffeic acid; Homogentisic acid; Esculin; Cinnamtannin 1; Baicalin; Naringin hydrate; Wogonine; Wogonine 7-o-beta-glucuronide; Curcumin; deltal-Tetrahydrocannabinolic acid; deltal-Tetrahydrocannabinol; (+-)-cis-4-Aminopermethrin; 3-(4-Aminophenoxy)benzy1(+−)-cis-3-(2,2-dichloroethenyl-2,2-dimethylcyclopropanecarboxylate; Permethrin; trans-Permethrin; cis-Permethrin; Cypermethrin; Phenothrin; Resmethrin; Cyfluthrin; trans-Permethrin acid Esfenvalerate; Fluvalinate; Fenpropathrin; cis-permethrin acid; 4-Phenoxybenzoyl alcohol; Diuron Derivative; 1-(3-Carboxypropyl)-3-(3,4-dichlorophenyl)-1-methylurea; Siduron; Terbuthiuron; Barban; acid trifluralin; 2,6-dinitro-N-propyl-N-(2-carboxyethy)-4-(trifluoromethyl)benzenamine; TR-13; 2-ethyl-7-nitro-1-propyl-5-(trifluoromethyl)-1H-benzimidazole; benefin; 2,6-dinitro-N-buty1-N-ethyl-4-(trifluoromethyl)benzenamine; TR-2; 2,6-dinitro-N-propyl-4-(trifluoromethyl)benzenamine; ethalfluaralin; 2,6-dinitro-N-ethyl-N-(2-methyl-2-propenyl)-4-(trifluoromethyl)benzenamine; TR-40; N-(2,6-dinitro-4-(trifluoromethyl)phenyl)-N-propylpropanamide; TR-15; 2-ethyl-4-nitro-6-(trifluoromethyl)-I H-benzimidazole; TR-3; 2,6-dinitro-4-(trifluoromethyl)benzenamine; TR-6; 3-nitro-5-(trifluoromethyl)-1,2-benzenediamine; TR-9; 5-(trifluoromethyl)-1,2,3-benzenetriamine; TR-21; 4-(dipropylamino)-3,5-dinitrobenzoic acid; TR-36M; 3-methoxy-2,6-dinitro-N,N-dipropyl-4-(trifluoromethyl)benzinamine; oryzalin; 3,5-dinitro-4-(dipropylamino)benzenesulfonamide; pendimethalin; 2,6-dinitro-N-(1-ethylpropyl)-3,4-dimethylbenzenamine; penta galloyl glucose; Pyrene Pyrene-1-carboxaldehyde; Phenanthrene; Benzo(a)pyrene; 3,4-Benzopyrene; Anthracene; 3,4-Benzopyrene; Acenapthene; Fluorene; Chrysene; 1,2-Benzphenanthrene; Benzo[g,h,i]perylene; Benzo[e]pyrene; Acenaphthylene; Fluoranthene; Benzo(j,k)fluorene; Indeno-1,2,3-cd-pyrene; 1,10-(1,2-Phenylene)pyrene; Benzo[a]anthracene; 1,2-Benzanthracene; Benzo(k)fluoranthene; Naphthalene; Benzo[a]fluoranthene; Dibenzo[ah]anthracene; 1,2:5,6-Dibenzanthracene; 2,3-Diaminonaphthalene; 2,6-Dinitroaniline; 17-beta-estradiol (ED); estra-1,3,5(10)-triene-3,17-beta-diol; Trifluralin derivative; 2,6-dinitro-4-tri-fluoromethylaniline; Trifluralin derivative; N-(2, 6-dinitro-4-trifluoromethylpheny)-6-aminohexanoic acid; Trifluralin derivative; N-(2, 6-dinitro-4-trifluoromethylphenyl)-N-methyl-6-aminohexanoic acid; Trifluralin derivative; N-(2,6-dinitro-4-trifluoromethylphenyl)-N-propyl-6-aminohexanoic acid; Trifluralin derivative; N-(2,6-dinitro-4-trifluoromethylphenyl)-6-aminohexanoic acid methyl ester; Trifluralin derivative; N-(2,6-dinitro-4-trifluoromethylphenyll)-6-aminohexanoic acid tert-butylester; Benfluralin; Ethalfluralin; Trifluralin derivative; 2,6-Dinitro-4-trifluoromethylphenol; Isopropalin; Aniline; 2-Hydroxybenzotrifluoride; N-propyl-6-aminohexanoic acid; N-methyl-6-aminohexanoic acid; MHPG Derivatives; D-MHPG (D-3-methoxy-4-hydroxyphenylglycol); MHPG Derivatives; L-MHPG (L-3-methoxy-4-hydroxyphenylglycol); MHPG Derivatives; DL-MHPG (DL-3-methoxy-4-hydroxyphenylglycol); Isomeric mixture of D-MHPG and L-MHPG forms; MHPG Derivatives; DL-MHPG-S04 (DL-3-methoxy-4-hydroxyphenylglycol-sulfate) Modification can include Isomeric mixture of D-MHPG-S04 and L-MHPG-S04 forms; Serotonin; 5-HT; 5-hydroxydopamine (5-4HDA); 3,4-dihydroxyphenylglycol (DOPEG); Dopamine; 4-(2-aminoethyl)pyrocatechol; 3-hydroxytyramine; 3,4-dihydroxyphenethy lamine; L-3,4-dihydroxyphenylalanine; L-DOPA; Vanillomandelic acid; DL-VMA; Homovanillic acid; Norepinephrine; DL-NE; D-Epinephrine; D-E; 3-methoxythyramine; MTA; 3-methoxytyrosine; MTyr; 3,4-dihydroxymandelic acid; DL-DOMA; 3,4-dihydroxyphenyl acetic acid; DOPAC; L-Phenylalanine; Tyramine; p-tyramine; 4-(2-Aminoethyl)phenol; D-Mandelic acid; Homocatechol; Octopamine; DL-Octopamine; Azinphos-Ethyl; S-(3,4-dihydro-4-oxobenzo[d]-[1,2,3]-triazin-3-ylmethyl) 0,0-diethyl phosphorodithioate; Phosmet; 0,0-dimethyl S-phthalimidomethyl phosphorodithioate; Folpet; N-[(Trichloromethyl)thio]phthalimide; Tetramethrin; (1-Cyclohexene-1,2-dicarboximido)methyl-2,2-dimethyl-3-(2-methylpropenyl)-cyclopropanecarboxylate; N-(bromomethyl)phthalimide; N-(Chloromethyl)benzazimide; 6-(N-phthalimidoy lmethylthio)hexanoic acid(MFH); Bromacil; 5-bromo-3-sec-butyl-6-methyluracil; Bromacil Derivative; 5-bromo-6-(hydroxymethyl)-3-(1-methylpropy)—2,4(1H,3H)-pyrimidineone; Bromacil Derivative; S-bromo-3-(2-methylpropyl-6-methyl-2,4(1H,3H)-pyrimidinedione; Metabolite of Bromacil; Bromacil Derivative; 3-hydroxy-1-methylpropyl-6-methyl-2,4(1H,3H)-pyrimidinedione (Modification Bromacil Metabolite); Bromacil Derivative; 6-methyl-3-(1-methylpropyl)-2,4(1H,3H)-pyrimidinedione (Modification Bromacil Metabolite); Terbacil Derivative; [S-chloro-3-(1,1-dimethylethyl)—6-(hydroxymethyl)-2,4(1H,3H)-pyrimidinedione; Terbacil; 3-tert-butyl-S-chloro-6-methyluracil; Bromacil Derivative; Ethyl-S-(S-Bromo-6-methyl-3-(1-methylpropy)-2,4(1H,3H)-pyrimidinedione-1-yl)hexanoate; Bromacil Derivative alkylated at N-1; Bromacil Derivative S-(S-Bromo-6-methyl-3-(1-methylpropyl)-2,4(1H,3H)-pyrimidinedione-I-yl)hexanoic Acid (Modification Bromacil Derivative alkylated at N-1) Bromacil Derivative; -Bromo-6-(Bromomethyl-3-(1-methylpropyl)-2,4(IH,3H)-pyrimidinedione (Modification Bromacil Derivative substituted at the 6-methyl position); Bromacil Derivative-[S-Bromo-3-(1-methylpropyl)-2,4(1H,3H)-pyrimidinedione-6-yl]-2-carboxylpropanoic Acid (Modification Bromacil Derivative substituted at the 6-methyl position); 3-[S-Bromo-3-(1-methylpropyl)-2,4(1H,3H)-pyrimidinedione-6-yl]propanoic Acid (Modification Bromacil Derivative substituted at the 6-methyl position); Bromacil Derivative S-Bromo-1,6-dimethyl-3-(1-methylpropyl)-2,4(1H,3H)-pyrimidinedione; Bromacil Derivative S-Bromo-I-butyl-6-methyl-3-(1-methylpropyl)-2,4(1H,3H)-pyrimidinedione; Butachlor; N-butoxymethyl-2-chloro-2′,6′-diethylacetanilide; Amidochlor; N-[(acetylamino)methyl]-2-chloro-N-(2,6-diethylpenyl)acetamide; Nicarbazin; N,N′-bis(4-nitrophenyl)-compound with 4,6-dimethyl-2(1H)-pyrimidinone(Modification (DNC+HDP)); 2-hydroxy-4,6-dimethylpyrimidine; HDP; Imazalil; [1-(beta-allyloxy-2,4-dichlorophenethyl)imidazole]; Imazalil Derivative; EIT-0073 (Modification Have a —O(CH2)S—COOH group instead of original —OCH2CH═CH2 group of imazalil); Penconazole; (RS)-1-(2,4-dichloro-β-propylphenethyl)-1H-1,2,4-triazole; Hexaconazole; (RS)-2-(2,4-dichlorophenyl)-1-(1H-1,2,4-triazol-1-yl)hexan-2-ol; Propiconazole; cis-trans-1-[2-(2,4-dichlorophenyl)-4-propyl-1,3-dioxolan-2-ylmethyl]-1H-1,2,4-triazole; Diclobutrazol; 2RS,3RS)-1-(2,4-dichlorophenyl)-4,4-dimethyl-2-(1H-1 2,4-triazol-1-yl)pentan-3-ol; Triflumizole; (E)-4-chloro-a,a,a-trifluoro-N-(1-imidazol-1-yl-2-propoxyethylidene)-o-toluidine; Imazalil Derivative; EIT-0183; Imazalil Derivative; EIT-0180; Imazalil Derivative; EIT-0111; Imazalil Derivative; EIT-0158; Imazalil Derivative; K-240; Chlorothalonil; tetrachloroisophthalonitrile Modification On benzene Ring R=CN, R2=Cl, R3=CN, R4=Cl, R5=Cl, R6=Cl); Chlorothalonil Derivative-2,4,5,6-tetrachloro-3-cyanobenzamide (Modification On benzene Ring R1=CONH2, R2=Cl, R3=CN, R4=Cl, R5=Cl, R6=Cl); Chlorothalonil Derivative-2,5,6-trichloro-4-hydroxyisophthalonitrile (Modification On benzene Ring R1=CN, R2=Cl, R3=CN, R4=OH, R 5=Cl, R6=Cl); 3-arbamyl-2,4,5-trichlorobenzoic acid (Modification On benzene Ring R1=CONH2, R2=Cl, R3=COOR, R4=H, R5=Cl, R6=Cl); Pentachloronitrobenzene (Modification On benzene Ring R1=N02, R2=Cl, R3=Cl, R4=Cl, R5=Cl, R6=Cl); Benzene hexachloride; Hexachlorobenzene; BHC; Lindane (Modification On benzene Ring R1=Cl, R2=Cl, R3=Cl, R4=Cl, R5=Cl, R6=Cl); 2,4,5,6-tetrachlorophenol (Modification On benzene Ring R1=OH, R2=Cl, R3=H, R4=Cl, R5=Cl, R6=Cl); Carbaryl Derivative; Ethylcarbamate (Modification R1=OCONHCH2CH3, R3=H); 1-Naphthol; 1-naphthaleneacetamide; -(1-naphthyl)acetamide; Carbaryl Derivative; 1-Methylcarbonate (Modification R1=OCOOCH3, R2=H; Carbaryl Derivative; 1-Ethylcarbonate (Modification R1=OCOOCH2CH3, R2=H); Carbaryl Derivative 2-Ethylcarbonate (Modification R1=H, R2=OCOOCH2CH3; Carbaryl Derivative; I-Ethylthiocarbonate (Modification R1=OCOSCH2CH3, R2=H); Carbaryl Derivative; 2-Ethylthiocarbonate (Modification R1=H, R2=OCOSCH2CH3); Naptalam; N-1-naphthylphthalamic acid; Carbaryl Derivative; 3-hydroxycarbaryl(Modification R1=OCONHCH3, R2=H, R3=OH, R4=H, R5=H); Carbaryl Derivative 4-hydroxycarbaryl (Modification R1=OCONHCH3, R2=H, R3=H, R4=OH, R5=H); Carbaryl Derivative S-hydroxycarbaryl (Modification R1=OCONHCH3, R2=H, R3=H, R4=H, R5=OH); Carbaryl Derivative; 1-(S-Carboxypentyl)-3-(1-naphthyl)urea (Modification R1=NHCONH(CH2)SCOOH, R2=H); (Structurally related s-triazines)-Aziprotryn; 4-azido-N-isopropyl-6-methylthio-1,3,S-triazin-2-ylamine (Modification R1=—SCH3, R2=—N3, R3=—CH(CH3)2); (Structurally related s-triazines); 2-(ethylamino)-4-(methylthio)-6-aminotriazine (Modification R1=—SCH3, R2=—NH—C2HS, R3=—NH2); (Structurally related s-triazines) 2-amino-4-(methylthio)-6-(isopropylamino)triazine (Modification R1=—SCH3, R2=—NH2, R3=—NH—CH(CH3)2); (Structurally related s-triazines) 2-amino-4-methoxy-6-(isopropylamino)triazine (Modification R1=—OCH3, R2=—NH2, R3=—NH—CH(CH3)2); TCP Derivative (3,5,6-trichloro-2-pyridinol Derivative); 3-(3,S-dichloro-6-hydroxy-2-pyridyl)thiopropanoic Acid; p-nitrosuccinanilic acid (PNA-S); PNA-S; PNA-C; p-nitro-cis-1,2-cyclohexanedicarboxanilic acid; Nitroaniline Derivative; 2-nitroaniline; o-Nitroaniline; Nitroaniline Derivative-3-nitroaniline; m-Nitroaniline; Nitroaniline Derivative-4-nitroaniline; p-Nitroaniline; Aeromatic Alcohols; 4-nitrobenzyl alcohol; Aeromatic Alcohols-4-nitrophenethyl alcohol; Aeromatic Alcohols 2-nitrobenzyl alcohol; Aeromatic Alcohols; 3-nitrobenzyl alcohol; Urea Derivative-I-benzyl-3-(4-nitrophenyl)urea; Urea Derivative-1-(3-chlorophenyl)-3-(2-methoxy-S-nitrophenyl)urea; Urea Derivative-1-(3-chlorophenyl)-3-(4-methoxy-3-nitrophenyl)urea; Urea Derivative-1-(4-chlorophenyl)-3-(4-nitropheny I)urea; Urea Derivative-(2-fluorophenyl)-3-(2-mehtoxy-4-nitrophenyl)urea; 1-(3-mehtoxyphenyl)-3-(3-nitrophenyl)urea; Carbofuran Derivative m Carbofuran-phenol; Carbofuran-hydroxy; Carbofuran-keto; Carbosulfan; 3-dihydro-2,2-dimethylbenzofuran-7-yl (dibutylaminothio)methylcarbamate; Benfuracarb; N-[2,3-dihydro-2,2-dimethylbenzofuran-7-yloxycarbonyl(methyl)aminothio]-N-isopropyl--alininate; Furathiocarb; 2,3-dihydro-2,2-dimethyl-7-benzofuranyl 2,4-dimethyl-S-oxo-6-oxa-3-thia-2,4-diazadecanoate; Carbofuran Derivative; 4-[[(2,3-Dihydro-2,2-dimethyl-7-benzofuranyloxy)carbonyl]-amino]butanoic Acid (BFNB) (Modification n=3 X=CH2); Endrin; nendrin; (1R,4S,4aS,SS,6S, 7R,8R,8aR)-1 2,3,4,10, 10-hexachloro-1,4,4a,5,6,7,8,8a-octahydro-6, 7-epoxy-1,4: 5,8-dimethanonaphthalene; Heptachlor; 1,4,5,6,7,8,8-heptachloro-3a,4, 7,7a-tetrahydro-4, 7-; Chlordane; 1,2,4,5,6,7,8,8-octachloro-2,3,3a,4, 7,7a-hexahydro-4, 7-methanoindene; Endosulfan (Modification isomer mix of alpha and beta forms); Endosulfan (Modification alpha isomeric form); Endosulfan (Modification beta isomeric form); Endosulfan Derivative; Endosulfan sulfate (Modification sulfate form); Endosulfan Derivative; Endosulfan diol; Diol metabolite of endosulfan; Endosulfan Derivative; Endosulfan ether (Modification ether metabolite of endosulfan); Endosulfan Derivative; hydroxy ether; hydroxy ether metabolite of endosulfan; Endosulfan Derivative; Endosulfan lactone (Modification lactone metabolite of endosulfan); Aldrin; Dieldrin; Fenvalerate isomers Modification 1S,2R isomer R:Ph); Fenvalerate isomers (Modification 1R,2S isomer R:Ph); Fenvalerate isomers (Modification 1R,2R isomer R:Ph); Fenvalerate isomers (Modification 1S,2R/S isomer R:Ph); Fenvalerate isomers (Modification 1R,2R/S isomer R:Ph); Fenvalerate isomers; fenvalerate (Modification 1 R/S,2R/S isomer R:Ph); Thiabendazole; 2-(thiazol-4-yl)benzimidazole; Thiabendazole Derivative; 5-hydroxythiabendazole (Modification 5-OH-TBZ); Thiabendazole Derivative; 5-NH2-TBZ; Thiabendazole Derivative; methyl benzimidazole carbamate; Albendazole; Mebendazole; Fenbendazole; Thiabendazole Derivative; 2-succinamidothiabendazole; Thiabendazole Derivative; 2-succinamidothiabendazole; Cambendazole; Fenvalerate Haptens; Cyano[3-(4-aminophenoxy)phenyl]methyl (S)-4-Chloro-alpha-(I-methylethyl)benzeneacetate (4-Aminoesfenvalerate); Fenvalerate Haptens; Benzyl 4-[3-[Cyano[(S)-2-(4-chlorophenyl)-3-methyl-1-oxobutanoxy]methyl]]phenoxy]benzenepropanoate; Fenvalerate Haptens; Benzyl 3-[Cyano[(S)-2-(4-chlorophenyl)-3-methyl-1-oxobutanoxy]methyl]]phenoxyacetate; Fenvalerate Haptens; 3-[Cyano[(S)-2-(4-chlorophenyl)-3-methyl-1-oxobutanoxy]methyl]]phenoxyacetic Acid; Fenvalerate Haptens; Benzyl 6-[3-[Cyano[(S)—2-(4-chlorophenyl)-3-methyl-1-oxobutanoxy]methyl]]phenoxy]hexanoate; Fenvalerate Haptens; 6-[3-[Cyano[(S)-2-(4-chlorophenyl)-3-methyl-1-oxobutanoxy]methyl]]phenoxy]hexanoic Acid Fenvalerate Haptens; 4-[3-[Cyano[(S)-2-(4-chlorophenyl)-3-methyl-1-oxobutanoxy]methyl]]phenoxy]benzenepropanoic Acid; (S)-fenvalerate Acid; (Structurally related s-triazines); atrazine mercapturate Modification R1=—SCH2CH(NHCOCH3)COOH, R2=—NHCH2CH3, R3=—NHCH(CH3)2; Fenthion Hapten; -Methyl 0-[3-methyl-4-(methylthio)phenyl] N-(3-carboxypropy ll)phosphoramidothioate Modification referred as Hapten B; Fenthion Derivative; Oxidized Fenthion; Fenthion Derivative; Oxidized Fenthion; pirimiphos-ethyl; 4-(Methylthio)-m-cresol; Chlorpyrifos Derivative; Chlorpyrifos-oxon; Fenchlorphos; 0,0-dimethyl 0-2,4,5-trichlorophenyl phosphorothioate; Trichloronate; 0-Ethyl 0-2,4,5-trichlorophenyl ethyl-phosphonothioate; Dichlofenthion; 0-2,4-dichlorophenyl 0,0-diethyl phosphorothioate; Parathion; 0,0-diethyl 0-4-nitrophenyl phosphorothioate; Thiophos; Chlorpyrifos Derivative Modification Synthesis of ARI is described; Chlorpyrifos Derivative; 0-Ethyl 0-(3,5,6-Trichloro-2-pyridyl) 0-(3-Carboxypropyl)Phosphorothioate; (PO); Chlorpyrifos Derivative-0-Ethyl 0-(3,5,6-Trichloro-2-pyridyl)N-(5-Carboxyethyl)Phosphoramidothioate; (PN1) (Modification Amide linkage of thiophosphate reagents); Chlorpyrifos Derivative; 0-Ethyl 0-(3,5,6-Trichloro-2-pyridyl)N-(2-Carboxyethyl)Phosphoramidothioate; (PN1) (Modification Amide linkage of suitable thiophosphate reagents); Triadimefon; (RS)-1-(4-chlorophenoxy)-3,3-dimethyl-1-(1H-1,2,4-triazol-1-yl)butan-2-one; GR151004; (4-[[5-[3-[2-(dimethylamino)ethyl]]-5-benzofuranyl]-3-pyridinyl]acetyl]morpholinedihydrochloride; Diflubenzuron; 1-(4-chlorophenyl)-3-(2,6-difluorobenzoy 1)urea; (Structurally related s-triazines)—SprAAT (Modification R1=SCH2CH2COOH, R2=NH2, R3=NH2); (Structurally related s-triazines); SBeAAT (Modification R1=S(C6H4)COOH, R2=NH2, R3=NH2); (Structurally related s-triazines); SAAT (Modification R1=SH, R2=NH2, R3=NH2); (Structurally related s-triazines); CDAT (Modification R1=Cl, R2=NH[C(O)CH3), R3=NH2); (Structurally related s-triazines)—CDET (Modification R1=Cl R2=NH[C(O)CH3), R 3=NH(CH2CH3); (Structurally related s-triazines)—CDIT (Modification R1=Cl R2=NH[C(O)CH3), R3=NH(CH(CH3)2)); (Structurally related s-triazines); CDDT (Modification R1=Cl R2=NH[C(O)CH3), R3=NH[C(O)CH3)); (Structurally related s-triazines)-ammeline; OAAT(Modification R1=OH R2=NH2, R3=NH2); (Structurally related s-triazines)-ammelide; OOAT (Modification R1=OH R2=OH, R3=NH2); (Structurally related s-triazines)-cyanuric acid; OOOT (Modification RI=OH R2=OH, R3=OH); (Structurally related s-triazines); melamine; AAAT (Modification R1=NH2 R2=NH2, R3=NH2); Structurally related s-triazines-N-isoropylammeline; OIAT (Modification R1=OH R2=NH[CH(CH3)2], R 3=NH2; Structurally related s-triazines-N-ethylammeline; OEAT (Modification R1=OH R2=NHCH2CH3, R3=NH2); Structurally related s-triazines; N-ethylammelide; OOET (Modification R1=OH R2=OH, R3=NHCH2CH3); Structurally related s-triazines)-cyromazine,CyPAAT (Modification R1=NH(C3H5) R2=NH2, R3=NH2); Structurally related s-triazines-diamino-s-triazine; HAAT(Modification R1=H R2=NIH2, R3=NH2); PCB congeners; 2,5,3′,4′-tetrachlorobiphenyl (Modification IUPAC no. 70); PCB congeners 2,4,5,3′,4′-pentachlorobiphenyl (Modification IUPAC no.: 118); PCB congeners-2,2′,5,5′-tetrachlorobiphenyl (Modification IUPAC no.: 52); PCB congeners; 6-[3,3′,4′-Trichlorobiphenyl-4-yl)oxy]hexanoic Acid; Metolazone; Brand Names: Mykrox; Zaroxolyn; Furfuryl benzoate; DDT Metabolites; DDA; Paraquat; 1,1′-dimethyl-4,4′-bipyridinium ion; Diethylcarbamazine; THP; 2,4,6-triphenyl-N-(4-hydroxyphenyl)-pyridinium; o-DNCP; -dinitrocarboxyphenol; PCB congeners; 3-chlorobiphenylol (Modification IUPAC No. 2); PCB congeners; 3,4′-dichlorobiphenyl (Modification IUPAC No. 13),PCB congeners; 3,5-dichlorobiphenyl (Modification IUPAC No. 14); PCB congeners; 3,4,5,3′,4′-pentachlorobiphenyl (Modification IUPAC No. 126); 2,3,3′,4′-tetrachlorobiphenyl (Modification IUPAC No. 56); 2′,3,4,5-tetrachlorobiphenyl (Modification IUPAC No. 76); 3,3′,5,5′-tetrachlorobiphenyl (Modification IUPAC No. 80); 2,4,5,2′,5′-pentachlorobiphenyl (Modification IUPAC No. 101); 2,3,3′,4,4′-pentachlorobiphenyl (Modification IUPAC No. 105); 2,3,6,3′,4′-pentachlorobiphenyl (Modification IUPAC No. 110); 3,3′,4,5,5′-pentachlorobiphenyl (Modification IUPAC No. 127); 3,4,5,3′,4′,5′-hexachlorobiphenyl (Modification IUPAC No. 169); 2,3,3′,4,4′,5-hexachlorobiphenyl (Modification IUPAC No. 156); 3,4,3′,4′-tetrabromobiphenyl; 3,4,5,3′ 4′,5′-hexabromobiphenyl; 2,4,5,2′,4′,5′-hexabromobiphenyl; Dibenzofurans and Dioxins; 2,3,7,8-tetrachlorobenzofuran; 2,3,7,8-tetrachlorodibenzo-p-dioxin; 3,4′,5-trichloro-4-biphenylol; 3,3′,5,5′-tetrachloro-4,4′-biphenyldiol; 3,4,3′ 4′-tetrachlorodiphenyl ether; 1-2-dichlorobenzene; 1,4-dichlorobenzene; 1,2,4-trichlorobenzene; 3,4-dichloroaniline; DDT Metabolites; 4,4′-DDT; 4,4′-DDD Retronecine; 3,4-dichlorobiphenyl Modification IUPAC No. 12; 3,4,3′-trichlorobiphenyl (Modification IUPAC No. 35); PCB Congeners; 3,4,4′-trichlorobiphenyl (Modification IUPAC No. 37); 3,4,3′,5-tetrachlorobiphenyl (Modification IUPAC No. 78); 3,4,3′,5′-tetrachlorobiphenyl (Modification IUPAC No. 79); 3,4,4′,5-tetrachlorobiphenyl (Modification IUPAC No. 81); DDT Metabolites; p,p′-DDT (Modification p,p′-dichlorodiphenyltrichloroethane); o,p′-DDT Modification o,p′-dichlorodiphenyltrichloroethane; p,p′-DDE Modification p,p′-DDE; o,p′-DDE Modification o,p′-; p,p′-DDD Modification p,p′-DDD; o,p′-DDD Modification o,p′-DDD; Dicofol; 4,4-dichloro-a-(trichloromethy I)benzhydrol; Cyprazine; 6-chloro-N-cyclopropyl-N′-(1-methylethyl)-1,3,5-triazine-2,4-diamine; Structurally related s-triazines; Dipropetryn; 6-(ethylthio)-N,N′-bis(1-methylethyl)-1,3,5-triazine-2,4-diamine; Trietazine; 6-chloro-N,N,N′-triethyl-1,3,5-triazine-2,4-diamine; 6-Hydroxyatrazine; hexazinone; 3-cyclohexyl-6-dimethylamino-1-methyl-1,3,5-triazine-2,4(1H,3H)-dione; TNT; 2,4,6-Trinitrotoluene; Tetraconazole (MI 4360); 1-[2-(2,4-dichlorophenyl)-3-(1, 1,2,2-tetrafluoroethoxy)propyl]-1H-1,2,4-triazole; DTP; 2-(2,4-dichlorophenyl)-3-(1H-1,2,4-triazol-1-yl)propanol; Imazalyl; fenarimol; (RS)-2,4′-dichloro-a-(pyrimidin-5-yl)benzhydryl alcohol; Lupanine metabolites; (+)-lupanine (Modification R=H); Lupanine metabolites; (+)-13-hydroxy lupanine (Modification R=OH); Lupanine metabolites; hemisuccinate ester of (+)-13-hydroxylupanine (Modification R=OCO—(CH2)2·COOH); Lupanine metabolites; cis-hexahydrophthalate ester of (+)-13-hydroxylupanine (Modification R OCO·C6H10·COOH); Lupanine metabolites; alpha-isolupanine; Lupanine metabolites;-hydroxylupanine; Sparteine; Cysteine; multiflorine; epilupinine; (Structurally related s-triazines); cyanazine acid Modification R1=Cl, R2=NHCH2CH3, R3=NHCCOOH(CH3)2; Structurally related s-triazines Modification R1=Cl, R2=NHCH2CH3, R3=NH(CH2)3000H; Structurally related s-triazines (Modification R1=Cl, R2=NHCH2CH3, R3=NHCH2COOH); (Structurally related s-triazines) (Modification RI=Cl, R2=NHCH2CH3, R3=NH(CH2)4000H); norflurazon; 4-chloro-5-(methylamino)-2-[3-(trifluoromethyl)phenyl]-3(2H)-pyridazinone; norflurazon derivative; desmethyl-norflurazon; metflurazon; -chloro-5-(dimethylamino)-2-[(3-trifluoromethyl)phenyl]-3(2H)-pyridazinone; Pyrazon; Chloridazon; 5-amino-4-chloro-2-phenyl-3(2H)-pyridazinone (active ingredient); dichlorophenyl-pyridazone; (Structurally related s-triazines) azidoatrazine (Modification R1=N3, R2=NHCH(CH3)2, R3=NHCH2CH3); ALACHLOR 2-chloro-2′,6′-diethyl-N-methoxymethylacetanilide; trichothecolone (Modification R1=H, R2=OH, R3=H, R4=0, R5=H); DON derivative; acetyl-T-2; DON derivative; T-2 tetrol tetraacetate; Chlorpyrifos derivatives; mono-dechloro-CP; Bromophos derivative; Bromophos-methyl; Bromophos derivative; Bromophos-ethyl dicapthon; -2-chloro-4-nitrophenyl 0,0-dimethyl phosphorothioate; tetrachlorvinphos; (Z)-2-chloro-1-(2,4,5-trichlorophenyl)vinyl dimethyl phosphate; triclopyr; 3,5,6-trichloro-2-pyridyloxyacetic acid; picloram; 4-amino-3,5,6-trichloropyridine-2-carboxylic acid; Formononetin; Biochanin A; 5; 7-dihydroxy-4′-methoxyisoflavone (Modification It is the 4′-methyl ether of genistein); equol; (7-hydroxy-3-(4′-hydroxyphenyl)-chroman; 2′ methoxyformononetin; Daidzein; 7-hydroxy-3-(4-hydroxyphenyl)-4H-1-benzopyran-4-one; geninstein; quercetin; 3,3′,4′,5,7-Pentahydroxyflavone; 3,5,7,3′,4′-Pentahydroxyflavone; matheucinol; coumestrol; (Structurally related s-triazines); Hydroxysimazine (Modification R1=OH, R2=NHCH2CH3, R3=NHCH2CH3; angustifoline; Alodan; 1-Methyl-4-phenyl-4-carboethoxypiperidine hydrochloride; Zearalenone; RAL; F-2 Toxin; Fenpropimorph; (RS)-cis-4-[3-(4-tert-butylphenyl)-2-methylpropyl]-2,6-dimethylmorpholine; Tridemorph; 2,6-dimethyl-4-tridecylmorpholine; 2,6-dimethylmorpholine; Amorolfine; Fenpropidin; (RS)-1-[3-(4-tert-buty 1phenyl)-2-methylpropyl]piperidine; (Structurally related s-triazines) (Modification R1=Cl, R2=Cl, R 3=NHCH2CH3; (Structurally related s-triazines) Modification R1=Cl, R2=Cl, R3=NHCH(CH3)2; (Structurally related s-triazines) Modification R1=Cl, R2=NHCH2CH3, R3=NH(CH2)5000H; (Structurally related s-triazines) Modification R1=Cl, R 2=NHCH(CH3)2, R3=NHCH2COOH; (Structurally related s-triazines) (Modification R1=C1, R2=NHCH(CH3)2, R3=NH(CH2)5000H); Structurally related s-triazines; cyanazine amide (Modification R1=C1, R2=NHCH2CH3, R3=NHCCONH2(CH3)2); hydroxycyanazine acid (Modification R1=OH, R2=NHCH2CH3, R3NHCCOOH(CH3)2); deethylsimazine (Modification R1=C1, R2=NH2, R3=NHCH2CH3); Albendazole sulfoxide; [5-(propylthionyl)-1H-benzimidazol-2-yl]-,methylester; Albendazole sulfone; 5(6)-alkylbenzimidazoles; 2-amino-5-(propylthio)benzimidazole; 5(6)-alkylbenzimidazoles; 2-amino-5-(propylsulfonyl)benzimidazole; oxibendazole; 5-propoxy-benzimidazole-2-methyl carbamate; 5(6)-arylbenzimidazoles; fenbendazole sulfone (Modification sulfone metabolite of fenbendazole); 5(6)-arylbenzimidazoles; 4′-hydroxyfenbendazole; 5(6)-arylbenzimidazoles; oxfendazole (Modification Oxfendazole is the sulfoxide metabolite of fenbendazole); 5(6)-arylbenzimidazoles; flubendazole; benzimidazole Metabolites; 2-aminobenzimidazole; benzimidazole Metabolites; 5-aminobenzimidazole; benzimidazole Metabolites; 2-acetylbenzimidazole; Benzophenone; Diphenylmethanone; phenyl ketone; Diphenyl ketone; Benzoylbenzene; Benzaldehyde; benzoic aldehyde; 4-Bromo-2,5-dichlorophenol; Acephate; O,S-dimethyl acetylphosphoramidothioate; methamidophos; O,S-dimethyl phosphoramidothioate; Dichlorvos; 2,2-dichlorovinyl dimethyl phosphate; Phenthoate; S-a-ethoxycarbonylbenzyl 0,0-dimethyl phosphorodithioate; EPN; Ethyl p-nitrophenyl thionobenzenephosphonate; Bioresmethrin; -benzyl-3-furylmethyl (1R,3R)-2,2-dimethyl-3-(2-methylprop-I-enyl)cyclopropanecarboxylate (Modification The unresolved isomeric mixture of this substance has the ISO common name resmethrin); flufenoxuron; 1-[4-(2-chloro-a,a,a-trifluoro-p-toly loxy)-2-fluorophenyl]-3-(2,6-difluorobenzoyl)urea; Amitrole; 1H-1,2,4-triazol-3-ylamine; molinate; S-ethyl azepane-1-carbothioate; molinate derivative (Modification S-2-carboxyethyl hexahydroazepine-1-carbothioate); molinate derivative (Modification S-5-carboxypentyl hexahydroazepine-1-carbothioate) molinate derivative (Modification molinate sulfone); molinate derivative (Modification S-(p-aminobenzyl) hexahydroazepine-1-carbothioate); molinate derivative (Modification S-2-(p-aminophenyl)ethyl hexahydroazepine-1-carbothioate); hexamethylenimine; thiobencarb (Bolero); butylate (Sutan); EPTC (Eptam); cycloate (Roneet); pebulate (Tillam); vernolate (Vernam); Aflatoxin Ml; AFMI (Modification AFMI); Aflatoxin B1; AFBI (Modification AFBI); Aflatoxin GI; AFGI (Modification AFGI); Aflatoxin M2; AFM2 (Modification AFM2); Aflatoxin B2; AFB2 (Modification AFB2); Aflatoxin G2; AFG2 (Modification AFG2); Aflatoxin B2alpha; AFB2alpha (Modification AFB2alpha); Aflatoxin G2alpha; AFG2alpha (Modification AFG2alpha); KB-6806; 6-amino-5-chloro-1-isopropyl-2-(4-methyl-1-piperazinyl) (Modification R1=NH2, R2=CH(CH3)2, R3=CH3); KB-6806 (Benzimidazole) Derivatives Modification R1=NH2, R2=CH2CH(CH3)2, R3=CH3; Hapten Name KB-6806 (Benzimidazole) Derivatives (Modification R1=NH2, R2=CH(CH2CH3)2, R3=CH3); KB-6806 (Benzimidazole) Derivatives (Modification R1=NHCOCH3, R2=CH(CH3)2, R3=CH3); KB-6806 (Benzimidazole) Derivatives (Modification R1=H, R2=CH(CH3)2, R3=CH3); KB-6806 (Benzimidazole) Derivatives (Modification R1=NH2, R2=CH(CH3)2, R3=CH3); KB-6806 (Benzimidazole) Derivatives Modification R1=NH2, R2=CH(CH3)2, R3==N(->O) CH3 (N-OXIDE); KB-6806 (Benzimidazole) Derivatives Modification R1=NH2, R2=CH(CH3)2, R3=H; KB-6806 (Benzimidazole) Derivatives Modification R1=NH2, R2=CH2CH3, R3=CH3; Aminopraoxon; phosphoric acid; O,O-diethyl O-(4-aminophenyl)ester,Methylparathion; phosphorothioic acid; O,O-dimethyl 0-(4-nitrophenyl)ester; Diethyl phenylphosphate; phenylphosphonic acid; O,O-diethyl ester; Diethyl phosphate; ethylphosphonic acid; 0,0-diethyl ester; p-Nitorphenyl phosphate; phosphonic acid; 0-(4-nitrophenyl)ester; Phorate; phosphorodithioic acid; O,O-diethyl S-[(ethylthio)methyl] ester; Ethion; bis(phosphorodithioic acid); S,S′-methylene O,O,O′,O′-tetraethyl ester; Carbophenthion; phosphorodithioic acid; O,O-diethyl S-[[(4-chlorophenyl)thio]methyl] ester; Disulfoton; phosphorodithioic acid; O,O-diethyl S-[(2-ethylthio)ethyl] ester; TS; N-[4-(Carboxymethyl)-2-thiazolyl)sulfanilamide; NS; N-(4-Nitrophenyl)sulfanilamide; Sulfamoxole; Sulfacetamide; DNP-SL; Spin labeIled dinitrophenyl (Modification The synthesis of DNP-SL has been described by Balakrishnan et al(1982) formula can be found in Anglister et al. (1984)); beta ecdysone; Benzimidazole Derivative; 5(6)-[Carboxypenty)thio]-2-(methoxycarbony)amino]-benzimidazole; 2-hydroxybiphenyl; HBP; Atrazine Caproic acid; Lysophosphatidic acid (LPA); I-acyl-2-hydroxy-sn-glycero-3-phosphate); berberine; Palmatine; 9-Acetylberberine; Corydaline; Coptisine; Berberrubine; 8-Oxoberbeine; Papaverine; Berberine Derivative; 9-O-carboxymethyl berberine; phencyclidine; 1-(1-phenylcyclohexyl)piperidine; Methoxychlor; Endosulfan Derivative; 4-Oxobutanoic Acid,4-(4,5,6, 7,8,8-Hexachloro-3a,4, 7,7a-tetrahydro-4, 7-methano-IH-indenyl-1-oxy); Endosulfan Derivative; 4-oxybutanoic Acid,4-(1,3,4,5,6,7,8-Octachloro-3a,4,7,7a-tetrahydro-4,7-methanoindanyl-2-oxy; Endosulfan Derivative (Modification Hemisuccinate of Endosulfan diol); Triazole Derivatives; 5-(3-Hydroxypropyl)-3-amino-2H-1,2,4-triazole; Triazole Derivatives; 5-(3-Hydroxypropyl)-3-(2-nitrophenylsulfenyl)amino-2H-1,2,4-triazole; Triazole Derivatives; 3-Amino-5-[(3-succinyloxy)propyl]-2H-1,2,4-triazole; Triazole Derivatives; 3-amino-1,2,4-triazole-5-thiol; Triazole Derivatives; 3-[(2-nitrophenylsulfenyl)amino-2H-1,2,4-triazole-5-thiol; Triazole Derivatives; 2H-1,2,4-triazole-5-thiol; Triazole Derivatives; 4-methyl-1,2,4-triazole-3-thiol; Triazole Derivatives; (1,2,4-triazol-2-yl)acetic acid; 1,2,4-triazole; 4-nitrophenyl 4′-carboxymethylphenyl phosphate; Triazole Derivative; 4-amino-1,2,4-triazole; Triazole Derivative; 3-acetamido-I H-1,2,4-triazole; Triazole Derivative; 3-amino-1,2,4-triazole-5-carboxylic acid hemihydrate; Triazole Derivative; 2-(4-chlorophenyl)-2-(1,2,4-triazol-1-yl)-methylhexanoic acid; succinic acid; Imidazole; L-histidine; L-glutamic acid; Permethrin derivative; 3-phenoxybenzyl 2,2-dimethylcyclopropatane-1,3-dicarboxylate; 3-phenoxybenzaldehyde; flucythrinate; Chrysanthemic acid; 2,4-Dinitrophenyl; DNP; Thiram Haptens; Disodium 4-[Carbodithioato(methyl)-amino]butanoate; Thiram Haptens 5,11-Dimethyl-6,10-dithioxo-7,9-dithia-5,11-diazadodecanoic Acid; Thiram Haptens; 2-{[(Dimethylamino)carbothioyL]sulfanyl}ethanoic Acid; Thiram Haptens; 4-{[(Dimethylamino)carbothioyl]sulfanyl} butanoic Acid; Thiram Haptens; 6-{[(Dimethylamino)carbothioyl]sulfanyl} hexanoic Acid; Thiram Haptens; 11-{[(Dimethylamino)carbothioyl]sulfanyl} undecanoic Acid; Thiram Haptens; 2-{[(Dimethylamino)carbothioyl]sulfanyl} ethanoic Acid; Thiram; Tetramethylthiurammonosulfide; Tetraethylthiuram disulfide; Dimethyldithiocarbamic acid sodium salt; Dimethyldithiocarbamic acid zinc salt; Diethyldithiocarbamic acid sodium salt; N,N,N′,N′-tetramethylthiourea; Nabam; Zineb; Maneb; Ethylenethiourea; Chlorpyrifos hapten; O,O Diethyl O-[3,5-Dichloro-6-[(2-carboxyethyl)thio]-2-pyridyl] Phosphorothioate; 2-Succinamidobenzimidazole; Methyl 2-Benzimidazolecarbamate; MBC; Benzimidazole; 2-benzimidazolylurea; succinamide; Ethyl carbamate; Urea; N-methylurea; N,N′-dimethylurea; Brevetoxin PbTx-3; Organophosphorous Haptens; O,O-Diethyl 0-(5-carboxy-2-fluorophenyl) phosphorothioate; Chlorpyrifos-ethyl; Anandamide hapten; N-Arachidonyl-7-amino-6-hydroxy-heptanoic acid; Anandamide; Arachidonic acid; Docosatetraenoyl ethanolamide; Dihomo-gamma-linolenyl ethanolamide; 2-Arachidonyl glycerol; 2-Arachidonyl glycerol ether; Stearoyl ethanolamide; Heptadecanoyl ethanolamide; Prostaglandin EI; 3-hydroxy-2-(3-hydroxy-1-octenyl)-5-oxocyclopentaneheptanoic acid; alprostadil; PGE 1; Prostaglandin D2; PGD2; Prostaglandin A2; PGA2; Prostaglandin B2; PGB2; Prostaglandin F2 alpha; 7-[3,5-dihydroxy-2-(3-hydroxy-1-octenyl)cyclopentyl]-5-heptenoic acid; dinoprost; PGF2alpha; Prostaglandin FI alpha; PGFlalpha; 6-keto-Prostaglandin FI alpha; 6-keto-PGFlalpha; 13,14-Dihydro-15-keto-Prostaglandin E2; 13,14-Dihydro-I 5-keto-PGE2; 13,14-Dihydro-15-keto-Prostaglandin F2alpha; 14-Dihydro-15-keto-PGF2alpha; 5alpha,7alpha-Dihydroxy-II-ketotetranorpostane-1,16-dioic acid; 15-keto-PGF2alpha; TXB2; Prostaglandin E2; 7-[3-hydroxy-2-(3-hydroxy-1-octenyl)-5-oxocyclopenty]-5-heptenoic acid; dinoprostone; PGE2; hCG-alpha-(59-92)-peptide (34 residues); Paraquat Derivative; Paraquat hexanoate (PQ-h); Monoquat; Diquat; 9, 10-dihydro-8a,10a-diazoniaphenanthrene; MPTP; 1-methyl-4-phenyl-1,2,5,6-tetrahydropyridine; 1,2-Naphthoquinone; N-Acetyl-S-(1,2-dihydroxy-4-naphthyl)cysteine; N-Acetyl-S-(1,4-dihydroxy-2-naphthyl)cysteine; N-Acetyl-S-(1,2-dihydroxy-I-hydroxy-1-naphthyl)cysteine; 2-Chloro-2′. 6′-diethylacetanilide(CDA) Hapten; 2-[2-Chloro-(2′-6′-diethyl)acetanilido]ethanoicAcid; 2-Chloro-2′. 6′-diethylacetanilide(CDA) Hapten; 2-[2-Chloro-(2′-6′-diethyl)acetanilido]butanoic Acid; 2-Chloro-2′. 6′-diethylacetanilide(CDA) Hapten; 5-(4-Chloroacetamido-3,5-diethyl)phenoxypentanoic Acid; CDA; 2-Chloro-2′.6′-diethylacetanilide; HDA; 2-Hydroxy-2′.6′-diethylacetanilide; 2,6-diethyl-aniline; Hydroxyalachlor; Alachlor ESA; Alachlor ethanesulfonic acid; Isoproturon Hapten; 3-(4-Isopropylphenyl)-1-carboxypropyl-1-methyl urea; chlorotoluron; 3-(3-chloro-p-tolyl)-1,1-dimethylurea; Metoxuron; 3-(3-chloro-4-methoxyphenyl)-1,1-dimethybrea; metamitron; 4-amino-4, 5-dihydro-3-methyl-6-phenyl-1,2,4-triazin-5-one; mecoprop; (RS)-2-(4-chloro-o-tolyloxy)propionic acid; propyzamide; 3,5-dichloro-N-(1,1-dimethylpropynyl)benzamide; Paraquat dichloride; MCPB; 4-(4-chloro-o-tolyloxy)butyric acid; Chlortoluron Hapten; N-(3-Chloro-4-methylphenyl)-N-methyl-N-carboxypropyl Urea; Metsulfuron; Methyl 2-[3-(4-methoxy-6-methyl-1,3,5-triazin-2-y I)ureidosulphonyl]benzoate; Captopril Haptens; Captopril-4-(Malelmidomethyl)-cyclohexane Carboxylic Acid(MCC); Captopril Haptens; Captopril Disulfide Modification; Mercaptoethanol-MCC; Mercaptoethanol-4-(Malelmidomethyl)-cyclohexane Carboxylic Acid Modification Captopril Haptens; Captopril without MCC; Aculeatiside A; Aculeatiside B; Solamargine; Solasonine; solanine-S; purapurine; Solasodine; Khasianine; Tomatine; lycopersicin; Tomatidine; 3-0-beta-D-Glucopyranosyl-solasodine; O-alpha-L--Rhamnosyl-1(1->2)-3-0-beta-D-glucopyranosyl-solasodine; 3-0-beta-D-Galacopyranosy 1-solasidine; 0-beta-D-Glucopyranosy 1-1(1->3)-3-0-beta-D-galacopyranosyl-solasodine; 12-Hydroxysolamargine; 12-Hydroxysolasonine; Isoanguivine; Solaverine I; Solaverine ll; Xylosyl-beta-solamargine; alpha-Solanine; alpha-Chaconine; Dioscine; Indole Derivatives; beta-Indole Acetic Acid; 2-Bromo-4,6-dinitroaniline; 2-Chloro-4,6-dinitroaniline; Tetryl; 2,4,6-trinitrophenyl-n-methylnitramine; nitramine; tetralite; tetril; 2-Amino-4,6-dinitrotoluene; 2,4-Dinitroaniline; 3,5-Dinitroaniline; 2-Amino-4,6-dinitrobenzoic acid; Disperse Blue 79; N-[5-[bis[2-(acetyloxy)ethyl]amino]-2-[(2-bromo-4,6-dinitrophenyl)azo]-4-ethoxyphenyl]acetamide; 1,3-Dinitrobenzene; 2,6-Dinitrotoluene; 4-Amino-2,6-dinitrotoluene; 1,3,5-Trinitrobenzene; Nicergoline; Ethylmorphine; 8-Didehydro-4,5-epoxy-3-ethoxy-17-methylmorphinan-6-ol; Dihydromorphine; Dihydrocodeine; dihydromorphinone; Hydromorphone; Dihydrocodeinone; Hydrocodone; Naltrexone; N-cyclopropylmethyl-14-hydroxydihydromorphinone; Dextromethorphan; (±)-3-Methoxy-1 7-methylmorphinan; Homatropine; Endorphins Modification Derivative Type: b-Endorphin; Met-enkephalin; DALEA; D-Ala(2)-D-Leu(5)-enkephalinamide; Vincristine; 22-Oxovincaleukoblastine; leurocristine; VCR; LCR; OCT; 22-Oxacalcitriol; OCT-3-HG; 22-oxacalcitriol-3-Hemiglutarate; 24(OH)OCT; 24(OH)-22-oxacalcitriol; 1,20(OH)2-hexanor-D3; Synephrine; Epinephrine; 4-[(1 R)-1-Hydroxy-2-(methylamino)ethyl]-1,2-benzenediol; Phenylephrine; Dopamine Derivative; 6-hydroxy dopamine; Tyramine derivative; 3-methoxy tyramine; Phenethylamine; Benzeneethanamine; PEA; m-tyramine; o-tyramine; dimethoxyphenethylamine; Thymidine glycol monophosphate; 5,6-Dihydroxythymidine monophosphate; Thymidine monophosphate; Thymidine glycol; Thymine glycol; 5,6-Dihydrothymidine; Thymidine; Thymine; 5-methyluracil; 2,4-dihydroxy-5-methylpyrimidine; AMP; Adenosine mono phosphate; CMP; Cytidine mono phosphate; Carbamazepine; 5-carbamoyl-5H-dibenz[b,f]azepine; Neopterin isomers; D-erythro-Neopterin; Neopterin isomers; L-erythro-Neopterin; Neopterin isomers; D-threo-Neopterin; Biopterin isomers; L-erythro-Biopterin; Biopterin isomers; D-erythro-Biopterin; Biopterin isomers; L-threo-Biopterin; Biopterin isomers; D-threo-Biopterin; Pterin-6-Carboxylic Acid; C7H5Ni03; Pterin; Thromboxane B2; (5Z,9alpha,13E,15S)-9,11,15-trihydroxythromboxa-5,13-dien-1-oic acid; 15 Ketoprostaglandin F2alpha; Fumonisin B1; macrofusine; FB1; Thyroliberin; TRH; thyrotropin-releasing factor; thyrotropin releasing hormone; TRF; protirelin; lopremone; Thyroliberin-OH; TRH-OH; Diketopiperazine; cyclo (H-P); TRH analogues; Methylated TRH; TRH analogues; TRH elongated peptides; TRH-Gly; TRH elongated peptides; TRH-Gly-Lys-Arg; TRH elongated peptides; TRH-Gly-Lys-Arg-Ala; TRH elongated peptides; P7 (Modification Q-H-P-G-L-R-F); TRH elongated peptides; P10 (Modification S-L-R-Q-H-P-G-L-R-F); TRH elongated peptides; Ps5 Modification pro-TRH[178-199]; TRH elongated peptides; TRH-Ps5 (Modification pro-TRH[172-199]; Hypothalmic peptide; LHRH; Cyanoginosin-LA; Cyanoginosin-LB; Cyanoginosin-LR; Cyanoginosin-LY; Cyanoginosin-AY; Cyanoginosin-FR; Cyanoginosin-YR; Ne-acetyllysine-containing peptide; Gly-Lys(Ac)-e-aminocaproic acid (Aca)-Cys; Benzoic Acid; Benzenecarboxylic acid; phenylformic acid; dracylic acid; m-hydroxybenzoic acid; 3-hydroxybenzoic acid; o-methoxybenzoic acid; 2-methoxybenzoic acid; o-toluic acid; 2-Methylbenzoic acid; o-chlorobenzoic acid; 2-chlorobenzoic acid; o-aminobenzoic acid; 2-aminobenzoic acid; thiosalicylic acid; 2-Mercaptobenzoic acid; o-sulfhydrylbenzoic acid; Salicylamide; 2-Hydroxybenzamide; Saligenin; saligenol; o-hydroxybenzyl alcohol; Salicyl alcohol; 2-cyanophenol; 2-hydroxyphenyl acetic acid; p-hydroxybenzoic acid; p-aminobenzoic acid; 4-Aminobenzoic acid; vitamin Bx; bacterial vitamin H1; p-toluic acid; p-methylamino benzoic acid; p-chlorosalicylic acid; 4-chloro-2-hydroxybenzoic acid; 2,4-dihydroxybenzoic acid; beta-Resorcylic Acid; 2,4-dihydroxybenzenecarboxylic acid; BRA; 4-aminosalicylic acid; 4-Amino-2-hydroxybenzoic acid; p-aminosalicylic acid; Gentisic Acid; 2,5-dihydroxybenzoic acid; 5-hydroxysalicylic acid; Picolinic acid; o-Pyridinecarboxylic acid; 2-Pyridinecarboxylic acid; picolinic acid N-oxide; 3-hydroxypicolinic acid; 2-hydroxynicotinic acid; 7-methylguanine; N2-Carboxymethyl-N7-methylguanine; 2-(7-methyl-6-oxo-6,7-dihydro-1H-purin-2-ylamino)acetic acid; 7-methylxanthine; 7-methyluric acid; 7-methyladenine; Guanine; 2-Amino-1,7-dihydro-6H-purin-6-one; 2-aminohypoxanthine; Adenine; 6-aminopurine; 6-amino-1H-purine; 6-amino-3H-purine; 6-amino-9H-purine; 7-(2-Carboxyethy I)guanine; 7-CEGua; 7-Ethylguanine; 2-amino-7-ethyl-1H-purin-6(7H)-one; 7-(2,3-Dihydroxypropyl)guanine; 2-amino-7-(2,3-dihydroxypropyl)-1H-purin-6(7H)-one; 7-(2-Hydroxyethyl)guanine; 2-amino-7-(2-hydroxyethyl)-1H-purin-6(7H)-one; 7-(2-[(2-Hydroxyethyl)amino]ethyl)-guanine; 2-amino-7-(2-(2-hydroxyethylamino)ethyl)-1H-purin-6(7H)-one; 7-Carboxymethylguanine; 2-(2-amino-6-oxo-1,6-dihydropurin-7-yl)acetic acid; fluorescein; urushiol; quinone; biotin; His-tags; FLAG-tags; Strep-tag; Myc-tag; HA-tag; Spot-tag; or NE-tag.

Exemplary antibodies with binding domains that bind haptens include 3-methylindole antibody; 3F12; 3-methylindole antibody; 4A1G; 3-methylindole antibody; 8F2; 3-methylindole antibody; 8H1; 3-methylindole antibody; Anit-Fumonisin BI antibody; 1,2-Naphthoquinone-antibody; 15-Acetyldeoxynivalenol antibody; 2-(2,4-dichlorophenyl)-3(1H-1,2,4-triazol-1-yl)propanol) antibody (DTP antibody); 22-oxacalcitriol antibody (As-1; 2 and 3); 24,25(OH)2D3) antibody (Abl 1); 24,25(OH)2D3) antibody (Ab3); 24,25(OH)2D3) antibody (Ab3-4); 2,4,5-Trichlorophenoxyacetic acid antibody; (2,4,5-Trichlorphenoxyacetic acid) antibody; 2,4,6-Trichlorophenol) antibody; 2,4,6-Trichlorophenol) antibody; 2,4,6-Trinitrotoluene(TNT) antibody; 2,4-Dichlorophenoxyacetic acid(MAb's B5/C3; E2/B5; E2/G2; F6/C10; and F6/E5); (2,4-Dichlorphenoxyacetic acid) antibody; 2-hydroxybiphenyl-antibody; 3,5,6-trichloro-2-pyridinol) antibody (LIB-MC2; LIB-MC3); (3,5,6-trichloro-2-pyridinol) antibody (LIB-MC2 MAb); 3-Acetyldeoxynivalenol(3-AcDON) antibody; 3-phenoxybenzoic acid (3-PBAc)-antibody; -4-Nitrophenol antibody; -nitrophenyl 4′-carboxymethylphenyl phosphate antibody; 7-(Carboxyethyl)guanine(7-CEGua) antibody (group specific for 7-meGua); 7-methylguanine(7-MEGua) antibody; BA antibody; Acephate antibody (Antiserum 8377); cetyllysine antibody (mAbs AL3D5; AL11; AKL3H6; AKL5C1); Aculaetiside-A antibody; Aflatoxin MI (AFMI)antibody (mAbs A1; N12; R16; FF32); gatharesinol antibody; gatharesinol antibody; Amidochlorantibody; mitrole antibody (la-BSA antibody); ampicillin antibody (AMPI 1 1D1 and AMPI II 3B5); nandamide antibody (9C11.C9C; 30G8.E6C; 7D2.E2b; 13C2 MAbs); atrazine antibody; trazine antibody; trazine antibody; Atrazine antibody; trazine antibody; trazine antibody; trazine antibody (4063-21-1 MAb cell line mAb and scAbs); trazine antibody (4D8 and 6C8 scAb); Atrazine antibody (C193); Atrazine antibody (In Rabbit/Sheep); Atrazine antibody (K4E7); Atrazine antibody (MAb: AM7B2. 1); Atrazine antibody (ScAb); Atrazine Mercapturic acid antibody; (Azinphos methyl) antibody (MAB's LIB-MFH14; LIB-MFH110); benalaxyl antibody; bensimidazolecarboxylic acid; benzimidazoles antibody (Ab 587); Benzo[a]pyrene antibody; Benzo(a)pyrene antibody (10C10 and 4D5 MAbs); Benzoylphenylurea)-antibody (mainly against Diflubenzuron); berberine antibody; beta Indole Acetic Acid antibody; Biopterin (L-erythro form) antibody; Brevetoxin PbTx-3-antibody; Bromacil antibody; Bromophos antibody; Bromophos ethyl antibody; Butachlor antibody; Captopril-MCC antibody; Carbamazepine (CBZ)-antibody; Carbaryl antibody; Carbaryl antibody (LIB-CNH32; LIB-CNH33,LIB-CNH36; LIB-CNH37; LIB-CNH45; LIB-CNA38); Carbaryl antibody (LIB/CNH-3.6 MAb); Carbofuran antibody (LIB-BFNB-52; LIB-BFNB-62; LIB-BFNB-67); Carbofuran antibody (LIB-BFNP21); CDA-antibody; CDA-antibody (2-[2-Chloro-(2′-6′-diethyl)acetanilido]butanoic Acid); CDA-antibody (2-[2-Chloro-(2′-6′-diethy)acetanilido] ethanoic Acid); CDA-antibody (5-(4-Chloroacetamido-3,5-diethyl)phenoxypentanoic Acid); ceftazidime antibody; chlorodiamino-s-triazine)antibody (CAAT) (PAb1-8); Chlorothalonil antibody; Chlorpyrifos antibody; Chlorpyrifos antibody; Chlorpyrifos antibody (LIB-AR1.1; LIB-AR1.4 Mabs); Chlorpyrifos antibody (LIB-C4); (chlorpyrifos) antibody (LIB-C4 MAb); Chlorpyrifos antibody (LIB-PN1 Mabs); Chlorpyrifos antibody (LIB-PN2 Mabs); Chlorpyrifos antibody (LIB-PO Mabs); Chlorsulfuron antibody; Chlorsulfuron antibody; Chlortoluron antibody (Antiserum); Cyanoginosin-LA antibody (mAbs 2B2-2; 2B2-7; 2B2-8; 2B2-9; 2B2-10; 2B5-5; 2B5-8; 2B5-14; 2B5-15; 2B5-23); D-3-methoxy-4-hydroxyphenylglycol) antibody; DDA antibody; DDTantibody (PAbs and MAbs); DDT Mabs (LIBI-11; LIB5-21; LIB5-25; LIB5-28; LIB5-212; LIB5-51; LIB5-52; LIB5-53); DEC antibody (diethylcarbamazine antibody); DEHA antibody; Delor 103) antibody; Deltamethrin antibody; Deltamethrin antibody (Del 01 to Del 12 MAbs and PAbs); deoxynivalenol (DON) antibody; Deoxynivalenol (DON) antibody; Dexamethasone antibody; Dexamethasone antibody; Dinitrophenyl(DNP)-antibody; dinitrophenyl spin labeled antibody (ANOI-ANI 2); Diuron Antibodies (MAb's: 21; 60; 195; 202; 275; 481; 488; 520); -D-MHPG antibody; DNC antibody; EB1089 antibody; ecdysone antibody; endosulfan antibody; Esfenvalerate antibody (Ab7588); estradiol antibody; Fenitrothion antibody (pAbs and mAbs); Fenpropimorph antibody; Fenthion antibody; Fenthion antibody; FITC antobodies (B13-DEI); Flucofuron antibody (F2A8/1/A4B3); flufenoxuron antibody; and Benzoylphenylurea)-antibody; Formononetin antibody; Furosemide antibody (Furo-26; Furo37; furo-72; Furn 73 Mabs); GR151004 antibody; hCG-alpha-peptide antibody (FA36; hydroxyatrazine antibody (HYB-283-2); Hydroxysimazine antibody; Imazalil antibody MoAb's(9C1-1-1; 9C5-1-1; 9C6-1-1; 9C8-1-1; 9C9-1-1; 9C12-1-1; 9C14-1-1; 9C16-1-11; 9C18-1-1; 9C19-1-1; 9E1-1; 9G2-1); Irgarol antibody; Isopentenyl adenosine antibody; Isoproturon antibody; AntiKB-6806 antiserum; -(+)lupanine antibody; Lysiohosphatidic (LPA) acid; M3G Ab1 and Ab2; M3G Ab1 and Ab2; MBC antibody (Anti2-succinamidobenzimidazole antiserum); Metanepharine antibody; (+)methamphetamine antibody; Methiocarb antibody (LIB-MXNB31; LIB-MXNB-33; LIB-MXNH14 and LIB-MXNH-15 MAbs); Metolachlor antibody; Metolachlor antibody; Metolachlor antibody (MAb 4082-25-4); Molinate antibody; monuron antibody; morphine-3-glucuronide(E3 scFv antibody); morphine antibody; Morphine antibody; Morphine antibody (mAbs 8.2. 1; 33.2.9; 35.4. 12; 39.3.9; 44.4.1; 76.7F.16; 83.3.10; 115.1.3; 124.2.2; 131.5.13; 158.1.3; 180.2.4); Neopterin (D-erythro form) antibody; Nicarbazin antibody (Nie 6; Nie 7; Nie 8; and Nie 9); Nicergoline antibody (Nic-1; Nic-2; Nic-3 & BNA-1; BNA-3); norflurazon antibody; NorMetanepharine antibody; (o-DNCP) antibody;-PIO antibody (TRH elongated peptide); Paraoxon antibody (BDI and CE3); Paraquat antibody; Paraquat antibody; Parathion-methyl antibody; PCB antibody (against 3,3′,4,4′-tetrachlorobiphenyl) MAb S2B1; pentachlorophenol antibody; Pentachlorophenol antibody; Pentachlorophenol antibody; permethrin antibody (Mabs Py-1; Py-3 and Py-4); Phencyclidine antibody (Mab 6B5 Fab); phenobarbital antibody; phenobarbital antibody; p.p′-DDT)-antibody (LIB-DDT-35 and LIB-DDT5-52); premethrin antibody (Ab549); Propoxur antibody (LIB-PRNPI 5; LIB-PRNP21; LIB-PRNB21; LIB-PRNB33); Prostaglandin E2-antibody; p-tyramine antibody; pyrene antibody; retronecine antibody; Retronecine antibody; salicylate antibody; Sennoside A antibody (MAb 6G8); Sennoside B antibody (MAb's: 7H12; 5G6; 5C7); Simizine antibody; Sulfonamides antibody (AntiTS); Sulocfuron antibody (S2B5/1/C3); sulphamethazine antibody (21C7); synephrine antibody; Thiabendazole antibody (antibody 300); Thiabendazole antibody (antibody 430 and 448); Thiram-antibody; THP antibody (7S and 19S); Thromboxane B2 antibody; thymidine glycol monophosphate antibody (mAb 2.6F.6B.6C);—Thyroliberin (TRH) antibody; TNT antibody (ABI and AB2 antiserum); Triadimefon antibody; triazine antibody (AMI B5.1); triazine antibody (AM5C5.3); triazine antibody (AM5D1.2); triazine antibody (AM7B2. 1); triazine antibody (SA5A1.1); Triazine serum (metryne); Triazine serum (trazine); Triazine serum (antisimazine); Triazine serum (antisimetryne); Trifluralin antibody; Trifluralin antibody; Vincristine antibody; Zearalenone antibody; Zeatin riboside antibody; E2 G2 and E4 C2; Fab Fragment K411B derived from MAb K4E7 (isotype IgG2b with k light chain); LIB-BFNP23 Mab; MAbs H-7 and H-9 (against O,O-diethyl OP pestides); MoAb 33A7-1-1; MoAb 33B8-1-1; MoAb 33C3-1-1; MoAb 3C10-1-1 and MoAb 3EI7-1-1; MoAb 45D6-5-1; MoAb 45E6-1-1; MoAb 45-1-1; Mutant (GlnL89Glu) in Fab Fragment K411B derived from MAb K4E7 (isotype IgG2b with k light chain); Mutant (GlnL89Glu/ValH37Ile/GluL3Val) in Fab Fragment K411B derived from MAb K4E7 (isotype IgG2b with k light chain); Mutant (GlnL89GIuNaIH37IIe/GluL3Val) in Fab Fragment K411B derived from MAb K4E7 (isotype IgG2b with k light chain); Mutant (GlnL89Glu/ValH37Ile) in Fab Fragment K411B derived from MAb K4E7 (isotype IgG2b with k light chain); Mutant (GlnL89GIuNaIH37IIe) in Fab Fragment K411B derived from MAb K4E7 (isotype IgG2b with k light chain); Mutant (GluH50Gln) in Fab Fragment K411B derived from MAb K4E7 (isotype IgG2b with k light chain); Mutant (GluH50X) in Fab Fragment K411B derived from MAb K4E7 (isotype igG2b with k light chain); Mutant (GLyHI OOaAla) in Fab Fragment K411B derived from MAb K4E7 (isotype IgG2b with k light chain); Mutant (GLyHI OOaSer) in Fab Fragment K411 B derived from MAb K4E7 (isotype IgG2b with k light chain); Mutant (HisH95Phe) in Fab Fragment K411 B derived from MAb K4E7 (isotype IgG2b with k light chain); Mutant (HisH95Tyr) in Fab Fragment K411B derived from MAb K4E7 (isotype IgG2b with k light chain); Mutant (PheL32Leu) in Fab Fragment K411B derived from MAb K4E7 (isotype IgG2b with k light chain); Mutant (TrpH33Phe,Tyr,Leu) in Fab Fragment K411B derived from MAb K4E7 (isotype IgG2b with k light chain); Mutant (Try196Phe) in Fab Fragment K411B derived from MAb K4E7 (isotype IgG2b with k light chain); Mutant (TryL96Phe) in Fab Fragment K411B derived from MAb K4E7 (isotype IgG2b with k light chain); Mutant (ValH37Ile) in Fab Fragment K411B derived from MAb K4E7 (isotype IgG2b with k light chain); Mutant (ValH37Ile) in Fab Fragment K411B derived from MAb K4E7 (isotype IgG2b with k light chain); P6A7 MAb; PNAS2 6/3 56(1)-1-5-1; PNAS2 6/3 56(1)-1-5-2; PNAS2 6/3 56(1)-1-10-4; PNAS2 6/3 56(1)--10-5 and PNAS2 6/3 56(1)-3-1-5; Alexa Fluor 405/Cascade Blue dye antibody; Alexa Fluor 488 dye antibody; BODIPY FL dye antibody; Dansyl antibody; Fluorescein/Oregon Green dye antibody; Lucifer yellow dye antibody; Tetramethylrhodamine and Rhodamine Red dye antibody; Texas Red and Texas Red-X dye antibody; Biotin antibody; Dinitrophenyl antibody and Nitrotyrosine antibody.

Exemplary scFv that bind hapten are provided in FIG. 33 and include forms of FITCE2 scFv, FITCE2 TyrH133Ala scFv, FITCE2 HisH131Ala scFv, FL (4M5.3) scFv, FL (4D5FIu) scFv, FL (4420) scFv, and DNP scFv.

In particular embodiments, the binding domain can target a small molecule ligand linked to a targeting moiety. In particular embodiments, a small molecule ligand includes a folate, DUPA, an NK-1R ligand, a CAIX ligand, a ligand of gamma glutamyl transpeptidase, an NKG2D ligand, or a CCK2R ligand, each of which is a small molecule ligand that binds specifically to cancer cells (i.e., the receptor for these ligands is overexpressed on cancers compared to normal tissues). In particular embodiments, the targeting moiety includes fluorescein, fluorescein isothiocyanate (FITC), NHS and/or fluorescein. In particular embodiments, the binding domain is specific for the targeting moiety. In particular embodiments, the binding domain includes an E2 anti-fluorescein antibody or antibody fragment.

(iii) Intracellular Signaling Domains. An intracellular component of a protein includes one or more intracellular signaling domains. In particular embodiments, the intracellular signaling domain generates a signal that promotes an immune effector function of a CAR modified cell. In particular embodiments, the intracellular signaling domain generates a stimulatory, co-stimulatory or inhibitory signal based on ligand binding to an engineered stimulatory, co-stimulatory or inhibitory activity-inducible fusion protein. Examples of immune effector function include cytolytic activity and helper activity, including the secretion of cytokines. Intracellular signaling domain signals can also lead to immune cell proliferation, activation, differentiation, and the like.

A signaling domain refers to the functional portion of a protein which acts by transmitting information within the cell to regulate cellular activity via defined signaling pathways by generating second messengers or functioning as effectors by responding to such messengers. Stimulation refers to a primary response induced by binding of a stimulatory molecule (e.g., a CAR) or co-stimulatory molecule with its cognate ligand, thereby mediating a signal transduction event, such as signal transduction via appropriate signaling domains of the CAR or engineered receptor protein. Stimulation can mediate altered expression of certain molecules.

An intracellular signaling domain can include the entire intracellular portion of the signaling domain or a functional fragment thereof. In particular embodiments, an intracellular signaling domain can include a primary intracellular signaling domain. In particular embodiments, primary intracellular signaling domains include those derived from the molecules responsible for primary stimulation, or antigen dependent stimulation. In particular embodiments, the intracellular signaling domain can include a costimulatory intracellular domain.

A primary intracellular signaling domain can include a signaling motif which is known as an immunoreceptor tyrosine-based activation motif or ITAM. Examples of ITAM containing primary cytoplasmic signaling sequences include those derived from CD3ζ, common FcR gamma (FCER1G), Fc gamma Rlla, FcR beta (Fc Epsilon R1b), CD3 gamma, CD3 delta, CD3 epsilon, CD79a, CD79b, DAP10, and DAP12.

In particular embodiments, a CD3ζ (CD247) stimulatory domain can include amino acid residues from the cytoplasmic domain of the T cell receptor zeta chain, or functional fragments thereof, that are sufficient to functionally transmit an initial signal necessary for cell activation. In particular embodiments, a CD3ζ stimulatory domain can include a human CD3ζ stimulatory domain or functional fragments thereof. In particular embodiments, a CD3ζ stimulatory domain includes SEQ ID NO: 121. In particular embodiments, a CD3ζ stimulatory domain is encoded by SEQ ID NO: 124. In particular embodiments, in the case of an intracellular signaling domain that is derived from a CD3ζ molecule, the intracellular signaling domain retains sufficient CD3ζ structure such that it can generate a signal under appropriate conditions.

In particular embodiments, the intracellular signaling domain can include a costimulatory intracellular domain. In particular embodiments, costimulatory intracellular signaling domains include those derived from molecules responsible for costimulatory signals, or antigen independent stimulation. In particular embodiments, a costimulatory intracellular signaling domain can be the intracellular portion of a costimulatory molecule. A costimulatory molecule refers to a cognate binding partner on an immune cell that specifically binds with a costimulatory ligand, thereby mediating a costimulatory response by the immune cell, such as proliferation. Costimulatory molecules include cell surface molecules other than antigen receptors or their ligands that contribute to an efficient immune response. A costimulatory molecule can be represented in the following protein families: TNF receptor proteins, immunoglobulin-like proteins, cytokine receptors, integrins, signaling lymphocytic activation molecules (SLAM proteins), and activating NK cell receptors. Examples of such molecules include: an MHC class I molecule, B and T cell lymphocyte attenuator (BTLA, CD272), a Toll ligand receptor, CD27, CD28, 4-1BB (CD137), OX40, GITR, CD30, CD40, ICOS (CD278), BAFFR, HVEM (LIGHTR), ICAM-1, lymphocyte function-associated antigen-1 (LFA-1; CD11a/CD18), CD2, CDS, CD7, CD287, LIGHT, NKG2C, NKG2D, SLAMF7, NKp80 (KLRF1), NKp30, NKp44, NKp46, CD160 (BY55), B7-H3 (CD276), CD19, CD4, CD8a, CD8P, IL2Rβ, IL2Rγ, IL7Rα, ITGA4, VLA1, CD49a, IA4, CD49d, ITGA6, VLA-6, CD49f, ITGAD, CD11d, ITGAE, CD103, ITGAL, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, and the like.

In particular embodiments, a costimulatory intracellular signaling domain includes 4-1BB (CD137, TNFRSF9). 4-1BB refers to a member of the tumor necrosis factor receptor (TNFR) superfamily. In particular embodiments, a 4-1BB costimulatory domain includes a human 4-1BB costimulatory domain or a functional fragment thereof. In particular embodiments, a 4-1BB costimulatory domain includes SEQ ID NO: 120. In particular embodiments, a 4-1BB costimulatory domain is encoded by SEQ ID NO: 123.

In particular embodiments, a costimulatory intracellular signaling domain includes CD28. CD28 is a T cell-specific glycoprotein involved in T cell activation, the induction of cell proliferation and cytokine production, and promotion of T cell survival. In particular embodiments, a CD28 costimulatory domain includes a human CD28 costimulatory domain or a functional fragment thereof. In particular embodiments, a human CD28 costimulatory domain includes SEQ ID NO: 180. In particular embodiments, a human CD28 costimulatory domain is encoded by SEQ ID NO: 182.

In particular embodiments, an intracellular signaling domain includes a combination of one or more stimulatory domains and one or more costimulatory domains described herein. In particular embodiments, an intracellular signaling domain includes a 4-1BB costimulatory domain and a CD3ζ stimulatory domain. In particular embodiments, an intracellular signaling domain including a 4-1BB costimulatory domain and a CD3ζ stimulatory domain is set forth in SEQ ID NO: 130. In particular embodiments, an intracellular signaling domain including a 4-1BB costimulatory domain and a CD3ζ stimulatory domain is encoded by a sequence set forth in SEQ ID NO: 132 or SEQ ID NO: 131.

Inhibitory immune cell molecules that can be engineered to include an intracellular hsp90 binding domain include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD80, CD86, CD160, 2B4, B7-H3 (CD276), B7-H4 (VTCN1), KIR, A2aR, MHC class I, MHC class II, GAL9, adenosine, and TGFR β.

(iv) Transmembrane Domains. Fusion proteins can be designed to include a transmembrane domain that links an extracellular component of the protein to an intracellular component of the protein when expressed. A transmembrane domain can anchor a protein to a cell membrane. A transmembrane domain can include one or more additional amino acids adjacent to the transmembrane region, e.g., one or more amino acids associated with the extracellular region of the protein from which the transmembrane was derived (e.g., 1, 2, 3, 4, 5, 6, 7, 8, 9, 10, 11, 12, 13, 14, 15 amino acids, or more of the extracellular region) and/or one or more additional amino acids associated with the intracellular region of the protein from which the transmembrane protein is derived (e.g., 1, 2, 3, 4, 5, 6, 7, 8, 9, 10, 11, 12, 13, 14, 15 amino acids, or more of the intracellular region). In particular embodiments, the transmembrane domain may be from the same protein that the signaling domain, costimulatory domain, or hinge domain is derived from. In particular embodiments, the transmembrane domain is not derived from the same protein that any other domain of a fusion protein is derived from. In particular embodiments, the transmembrane domain can be selected or modified by amino acid substitution to avoid binding of or to minimize interactions with other domains in the fusion protein.

In particular embodiments, a transmembrane domain has a three-dimensional structure that is thermodynamically stable in a cell membrane, and generally ranges in length from 15 to 30 amino acids. The structure of a transmembrane domain can include an alpha helix, a beta barrel, a beta sheet, a beta helix, or any combination thereof.

The transmembrane domain may be derived either from a natural or from a recombinant source. Where the source is natural, the domain may be derived from any membrane-bound or transmembrane protein. In particular embodiments, the transmembrane domain is capable of signaling to the intracellular domain(s) whenever a fusion protein having an extracellular ligand binding domain has bound to a target. In particular embodiments, a transmembrane domain may include at least the transmembrane region(s) of: the a, s, or (chain of the T-cell receptor; CD28; CD27; CD3E; CD45; CD4; CD5; CD8; CD9; CD16; CD22; CD33; CD37; CD64; CD80; CD86; CD134; CD137; and/or CD154. In particular embodiments, a transmembrane domain may include at least the transmembrane region(s) of: KIRDS2; OX40; CD2; LFA-1; ICOS; 4-1 BB; GITR; CD40; BAFFR; HVEM; SLAMF7; NKp80; NKp44; NKp30; NKp46; CD160; CD19; IL2Rβ; IL2Rγ; IL7Ra; ITGA1; VLA1; CD49a; ITGA4; IA4; CD49D; ITGA6; VLA-6; CD49f; ITGAD; CDI Id; ITGAE; CD103; ITGAL; CDI la; ITGAM; CDI Ib; ITGAX; CDI Ic; ITGB1; CD29; ITGB2; CD18; ITGB7; TNFR2; DNAM1; SLAMF4; CD84; CD96; CEACAM1; CRT AM; Ly9; CD160; PSGL1; CD100; SLAMF6 (NTB-A, Ly108); SLAM; BLAME; SELPLG; LTBR; PAG/Cbp; NKG2D; and/or NKG2C. In particular embodiments, a transmembrane domain may include a transmembrane domain from CD28 or the CD8a chain. In particular embodiments, a CD8 transmembrane domain includes SEQ ID NO: 119 or 128 and/or is encoded by SEQ ID NO: 122.

In particular embodiments, the transmembrane domain can include predominantly hydrophobic residues such as leucine and valine. In particular embodiments, the transmembrane domain can include a triplet of phenylalanine, tryptophan and valine found at each end of the transmembrane domain. In particular embodiments, a CD28 or CD8 hinge is juxtaposed on the extracellular side of the transmembrane domain.

(v) Linkers. As used herein, a linker can be any portion of a fusion protein that serves to connect two subcomponents or domains of the fusion protein. In particular embodiments, linkers can provide flexibility for different components of the fusion protein. Linkers in the context of linking VH and VL of antibody derived binding domains of scFv are described above. Linkers can also include spacer regions and junction amino acids. In certain examples, when a more rigid linker is required, proline-rich linkers can be used.

Spacer regions are a type of linker region that are used to create appropriate distances and/or flexibility from other linked components.

In particular embodiments, the length of a spacer region can be customized for individual purposes. For example, a spacer region can be customized for individual cellular markers on targeted cells to optimize cell recognition and destruction following fusion protein binding. In certain examples, the spacer can be of a length that provides for increased responsiveness of a CAR expressing cell following antigen binding, as compared to in the absence of the spacer. In particular embodiments, a spacer region length can be selected based upon the location of a cellular marker epitope, affinity of a binding domain for the epitope, and/or the ability of the CAR modified cells to destroy target cells ex vivo and/or in vivo in response to cellular marker recognition. Spacer regions can also allow for high expression levels in CAR modified cells. In particular embodiments, an extracellular spacer region of a CAR is located between a transmembrane domain and the extracellular binding domain.

Exemplary spacers include those having 10 to 250 amino acids, 10 to 200 amino acids, 10 to 150 amino acids, 10 to 100 amino acids, 10 to 50 amino acids, or 10 to 25 amino acids. In particular embodiments, a spacer region is 12 amino acids, 20 amino acids, 21 amino acids, 26 amino acids, 27 amino acids, 45 amino acids, or 50 amino acids. In particular embodiments, a long spacer is greater than 119 amino acids, an intermediate spacer is 13-119 amino acids, and a short spacer is 10-12 amino acids.

In particular embodiments, a spacer region includes an immunoglobulin hinge region. An immunoglobulin hinge region may be a wild-type immunoglobulin hinge region or an altered wild-type immunoglobulin hinge region. In particular embodiments, an immunoglobulin hinge region is a human immunoglobulin hinge region. An immunoglobulin hinge region may be an IgG, IgA, IgD, IgE, or IgM hinge region. An IgG hinge region may be an IgG1, IgG2, IgG3, or IgG4 hinge region. In particular embodiments, the spacer region can include all or a portion of a hinge region sequence from IgG1, IgG2, IgG3, IgG4 or IgD alone or in combination with all or a portion of a CH2 region; all or a portion of a CH3 region; or all or a portion of a CH2 region and all or a portion of a CH3 region. As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

Exemplary spacers include IgG4 hinge alone, IgG4 hinge linked to CH2 and CH3 domains, or IgG4 hinge linked to the CH3 domain. In particular embodiments, the spacer includes an IgG4 linker as set forth in SEQ ID NOs: 78 or 80. Hinge regions can be modified to avoid undesirable structural interactions such as dimerization with unintended partners. Other examples of hinge regions that can be used in fusion proteins described herein include the hinge region present in extracellular regions of type 1 membrane proteins, such as CD8a, CD4, CD28, and CD7, which may be wild-type or variants thereof. In particular embodiments, a hinge includes a CD8a hinge set forth in SEQ ID NO: 129.

In particular embodiments, a spacer region includes a hinge region of a type II C-lectin interdomain (stalk) region or a cluster of differentiation (CD) molecule stalk region. A “stalk region” of a type II C-lectin or CD molecule refers to the portion of the extracellular domain of the type II C-lectin or CD molecule that is located between the C-type lectin-like domain (CTLD; e.g., similar to CTLD of natural kiIler cell receptors) and the hydrophobic portion (transmembrane domain).

For example, the extracellular domain of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al., Immunity 10:15, 1999; for descriptions of other stalk regions, see also Beavil et al., Proc. Nat'l. Acad. Sci. USA 89:153, 1992; and Figdor et al., Nat. Rev. Immunol. 2:11, 2002). These type II C-lectin or CD molecules may also have junction amino acids between the stalk region and the transmembrane region or the CTLD. In another example, the 233 amino acid human NKG2A protein (UniProt ID P26715.1) has a hydrophobic portion (transmembrane domain) ranging from amino acids 71-93 and an extracellular domain ranging from amino acids 94-233. The CTLD includes amino acids 119-231 and the stalk region includes amino acids 99-116, which may be flanked by additional junction amino acids. Other type II C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

Junction amino acids can be a linker which can be used to connect the sequences of fusion protein domains when the distance provided by a spacer is not needed and/or wanted. In particular embodiments, junction amino acids are short amino acid sequences that can be used to connect intracellular signaling domains. In particular embodiments, junction amino acids are 9 amino acids or less.

Junction amino acids can be a short oligo- or protein linker, preferably between 2 and 9 amino acids (e.g., 2, 3, 4, 5, 6, 7, 8, or 9 amino acids) in length to form the linker. In particular embodiments, a glycine-serine doublet can be used as a suitable junction amino acid linker. In particular embodiments, a single amino acid, e.g., an alanine, a glycine, can be used as a suitable junction amino acid. CAR constructs disclosed herein can additionally utilize Gly3 as a junction amino acid sequence.

(vi) Tags and Selectable Markers. In particular embodiments, a fusion protein can include one or more tags and/or be expressed with one more selectable markers. Exemplary tags include His tag, Flag tags, Xpress tag, Avi tag, Calmodulin binding peptide (CBP) tag, Polyglutamate tag, HA tags, Myc tag, Strep tag (which refers to the original STREP® tag, STREP® tag II (IBA Institut fur Bioanalytik, Germany); see, e.g., U.S. Pat. No. 7,981,632), Softag 1, Softag 3, and V5. See FIG. 33 for exemplary sequences.

Conjugate binding molecules that specifically bind tag sequences disclosed herein are commercially available. For example, His tag antibodies are commercially available from suppliers including Life Technologies, Pierce Antibodies, and GenScript. Flag tag antibodies are commercially available from suppliers including Pierce Antibodies, GenScript, and Sigma-Aldrich. Xpress tag antibodies are commercially available from suppliers including Pierce Antibodies, Life Technologies, and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal, and Abcam. Myc tag antibodies are commercially available from suppliers including Santa Cruz Biotechnology, Abcam, and Cell Signal. Strep tag antibodies are commercially available from suppliers including Abcam, Iba, and Qiagen.

In particular embodiments, one or more transduction markers can be co-expressed with the fusion protein, for example, using a skipping element or IRES site that allows expression of the transduction marker and other components of the fusion protein as distinct molecules. Exemplary self-cleaving polypeptides include 2A peptides from porcine teschovirus-1 (P2A), Thosea asigna virus (T2A), equine rhinitis A virus (E2A), and foot-and-mouth disease virus (F2A) (see, e.g., FIG. 33).

In particular embodiments, the transduction marker can include any cell surface displayed marker that can be detected with an antibody that binds to that marker and allows sorting of cells that have the marker. In particular embodiments, the transduction marker can include the magnetic sortable marker streptavidin binding peptide (SBP) displayed at the cell surface by a truncated Low Affinity Nerve Growth Receptor (LNGFRF) and one-step selection with streptavidin-conjugated magnetic beads (Matheson et al. (2014) PloS one 9(10): e111437) or a truncated human epidermal growth factor receptor (EGFR) (tEGFR; see Wang et al., Blood 118: 1255, 2011).

In some alternatives, the transduction marker is a truncated EGFR (EGFRt), a truncated Her2 (Her2), a truncated Her2 (Her2tG), a truncated CD19 (CD19t), or the transduction marker DHFRdm.

Transduction markers can include any suitable fluorescent protein including: blue fluorescent proteins (e.g., BFP, eBFP, eBFP2); cyan fluorescent proteins (e.g., eCFP, Cerulean, CyPet); green fluorescent proteins (e.g., GFP-2, tagGFP, turboGFP, eGFP,); orange fluorescent proteins (e.g., mOrange, mKO, Kusabira-Orange); red fluorescent proteins (e.g., mKate, mPlum, DsRed monomer, mCherry, mRFP1, DsRed-Express); yellow fluorescent proteins (e.g., YFP, eYFP, Citrine, Venus); and any other suitable fluorescent proteins, including, for example, firefly luciferase. In particular embodiments, the transduction marker includes a BFP as shown in FIG. 33. See also Heim and Tsien (1996) Current Biology, 6(2): 178-182; Yang et al. (1998) Journal of Biological Chemistry, 273(14): 8212-8216; Ai et al. (2007) Biochemistry, 46(20): 5904-5910; and Constantini et al. (2015) Nature Communications, 6(1): 7670).

(vii) Additional Transmembrane Receptors. The current disclosure also includes stimulatory, co-stimulatory and inhibitory immune receptors engineered to be activity-inducible based on the presence of an intracellular hsp90 binding domain.

Stimulatory receptors include, for example, CD3.

Co-stimulatory immune cell molecules that can be engineered to include an intracellular hsp90 binding domain include 4-1EE, OX40, CD40, CD30, CD27, DR3, SLAMF1, ICOS, GITR, CD25, CD28, CD79A, CD79B, CD226, CARD11, DAP10, DAP12, DR3, FcRα, FcRβ, FcRγ, Fyn, Lck, LAT, LRP, LIGHT, NKG2D, NOTCH1, NOTCH2, NOTCH3, NOTCH4, ROR2, Ryk, Slp76, pTa, TCRα, TCRβ, TIM1, TRIM, Zap70, and PTCH2. Inhibitory immune cell molecules that can be engineered to include an intracellular hsp90 binding domain include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD80, CD86, CD160, 2B4, B7-H3 (CD276), B7-H4 (VTCN1), KIR, A2aR, MHC class I, MHC class II, GAL9, adenosine, and TGFR β. Certain embodiments include an EBD linked to the intracellular signaling domain of 4-1 BB, OX40, CD40, CD30, CD27, DR3, SLAMF1, ICOS, GITR, CD25, CD28, CD79A, CD79B, CD226, CARD11, DAP10, DAP12, DR3, FcRα, FcRβ, FcRγ, Fyn, Lck, LAT, LRP, LIGHT, NKG2D, NOTCH1, NOTCH2, NOTCH3, NOTCH4, ROR2, Ryk, Slp76, pTα, TCRα, TCRβ, TIM1, TRIM, Zap70, PTCH2, PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD80, CD86, CD160, 2B4, B7-H3 (CD276), B7-H4 (VTCN1), KIR, A2aR, MHC class I, MHC class II, GAL9, adenosine, or TGFR β.

(viii) Cells Genetically Modified to Express Activity-Inducible Fusion Proteins. The present disclosure includes cells genetically modified to express an activity-inducible fusion protein. As used herein, the term “genetically modified” or “genetically engineered” refers to the addition of extra genetic material in the form of DNA or RNA into the total genetic material in a cell. The terms “genetically modified cells” and “modified cells” are used interchangeably. In particular embodiments, a cell genetically modified to express an activity-inducible fusion protein includes an immune effector cell. An “immune effector cell” includes any cell of the immune system that has one or more effector functions (e.g., cytotoxic cell killing activity, secretion of cytokines, induction of antibody-dependent cell cytotoxicity (ADCC) and/or complement-dependent cytotoxicity (CDC). Immune effector cells are a subtype of immune cells.

Immune cells of the disclosure can be autologous/autogeneic (“self”) or non-autologous (“non-self,” e.g., allogeneic, syngeneic or xenogeneic). “Autologous” refers to cells from the same subject. “Allogeneic” refers to cells of the same species that differ genetically to a cell in comparison. “Syngeneic” refers to cells of a different subject that are genetically identical to the cell in comparison. “Xenogeneic” refers to cells of a different species to the cell in comparison. In particular embodiments, modified cells of the disclosure are autologous or allogeneic.

In particular embodiments, genetically modified cells include lymphocytes. In particular embodiments, genetically modified cells include T cells, B cells, natural kiIler (NK) cells, monocytes/macrophages, or HSPC.

Most T cells have a T-cell receptor (TCR) composed of two separate peptide chains (the α- and β-TCR chains). γd T cells represent a small subset of T cells that possess a distinct T cell receptor (TCR) made up of one γ-chain and one d-chain.

CD3 is expressed on all mature T cells. T cells can further be classified into cytotoxic T cells (CD8+ T cells, also referred to as CTLs) and helper T cells (CD4+ T cells).

Cytotoxic T cells destroy virally infected cells and tumor cells and are also implicated in transplant rejection. These cells recognize their targets by binding to antigen associated with MHC class I, which is present on the surface of nearly every cell of the body.

Central memory T cells (TCM) refer to antigen experienced CTL that express CD62L or CCR7 and CD45RO and does not express or has decreased expression of CD45RA as compared to naive cells.

Effector memory T cells (TEM) refer to an antigen experienced T-cell that does not express or has decreased expression of CD62L as compared to central memory cells and does not express or has decreased expression of CD45RA as compared to a naive cell. In particular embodiments, effector memory T cells are negative for expression of CD62L and CCR7, compared to naive cells or central memory cells, and have variable expression of CD28 and CD45RA. Effector T cells are positive for granzyme B and perforin as compared to memory or naive T cells.

Helper T cells assist other immune cells such as activating of cytotoxic T cells and macrophages and facilitating the maturation of B cells, among other functions. Helper T cells become activated when they are presented with peptide antigens by MHC class II molecules that are expressed on the surface of antigen presenting cells (APCs). Once activated, they divide rapidly and secrete cytokines that regulate or assist in the active immune response.

Natural kiIler T (NKT) cells are a subset of T cells that co-express an ap T-cell receptor, but also express a variety of molecular markers that are typically associated with natural kiIler cells, such as NK1.1 (CD161), CD16, and/or CD56.

Natural kiIler cells (also known as K cells and kiIler cells) express CD8, CD16 and CD56 but do not express CD3. NK cells also express activating receptors such as NKp46 and inhibitory receptors such as NKG2A that regulate NK cell cytotoxic function against tumor and virally infected cells.

Tumor-infiltrating lymphocytes (TILs) refers to immune cells that have moved from the blood into a tumor and can function to recognize and kill cancer cells. Marrow-infiltrating lymphocytes (MILs) are antigen-experienced immune cells that travel to and remain in the bone marrow. Mucosal-associated invariant T (MAIT) cells are innate-like T cells which are found in the mucosa, blood, and secondary lymphoid organs (SLO), and display effector phenotype. MAIT cells display a semi-invariant T cell receptor (TCR) and are restricted by the major histocompatibility complex related molecule, MR1.

Macrophages (and their precursors, monocytes) reside in every tissue of the body where they engulf apoptotic cells, pathogens and other non-self-components. Monocytes/macrophages express CD11b, F4/80, CD68, CD11c, IL-4Rα, and/or CD163.

Immature dendritic cells (i.e., pre-activation) engulf antigens and other non-self-components in the periphery and subsequently, in activated form, migrate to T cell areas of lymphoid tissues where they provide antigen presentation to T cells. Dendritic cells express CD1a, CD1b, CD1c, CD1d, CD21, CD35, CD39, CD40, CD86, CD101, CD148, CD209, and DEC-205.

Hematopoietic stem cells (HSC) refer to undifferentiated hematopoietic cells that are capable of self-renewal and differentiation into all other hematopoietic cell types. HSC are CD34+.

Hematopoietic progenitor cells (HPC) are derived from HSC and are capable of further differentiation into mature cell types. HPC can self-renew or can differentiate into (i) myeloid progenitor cells which ultimately give rise to monocytes and macrophages, neutrophils, basophils, eosinophils, erythrocytes, megakaryocytes/platelets, or dendritic cells; or (ii) lymphoid progenitor cells which ultimately give rise to T cells, B cells, and NK cells. HPC are CD24^{lo} Lin⁻ CD117⁺.

HSPC refer to a cell population having HSC and HPC. HSPC cell populations can be positive for CD34, CD43, CD45RO, CD45RA, CD59, CD90, CD109, CD117, CD133, CD166, HLA DR, or a combination thereof.

Induced pluripotent stem cells (iPSCs) refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by introducing or contacting with reprogramming factors.

(ix) Methods to Modify Cells Ex Vivo and In Vivo. The present disclosure provides methods for collecting, enriching for, culturing, and modifying cells to express an activity-inducible fusion protein (e.g. a CAR) ex vivo and/or genetically modifying immune cells in vivo utilizing cell-targeted delivery methods.

In particular embodiments, lymphocytes are isolated from a sample such as blood or a blood-derived sample, an apheresis or a leukapheresis product. Exemplary samples include whole blood, peripheral blood mononuclear cells (PBMCs), bone marrow, thymus, cancer tissue, lymphoid tissue, spleen, or other appropriate sources.

Sources of HSPC include, for example, peripheral blood (see U.S. Pat. Nos. 5,004,681; 7,399,633; and U.S. Pat. No. 7,147,626; Craddock, et al., 1997, Blood 90(12):4779-4788; Jin, et al., 2008, Journal of Translational Medicine 6:39; Pelus, 2008, Curr. Opin. Hematol. 15(4):285-292; Papayannopoulou, et al., 1998, Blood 91(7):2231-2239; Tricot, et al., 2008, Haematologica 93(11):1739-1742; and Weaver et al., 2001, Bone Marrow Transplantation 27(2):S23-S29).

Methods regarding collection, anti-coagulation and processing, etc. of blood samples can be found in, for example, Alsever, et al., 1941, N.Y. St. J. Med. 41:126; De Gowin, et al., 1940, J. Am. Med. Ass. 114:850; Smith, et al., 1959, J. Thorac. Cardiovasc. Surg. 38:573; Rous and Turner, 1916, J. Exp. Med. 23:219; and Hum, 1968, Storage of Blood, Academic Press, New York, pp. 26-160.

In particular embodiments, collected cells are washed, centrifuged, and/or incubated in the presence of one or more reagents, for example, to remove unwanted components, enrich for desired components, lyse or remove cells sensitive to particular reagents. The isolation can include one or more of various cell preparation and separation steps, including separation based on one or more properties, such as size, density, sensitivity or resistance to particular reagents, and/or affinity, e.g., immunoaffinity, to antibodies or other binding partners.

In particular embodiments, one or more of the cell populations enriched, isolated and/or selected from a sample by the provided methods are cells that are positive for (marker+) or express high levels (marker^{hi}) of one or more particular markers, such as surface markers, or that are negative for (marker−) or express relatively low levels (marker^{lo}) of one or more markers.

In particular embodiments, T cells can be isolated from peripheral blood mononuclear cells (PBMCs) by lysing the red blood cells and depleting the monocytes, for example, by centrifugation through a PERCOLL™ gradient. In particular embodiments, a specific subpopulation of T cells, expressing CD3, CD28, CD4, CD8, CD45RA, and CD45RO is further isolated by positive or negative selection techniques. In particular embodiments, cell sorting and/or selection occurs via negative magnetic immunoadherence or flow cytometry using a cocktail of monoclonal antibodies directed to cell surface markers present on the cells negatively selected. For example, to enrich for CD4⁺ cells by negative selection, a monoclonal antibody cocktail that typically includes antibodies to CD14, CD20, CD11b, CD16, HLA-DR, and CD8 can be used.

Following isolation and/or enrichment, cells can be expanded to increase the number of cells. In particular embodiments, T cells can be activated and expanded before or after genetic modification to express a CAR, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and US 2006/0121005.

Generally, the T cells are expanded by contact with a surface having attached thereto an agent that stimulates a CD3 TCR complex associated signal and a ligand that stimulates a co-stimulatory molecule on the surface of the T cells. In particular embodiments, PBMCs or isolated T cells are contacted with a stimulatory agent and costimulatory agent, such as anti-CD3 and anti-CD28 antibodies, generally attached to a bead or other surface, in a culture medium with appropriate cytokines (see Berg et al., Transplant Proc. 30(8):3975-3977, 1998; Haanen et al., J. Exp. Med. 190(9): 13191328, 1999; Garland et al., J. Immunol Meth. 227(1-2):53-63, 1999). In particular embodiments, the T cells may be activated and stimulated to proliferate with feeder cells and appropriate antibodies and cytokines using methods such as those described in U.S. Pat. Nos. 6,040,177; 5,827,642; and WO 2012/129514.

In particular embodiments, artificial APC (aAPC) can be made by engineering K562, U937, 721.221, T2, and C1R cells to direct the stable expression and secretion of a variety of co-stimulatory molecules and cytokines. aAPCs are described in WO 03/057171 and US 2003/0147869.

In particular embodiments, HSPCs can be isolated and/or expanded following methods described in, for example, U.S. Pat. Nos. 7,399,633; 5,004,681; US 2010/0183564; WO2006/047569; WO2007/095594; WO 2011/127470; and WO 2011/127472; Vamum-Finney, et al., 1993, Blood 101:1784-1789; Delaney, et al., 2005, Blood 106:2693-2699; Ohishi, et al., 2002, J. Clin. Invest. 110:1165-1174; Delaney, et al., 2010, Nature Med. 16(2): 232-236; and Chapter 2 of Regenerative Medicine, Department of Health and Human Services, August 2006, and the references cited therein. The collection and processing of other cell types described herein are known by one of ordinary skill in the art.

In particular embodiments, the isolating, incubating, expansion, and/or engineering steps are carried out in a sterile or contained environment and/or in an automated fashion, such as controlled by a computer attached to a device in which the steps are performed. Final formulation of modified cells into modified formulations for administration is described elsewhere herein.

Targeted viral vectors and/or nanoparticles can also be used to genetically-modify immune cells in vivo or ex vivo. Viral vectors that can be used to deliver fusion protein-encoding genes to cells are described elsewhere herein, and numerous targeted (e.g., pseudotyped) viral vectors are known in the art.

Exemplary cell-targeted nanoparticles include a cell targeting ligand (e.g., CD3, CD4, CD8, CD34) on the surface of the nanoparticle wherein the cell targeting ligand results in selective uptake of the nanoparticle by a selected cell type. The nanoparticle then delivers gene modifying components that result in expression of the activity-inducible fusion protein.

Exemplary nanoparticles include liposomes (microscopic vesicles including at least one concentric lipid bilayer surrounding an aqueous core), liposomal nanoparticles (a liposome structure used to encapsulate another smaller nanoparticle within its core); and lipid nanoparticles (liposome-like structures that lack the continuous lipid bilayer characteristic of liposomes). Other polymer-based nanoparticles can also be used as well as porous nanoparticles constructed from any material capable of forming a porous network. Exemplary materials include metals, transition metals and metalloids (e.g., lithium, magnesium, zinc, aluminum and silica).

For in vivo delivery and cellular uptake, nanoparticles can have a neutral or negatively-charged coating and a size of 130 nm or less. Dimensions of the nanoparticles can be determined using, e.g., conventional techniques, such as dynamic light scattering and/or electron microscopy.

(x) Production of Activity-Inducible Fusion Proteins. An activity-inducible fusion protein according to the present disclosure can be produced by any methods known in the art. In particular embodiments, an activity-inducible fusion protein is produced using recombinant DNA techniques. A nucleic acid encoding the several regions of the activity-inducible fusion protein can be prepared and assembled into a complete coding sequence by standard techniques of molecular cloning. The resulting coding regions can be inserted into an expression vector and used to transform a cell or cell line.

The term “gene” refers to a nucleic acid sequence (used interchangeably with polynucleotide or nucleotide sequence) that encodes an activity-inducible fusion protein, components of an activity-inducible fusion protein, or a molecule co-expressed with a an activity-inducible fusion protein as described herein. This definition includes various sequence polymorphisms, mutations, and/or variants wherein such alterations do not substantially affect the function of the encoded protein. The term “gene” may include not only coding sequences but also regulatory regions such as promoters, enhancers, and termination regions. Gene sequences encoding a molecule can be DNA or RNA that directs the expression of the activity-inducible fusion protein. These nucleic acid sequences may be a DNA strand sequence that is transcribed into RNA or an RNA sequence that is translated into protein.

“Encoding” refers to the property of specific sequences of nucleotides in a gene, such as a complementary DNA (cDNA), or a messenger RNA (mRNA), to serve as templates for synthesis of other macromolecules such as a defined sequence of amino acids. Thus, a gene codes for a protein if transcription and translation of mRNA corresponding to that gene produces the protein in a cell or other biological system. A “gene encoding a protein” includes all nucleotide sequences that are degenerate versions of each other and that code for the same amino acid sequence or amino acid sequences of substantially similar form and function.

Polynucleotide gene sequences encoding more than one portion of an expressed activity-inducible fusion protein can be operably linked to each other and relevant regulatory sequences. For example, there can be a functional linkage between a regulatory sequence and an exogenous nucleic acid sequence resulting in expression of the latter. For another example, a first nucleic acid sequence can be operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary or helpful, join coding regions, into the same reading frame.

In an exemplary nucleic acid construct employed in the present disclosure, the promoter is operably linked to the nucleic acid sequence encoding an activity-inducible fusion protein, i.e., they are positioned so as to promote transcription of mRNA from the DNA encoding the activity-inducible fusion protein. The promoter can be of genomic origin or synthetically generated. The promoters may or may not be associated with enhancers, wherein the enhancers may be naturally associated with the particular promoter or associated with a different promoter. A variety of promoters for use in cells are well-known in the art (e.g., a CD4 promoter). The promoter can be constitutive or inducible, where induction is associated with a specific cell type or a specific stage of development, for example. Alternatively, a number of well-known viral promoters are also suitable. Promoters of interest include: a viral simian virus 40 (SV40) (e.g., early or late) promoter; a Moloney murine leukemia virus (MoMLV) long terminal repeat (LTR) promoter; a Rous sarcoma virus (RSV) LTR promoter; a herpes simplex virus (HSV) (thymidine kinase) promoter; a glyceraldehyde 3-phosphate dehydrogenase (GAPDH) promoter; heat shock protein 70 kDa (HSP70) promoter; a Ubiquitin C (UBC) promoter; or a phosphoglycerate kinase-1 (PGK) promoter.

In certain embodiments, a signal sequence directing the activity-inducible fusion protein to the surface membrane can be used and can include an endogenous signal sequence of the N-terminal component of the activity-inducible fusion protein. Optionally, in some instances, it may be desirable to exchange this sequence for a different signal sequence. However, the signal sequence selected should be compatible with the secretory pathway of the activity-inducible fusion protein expressing cells so that the activity-inducible fusion protein is presented on the surface of its expressing cell. Particular embodiments disclosed herein utilize a GM-CSF signal peptide or a CD8 signal peptide.

Similarly, a termination region may be provided by the naturally occurring or endogenous transcriptional termination region of the nucleic acid sequence encoding the C-terminal component of the activity-inducible fusion protein. Alternatively, the termination region may be derived from a different source. For the most part, the source of the termination region is generally not considered to be critical to the expression of a recombinant protein and a wide variety of termination regions can be employed without adversely affecting expression.

As will be appreciated by one of skill in the art, in some instances, a few amino acids at the ends of the binding domain in an activity-inducible fusion protein (e.g., a CAR) can be deleted, usually not more than 10, more usually not more than 5 residues, for example. Also, it may be desirable to introduce a small number of amino acids at the borders, usually not more than 10, more usually not more than 5 residues. The deletion or insertion of amino acids may be as a result of the needs of the construction, providing for convenient restriction sites, ease of manipulation, improvement in levels of expression, or the like. In addition, the substitute of one or more amino acids with a different amino acid can occur for similar reasons.

In any of the embodiments described herein, a polynucleotide can include a sequence that encodes a self-cleaving polypeptide between the polynucleotide segment encoding the activity-inducible fusion protein and a polynucleotide encoding a selection (e.g., transduction) marker (e.g., EGFRt, Her2tG, CD19t, or DHFRdm). Exemplary nucleic acid sequences encoding 2A peptides are set forth in, for example, Kim et al. (PLOS One 6:e18556 (2011)) and Donnelly et al. (J. Gen. Virol. 82:1027-1041 (2001)).

Desired genes encoding activity-inducible fusion proteins can be introduced into cells by any method known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, mammalian artificial chromosomes (Vos, 1998, Curr. Op. Genet. Dev. 8:351-359), liposomes (Tarahovsky and Ivanitsky, 1998, Biochemistry (Mosc) 63:607-618), ribozymes (Branch and Klotman, 1998, Exp. Nephrol. 6:78-83), triplex DNA (Chan and Glazer, 1997, J. Mol. Med. 75:267-282), etc. Numerous techniques are known in the art for the introduction of foreign genes into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the gene to the cell, so that the gene is expressed by the cell and, in certain instances, preferably heritable and expressed in its cell progeny.

In particular embodiments, a gene encoding an activity-inducible fusion protein can be introduced into cells in a vector. A “vector” is a nucleic acid molecule that is capable of transporting another nucleic acid. Vectors may be, e.g., plasmids, cosmids, viruses, or phage. An “expression vector” is a vector that is capable of directing the expression of a protein encoded by one or more genes carried by the vector when it is present in the appropriate environment.

Viral vectors can be derived from numerous viruses. “Lentivirus” refers to a genus of retroviruses that are capable of infecting dividing and non-dividing cells and typically produce high viral titers. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

Additional examples of viral vectors include those derived from foamy viruses, adenoviruses (e.g., adenovirus 5 (Ad5), adenovirus 35 (Ad35), adenovirus 11 (Ad11), adenovirus 26 (Ad26), adenovirus 48 (Ad48) or adenovirus 50 (Ad50)), adeno-associated virus (AAV; see, e.g., U.S. Pat. No. 5,604,090; Kay et al., 2000; Nakai et al., z1998), alphaviruses, cytomegaloviruses (CMV), flaviviruses, herpes viruses (e.g., herpes simplex), influenza viruses, papilloma viruses (e.g., human and bovine papilloma virus; see, e.g., U.S. Pat. No. 5,719,054), poxviruses, vaccinia viruses, etc. See Kozarsky and Wilson, 1993; Rosenfeld, et al., 1991; Rosenfeld, et al., 1992; Mastrangeli, et al., 1993; Walsh, et al., 1993; and Lundstrom, 1999. Examples include modified vaccinia Ankara (MVA) and NYVAC, or strains derived therefrom. Other examples include avipox vectors, such as a fowlpox vectors (e.g., FP9) or canarypox vectors (e.g., ALVAC and strains derived therefrom). For additional information regarding viral vectors for gene delivery, see Kozarsky and Wilson, 1993, Current Opinion in Genetics and Development 3:499-503, Rosenfeld, et al., 1991, Science 252:431-434; Rosenfeld, et al., 1992, Cell 68:143-155; Mastrangeli, et al., 1993, J. Clin. Invest. 91:225-234; Walsh, et al., 1993, Proc. Soc. Exp. Bioi. Med. 204:289-300; and Lundstrom, 1999, J. Recept. Signal Transduct. Res. 19: 673-686; Miller, et al., 1993, Meth. Enzymol. 217:581-599); Naldini et al. (1996) Science 272(5259): 263-267; Naldini et al. (1996) Proceedings of the National Academy of Sciences 93(21): 11382-11388; Zufferey et al. (1997) Nature biotechnology 15(9): 871-875; Dull et al. (1998) Journal of virology 72(11): 8463-8471; U.S. Pat. Nos. 6,013,516; and 5,994,136).

Targeted genetic engineering approaches may also be utilized. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. ZFNs are a class of site-specific nucleases engineered to bind and cleave DNA at specific positions. For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. For additional information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14(1):49-55; Beurdeley et al., Nat Commun, 2013, 4: 1762; Scharenberg et al., Curr Gene Ther, 2013, 13(4):291-303; Gaj et al., Nat Methods, 2012, 9(8):805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

Cells that have been successfully genetically modified to express an activity-inducible fusion protein ex vivo can be sorted based on, for example, expression of a transduction marker, and further processed.

(xi) Modified Formulations, Modifying Formulations, and Drug Compositions. Formulations described herein can include ex vivo genetically modified cells (i.e., modified formulations) or can include viral vectors or nanoparticles that result in in vivo genetic modification of cells to express a CAR (modifying formulations). As used herein, compositions include a drug molecule that binds an hsp90 binding domain present on an expressed activity-inducible fusion protein and/or results in a conformation change of the activity-inducible fusion protein such that intracellular signaling occurs upon ligand binding.

A “pharmaceutical” formulation or composition includes an active compound for administration (e.g., a genetically modified cell, viral vector, nanoparticle, or drug molecule) within a pharmaceutically-acceptable carrier.

The phrase “pharmaceutically acceptable” refer to those compounds, materials, and/or dosage forms which are, within the scope of sound medical judgment, suitable for use in contact with the tissues of human beings and animals without excessive toxicity, commensurate with a reasonable benefit/risk ratio. In certain instances, pharmaceutically-acceptable carriers have been approved by a relevant regulatory agency (e.g., the United States Food and Drug Administration (US FDA)).

Depending on the context and active compound for delivery, “pharmaceutically acceptable carriers” includes any adjuvant, excipient, glidant, diluent, preservative, dye/colorant, surfactant, wetting agent, dispersing agent, suspending agent, stabilizer, isotonic agent, solvent, surfactant, or emulsifier which meets the requirements noted above. Exemplary pharmaceutically acceptable carriers are disclosed in Remington's Pharmaceutical Sciences, 18th Ed. Mack Printing Company, 1990. Moreover, formulations and compositions can be prepared to meet sterility, pyrogenicity, general safety, and purity standards as required by the US FDA Office of Biological Standards and/or other relevant foreign regulatory agencies.

Exemplary pharmaceutically-acceptable carriers include saline, buffered saline, physiological saline, water, Hanks' solution, Ringer's solution, Nonnosol-R (Abbott Labs), PLASMA-LYTE A® (Baxter Laboratories, Inc., Morton Grove, IL), glycerol, ethanol, and combinations thereof. In particular embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols, amino acids, organic sugars or sugar alcohols, PEG, sulfur-containing reducing agents, bovine serum albumin, gelatin or immunoglobulins, polyvinylpyrrolidone, and saccharides.

Where necessary or beneficial, formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens, catechol, resorcinol, cyclohexanol, and 3-pentanol.

Therapeutically effective amounts of cells within modified formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In modified formulations disclosed herein, cells are generally in a volume of a liter or less, 500 ml or less, 250 ml or less, or 100 ml or less. Hence the density of administered cells is typically greater than 10⁴ cells/ml, 10⁷ cells/ml, or 10⁸ cells/ml.

Therapeutically effective amounts of active ingredients (vectors, nanoparticles) within modifying formulations can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts of drug molecules within compositions can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

In particular embodiments, modified formulations can include one or more genetically modified cell type (e.g., modified T cells, NK cells, or stem cells) or genetically modified cells that express one or more activity-inducible fusion protein types. The different populations of genetically modified cells can be provided in different ratios. Further, modifying formulations can deliver nucleic acids resulting in the genetic modification of more than one cell type and/or the expression of different activity-inducible fusion proteins.

Certain modified formulations include immune cells that express more than one CAR type. For example, an immune cell (e.g., a T cell) can be modified to express different CAR wherein the different CAR have different ligand binding domains. The different ligand binding domains can bind different epitopes on a same cancer antigen or can bind different cancer antigens. In this scenario, the CAR that bind different epitopes or antigens can also include different hsp90 binding domains, so that their activation states can be individually controlled by administration of different drug molecules (e.g., small molecule estrogen analogs).

Certain formulations can result in the expression of multiple activity-inducible fusion proteins, wherein the activity-inducible fusion proteins can be individually activated or inactivated through inclusion of different EBD. The different EBD can include EBD (E353A), activatable by the administration of ES8, EBD (L384M, M421G, G521R), activatable by the administration of CMP8, and EBD ERT2, activatable by the administration of 4-OHT. Numerous additional combinations are possible, based on the content of the current disclosure.

Exemplary combinations of cancer antigens bound by immune cells expressing different CAR and hsp90 binding domains include (i) CD19, CD22, and/or BAFF-R; (ii) CD19 and CD22; (iii) Her2, B7H3, EGFR, and/or IL13Ra2; and (iv) CD33 and CD123. Other relevant groupings of cancer antigens by cancer type are described elsewhere within this disclosure. Different EBD/drug molecule combinations include EBD (E353A) with ES8, EBD (L384M, M421G, G521R) with CMP8, and EBD with G400V, M543A, and L544A mutations (ERT2) with 4-OHT. This approach of utilizing different binding domains with different hsp90 binding domains is referred to herein as a CAR combination therapy.

In certain examples, formulations result in the expression of a CAR and a co-stimulatory immune molecule (e.g., CD28, 4-1BB, OX40, ICOS) wherein the CAR and the co-stimulatory immune molecule each include a different hsp90 binding domain. In certain examples, formulations result in the expression of a CAR and a two different co-stimulatory immune molecules (e.g., CD28, 4-1BB, OX40, ICOS) wherein the CAR and the different co-stimulatory immune molecules each include a different hsp90 binding domain. In certain examples, formulations result in the expression of two CAR types and a two different co-stimulatory immune molecules (e.g., CD28, 4-1EE, OX40, ICOS) wherein the two CAR types have different binding domains and different hsp90 binding domains while the different co-stimulatory immune molecules each include the same hsp90 binding domain. In certain examples, formulations result in the expression of two CAR types and a two different co-stimulatory immune molecules (e.g., CD28, 4-1BB, OX40, ICOS) wherein the two CAR types have different binding domains and different hsp90 binding domains and the different co-stimulatory immune molecules each include a different hsp90 binding domain. In this example, the hsp90 binding domains of the co-stimulatory molecules can match that of a CAR or be distinct from the hsp90 binding domains of the CAR.

Modified formulations can also include different immune cells expressing different CAR. For example, individually modified immune cells express only one type of CAR but are formulated with immune cells modified to express different types of CAR (e.g., different ligand binding domains associated with different EBD/drug molecule combinations). The immune cells can be of the same type (all T cells) or can include a mixture of different types (e.g., T cells, NK cells, and/or HSPC).

Modifying formulations can also be prepared to lead to in vivo populations of immune cells having these characteristics (e.g., expression of different CAR types by a single immune cell; expression of a different CAR types by different immune cells; expression of a same CAR type by different types of immune cells; and/or expression of different CAR types by different types of immune cells; inclusion of activity-inducible co-stimulatory or inhibitory molecules).

Formulations and compositions can be prepared for administration by, e.g., injection, infusion, perfusion, lavage, or ingestion. The formulations and compositions can further be formulated for bone marrow, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous injection.

In some instances, it can be useful to cryopreserve modified cell formulations of the disclosure. As used herein, “cryopreserving,” refers to the preservation of cells by cooling to sub-zero temperatures, such as (typically) 77 K or −196° C. (the boiling point of liquid nitrogen). Cryoprotective agents are often used at sub-zero temperatures to ameliorate or prevent cell damage due to freezing at low temperatures or warming to room temperature. Cryoprotective agents and optimal cooling rates can protect against cell injury. Cryoprotective agents which can be used include dimethyl sulfoxide (DMSO) (Lovelock and Bishop, Nature, 1959; 183: 1394-1395; Ashwood-Smith, Nature, 1961; 190: 1204-1205), glycerol, polyvinylpyrrolidine (Rinfret, Ann. N.Y. Acad. Sci., 1960; 85: 576), and polyethylene glycol (Sloviter and Ravdin, Nature, 1962; 196: 48). In particular embodiments, the cooling rate is 1° to 3° C./minute. After at least two hours, the cells reach a temperature of −80° C. and can be placed directly into liquid nitrogen (−196° C.) for permanent storage such as in a long-term cryogenic storage vessel.

(xii) Methods of Use. Methods disclosed herein include treating subjects (humans, veterinary animals (dogs, cats, reptiles, birds, etc.) livestock (horses, cattle, goats, pigs, chickens, etc.) and research animals (monkeys, rats, mice, fish, etc.) with (i) modified formulations and/or modifying formulations, and (ii) drug compositions disclosed herein. Treating subjects includes delivering therapeutically effective amounts. Therapeutically effective amounts include those that provide effective amounts, prophylactic treatments and/or therapeutic treatments without undue toxicity.

An “effective amount” is the amount of a formulation or composition necessary to result in a desired physiological effect. Effective amounts are often administered for research purposes.

Effective amounts disclosed herein can cause chromium or cytokine release in an assay of cell activation.

A “prophylactic treatment” includes a treatment administered to a subject who does not display signs or symptoms of a condition (e.g., cancer or an infection) or displays only early signs or symptoms of the condition such that treatment is administered for the purpose of diminishing or decreasing the risk of developing the condition further. Thus, a prophylactic treatment functions as a preventative treatment against a condition. In particular embodiments, prophylactic treatments reduce, delay, or prevent the worsening of a condition.

A “therapeutic treatment” includes a treatment administered to a subject who displays symptoms or signs of a condition and is administered to the subject for the purpose of diminishing or eliminating those signs or symptoms of the condition. The therapeutic treatment can reduce, control, or eliminate the presence or activity of the condition and/or reduce control or eliminate side effects of the condition.

Function as an effective amount, prophylactic treatment or therapeutic treatment are not mutually exclusive, and in particular embodiments, administered dosages may accomplish more than one treatment type.

Therapeutically effective amounts can be achieved by administering single or multiple doses during the course of a treatment regimen (e.g., daily, every other day, every 3 days, weekly, every 2 weeks, monthly, every 2 months, every 4 months, every 6 months, yearly, etc.).

As indicated, the formulations and compositions can be administered by injection, transfusion, implantation or transplantation. Modifying formulations and drug compositions can also be administered orally or via inhalation. In particular embodiments, formulations and compositions are administered parenterally. The phrases “parenteral administration” and “administered parenterally” refer to modes of administration other than enteral and topical administration, usually by injection, and includes, intravascular, intravenous, intramuscular, intraarterial, intrathecal, intracapsular, intratumoral, intraperitoneal, and subcutaneous, injection and infusion. In particular embodiments, the formulations and compositions described herein are administered to a subject by direct injection into a tumor, lymph node, or site of disease. In particular embodiments, drug compositions are administered orally.

In certain examples the disclosure provides methods of performing cellular immunotherapy in a subject having a disease or disorder comprising: administering a modified or modifying formulation that results in in vivo expression of a CAR whose activation state is constitutively “OFF”. In these examples, the methods further include administering a drug composition that allows activation of the CAR upon antigen binding. In some alternatives, the drug composition is delivered prior to, at the same time as the modified or modifying formulation, or at later time points after the modified or modifying formulation has been administered.

In some alternatives, the drug composition is administered with the modified or modifying formulation, and if a toxic effect of the formulation is observed the drug composition is withdrawn until the toxic effects diminish. After the symptoms of toxicity diminish, the drug composition can be administered again.

In certain examples the disclosure provides methods of performing cellular immunotherapy in a subject having a disease or disorder comprising: administering a modified or modifying formulation that results in in vivo expression of at least two types of CAR whose activation state is constitutively “OFF”. The two types of CAR bind different cancer antigens and have different hsp90 binding domains that bind different drug molecules. In these examples, the methods further include selectively administering one or more of the different drug molecule compositions to selectively allow activation of different CAR upon antigen binding. In some alternatives, one or more of the drug compositions are delivered prior to, at the same time as the modified or modifying formulation, or at later time points after the modified or modifying formulation has been administered.

In certain examples the disclosure provides methods of performing cellular immunotherapy in a subject having a disease or disorder comprising: administering a modified or modifying formulation that results in in vivo expression of at least one CAR whose activation state is constitutively “OFF” and at least one co-stimulatory molecule whose activation state is constitutively “OFF”. The CAR and the co-stimulatory molecule have different hsp90 binding domains that bind different drug molecules. In these examples, the methods further include selectively administering one or more of the different drug molecule compositions to selectively allow activation of the CAR upon antigen binding and/or the co-stimulatory molecule. In some alternatives, one or more of the drug compositions are delivered prior to, at the same time as the modified or modifying formulation, or at later time points after the modified or modifying formulation has been administered.

In some alternatives, one or more drug compositions are administered with the modified or modifying formulation, and if a toxic effect of the formulation is observed one or more of the drug compositions is withdrawn until the toxic effects diminish. After the symptoms of toxicity diminish, one or more drug compositions can be administered again. Toxicity can be observed based on, for example, levels of TNFα or IFNγ that exceed a clinically-relevant threshold.

In some alternatives, the drug composition(s) is administered with the modified or modifying formulation but once the subject has a decrease in cancer cells or virally-infected cells, the drug composition is not administered for a period of time to allow the modified cells to rest. Administration of the drug composition can also be stopped when a cancer is in remission or an infection has been cleared.

In other alternatives, a method comprises administering to the subject a genetically modified cytotoxic T lymphocyte cell preparation that provides a cellular immune response, wherein the cytotoxic T lymphocyte cell preparation comprises CD8+ T cells that express a CAR comprising a ligand binding domain, wherein the ligand binding domain is specific for a ligand, wherein the ligand is a tumor specific molecule, viral molecule, or any other molecule expressed on a target cell population, wherein the ligand can elicit recognition, modulation, inhibition, and/or elimination by a lymphocyte; a spacer domain; a transmembrane domain; and an intracellular signaling domain under the control of an hsp90 binding domain as described herein, and/or a genetically modified helper T lymphocyte cell preparation that elicits direct tumor recognition and enhances the genetically modified cytotoxic T lymphocyte cell preparations ability to mediate a cellular immune response, wherein the helper T lymphocyte cell preparation comprises CD4+ T cells express a CAR comprising a ligand binding domain, wherein the ligand binding domain is specific for a ligand, wherein the ligand is a tumor specific molecule, viral specific molecule, or any other molecule expressed on a target cell population, wherein the ligand can elicit recognition, modulation, inhibition, and/or elimination by a lymphocyte; a spacer domain; a transmembrane domain; and an intracellular signaling domain under control of an hsp90 binding domain as described herein and administering the drug composition that allows activation of the CAR.

Cancers that can be treated by modified or modifying formulations and drug compositions disclosed herein include: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B cell lymphoma, T cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma. Other exemplary cancers that can be treated according to the disclosure include hematopoietic tumors of lymphoid lineage, for example T cell and B cell tumors, including: T cell disorders such as T-prolymphocytic leukemia (T-PLL), including of the small cell and cerebriform cell type; large granular lymphocyte leukemia (LGL) of the T cell type; Sezary syndrome (SS); adult T cell leukemia lymphoma (ATLL); hepatosplenic T cell lymphoma; peripheral/post-thymic T cell lymphoma (pleomorphic and immunoblastic subtypes); angioimmunoblastic T cell lymphoma; angiocentric (nasal) T cell lymphoma; anaplastic (Ki 1+) large cell lymphoma; intestinal T cell lymphoma; and T-lymphoblastic lymphoma/leukemia (T-Lbly/T-ALL).

CAR with ligand binding domains that bind the following exemplary cancer antigens can be selected based on the cancer experienced by a subject: bladder cancer antigens: MUC16, PD-L1, EGFR; breast cancer antigens: HER2, ERBB2, ROR1, PD-L1, EGFR, MUC16, FOLR, CEA; cholangiocarcinoma antigens: mesothelin, PD-L1, EGFR; colorectal cancer antigens: CEA, PD-L1, EGFR; glioblastoma antigens: EGFR variant Ill (EGFRvIII), IL13Ra2; lung cancer antigens: ROR1, PD-L1, EGFR, mesothelin, MUC16, FOLR, CEA, CD56; Merkel cell carcinoma antigens: CD56, PD-L1, EGFR; mesothelioma antigens: mesothelin, PD-L1, EGFR; neuroblastoma antigens: ROR1, glypican-2, CD56, disialoganglioside, PD-L1, EGFR; ovarian cancer antigens: EpCam, L1-CAM, MUC16, folate receptor (FOLR), Lewis Y, ROR1, mesothelin, WT-1, PD-L1, EGFR, CD56; melanoma antigens: Tyrosinase related protein 1 (TYRP1/gp75); GD2, PD-L1, EGFR; multiple myeloma antigens: B-cell maturation antigen (BCMA), PD-L1, EGFR; pancreatic cancer antigens: mesothelin, CEA, CD24, ROR1, PD-L1, EGFR, MUC16; prostate cancer antigens: PSMA, WT1, Prostate Stem Cell antigen (PSCA), SV40 T, PD-L1, EGFR; renal cell carcinoma antigens: carboxy-anhydrase-IX (CAIX); PD-L1, EGFR; and stem cell cancer antigens: CD133, PD-L1, EGFR. Other examples are known to those of ordinary skill in the art. These groupings can be utilized to create CAR combination therapies as described elsewhere herein. CAR combination therapies can include activity-inducible co-stimulatory and/or inhibitory molecules.

Particular CAR combination therapies include CAR with binding domains that bind (i) CD19, CD22, and/or BAFF-R (e.g., CD19 and CD22) for the treatment of acute lymphoblastic leukemia (ALL); (ii) Her2, B7H3, EGFR, and/or IL13Ra2 for the treatment of brain tumors; and (iii) CD33 and CD123 for the treatment of acute myeloid leukemia (AML). Drug molecules to selectively allow for activation of CAR expressing ligand binding domains for these antigens include ES8, CMP8, and 4-OHT.

In certain examples, a cancerous sample from a subject can be characterized for the presence of certain biomarkers or cell surface markers. For example, breast cancer cells from a subject can be positive or negative for each of Her2Neu, Estrogen receptor, and/or the Progesterone receptor. A tumor antigen or cell surface molecule that is found on the individual subject's tumor cells as well as a CAR with a binding domain that binds the antigen is selected. Combinations may also be selected to create a CAR combination therapy.

In particular embodiments, therapeutically effective amounts of formulations and drug compositions provide anti-cancer effects. Anti-cancer effects include a decrease in the number of malignant cells, decrease in the number of metastases, a decrease in tumor volume, an increase in life expectancy, induced chemo- or radio-sensitivity in cancer cells, inhibited angiogenesis near cancer cells, inhibited cancer cell proliferation, inhibited tumor growth, prevented or reduced metastases, prolonged subject life, reduced cancer-associated pain, and/or reduced relapse or re-occurrence of cancer following treatment.

Infections that can be treated by disclosed formulations and compositions include bacterial, viral, fungal, parasitic, and arthropod infections. In particular embodiments, the infections are chronic. In particular embodiments, bacterial infections can include infections caused by Staphylococcus spp., Streptococcus spp., Campylobacter jejuni, Clostridium botulinum, Clostridium difficile, Escherichia coli, Listeria monocytogenes, Salmonella, Vibrio, Chlamydia trachomatis, Neisseria gonorrhoeae, and Treponema pallidum. In particular embodiments, viral infections can include infections caused by rhinovirus, influenza virus, respiratory syncytial virus (RSV), coronavirus (e.g., MERS, SARS, SARS-CoV-2), herpes simplex virus-1 (HSV-1), varicella-zoster virus (VZV), hepatitis A, norovirus, rotavirus, human papillomavirus (HPV), hepatitis B, human immunodeficiency virus (HIV), herpes simplex virus-2 (HSV-2), Epstein-Barr virus (EBV), West Nile virus (WNV), enterovirus, hepatitis C, human T-lymphotrophic virus-1 (HTLV-1), and Merkel cell polyomavirus (MCV). In particular embodiments, fungal infections can include infections caused by Trychophyton spp. and Candida spp. In particular embodiments, parasitic infections can include infections caused by Giardia, toxoplasmosis, E. vermicularis, Trypanosoma cruzi, Echinococcosis, Cysticercosis, Toxocariasis, Trichomoniasis, and Amebiasis. In particular embodiments, arthropod infections can include infections spread by arthropods infected with viruses or bacteria, including California encephalitis, Chikungunya, dengue, Eastern equine encephalitis, Powassan, St. Louis encephalitis, West Nile, Yellow Fever, Zika, Lyme disease, and babesiosis.

In particular embodiments, therapeutically effective amounts of formulations and drug compositions provide anti-infection effects. Anti-infection effects include a decrease in: the amount or level of infective pathogen, fatigue, loss of appetite, weight loss, fevers, night sweats, chills, aches and pains, diarrhea, bloating, abdominal pain, skin rashes, coughing, and/or a runny nose.

In particular embodiments, administration of drug compositions is stopped to provide an anti-side effect effect. An anti-side effect effect can reduce or eliminate a negative effect of formulation administration such as engraftment-induced cytokine storm (cytokine release syndrome), tumor lysis syndromes (TLS) or B cell cytopenia.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of disease, stage of disease, previous or concurrent therapeutic interventions, idiopathy of the subject and route of administration.

Therapeutically effective amounts of modified formulations to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

Useful doses to administer within modifying formulations or drug compositions can range from, for example, 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

(xiii) Kits. The current disclosure also includes kits assembled with materials useful to practice aspects of the disclosure. The kits can include, for example, cells (e.g., immune cells), nucleic acids encoding an activity-inducible fusion protein (e.g., a CAR, a co-stimulatory molecule and/or an inhibitory molecule), transfection reagents, assay reagents, drug molecules, buffers, cell nutrients and expansion media, cell sorting molecules (e.g., Dynabeads), tubes, wells, and small molecule estrogen analogs (e.g., tamoxifen, 4-OHT, ES8, CMP8).

(xiv) hsp90 Clients. Activity-inducible fusion proteins disclosed herein can include a binding domain derived from an alternative hsp90 client molecule or domain thereof. Hsp90 client proteins described herein are grouped according to transcription factors, kinases, and “other” as denoted in picard.ch/downloads/hsp90 interactors.

Examples of hs90 client transcription factors include 12(S)-HETE receptor; AF9/MLLT3; all vertebrate steroid receptors (GR, MR, ERa, ERP, PR, AR); AGL24; ATF3; BBX; BCL-6; Bclaf1; BES1; BrZ7; BZR1; C20orf194; CAR; CEBPE; Cwt1; CXXC1; cytoplasmic v-erbA; DLX6; DMRTA1; EcR; FOXD4L6; FOXM1; FOXP2; GTF21RD2; Hap1; HCFC1; HMGA1, HMGA2; HNF4A; HP1BP3; HSF-1; HsfA1, HsfA2, HsfB1; IRF2; IRF3; ISX; LFY; MAFG; Mal63; MaIR; MAX; Met1; MeWRKY20; MKX; mod(mdg4); c-Myc; Nanog; NFIC; FRKB; Notch1 (ICN1); NR1H3; NR112; Oct4; p53; p73; PAS family members: Dioxin receptor (=AhR), Sim, HIF-1α, HIF-2a, HIF-3a; PCGF6; POGK; PPARα, PPARβ, PPARγ; PRDM1; PREB; PXR; REST; REV-ERBα; RImA (of Aspergillus); SETDB1; SIM2; SLFN11; SOC1; SOX11; Sp1; SREBF1; SREBP1; SREBP2; Stat2; Stat3 (also in caveolin-1 complexes in rafts); Stat5; SUP; TADA2A; TBX22; TCF25; TDP-43; TEAD2; TFDP3; THAP4; TonEBP/OREBP; TRIM32; Tup1; Twist1; Ure2; USP1; VDR; VP16; water mold Achlya steroid (antheridiol) receptor; WT1; ZBED4; ZBTB17; ZBTB20; ZC3H7B; ZNF215; ZNF509; and ZNF74.

Examples of hs90 client kinases include ACVR1B; ACVR1C; ACVR2B; Akt/PKB; AKT2; ALK; ALK1, ALK5; ALPK1; AMHR2; AMPKa, AMPKy; ARAF; ASK1; ATM; AURKC; Aurora B; AXL; Bcr-Abl; BCR-FGFR1; BGLF4 of EBV; BLK; BMPR1A; BMX; BTK; c-Abl; c-Kit; c-Mos; CAMK1G; CAMK2A; CAMK2B; CAMK2D; CAMK2G; CAMK4; CAMKK1; CAMKK2; CAMKV; casein kinase Ila catalytic subunit; Cdc2 (=Cdk1); CDK11B; CDK14; CDK15; CDK18; Cdk2, Cdk4, Cdk6, Cdk9, Cdk11; CDK3; CheA (E. coli); Chk1; Cla4; CLK2; CLK3; Cot=Tpl-2; CSF1R; CSNK1A1; DCLK2; DDR1; DDR2; Death-associated kinases DAPK, DAPK2, DAPK3; DLK; DMPK; DYRK1B; DYRK2; DYRK4; eEF-2 kinase; EGF receptor (mutant and wt); elF2-a kinases HRI, Gcn2, Perk, PKR; Eml4-Alk; EPHA1; EphA2; EPHA4; EPHB1; EPHB6; ErbB2; ERBB3; ERBB4; ERK5; FASTK; FGFR1; FGFR3 and FGFR4; Flt3; FLT4; FOP2-FGFR1; FRK; Fused; FYN; Gal1; GRK2 and GRK6; GRK4; GRK7; GSK3A; GSK3R; HCK; HER3; HIPK4; HopBF1 effectors; ICK; INSRR; Insulin receptor; Insulin-like growth factor 1 receptor; Integrin-linked kinase; IP6K2; IRAK-1; IRAK2; IRAK3; Ire1a; ITK; IKB kinases (IKK) α, β, γ, ε; JAK1; JNK; KSR; LATS1, LATS2; LCK; LIMK1; LIMK2; Lkb1; LMTK3; LRRK2; LYN; MAP2K5; MAP2K7; MAP3K12; MAP3K15; MAP3K2; MAP3K6; MAP3K9; MAP4K1; MAP4K2; MAP4K4; MAPKKK (MEKK) YODA; MAPK15; MAPK4; MAPK6; MAPK7; MAST1; MAST2; MATK; MEK; MEKK1 and MEKK3; MERTK; MET; Mik1; MINK1; MLK3; MLKL; MOK, MAK, MRK; MpkA (of Aspergillus); Mps1; mTOR; MUSK; MYLK2; MYLK3; MYLK4; NEK11; NEK8; NEK9; NIK; NPM-Alk; NPR2; NTRK1; NTRK2; NTRK3; NUAK2; Nucleophosmin-Anaplastic Lymphoma Kinase; p38; p90RSK; PAK6; PASK; Pbs2; PDGFRB; PDIK1L; PDK1; PGK1; PI4KIIP; Pim-1; PIM2; PIM3; Pink1; PKCA, PKCEand other PKCs; PKM2; PKN1; PKN2; platelet-derived growth factor receptor a; Plk1; Plk3; Pnck; pp60v-src, c-src; PRKAA2; PRKACB; PRKCA; PRKCB; PRKCG; PRKCH; PRKCI; PRKCQ; PRKCZ; PRKD1; PRKD2; PRKDC; PRKG2; PRKX; PRKY; PSKH1; PSKH2; PTK2; PTK2B; PTK6; PTK6; Raf-1, B-Raf, Ste11; RET; RET/PTC1; RIP1; RIP3; Ron; ROR2; RPS6KA1; RPS6KA2; RPS6KA3; RPS6KA5; RPS6KA6; RPS6KB1; RPS6KC1; RPS6KL1; Ryk; SGK-1; SGK2; SGK223; SGK3; Slt2; src related tyrosine kinases: fer, fes, fgr, fps, Ick, yes; SRPK1; SRPK3; SSCMK1; STK32B; STK32C; STK33; STK38; STK38L; STYK1; SYK; TAK1; TAOK3; TBK1; TESK1; TESK2; TGFP receptors I and II; TIE1; TNK1; TNK2; TNNI3K; TP53RK; TrkAl and Ill; TrkB; TSSK1B; TSSK2; TSSK3; TSSK4; TSSK6; Tyk2; TYRO3; Ulk1; VEGFR1, VEGFR2; Wee1, Swe1; WNK4; and ZAP-70.

Examples of hs90 client molecules denoted as “other” include Act1 (=TRAF3IP2); Adenosine A2A receptor; a2C adrenergic receptor; AID; AIP56; Aldo-keto reductase 1B10; ANAPC2; ANKMY2; Annexin A2; ANP receptor; ANP32C/D; Apaf-1; apoB; APOBEC-3B, -3C, -3G; Arb1; ARD1; Argonaute-1 (Ago1); Argonaute-2 (=Ago2=GERp95); Argonaute-4 (Ago4); ARMC5; ArtAB; ASB17; ASB2; ASB3; ASB4; ASB6; ATG8 (GABARAP) proteins; Axin 1; BCAP (PIK3AP1); BALF5 of EBV; Bcl-2; Bcl-xL; Beclin 1; Bid; BIN2; BLM helicase; Bms1; BPIFB4; BRAT1; BRCA1; BRCA2; BRMS1; BTRC; c-IAP1; calcineurin (Cna2; catalytic subunit); calmodulin; calmodulin methyltransferase; calpain-1; calponin; CARM1; Caspase-8; p-catenin; CB2 cannabinoid receptor; Ccp1; CCDCl17; CD38 type Ill; CD79a; Cdc13; Cdc14; Cdc25a and Cdc25c; Cdk5 activator p35; CPEB1, CPEB2, CPEB3; CFTR (nascent and mutant polypeptide); ChAT; CheZ (E. coli); ChIl1; Chronophin; Cineole synthase 1; Clathrin heavy chain; CLC-1 chloride channel; CLC-2 chloride channel; Clostridium toxin CDT; Clostridium toxin iota; Clusterin; COG complex; COI1; Complement C9; Cry toxins; CTA1=CtxA1; Ctf13/Skp1 component of CBF3; CUL1; CUL2; CUL3; CUL4A; CUL4B; Cup; cyclin B; cyclophilin D (mitochondrial); Cyr1; cytoskelettal proteins: actin, tubulin (including ciliary P4-tubulin), myosin (including Myo3B); DBC2; DEDD; Dengue virus protein E; Dengue virus proteins NS1/2B/3/4B/5; DET1; Diphtheria toxin A; DNA helicase Ss12; DNA polymerase a; DNA polymerase λ; DNA polymerase η; DnaA (E. coli); DNMT1; Dsn1; DTX4; E6\hat{ }E7; EBAX-1; Emc2; ENC1; eNOS, nNOS (?); ether-a-gogo-related potassium channel (ERG=HERG=KCNH2); EZH2; F1FO-ATP synthase; FANCA; FBXL12; FBXL13; FBXL14; FBXL15; FBXL18; FBXL2; FBXL3; FBXL6; FBXL8; FBXO10; FBXO17; FBXO18; FBXO24; FBXO25; FBXO27; FBXO28; FBXO3; FBXO34; FBXO38; FBXO4; FBXO40; FBXO6; FBXO9; FBXW11; FBXW2; FBXW5; FBXW7; FGAMS; Fibronectin; FliN, Flil (E. coli); FLIPs and FLIP_{L}; Folliculin; free Py subunit of G protein; FtsZ; G2E3; GAN; Gln1; GLT-1; GluR1; glutathione S-transferase subunit 3 (KS type); Guanylate cyclase, soluble; Gao, Ga₁₂; Glucocerebrosidase; GREB1; HAX-1; HDAC1; HDAC6; HECTD3; Hepatitis B virus core protein; Hepatitis C virus protein NS3; Hepatitis E virus capsid protein; HERC4; HERC6; Histones H1, H2A, H2B, H3 and H4; HMGCR; Hsp27; Humanin; Huntingtin; Importin 4 (IP04); Importin α1; Importin-α6 (KPNA5); Ino80; Inositol 1,4,5-trisphosphate receptor 3; Integrin α2; Integrin α4; Integrin aL; IL-1p; IRS-2; Japanese encephalitis virus E protein; JlpA; KAP1; KAT5; KBTBD4; KBTBD7; KCNA5; KCNA6; KCNG1; KCNS3; KCNQ4; KCTD8; KDM3A/JMJD1A; KDM4B/JMJD2B; KEAP1; KIAA0317; Kir6.2; KLHL1; KLHL10; KLHL13; KLHL14; KLHL15; KLHL22; KLHL23; KLHL25; KLHL26; KLHL29; KLHL32; KLHL34; KLHL36; KLHL38; KLHL6; knob complexes (in the membrane of Plasmodium-infected erythrocytes); KSHV K1; KSR1; KSR2; L protein of HRSV; Lamin A/C; LAMP-2A; LANA of KS-HSV; LAP; LARP4B; Legumain; LGALS3BP; LIS1; LNX1; LOC440248; LOX1 (OLR1); LOXL2; Lp11 (S. aureus); LRP1 (=CD91); LRP5; LRSAM1; LSD1; LSM8; macromolecular aminoacyl-tRNA synthetase complex; Macrophage scavenger receptor; MAP1B; MARCH9; Mdm2; MDM4; MeCatalase1; Mg²⁺-dependent phosphatidate phosphohydrolase; MIF; misfolded VHL; MMP2, MMP3, MMP9; p-opioid receptor; MRE11/Rad50/NBS1 (MRN) complex; MRP1; Msps/XMAP215/ch-TOG; MTA1; MTG8; MUC1; Myoglobin; N-myc downstream-regulated gene 1 (NRDG1); N-WASP; Na⁺-K⁺—Cl⁻ cotransporter 1; NadA; NAP1; NB-LRR proteins: RPM1 and RPS2, Nod1, Nod2, NALP2, NALP3, NALP4, NALP12, IPAF, RPP4; NCC; NDRG2; NELF-E; Nervous necrosis virus capsid protein; Neuraminidase; NeuropeptideY; NHE1; NHLRC1; Nibrin; NleH1 and NleH2; NMNAT2; Norovirus capsid protein VP1; Nox1, Nox2, Nox3, Nox5; NS1; Ns|1; nsP3 and nsP4 of Chikungunya virus; Nucleoprotein (NP) of MERS-CoV; Nup62; OGT; OsCERK1; P protein (rabies virus); P1 (picornaviral capsid precursor protein P1); p14ARF; P2X₇ purinergic receptor; p300; P450 CYP2E1; PARK2; PARK7 (DJ-1); PB1 and PB2 subunits of influenza RNA pol.; PCGF1; PCGF3; PCNA; Peli1; perilipin; PfCRT; PIDD; Piwi; PIWIL2; PLCγ; PLN; polysomal ribonuclease 1 (PMR1); PPAT; PRDM14; PRMT5; pro-Dcp1; prolactin receptor; prostacyclin synthase; proteasome; PRPF8; PRPF19; PTPN22; Ptx; R-protein 1-2; R2TP complex through Pih1; Rab-aGDI; Rab3a; Rab11a; RAB40A; Rac/Rop GTPase Rac1 (rice); Rac1; Rad51; Rad52; RAG1; Ral-binding protein 1 (RaIBP1); RanBP9; Rapsyn; Raptor; RCBTB1; RCBTB2; reovirus protein 61; REV1; reverse transcriptase of hepatitis B virus; RFWD3; RGS11; RGS6; RGS7; RGS9; RHOBTB1; ribosomal protein L2 (E. coli); ribosomal proteins S3 and S6; ricin catalytic A chain; RIG-I; RNA-dep. RNA polymerase (of Bamboo mosaic virus); RNF10; RNF111; RNF19B; RNF40; RNGTT; Rnr4; Rpb1; SCAP; SDF2; SENP3; SERCA2a; SERT (SLC6A4); SF3B3; SH3RF2; Sicily; SIR2 (SIR2RP1 in Leishmania); SIRT1; SIRT2; SKP2; SKP2 complexes; SLC6A14; SMYD1, SMYD2, SMYD3; snoRNP complexes; SNRNP200; SOCS6; SPSB1; SPSB3; SREC-l; STING; SUR1 (subunit of β-cell ATP-sensitive potassium channel); survivin; SV40 large T-antigen; Swr1; α-synuclein; Tab2/3; Tas3; Tau protein; Tax; TCL1A; telomerase; TFR1; thiopurine S-methyltransferase; thrombin receptor (PAR-1); thromboxane synthase; TilS; TIMP2; TIR1; Tissue plasminogen activator (tPA); Titin; TLR4/MD-2 complex; TLR7; TLR9; Tm-2²; TNFAIP3; TOM40; TRIM10; TRIM17; TRIM2; TRIM36; TRIM37; TRIM41; TRIM49; TRIM56; TRIM7; TRIM73; TRIM74; TRIM8; Triosephosphate isomerase; Trithorax (and ortholog MLL); Trx1; TrxR; TSG101; Tyrosine hydroxylase; UCH-L1; UHRF1; Ulp1; uPA; Ura2; URI complex; Uroporphyrinogen decarboxylase (HemE) [in cyanobacteria]; Us11 (of HSV-1); USP19; Utp21; Vaccinia core protein 4a; vFLIP (of KSHV); Vimentin; VIP1; VPS18; VPS41; WASF3; WSB2; WTAP; WWP1; XPO1; XPORT; XRCC1; ZEITLUPE; and ZMYND10.

(xv) Variants. Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32).

Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

Functional variants include one or more residue additions or substitutions that do not substantially impact the physiological effects of the protein. Functional fragments include one or more deletions or truncations that do not substantially impact the physiological effects of the protein. A lack of substantial impact can be confirmed by observing experimentally comparable results in an activation study or a binding study. Functional variants and functional fragments of intracellular domains (e.g., intracellular signaling domains) transmit activation or inhibition signals comparable to a wild-type reference when in the activated state of the current disclosure. Functional variants and functional fragments of binding domains bind their cognate antigen or ligand at a level comparable to a wild-type reference.

In particular embodiments, a binding domain VH region can be derived from or based on a VH of a known antibody and can optionally contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of the known antibody. An insertion, deletion or substitution may be anywhere in the VH region, including at the amino- or carboxy-terminus or both ends of this region, provided that each CDR includes zero changes or at most one, two, or three changes and provided a binding domain containing the modified VH region can still specifically bind its target with an affinity similar to the wild type binding domain.

In particular embodiments, a VL region in a binding domain is derived from or based on a VL of a known antibody and optionally contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the known antibody. An insertion, deletion or substitution may be anywhere in the VL region, including at the amino- or carboxy-terminus or both ends of this region, provided that each CDR includes zero changes or at most one, two, or three changes and provided a binding domain containing the modified VL region can still specifically bind its target with an affinity similar to the wild type binding domain.

These considerations similarly apply to TCR chains and other derived binding domains.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

### (xvi) Exemplary Embodiments

1. An activity-inducible fusion protein including an hsp90 binding domain.

2. The activity-inducible fusion protein of embodiment 1, wherein the hsp90 binding domain binds hsp90 with a lower affinity than it binds a drug molecule.

3. The activity-inducible fusion protein of embodiments 1 or 2, wherein the hsp90 binding domain includes a hormone binding domain.

4. The activity-inducible fusion protein of embodiment 3, wherein the hormone binding domain is an engineered estrogen receptor binding domain (EBD).

5. The activity-inducible fusion protein of embodiment 4, wherein the EBD includes the binding domain portion of the estrogen receptor and a set of mutations selected from G521R; E353A; L384M and M421G; L384M, M421G, and G521R; or G400V, M543A, and L544A.

6. The activity-inducible fusion protein of embodiment 5, wherein the EBD has the sequence as set forth in SEQ ID NO: 4, SEQ ID NO: 6, SEQ ID NO: 9, SEQ ID NO: 11, or SEQ ID NO: 13.

7. The activity-inducible fusion protein of any of embodiments 2-6, wherein the drug molecule includes a small molecule estrogen analog.

8. The activity-inducible fusion protein of embodiment 7, wherein the small molecule estrogen analog includes tamoxifen, a salt of tamoxifen, a metabolite of tamoxifen, or a compound that is structurally similar to tamoxifen.

9. The activity-inducible fusion protein of embodiments 7 or 8, wherein the small molecule estrogen analog includes tamoxifen, 4-OHT, (Z)-endoxifen, ES8, or CMP8.

10. The activity-inducible fusion protein of any of embodiments 1-9, wherein the activity-inducible fusion protein is a chimeric antigen receptor (CAR) that when expressed by a cell includes:
an extracellular component and an intracellular component linked by a transmembrane domain, wherein
   the extracellular component includes a ligand binding domain and
   the intracellular component includes an intracellular signaling domain and the hsp90 binding domain.

11. The activity-inducible fusion protein of embodiment 10, wherein the ligand binding domain binds a cancer antigen or a viral antigen.

12. The activity-inducible fusion protein of embodiments 10 or 11, wherein the ligand binding domain includes an scFv that binds HER2, CE7, hB7H3, EGFR, EGFRvIII, CD19, CD20, CD22, EphA2, IL13Ra2, L1CAM, oaGD2, B7H3, CD33, Mesothelin, ROR1, FITC or VAR2CSA.

13. The activity-inducible fusion protein of embodiment 12, wherein the scFv has a sequence as set forth in SEQ ID NO: 16, SEQ ID NO: 18, SEQ ID NO: 19, SEQ ID NO: 20, SEQ ID NO: 21, SEQ ID NO: 23, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 342, or SEQ ID NO: 43.

14. The activity-inducible fusion protein of embodiment 10, wherein the ligand binding domain binds an immune cell antigen.

15. The activity-inducible fusion protein of embodiment 14, wherein the immune cell antigen is expressed by a B cell, a T cell, a natural kiIler cell, a natural kiIler T cell, a MAIT cell, a myeloid cell, a macrophage, a monocyte, or a dendritic cell.

16. The activity-inducible fusion protein of embodiment 10, wherein the ligand binding domain binds a hapten.

17. The activity-inducible fusion protein of embodiment 16, wherein the hapten includes fluorescein, urushiol, quinone, biotin, or dinitrophenol.

18. The activity-inducible fusion protein of embodiments 16 or 17, wherein the ligand binding domain is an scFv having the sequence as set forth in SEQ ID NO: 44, SEQ ID NO: 45, SEQ ID NO: 46, SEQ ID NO: 47, SEQ ID NO: 48, SEQ ID NO: 49, SEQ ID NO: 51, SEQ ID NO: 53, SEQ ID NO: 60, SEQ ID NO: 61, SEQ ID NO: 62, SEQ ID NO: 63, SEQ ID NO: 64, or SEQ ID NO: 65.

19. The activity-inducible fusion protein of any of embodiments 10-18, wherein the extracellular component further includes a spacer region.

20. The activity-inducible fusion protein of embodiment 19, wherein the spacer region includes an IgG4 hinge.

21. The activity-inducible fusion protein of any of embodiments 10-20, wherein the intracellular signaling domain includes a CD3ζ signaling domain.

22. The activity-inducible fusion protein of any of embodiments 10-21, wherein the intracellular signaling domain includes a 4-1 BB signaling domain.

23. The activity-inducible fusion protein of any of embodiments 10-22, wherein the intracellular signaling domain includes a CD3ζ signaling domain and a 4-1BB signaling domain.

24. The activity-inducible fusion protein of any of embodiments 10-23, further including a 1 Gly, 2 Gly, or 3Gly junction amino acid adjacent to the hsp90 binding domain or no linker adjacent to the hsp90 binding domain.

25. The activity-inducible fusion protein of embodiment 24, wherein the 1, 2, or 3 Gly junction amino acid, if present, is 5′ of the hsp90 binding domain and 3′ of the signaling domain.

26. The activity-inducible fusion protein of any of embodiments 10-25, wherein the hsp90 binding domain is 3′ of the signaling domain.

27. The activity-inducible fusion protein of any of embodiments 10-25, wherein the hsp90 binding domain is 5′ of the signaling domain and 3′ of the transmembrane domain.

28. The activity-inducible fusion protein of any of embodiments 23-25, wherein the hsp90 binding domain is 5′ of the CD3ζ portion of the signaling domain and 3′ of the 4-1EE portion of the signaling domain.

29. The activity-inducible fusion protein of any of embodiments 10-25, wherein the intracellular component lacks linkers and junction amino acids.

30. The activity-inducible fusion protein of any of embodiments 10-29, wherein the transmembrane domain includes a CD28 transmembrane domain.

31. The activity-inducible fusion protein of any of embodiments 10-30, having the protein sequence as set forth in SEQ ID NO: 118.

32. The activity-inducible fusion protein of any of embodiments 1-31, wherein the hsp90 binding domain is linked to the intracellular portion of a co-stimulatory immune molecule or an inhibitory immune molecule.

33. The activity-inducible fusion protein of embodiment 32, wherein the co-stimulatory immune molecule includes 4-1BB, OX40, CD40, CD30, CD27, DR3, SLAMF1, ICOS, GITR, CD25, CD28, CD79A, CD79B, CD226, CARD11, DAP10, DAP12, DR3, FcRα, FcRβ, FcRγ, Fyn, Lck, LAT, LRP, LIGHT, NKG2D, NOTCH1, NOTCH2, NOTCH3, NOTCH4, ROR2, Ryk, Slp76, pTα, TCRα, TCRβ, TIM1, TRIM, Zap70, or PTCH2.

34. The activity-inducible fusion protein of embodiment 32, wherein the inhibitory immune molecule includes PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD80, CD86, CD160, 2B4, B7-H3 (CD276), B7-H4 (VTCN1), KIR, A2aR, MHC class I, MHC class II, GAL9, adenosine, or TGFR β.

35. A nucleotide encoding an activity-inducible fusion protein of any of embodiments 1-34.

36. The nucleotide of embodiment 35, including the coding sequence as set forth in SEQ ID NO: 7, SEQ ID NO: 14, or SEQ ID NO: 15.

37. The nucleotide of embodiment 36, further including the coding sequence as set forth in SEQ ID NO: 17.

38. The nucleotide of embodiments 36 or 37, further including the coding sequence as set forth in SEQ ID NO: 79.

39. The nucleotide of any of embodiments 36-39, further including the coding sequence as set forth in SEQ ID NO: 124.

40. The nucleotide of any of embodiments 36-39, further including the coding sequence as set forth in SEQ ID NO: 123.

41. The nucleotide of any of embodiments 36-40, further including the coding sequence as set forth in SEQ ID NO: 122.

42. The nucleotide of any of embodiments 35-41, having the sequence as set forth in SEQ ID NO: 125.

43. A cell genetically modified to express an activity-inducible fusion protein of any of embodiments 1-34.

44. The cell of embodiment 43, genetically modified to express at least two types of an activity-inducible fusion protein of any of embodiments 1-29, wherein the two types have different hsp90 binding domains that bind different drug molecules.

45. The cell of embodiment 44, wherein the two types further have different ligand binding domains.

46. The cell of embodiment 45, wherein the different ligand binding domains bind different antigens.

47. The cell of embodiment 46, wherein the different antigens are associated with a common cancer, a common infection, a common immune cell type, or a common hapten or are associated with different cancers, different infections, different immune cell types, or different haptens.

48. The cell of embodiment 47, wherein the common cancer type is acute lymphoblastic leukemia (ALL), acute myeloid leukemia (AML), or a brain cancer.

49. The cell of any of embodiments 43-48, wherein the cell is a T cell or a natural kiIler (NK) cell.

50. The cell of embodiment 49, wherein the T cell is a CD4+ or a CD8+ T cell.

51. The cell of any of embodiments 43-48, wherein the cell is an induced pluripotent stem cell (iPSC), a tumor-infiltrating lymphocyte (TIL), a marrow-infiltrating lymphocyte (MIL), a natural kiIler T cell (NKT), a mucosal-associated invariant T (MAIT) cell, a dendritic cell, a monocyte or a macrophage.

52. A system for controlling the activation state of an activity-inducible fusion protein in vivo including:
the activity-inducible fusion protein wherein the activity-inducible fusion protein includes an hsp90 binding domain that binds a drug molecule; and
the drug molecule.

53. The system of embodiment 52, wherein the hsp90 binding domain binds hsp90 with a lower affinity than it binds the drug molecule.

54. The system of embodiments 52 or 53, wherein the hsp90 binding domain includes a hormone binding domain.

55. The system of embodiment 54, wherein the hormone binding domain includes an engineered estrogen receptor binding domain (EBD).

56. The system of embodiment 55, wherein the EBD includes the binding domain portion of the estrogen receptor and a set of mutations selected from G521R; E353A; L384M and M421G; L384M, M421G, and G521R; or G400V, M543A, and L544A.

57. The system of embodiments 55 or 56, wherein the EBD has the sequence as set forth in SEQ ID NO: 4, SEQ ID NO: 6, SEQ ID NO: 9, SEQ ID NO: 11, or SEQ ID NO: 13.

58. The system of any of embodiments 52-57, wherein the drug molecule includes a small molecule estrogen analog.

59. The system of embodiment 58, wherein the small molecule estrogen analog includes tamoxifen, a salt of tamoxifen, a metabolite of tamoxifen, or a compound that is structurally similar to tamoxifen.

60. The system of embodiments 58 or 59, wherein the small molecule estrogen analog includes tamoxifen, 4-OHT, (Z)-endoxifen, ES8, or CMP8.

61. The system of any of embodiments 52-60, wherein the activity-inducible fusion protein is a CAR and wherein when expressed by a cell the CAR includes
an extracellular component and an intracellular component linked by a transmembrane domain, wherein
the extracellular component includes a ligand binding domain and
the intracellular component includes an intracellular signaling domain and the hsp90 binding domain.

62. The system of embodiment 61, wherein the ligand binding domain binds a cancer antigen or a viral antigen.

63. The system of embodiments 61 or 62, wherein the ligand binding domain includes an scFv that binds HER2, CE7, hB7H3, EGFR, EGFRvIII, CD19, CD20, CD22, EphA2, IL13Ra2, L1CAM, oaGD2, B7H3, CD33, Mesothelin, ROR1, FITC or VAR2CSA.

64. The system of embodiment 63, wherein the scFv has a sequence as set forth in SEQ ID NO: 16, SEQ ID NO: 18, SEQ ID NO: 19, SEQ ID NO: 20, SEQ ID NO: 21, SEQ ID NO: 23, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, or SEQ ID NO: 43.

65. The system of embodiment 61, wherein the ligand binding domain binds an immune cell antigen.

66. The system of embodiment 65, wherein the immune cell antigen is expressed by a B cell, a T cell, a natural kiIler cell, a natural kiIler T cell, a MAIT cell, a myeloid cell, a macrophage, a monocyte, or a dendritic cell.

67. The system of embodiment 61, wherein the ligand binding domain binds a hapten.

68. The system of embodiment 67, wherein the hapten comprises fluorescein, urushiol, quinone, biotin, or dinitrophenol.

69. The system of embodiments 67 or 68, wherein the ligand binding domain is an scFv having the sequence as set forth in SEQ ID NO: 44, SEQ ID NO: 45, SEQ ID NO: 46, SEQ ID NO: 47, SEQ ID NO: 48, SEQ ID NO: 49, SEQ ID NO: 51, SEQ ID NO: 53, SEQ ID NO: 60, SEQ ID NO: 61, SEQ ID NO: 62, SEQ ID NO: 63, SEQ ID NO: 64, or SEQ ID NO: 65.

70. The system of any of embodiments 61-69, wherein the extracellular component further includes a spacer region.

71. The system of embodiment 70, wherein the spacer region includes an IgG4 hinge.

72. The system of any of embodiments 61-71, wherein the intracellular signaling domain includes a CD3ζ signaling domain.

73. The system of any of embodiments 61-72, wherein the intracellular signaling domain includes a 4-1 BB signaling domain.

74. The system of any of embodiments 61-73, wherein the intracellular signaling domain includes a CD3ζ signaling domain and a 4-1 BB signaling domain.

75. The system of any of embodiments 61-74, further including a 1 Gly, 2 Gly, or 3Gly junction amino acid adjacent to the hsp90 binding domain or no linker adjacent to the hsp90 binding domain.

76. The system of embodiment 75, wherein the 1, 2, or 3 Gly junction amino acid, if present, is 5′ of the hsp90 binding domain and 3′ of the signaling domain.

77. The system of any of embodiments 61-76, wherein the hsp90 binding domain is 3′ of the signaling domain.

78. The system of any of embodiments 61-76, wherein the hsp90 binding domain is 5′ of the signaling domain and 3′ of the transmembrane domain.

79. The system of any of embodiments 74-76, wherein the hsp90 binding domain is 5′ of the CD3ζ portion of the signaling domain and 3′ of the 4-1BB portion of the signaling domain.

80. The system of any of embodiments 61-79, wherein the intracellular component lacks linkers and junction amino acids.

81. The system of any of embodiments 61-80, wherein the transmembrane domain includes a CD28 transmembrane domain.

82. The system of any of embodiments 61-81, wherein the CAR has the protein sequence as set forth in SEQ ID NO: 118. 83. The system of any of embodiments 52-82, wherein the activity-inducible fusion protein is a co-stimulatory immune molecule or an inhibitory immune molecule.

84. The system of embodiment 83, wherein the co-stimulatory immune molecule includes 4-1EE, OX40, CD40, CD30, CD27, DR3, SLAMF1, ICOS, GITR, CD25, CD28, CD79A, CD79B, CD226, CARD11, DAP10, DAP12, DR3, FcRα, FcRβ, FcRγ, Fyn, Lck, LAT, LRP, LIGHT, NKG2D, NOTCH1, NOTCH2, NOTCH3, NOTCH4, ROR2, Ryk, Slp76, pTα, TCRα, TCRβ, TIM1, TRIM, Zap70, or PTCH2.

85. The system of embodiment 83, wherein the inhibitory immune molecule includes PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD80, CD86, CD160, 2B4, B7-H3 (CD276), B7-H4 (VTCN1), KIR, A2aR, MHC class I, MHC class II, GAL9, adenosine, or TGFR β.

86. The system of any of embodiments 52-85, including at least two types of an activity-inducible fusion protein, wherein the two types have different hsp90 binding domains that bind different drug molecules.

87. The system of embodiment 86, wherein the two types further have different ligand binding domains.

88. The system of embodiment 87, wherein the different ligand binding domains bind different antigens.

89. The system of embodiment 88, wherein the different antigens are associated with a common cancer, a common infection, a common immune cell type, or a common hapten or are associated with different cancers, different infections, different immune cell types, or different haptens.

90. The system of embodiment 89, wherein the common cancer type is acute lymphoblastic leukemia (ALL), acute myeloid leukemia (AML), or a brain cancer.

91. The system of any of embodiments 52-90, formulated for administration to a subject.

92. The system of embodiment 91, wherein the formulated system includes
(i) ex vivo manufactured cells expressing the activity-inducible fusion protein formulated into a pharmaceutically acceptable carrier to create a modified formulation; and/or
(ii) cell-targeted viral vectors and/or a cell-targeted nanoparticles formulated into a pharmaceutically acceptable carrier to create a modifying formulation wherein the cell-targeted viral vectors and/or a cell-targeted nanoparticles include gene-modifying components that result in expression of the activity-inducible fusion protein in vivo by the targeted cell following administration; and
(iii) the drug molecule formulated into a pharmaceutically acceptable carrier to create a drug composition.

93. A method of treating a subject in need thereof including administering a system of embodiment 92 to the subject, thereby treating the subject.

94. The method of embodiment 93, wherein the administering includes administering the modified formulation and the drug composition.

95. The method of embodiment 93, wherein the administering includes administering the modifying formulation and the drug composition.

96. The method of any of embodiments 93-95, wherein the method further includes stopping administration of the drug molecule to reduce a side effect of system administration.

97. The method of any of embodiments 93-96, wherein the method further includes stopping administration of the drug molecule when the subject is no longer in need thereof.

98. The method of any of embodiments 93-97, wherein the subject is in need thereof due to cancer or a viral infection.

99. The method of any of embodiments 93-98, wherein the system includes at least two types of an activity-inducible fusion protein, wherein the two types have different hsp90 binding domains that bind different drug molecules.

100. The method of embodiment 99, including administering at least two types of drug molecules wherein one of the at least two types binds the hsp90 binding domain of one activity-inducible fusion protein of the system and wherein one of the at least two types binds the hsp90 binding domain of a different activity-inducible fusion protein of the system.

101. The method of embodiment 100, wherein the method further includes stopping administration of at least one of the drug molecules.

102. The method of embodiments 100 or 101, wherein the method further includes stopping administration of all of the drug molecules.

103. The method of any of embodiments 99-103, wherein the two types further have different ligand binding domains.

104. The method of embodiment 103, wherein the different ligand binding domains bind different antigens.

105. The method of embodiment 104, wherein the different antigens are associated with a common cancer, a common infection, a common immune cell type, or a common hapten or are associated with different cancers, different infections, different immune cell types, or different haptens.

106. The method of embodiment 105, wherein the common cancer type is acute lymphoblastic leukemia (ALL), acute myeloid leukemia (AML), or a brain cancer.

107. The activity-inducible fusion protein of any of embodiments 1-34, wherein the activity-inducible fusion protein does not include a degron sequence.

108. The activity-inducible fusion protein of any of embodiments 1-34 or 107, wherein activity is induced within 1 hour of administration of a drug molecule that binds the HSP binding domain.

109. The activity-inducible fusion protein of any of embodiments 1-34 or 107, wherein activity is induced within 30 minutes of administration of a drug molecule that binds the HSP binding domain.

110. The activity-inducible fusion protein of any of embodiments 1-34 or 107, wherein activity is induced within 15 minutes of administration of a drug molecule that binds the HSP binding domain.

111. The activity-inducible fusion protein of any of embodiments 1-34 or 107, wherein activity is induced within 5 minutes of administration of a drug molecule that binds the HSP binding domain.

(xvii) Experimental Example describing FIGS. 9-17. Further assessment of the CAR-EBD constructs examined the positioning of the EBD domain in the CAR construct as well as the linker used to connect the EBD domain to the CAR construct. CAR-EBD constructs containing a glycine linker (also referred to herein as a junction amino acid) consisting of one, two, or three glycine positioned after (i.e, 3′) CD3zeta were examined (data shown). CAR-EBD constructs with no glycine linkers and constructs that position the EBD domain between 4-1BB and CD3zeta with surrounding 3 glycine linkers or fused (no glycine linkers) have also been designed. Examples include:
huCD19 CAR-Gly1-EBD-P2A-DHFRdm-Her2tG;
huCD19 CAR-Gly2-EBD-P2A-DHFRdm-Her2tG;
huCD19 CAR-Gly3-EBD-P2A-DHFRdm-Her2tG;
huCD19 CAR-EBD-P2A-DHFRdm-Her2tG;
EGFR806scFv-IgG4hinge-CD28tm-4-1 BB-Gly3-EBD-Gly3-CD3zeta-P2A-DHFRdm-CD19t; and
EGFR806scFv-IgG4hinge-CD28tm-4-1 BB-EBD-CD3zeta-P2A-DHFRdm-CD19t.

The EBD can be, for example, EBD(4-OHT), EBD(CMP8), or EBD(ES8).

Data for 2 different EBD mutants are presented within FIGS. 9-17:
EBD(4-OHT) (SEQ ID NO: 13): Responsive to 4-OHT and (Z)-endoxifen; and
EBD(CMP8) (SEQ ID NO: 11): Responsive to 4-OHT, (Z)-endoxifen, and CMP8.

FIG. 9 (top) depicts a schematic of Jurkat iSynPro eGFP:ffluc reporter cells. Jurkat iSynPro eGFP:ffluc reporter cells feature a Jurkat cell line lentivirally transduced to stably express a inducible synthetic promoter (iSynPro WO2018213332A1) regulating the expression of an enhanced green fluorescent protein (eGFP) fused to bioluminescent reporter firefly luciferase (ffluc). This line was then clonally selected by limiting dilution. Upon T cell activation, the Jurkat iSynPro eGFP:ffluc line will both induce GFP expression detectable by flow cytometry and allow for the measurement of luciferase activity by using the addition of substrate D-luciferin to catalyze the luciferase reaction.

Jurkat iSynPro eGFP:ffluc assays are also depicted in FIG. 9 (bottom). A clonal Jurkat iSynPro eGFP:ffluc reporter cell line was transduced with either the huCD19 CAR, the huCD19 EBD(4-OHT) CAR constructs with different glycine linkers (Gly1-3), the B7H3 CAR, the B7H3 EBD(4-OHT) CAR, or the B7H3 EBD(CMP8) CAR, and MTX selected if possible. 24-hours prior to assays, the transduced T cells were co-cultured with target cell lines (K562+CD19 for huCD19 CARs or K562 Parental for B7H3 CARs), and various concentrations of drug (4-OHT or CMP8). At the 24 hr timepoint, cells were collected and evaluated by flow cytometry. Cells were initially stained for CD33 to gate out target cell lines before they were evaluated for GFP expression by positivity and/or mean fluorescence intensity (MFI). Cell lines were stained for their transduction marker confirming expression of the various CAR-EBD constructs.

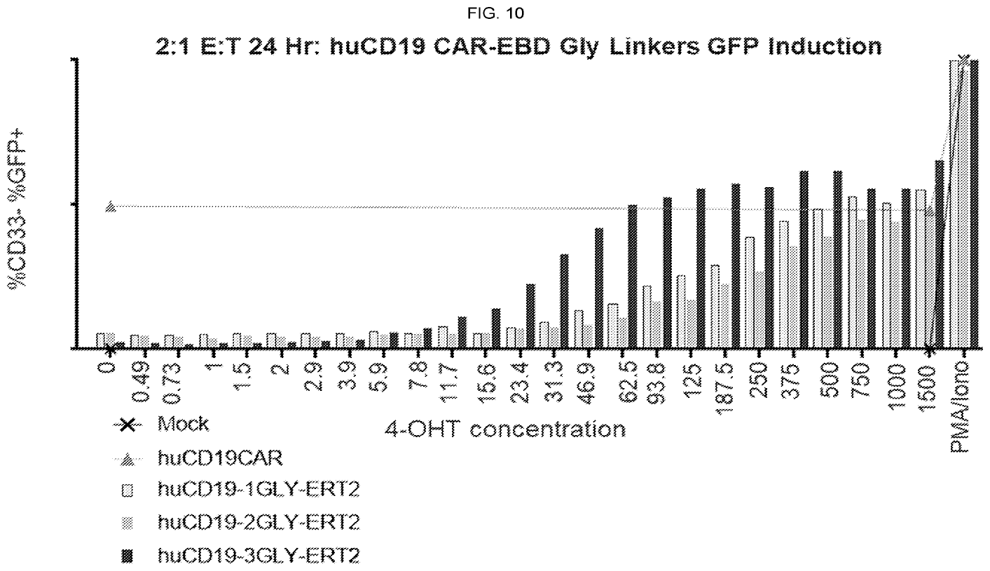

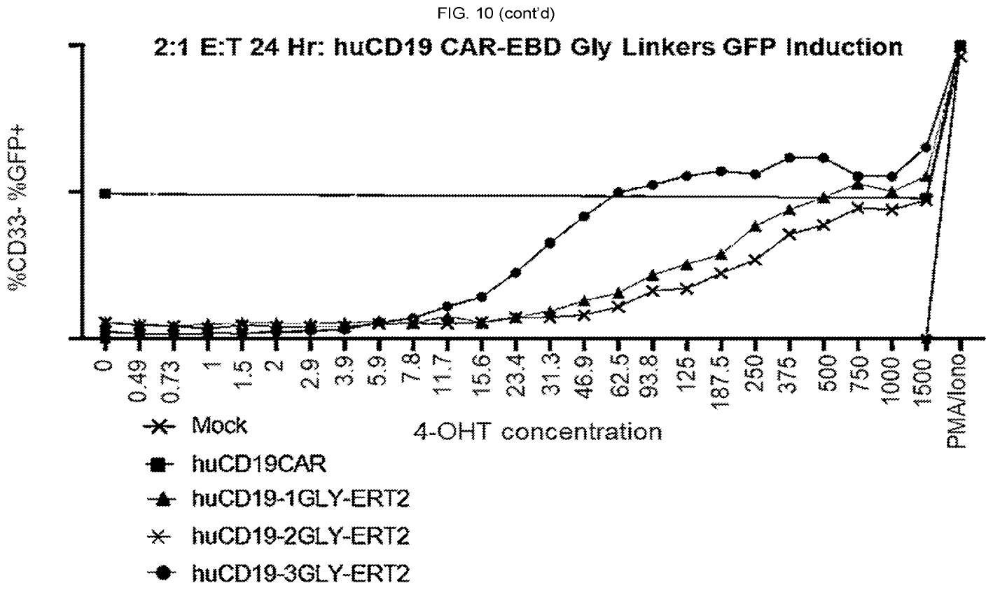

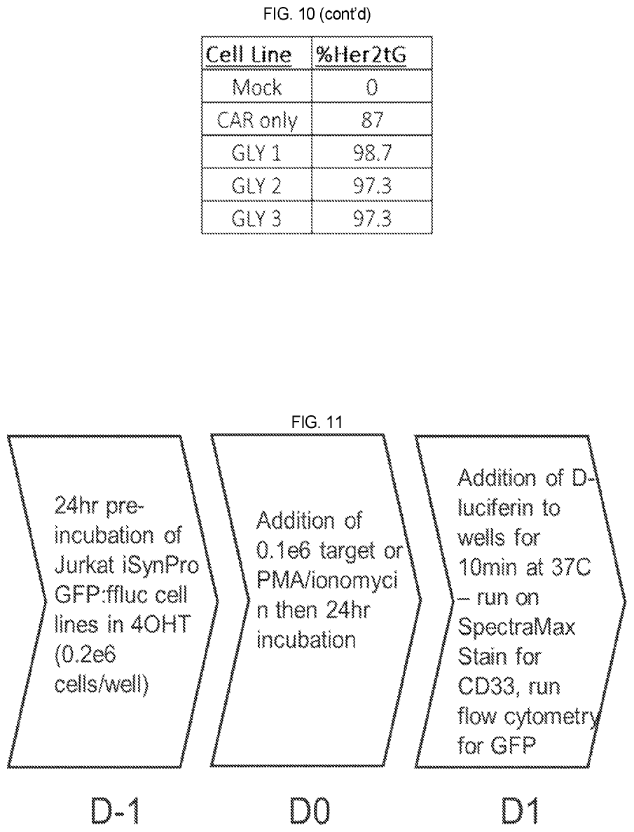

FIGS. 10, 14, and 15 provide results of huCD19-EBD(4-OHT) CAR glycine linker studies. Evaluation of activation curve by expression of GFP for the huCD19-EBD(4-OHT) CARs with different glycine linkers show drug depending activation (On/Off state) as well as the effects different linkers can have on the inducibility and leaky construct expression. For example, the Gly3 linker was observed to “turn on” at lower levels of drug and reach maximal induction at lower levels of drug compared to the Gly1 and 2 mutants in this study.

This experiment was repeated with a more dynamic range of drug concentrations with the addition of the tamoxifen metabolite (Z)-endoxifen, and a control group of drug with no tumor cells to examine the potential of activation in the absence of target. Evaluation of mean fluorescence intensity (MFI) show an on/off state for both 4-OHT and (Z)-endoxifen for all Gly linker constructs with similar activation curves.

FIGS. 11-13, 16 and 17 depict results of different B7H3-EBD CAR mutant studies. A first study was performed the confirm functionality of the B7H3 CAR ERT2 (EBD(4-OHT)) mutant responsive to 4-OHT or the EBD(CMP8) mutant responsive to both 4-OHT and CMP8 (EBD[L348M, M431G, G521R] mutant). 0, 500, and 1000 nM drug concentrations were chosen. A clear on/off state was observed and responsiveness to 4-OHT was shown for both EBD mutants (EBD(4-OHT) and EBD(CMP8). In addition, response to CMP8 was only observed with the CMP8 responsive EBD(CMP8) mutant (L348M, M431G, G521R]), and not with the EBD(4-OHT) mutant (not shown).

Following this study, a larger activation curve study was performed. For the B7H3-EBD(4-OHT) CAR a clear on/off state was observed with drug dependent activation. However, for the CMP8 responsive EBD(CMP8) mutant, induction of both 4-OHT and CMP8 expression was still observed even at 0.49 nM of drug.

The larger activation curve study was repeated with a more dynamic range of drug concentrations with the addition of the tamoxifen metabolite (Z)-endoxifen. Evaluation of mean fluorescence intensity (MFI) confirms the response of the CMP8 (EBD(CMP8) [L348M, M431G, G521R]) mutant to 4-OHT and CMP8 along with demonstrating that (Z)-endoxifen can also induce the system. Examination of the EBD(4-OHT) responsive mutant confirms the ability of 4-OHT and (Z)-endoxifen to induce the system, but not CMP8.

In these studies, all constructs show an on/off state for their respective drugs.

(xviii) Closing Paragraphs. The nucleic acid and amino acid sequences provided herein are shown using letter abbreviations for nucleotide bases and amino acid residues, as defined in 37 C.F.R. § 1.822 and set forth in the tables in WIPO Standard ST.25 (1998), Appendix 2, Tables 1 and 3. Only one strand of each nucleic acid sequence is shown, but the complementary strand is understood as included in embodiments where it would be appropriate.

The terms “specific binding affinity” or “specifically binds” or “specific binding” or “specifically targets” as used herein, describe binding of one molecule to another at greater binding affinity than background binding. A binding domain (e.g., of a CAR including a binding domain) “specifically binds” to a target molecule if it binds to or associates with a target molecule with an affinity or Ka (i.e. an equilibrium association constant of a particular binding interaction with units of 1/M) of, for example, greater than or equal to 10⁵ M⁻¹. In particular embodiments, a binding domain (or CAR) binds to a target with a Ka greater than or equal to 10⁶ M⁻¹, 107 M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains refers to those binding domains with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater.

Alternatively, affinity may be defined as an equilibrium dissociation constant (Kd) of a particular binding interaction with units of M (e.g., 10⁻⁵ M to 10⁻¹³ M, or less). Affinities of binding domains and CAR proteins according to the present disclosure can be readily determined using conventional techniques, e.g., by competitive ELISA (enzyme-linked immunosorbent assay), or by binding association, or displacement assays using labeled ligands, or using a surface-plasmon resonance device such as the Biacore T100, which is available from Biacore, Inc., Piscataway, N.J., or optical biosensor technology such as the EPIC system or EnSpire that are available from Corning and Perkin Elmer respectively (see also, e.g., Scatchard et al. (1949) Ann. N.Y. Acad. Sci. 51:660; U.S. Pat. Nos. 5,283,173; 5,468,614).

In particular embodiments, the affinity of specific binding is 2 times greater than background binding, 5 times greater than background binding, 10 times greater than background binding, 20 times greater than background binding, 50 times greater than background binding, 100 times greater than background binding, or 1000 times greater than background binding or more.

“Derived from” as used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3ζ molecule, the intracellular signaling domain retains sufficient CD3ζ structure such that is has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3ζ sequence and delete unwanted sequence, or impose mutations, to arrive at the intracellular signaling domain.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to activate a cell expressing an activity-inducible fusion protein in the presence of its relevant drug molecule and relevant physiological condition (e.g., antigen binding for a CAR; ligand binding for a co-stimulatory or inhibitory molecule).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 19% of the stated value; ±18% of the stated value; 17% of the stated value; 16% of the stated value; ±15% of the stated value; 14% of the stated value; ±13% of the stated value; 12% of the stated value; 11% of the stated value; 10% of the stated value; 9% of the stated value; 8% of the stated value; 7% of the stated value; ±6% of the stated value; 5% of the stated value; 4% of the stated value; ±3% of the stated value; 2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A chimeric antigen receptor (CAR) that when expressed by a cell comprises:
an extracellular component and an intracellular component linked by a transmembrane domain, wherein
the extracellular component comprising a ligand binding domain that binds a cancer antigen or a viral antigen and
the intracellular component comprising an intracellular signaling domain and an estrogen binding domain having a set of mutations selected from
G521R;
E353A;
L384M and M421G;
L384M, M421G, and G521R; and
G400V, M543A, and L544A.

2. An activity-inducible fusion protein comprising an hsp90 binding domain and an intracellular signaling domain of (i) a co-stimulatory immune molecule or (ii) an inhibitory immune molecule.

3. The activity-inducible fusion protein of claim 2, wherein the hsp90 binding domain binds a drug molecule.

4. The activity-inducible fusion protein of claim 3, wherein the hsp90 binding domain binds hsp90 with a lower affinity than it binds the drug molecule.

5. The activity-inducible fusion protein of claim 2, wherein the hsp90 binding domain comprises a hormone binding domain.

6. The activity-inducible fusion protein of claim 5, wherein the hormone binding domain is an engineered estrogen receptor binding domain (EBD).

7. The activity-inducible fusion protein of claim 6, wherein the engineered EBD comprises a binding domain portion of the estrogen receptor and a set of mutations selected from G521R; E353A; L384M and M421G; L384M, M421G, and G521R; or G400V, M543A, and L544A.

8. The activity-inducible fusion protein of claim 7, wherein the engineered EBD has the sequence as set forth in SEQ ID NO: 4, SEQ ID NO: 6, SEQ ID NO: 9, SEQ ID NO: 11, or SEQ ID NO: 13.

9. The activity-inducible fusion protein of claim 3, wherein the drug molecule comprises a small molecule estrogen analog.

10. The activity-inducible fusion protein of claim 9, wherein the small molecule estrogen analog comprises tamoxifen, a salt of tamoxifen, a metabolite of tamoxifen, or a compound that is structurally similar to tamoxifen.

11. The activity-inducible fusion protein of claim 9, wherein the small molecule estrogen analog comprises tamoxifen, 4-OHT, Endoxifen, ES8, or CMP8.

12. The activity-inducible fusion protein of claim 2, wherein the activity-inducible fusion protein is a chimeric antigen receptor (CAR) that when expressed by a cell comprises:
an extracellular component and an intracellular component linked by a transmembrane domain, wherein
the extracellular component comprises a ligand binding domain and
the intracellular component comprises the intracellular signaling domain of the co-stimulatory immune molecule and the hsp90 binding domain.

13. The activity-inducible fusion protein of claim 12, wherein the ligand binding domain binds a cancer antigen or a viral antigen.

14. The activity-inducible fusion protein of claim 12, wherein the ligand binding domain comprises an scFv that binds HER2, CE7, hB7H3, EGFR, EGFRvIII, CD19, CD20, CD22, EphA2, IL13Ra2, L1CAM, oaGD2, B7H3, CD33, Mesothelin, ROR1, FITC or VAR2CSA.

15. The activity-inducible fusion protein of claim 14, wherein the scFv has a sequence as set forth in SEQ ID NO: 16, SEQ ID NO: 18, SEQ ID NO: 19, SEQ ID NO: 20, SEQ ID NO: 21, SEQ ID NO: 23, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 342, or SEQ ID NO: 43.

16. The activity-inducible fusion protein of claim 12, wherein the ligand binding domain binds an immune cell antigen.

17. The activity-inducible fusion protein of claim 16, wherein the immune cell antigen is expressed by a B cell, a T cell, a natural kiIler cell, a natural kiIler T cell, a MAIT cell, a myeloid cell, a macrophage, a monocyte, or a dendritic cell.

18. The activity-inducible fusion protein of claim 12, wherein the ligand binding domain binds a hapten.

19. The activity-inducible fusion protein of claim 18, wherein the hapten comprises fluorescein, urushiol, quinone, biotin, or dinitrophenol.

20. The activity-inducible fusion protein of claim 18, wherein the ligand binding domain is an scFv having the sequence as set forth in SEQ ID NO: 44, SEQ ID NO: 45, SEQ ID NO: 46, SEQ ID NO: 47, SEQ ID NO: 48, SEQ ID NO: 49, SEQ ID NO: 51, SEQ ID NO: 53, SEQ ID NO: 60, SEQ ID NO: 61, SEQ ID NO: 62, SEQ ID NO: 63, SEQ ID NO: 64, or SEQ ID NO: 65.

21. The activity-inducible fusion protein of claim 12, wherein the extracellular component further comprises a spacer region.

22. The activity-inducible fusion protein of claim 21, wherein the spacer region comprises an IgG4 hinge.

23. The activity-inducible fusion protein of claim 12, wherein the intracellular component further comprises a CD3ζ intracellular signaling domain.

24. The activity-inducible fusion protein of claim 12, wherein the intracellular signaling domain comprises a 4-1 BB intracellular signaling domain.

25. The activity-inducible fusion protein of claim 12, wherein the intracellular signaling domain comprises a 4-1 BB intracellular signaling domain and wherein the intracellular component further comprises a CD3ζ intracellular signaling domain.

26. The activity-inducible fusion protein of claim 12, further comprising a 1 Gly, 2 Gly, or 3Gly junction amino acid adjacent to the hsp90 binding domain or no linker or junction amino acid adjacent to the hsp90 binding domain.

27. The activity-inducible fusion protein of claim 26, wherein the 1, 2, or 3 Gly junction amino acid, if present, is 5′ of the hsp90 binding domain and 3′ of the intracellular signaling domain.

28. The activity-inducible fusion protein of claim 12, wherein the hsp90 binding domain is 3′ of the intracellular signaling domain.

29. The activity-inducible fusion protein of claim 12, wherein the hsp90 binding domain is 5′ of the intracellular signaling domain and 3′ of the transmembrane domain.

30. The activity-inducible fusion protein of claim 25, wherein the hsp90 binding domain is 5′ of the CD3ζ intracellular signaling domain and 3′ of the 4-1BB intracellular signaling domain.

31. The activity-inducible fusion protein of claim 12, wherein the intracellular component lacks linkers and junction amino acids.

32. The activity-inducible fusion protein of claim 12, wherein the transmembrane domain comprises a CD28 transmembrane domain.

33. The activity-inducible fusion protein of claim 12, having the protein sequence as set forth in SEQ ID NO: 118.

34. The activity-inducible fusion protein of claim 2, wherein the activity-inducible fusion protein does not comprise a degron sequence.

35. The activity-inducible fusion protein of claim 2, wherein the co-stimulatory immune molecule comprises 4-1EE, OX40, CD40, CD30, CD27, DR3, SLAMF1, ICOS, GITR, CD25, CD28, CD79A, CD79B, CD226, CARD11, DAP10, DAP12, DR3, FcRα, FcRβ, FcRγ, Fyn, Lck, LAT, LRP, LIGHT, NKG2D, NOTCH1, NOTCH2, NOTCH3, NOTCH4, ROR2, Ryk, SIp76, pTα, TCRα, TCRβ, TIM1, TRIM, Zap70, or PTCH2.

36. The activity-inducible fusion protein of claim 2, wherein the inhibitory immune molecule comprises PD1, PD-L1, PD-L2, CTLA4, TIM3, CEACAM, LAG3, VISTA, BTLA, TIGIT, LAIR1, CD80, CD86, CD160, 2B4, B7-H3 (CD276), B7-H4 (VTCN1), KIR, A2aR, MHC class I, MHC class II, GAL9, adenosine, or TGFR β.

37. An activity-inducible fusion protein comprising an hsp90 binding domain wherein the activity-inducible fusion protein lacks a degron sequence.

38. The activity-inducible fusion protein of claim 37, wherein the hsp90 binding domain binds hsp90 with a lower affinity than it binds a drug molecule.

39. The activity-inducible fusion protein of claim 37, wherein the hsp90 binding domain comprises a hormone binding domain.

40. The activity-inducible fusion protein of claim 39, wherein the hormone binding domain is an engineered estrogen receptor binding domain (EBD).

41. The activity-inducible fusion protein of claim 40, wherein the engineered EBD comprises a binding domain portion of the estrogen receptor and a set of mutations selected from G521 R; E353A; L384M and M421G; L384M, M421G, and G521R; or G400V, M543A, and L544A.

42. The activity-inducible fusion protein of claim 41 wherein the engineered EBD has the sequence as set forth in SEQ ID NO: 4, SEQ ID NO: 6, SEQ ID NO: 9, SEQ ID NO: 11, or SEQ ID NO: 13.

43. The activity-inducible fusion protein of claim 38, wherein the drug molecule comprises a small molecule estrogen analog.

44. The activity-inducible fusion protein of claim 43, wherein the small molecule estrogen analog comprises tamoxifen, a salt of tamoxifen, a metabolite of tamoxifen, or a compound that is structurally similar to tamoxifen.

45. The activity-inducible fusion protein of claim 43, wherein the small molecule estrogen analog comprises tamoxifen, 4-OHT, Endoxifen, ES8, or CMP8.

46. A nucleotide encoding an activity-inducible fusion protein of claim 2.

47. The nucleotide of claim 46, comprising the coding sequence as set forth in SEQ ID NO: 7, SEQ ID NO: 14, or SEQ ID NO: 15.

48. The nucleotide of claim 46, further comprising the coding sequence as set forth in SEQ ID NO: 17.

49. The nucleotide of claim 46, further comprising the coding sequence as set forth in SEQ ID NO: 79.

50. The nucleotide of claim 46, further comprising the coding sequence as set forth in SEQ ID NO: 124.

51. The nucleotide of claim 46, further comprising the coding sequence as set forth in SEQ ID NO: 123.

52. The nucleotide of claim 46, further comprising the coding sequence as set forth in SEQ ID NO: 122.

53. The nucleotide of claim 46, having the sequence as set forth in SEQ ID NO: 125.

54. A cell genetically modified to express an activity-inducible fusion protein of claim 2.

55. The cell of claim 54, genetically modified to express at least two types of an activity-inducible fusion protein of claim 1, wherein the at least two types have different hsp90 binding domains that bind different drug molecules.

56. The cell of claim 55, wherein the at least two types further have different ligand binding domains.

57. The cell of claim 56, wherein the different ligand binding domains bind different antigens.

58. The cell of claim 57, wherein the different antigens are associated with a common cancer, a common infection, a common immune cell type, or a common hapten or are associated with different cancers, different infections, different immune cell types, or different haptens.

59. The cell of claim 58, wherein the common cancer is acute lymphoblastic leukemia (ALL), acute myeloid leukemia (AML), or a brain cancer.

60. The cell of claim 54, wherein the cell is a T cell or a natural kiIler (NK) cell.

61. The cell of claim 60, wherein the T cell is a CD4+ or a CD8+ T cell.

62. The cell of claim 54, wherein the cell is an induced pluripotent stem cell (iPSC), a tumor-infiltrating lymphocyte (TIL), a marrow-infiltrating lymphocyte (MIL), a natural kiIler T cell (NKT), a mucosal-associated invariant T (MAIT) cell, a dendritic cell, a monocyte or a macrophage.

63. A system for controlling the activation state of an activity-inducible fusion protein in vivo comprising:
the activity-inducible fusion protein wherein the activity-inducible fusion protein comprises an hsp90 binding domain that binds a drug molecule and an intracellular signaling domain of a co-stimulatory immune molecule or an inhibitory immune molecule; and
the drug molecule.

64. The system of claim 63, wherein the hsp90 binding domain binds hsp90 with a lower affinity than it binds the drug molecule.

65. The system of claim 63, wherein the hsp90 binding domain comprises a hormone binding domain.

66. The system of claim 65, wherein the hormone binding domain comprises an engineered estrogen receptor binding domain (EBD).

67. The system of claim 66, wherein the engineered EBD comprises a binding domain portion of the estrogen receptor and a set of mutations selected from G521R; E353A; L384M and M421G; L384M, M421G, and G521R; or G400V, M543A, and L544A.

68. The system of claim 66, wherein the engineered EBD has the sequence as set forth in SEQ ID NO: 4, SEQ ID NO: 6, SEQ ID NO: 9, SEQ ID NO: 11, or SEQ ID NO: 13.

69. The system of claim 63, wherein the drug molecule comprises a small molecule estrogen analog.

70. The system of claim 69, wherein the small molecule estrogen analog comprises tamoxifen, a salt of tamoxifen, a metabolite of tamoxifen, or a compound that is structurally similar to tamoxifen.

71. The system of claim 69, wherein the small molecule estrogen analog comprises tamoxifen, 4-OHT, Endoxifen, ES8, or CMP8.

72. The system of claim 63, wherein the activity-inducible fusion protein is a CAR and wherein when expressed by a cell the CAR comprises
an extracellular component and an intracellular component linked by a transmembrane domain, wherein
the extracellular component comprises a ligand binding domain and
the intracellular component comprises the intracellular signaling domain of the co-stimulatory immune molecule and the hsp90 binding domain.

73. The system of claim 72, wherein the ligand binding domain binds a cancer antigen or a viral antigen.

74. The system of claim 72, wherein the ligand binding domain comprises an scFv that binds HER2, CE7, hB7H3, EGFR, EGFRvIII, CD19, CD20, CD22, EphA2, IL13Ra2, L1CAM, oaGD2, B7H3, CD33, Mesothelin, ROR1, FITC or VAR2CSA.

75. The system of claim 74, wherein the scFv has a sequence as set forth in SEQ ID NO: 16, SEQ ID NO: 18, SEQ ID NO: 19, SEQ ID NO: 20, SEQ ID NO: 21, SEQ ID NO: 23, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, or SEQ ID NO: 43.

76. The system of claim 72, wherein the ligand binding domain binds an immune cell antigen.

77. The system of claim 76, wherein the immune cell antigen is expressed by a B cell, a T cell, a natural kiIler cell, a natural kiIler T cell, a MAIT cell, a myeloid cell, a macrophage, a monocyte, or a dendritic cell.

78. The system of claim 72, wherein the ligand binding domain binds a hapten.

79. The system of claim 78, wherein the hapten comprises fluorescein, urushiol, quinone, biotin, or dinitrophenol.

80. The system of claim 78, wherein the ligand binding domain is an scFv having the sequence as set forth in SEQ ID NO: 44, SEQ ID NO: 45, SEQ ID NO: 46, SEQ ID NO: 47, SEQ ID NO: 48, SEQ ID NO: 49, SEQ ID NO: 51, SEQ ID NO: 53, SEQ ID NO: 60, SEQ ID NO: 61, SEQ ID NO: 62, SEQ ID NO: 63, SEQ ID NO: 64, or SEQ ID NO: 65.

81. The system of claim 72, wherein the extracellular component further comprises a spacer region.

82. The system of claim 81, wherein the spacer region comprises an IgG4 hinge.

83. The system of claim 72, wherein the intracellular component further comprises a CD3ζ intracellular signaling domain.

84. The system of claim 72, wherein the intracellular signaling domain comprises a 4-1EE intracellular signaling domain.

85. The system of claim 72, wherein the intracellular signaling domain comprises a 4-1EE intracellular signaling domain and wherein the intracellular component further comprises a CD3ζ intracellular signaling domain.

86. The system of claim 72, further comprising a 1 Gly, 2 Gly, or 3 Gly junction amino acid adjacent to the hsp90 binding domain or no linker or junction amino acid adjacent to the hsp90 binding domain.

87. The system of claim 86, wherein the 1, 2, or 3 Gly junction amino acid, if present, is 5′ of the hsp90 binding domain and 3′ of the intracellular signaling domain.

88. The system of claim 63, wherein the hsp90 binding domain is 3′ of the intracellular signaling domain.

89. The system of claim 63, wherein the hsp90 binding domain is 5′ of the intracellular signaling domain and 3′ of the transmembrane domain.

90. The system of claim 85, wherein the hsp90 binding domain is 5′ of the CD3ζ intracellular signaling domain and 3′ of the 4-1 BB intracellular signaling domain.

91. The system of claim 63, wherein the intracellular component lacks linkers and junction amino acids.

92. The system of claim 72, wherein the transmembrane domain comprises a CD28 transmembrane domain.

93. The system of claim 72, wherein the CAR has the protein sequence as set forth in SEQ ID NO: 118.

94. The system of claim 63, wherein the activity-inducible fusion protein lacks a degron sequence.

95. The system of claim 63, wherein the co-stimulatory immune molecule comprises 4-1 BB, OX40, CD40, CD30, CD27, DR3, SLAMF1, ICOS, GITR, CD25, CD28, CD79A, CD79B, CD226, CARD11, DAP10, DAP12, DR3, FcRα, FcRβ, FcRγ, Fyn, Lck, LAT, LRP, LIGHT, NKG2D, NOTCH1, NOTCH2, NOTCH3, NOTCH4, ROR2, Ryk, Slp76, pTα, TCRα, TCRβ, TIM1, TRIM, Zap70, or PTCH2.

96. The system of claim 63, wherein the inhibitory immune molecule comprises PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD80, CD86, CD160, 2B4, B7-H3 (CD276), B7-H4 (VTCN1), KIR, A2aR, MHC class I, MHC class II, GAL9, adenosine, or TGFR β.

97. The system of claim 63, comprising at least two types of an activity-inducible fusion protein, wherein the at least two types have different hsp90 binding domains that bind different drug molecules.

98. The system of claim 97, wherein the at least two types further have different ligand binding domains.

99. The system of claim 97, wherein the different ligand binding domains bind different antigens.

100. The system of claim 99, wherein the different antigens are associated with a common cancer, common cancer, a common infection, a common immune cell type, or a common hapten or are associated with different cancers, different infections, different immune cell types, or different haptens.

101. The system of claim 100, wherein the common cancer is acute lymphoblastic leukemia (ALL), acute myeloid leukemia (AML), or a brain cancer.

102. A system for controlling the activation state of an activity-inducible fusion protein in vivo comprising:
an activity-inducible fusion protein wherein the activity-inducible fusion protein comprises an hsp90 binding domain that binds a drug molecule and wherein the activity-inducible fusion protein does not include a degron sequence; and
the drug molecule.

103. The system of claim 102, wherein the hsp90 binding domain binds hsp90 with a lower affinity than it binds the drug molecule.

104. The system of claim 102, wherein the hsp90 binding domain comprises a hormone binding domain.

105. The system of claim 104, wherein the hormone binding domain comprises an engineered estrogen receptor binding domain (EBD).

106. The system of claim 105, wherein the engineered EBD comprises a binding domain portion of the estrogen receptor and a set of mutations selected from G521R; E353A; L384M and M421G; L384M, M421G, and G521R; or G400V, M543A, and L544A.

107. The system of claim 105, wherein the engineered EBD has the sequence as set forth in SEQ ID NO: 4, SEQ ID NO: 6, SEQ ID NO: 9, SEQ ID NO: 11, or SEQ ID NO: 13.

108. The system of claim 102, wherein the drug molecule comprises a small molecule estrogen analog.

109. The system of claim 108, wherein the small molecule estrogen analog comprises tamoxifen, a salt of tamoxifen, a metabolite of tamoxifen, or a compound that is structurally similar to tamoxifen.

110. The system of claim 108, wherein the small molecule estrogen analog comprises tamoxifen, 4-OHT, Endoxifen, ES8, or CMP8.

111. The system of claim 63, formulated for administration to a subject.

112. The system of claim 111, wherein the formulated system comprises
(i) ex vivo manufactured cells expressing the activity-inducible fusion protein formulated into a pharmaceutically acceptable carrier to create a modified formulation; and/or
(ii) cell-targeted viral vectors and/or a cell-targeted nanoparticles formulated into a pharmaceutically acceptable carrier to create a modifying formulation wherein the cell-targeted viral vectors and/or the cell-targeted nanoparticles comprise gene-modifying components that result in expression of the activity-inducible fusion protein in vivo by the targeted cell following administration; and
(iii) the drug molecule formulated into a pharmaceutically acceptable carrier to create a drug composition.

113. A method of treating a subject in need thereof comprising administering a system of claim 112 to the subject, thereby treating the subject.

114. The method of claim 113, wherein the administering comprises administering the modified formulation and the drug composition.

115. The method of claim 113, wherein the administering comprises administering the modifying formulation and the drug composition.

116. The method of claim 113, wherein the method further comprises stopping administration of the drug molecule to reduce a side effect of system administration.

117. The method of claim 113, wherein the method further comprises stopping administration of the drug molecule when the subject is no longer in need thereof.

118. The method of claim 113, wherein the subject is in need thereof due to cancer or a viral infection.

119. The method of claim 113, wherein the system comprises at least two types of an activity-inducible fusion protein, wherein the at least two types have different hsp90 binding domains that bind different drug molecules.

120. The method of claim 119, comprising administering at least two types of drug molecules wherein one of the at least two types binds the hsp90 binding domain of one activity-inducible fusion protein of the system and wherein one of the at least two types binds the hsp90 binding domain of a different activity-inducible fusion protein of the system.

121. The method of claim 120, wherein the method further comprises stopping administration of at least one of the drug molecules.

122. The method of claim 120, wherein the method further comprises stopping administration of all of the drug molecules.

123. The method of claim 119, wherein the at least two types further have different ligand binding domains.

124. The method of claim 123, wherein the different ligand binding domains bind different antigens.

125. The method of claim 124, wherein the different antigens are associated with a common cancer, common cancer, a common infection, a common immune cell type, or a common hapten or are associated with different cancers, different infections, different immune cell types, or different haptens.

126. The method of claim 125, wherein the common cancer type is acute lymphoblastic leukemia (ALL), acute myeloid leukemia (AML), or a brain cancer.

127. An activity-inducible fusion protein comprising an hsp90 binding domain wherein activity is induced within 1 hour of administration of a drug molecule that binds the hsp90 binding domain.

128. The activity-inducible fusion protein of claim 127, wherein the activity-inducible fusion protein does not comprise a degron sequence.

129. The activity-inducible fusion protein of claim 127, wherein the hsp90 binding domain binds hsp90 with a lower affinity than it binds a drug molecule.

130. The activity-inducible fusion protein of claim 127, wherein the hsp90 binding domain comprises a hormone binding domain.

131. The activity-inducible fusion protein of claim 130, wherein the hormone binding domain is an engineered estrogen receptor binding domain (EBD).

132. The activity-inducible fusion protein of claim 131, wherein the EBD comprises the binding domain portion of the estrogen receptor and a set of mutations selected from G521R; E353A; L384M and M421G; L384M, M421G, and G521R; or G400V, M543A, and L544A.

133. The activity-inducible fusion protein of claim 132, wherein the EBD has the sequence as set forth in SEQ ID NO: 4, SEQ ID NO: 6, SEQ ID NO: 9, SEQ ID NO: 11, or SEQ ID NO: 13.

134. The activity-inducible fusion protein of claim 128, wherein the drug molecule comprises a small molecule estrogen analog.

135. The activity-inducible fusion protein of claim 134, wherein the small molecule estrogen analog comprises tamoxifen, a salt of tamoxifen, a metabolite of tamoxifen, or a compound that is structurally similar to tamoxifen.

136. The activity-inducible fusion protein of claim 134, wherein the small molecule estrogen analog comprises tamoxifen, 4-OHT, Endoxifen, ES8, or CMP8.

137. The activity-inducible fusion protein of claim 127, wherein the activity-inducible fusion protein is a chimeric antigen receptor (CAR) that when expressed by a cell comprises:
an extracellular component and an intracellular component linked by a transmembrane domain, wherein
the extracellular component comprises a ligand binding domain and
the intracellular component comprises an intracellular signaling domain and the hsp90 binding domain.

138. The activity-inducible fusion protein of claim 137, wherein the ligand binding domain binds a cancer antigen or a viral antigen.


SEQUENCE LISTING (308 sequences)

SEQ ID NO: 1 - protein, 595 aa, Homo sapiens [sequence omitted]

SEQ ID NO: 2 - protein, 314 aa, Artificial Sequence
  EBD with Optional N- and C-Terminal Truncation
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 3 - protein, 595 aa, Artificial Sequence
  Estrogen Receptor with G521R mutation
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 4 - protein, 314 aa, Artificial Sequence
  EBD with G521R mutation
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 5 - protein, 595 aa, Artificial Sequence
  Estrogen Receptor with E353A mutation
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRALVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 6 - protein, 314 aa, Artificial Sequence
  EBD (E353A) Pairs with estrogen analog ES8
SAGDMRAANLWPSPLMIKRSKKNSLALSLTADQMVSALLDAEPPILYSEYDPTRPFSEASMMGLLTNLADRALVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 7 - DNA, 942 bp, Artificial Sequence
  EBD (E353A) Coding Sequence
tctgctggcgatatgagagccgctaacctgtggccttctccactgatgatcaagcggagcaagaagaactctctggccctgtctctgaccgccgaccagatggtttctgctctgctggatgccgagcctcctatcctgtactccgagtacgaccccaccagaccttttagcgaggccagcatgatgggcctgctgaccaatctggctgacagagccctggtgcacatgatcaactgggccaagcgcgtgcccggctttgtcgatctgacactgcacgaccaagtgcatctgctggaatgcgcctggctggaaattctgatgatcggcctcgtgtggcggagcatggaacaccctggaaaactgctgttcgcccctaacctgctgctcgacagaaaccagggcaaatgcgtggaaggcatggtggaaatcttcgatatgctgctggccacctccagccggttccggatgatgaatctccagggcgaagagttcgtgtgcctgaagtccatcatcctgctgaatagcggcgtgtacacattcctgagcagcaccctgaaatccctggaagagaaggaccacatccaccgggtgctcgacaagatcaccgacacactgatccacctgatggccaaggccggactgactctgcagcagcaacatcagagactggcccagctgctgctcatcctgagccacatcagacacatgagcaacaaaggcatggaacatctgtacagcatgaagtgcaagaacgtggtgcccctgtacgacctgctcctggaaatgctggacgcccacagactgcacgcccctacatctagaggtggcgcctctgtggaagagacagaccagtctcacctggccacagccggcagcacaagctctcattccctgcagaagtactacatcaccggcgaggccgagggatttcctgccacagtt

SEQ ID NO: 8 - protein, 595 aa, Artificial Sequence
  Estrogen Receptor with L384M and M421G mutations
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 9 - protein, 314 aa, Artificial Sequence
  EBD with L384M and M421G mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 10 - protein, 595 aa, Artificial Sequence
  Estrogen Receptor with L384M, M421G, and G521R mutations
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 11 - protein, 314 aa, Artificial Sequence
  EBD (L384M, M421G, G521R) pairs with CMP8
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 12 - protein, 595 aa, Artificial Sequence
  Estrogen receptor (G400V, M543A, and L544A mutations)
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 13 - protein, 314 aa, Artificial Sequence
  ERT2
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 14 - DNA, 942 bp, Artificial Sequence
  ERT2
tcagcgggtgacatgcgagctgccaatctctggccgtcacctttgatgattaaacgatcaaagaaaaatagcctggctctgagcttgacggctgatcaaatggtaagtgcactgctcgacgccgaaccgccgatcctgtattccgagtatgaccccacacggccattctccgaagccagtatgatgggacttcttaccaaccttgcagatcgcgaactcgtacatatgataaattgggcgaagcgcgttccagggttcgtcgacctcacgttgcatgatcaggtccatctgctggaatgcgcgtggttggaaatcttgatgattggtttggtctggcgatctatggagcatccggtgaaacttcttttcgcgcccaatcttcttttggatcgcaatcaagggaaatgcgtggaaggaatggtcgaaatttttgatatgcttctcgcgaccagctcccgattcaggatgatgaatttgcagggtgaagagtttgtatgtcttaagtctattatattgctcaactccggcgtttatacattcctttctagcacacttaaatcactcgaggaaaaggaccacatccaccgggttttggataaaatcactgatacactgattcatttgatggccaaggccggactcacactccaacaacagcaccaacggcttgcacagctcctcctcatactttctcatataaggcacatgtcaaataagggaatggagcacctgtactccatgaagtgtaaaaatgtagtccctctgtatgatctgctccttgaggctgcagacgcgcatagactgcacgccccaacgtcacgagggggggcctcagtggaagagacagatcagagccatctcgcaaccgcaggatctacatccagccactctcttcagaaatactatataactggcgaagccgaagggttccctgccaccgtt

SEQ ID NO: 15 - protein, 942 aa, Artificial Sequence
  EBD (L384M, M421G, G521R)
TCTGCTGGCGATATGAGAGCCGCTAACCTGTGGCCTTCTCCACTGATGATCAAGCGGAGCAAGAAGAACTCTCTGGCCCTGTCTCTGACCGCCGACCAGATGGTTTCTGCTCTGCTGGATGCCGAGCCTCCTATCCTGTACTCCGAGTACGACCCCACCAGACCTTTTAGCGAGGCCAGCATGATGGGCCTGCTGACCAATCTGGCCGACAGAGAACTGGTGCACATGATCAACTGGGCCAAGCGCGTGCCCGGCTTTGTCGATCTGACACTGCACGACCAAGTGCATCTGCTGGAATGCGCCTGGATGGAAATCCTGATGATCGGCCTCGTGTGGCGGAGCATGGAACACCCTGGAAAACTGCTGTTCGCCCCTAACCTGCTGCTCGACAGAAACCAGGGCAAATGCGTGGAAGGCGGCGTGGAAATCTTCGATATGCTGCTGGCCACCTCCAGCCGGTTCCGGATGATGAATCTCCAGGGCGAAGAGTTCGTGTGCCTGAAGTCCATCATCCTGCTGAATAGCGGCGTGTACACATTCCTGAGCAGCACCCTGAAATCCCTGGAAGAGAAGGACCACATCCACCGGGTGCTCGACAAGATCACCGACACACTGATCCACCTGATGGCCAAGGCCGGACTGACTCTGCAGCAGCAACATCAGAGACTGGCCCAGCTGCTGCTCATCCTGAGCCACATCAGACACATGAGCAACAAGCGGATGGAACATCTGTACTCCATGAAGTGCAAGAACGTGGTGCCCCTGTACGACCTGCTCCTGGAAATGCTGGACGCCCACAGACTGCACGCCCCTACATCTAGAGGTGGCGCCTCTGTGGAAGAGACAGACCAGTCTCACCTGGCCACAGCCGGCAGCACAAGCTCTCATTCCCTGCAGAAGTACTACATCACCGGCGAGGCCGAGGGCTTTCCAGCCACAGTT

SEQ ID NO: 16 - protein, 264 aa, Artificial Sequence
  EGFRVIII (806) scFv
MLLLVTSLLLCELPHPAFLLIPDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGSTSGSGKPGSGEGSTKGDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKR

SEQ ID NO: 17 - DNA, 787 bp, Artificial Sequence
  EGFRVIII (806) scFv
atgctgctgctggtgaccagcctgctgctgtgcgagctgccccaccccgcctttctgctgatcccccaggtgcagctgcagcagcctggcgccgagctggtgaagccaggcgccagcgtgaagctgtcctgcaaggccagcggctacaccttcaccggctactggatgcactgggtgaagcagagacccggccacggcctggaatggatcggcgagatcaaccccagcaacggccggaccaactacaacgagcggttcaagagcaaggccaccctgaccgtggacaagagcagcaccaccgccttcatgcagctgtccggcctgaccagcgaggacagcgccgtgtacttctgcgccagggactactacggcaccagctacaacttcgactactggggccagggcaccacactgaccgtgagcagcggcggagggggctctggcggcggaggatctgggggagggggcagcgacatccagatgacccagagcagcagcagcttcagcgtgagcctgggcgaccgggtgaccatcacctgtaaggccaacgaggacatcaacaaccggctggcctggtatcagcagacccccggcaacagccccaggctgctgatcagcggcgccaccaacctggtgaccggcgtgcccagccggtttagcggcagcggctccggcaaggactacaccctgaccatcacaagcctgcaggccgaggacttcgccacctactactgccagcagtactggtccacccccttcaccttcggcagcggcaccgagctggaaa

SEQ ID NO: 18 - protein, 265 aa, Artificial Sequence
  EGFR scFv
MLLLVTSLLLCELPHPAFLLIPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRT

SEQ ID NO: 19 - protein, 268 aa, Artificial Sequence
  huCD19 (G01S) scFv
MLLLVTSLLLCELPHPAFLLIPEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSGGGGSGGGGSGGGGSQSALTQPRSVSGFPGQSVTISCTGTTSDDVSWYQQHPGKAPQLMLYDVSKRPSGVPHRFSGSRSGRAASLIISGLQTEDEADYFCSSYAGRYNSVLFGGGTKLTVL

SEQ ID NO: 20 - protein, 267 aa, Artificial Sequence
  muCD19 (FMC63) scFv
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 21 - protein, 244 aa, Artificial Sequence
  CD19 scFv
DIQMTQTTSSLSALGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 22 - DNA, 735 bp, Artificial Sequence
  CD19 scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 23 - protein, 266 aa, Artificial Sequence
  CD20 (Leu 16) scFv
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 24 - DNA, 798 bp, Artificial Sequence
  CD20 scFv
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctca

SEQ ID NO: 25 - protein, 270 aa, Artificial Sequence
  CD22 (m971) scFv
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKRT

SEQ ID NO: 26 - protein, 316 aa, Artificial Sequence
  B7H3 (hBRCA84D) scFv
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITGQPMTFPPEALWVTVGLSVCLIALLVALAFVCWRKIKQSCEEENAGAEDQDGEGEGSKTALQPLKHSDSKEDDGQEIA

SEQ ID NO: 27 - protein, 264 aa, Artificial Sequence
  L1CAM (CE7) scFv
MLLLVTSLLLCELPHPAFLLIPQVQLQQPGAELVKPGASVKLSCKASGYTFTGYWMHWVKQRPGHGLEWIGEINPSNGRTNYNERFKSKATLTVDKSSTTAFMQLSGLTSEDSAVYFCARDYYGTSYNFDYWGQGTTLTVSSGGGGSGGGGSGGGGSDIQMTQSSSSFSVSLGDRVTITCKANEDINNRLAWYQQTPGNSPRLLISGATNLVTGVPSRFSGSGSGKDYTLTITSLQAEDFATYYCQQYWSTPFTFGSGTELEIK

SEQ ID NO: 28 - protein, 259 aa, Artificial Sequence
  EphA2 (2A4) scFv
MLLLVTSLLLCELPHPAFLLIPQVQLLESGGGLVQPGGSLRLSCAASGFTFSSYTMSWVRQAPGQALEWMGTISSRGTYTYYPDSVKGRFTISRDNAKNSLYLQMNSLRAEDTAVYYCAREAIFTHWGRGTLVTVSSGGGGSGGGGSGGGGSDIQLTQSPSSLSASVGDRVTITCKASQDINNYHSWYQQKPGQAPRLLIYRANRLVDGVPDRFSGSGYGTDFTLTINNIESEDAAYYFCLKYNVFPYTFGQGTKVEIK

SEQ ID NO: 29 - protein, 259 aa, Artificial Sequence
  EpHA2 (4H5) scFv
MLLLVTSLLLCELPHPAFLLIPQVQLLESGGGLVQPGGSLRLSCAASGFTFSSYTMSWVRQAPGQALEWMGTISSGGTYTYYPDSVKGRFTISRDNAKNSLYLQMNSLRAEDTAVYYCAREAIFTYWGRGTLVTVSSGGGGSGGGGSGGGGSDIQLTQSPSSLSASVGDRVTITCKASQDINNYLSWYQQKPGQAPRLLIYRANRLVDGVPDRFSGSGYGTDFTLTINNIESEDAAYYFCLKYDVFPYTFGQGTKVEIK

SEQ ID NO: 30 - protein, 273 aa, Artificial Sequence
  FITC (E2) scFv
MLLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVS

SEQ ID NO: 31 - protein, 127 aa, Artificial Sequence
  GD2 (hu3F8) scFv
MLLLVTSLLLCELPHPAFLLIPSIVMTQTPKFLLVSAGDRVTITCKASQSVSNDVTWYQQKAGQSPKLLIYSASNRYSGVPDRFTGSGYGTAFTFTISTVQAEDLAVYFCQQDYSSFGGGTKLEIKR

SEQ ID NO: 32 - protein, 267 aa, Artificial Sequence
  Her2 (Herceptin) scFv
MLLLVTSLLLCELPHPAFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 33 - protein, 240 aa, Artificial Sequence
  IL13Ra2 (hu08) VlVh scFv
MLLLVTSLLLCELPHPAFLLIPDIQMTQSPSSLSASVGDRVTITCKASQDVGTAVAWYQQKPGKAPKLLIYSASYRSTGVPSRFSGSGSGTDFTLTISSLQPEDFATYYCQHHYSAPWTFGGGTKVEIKGGGGSGGGGSGGGGSEVQLVESGGGLVQPGGSLRLSCAASGFTFSRNGMSWVRQAPGKGLEWVATVSSGGSYIYYADSVKGRFTISRDNAKNSLYLQMNSLRAEDTAVYYC

SEQ ID NO: 34 - protein, 269 aa, Artificial Sequence
  IL13Ra2 hu08 VhVl scFv
MLLLVTSLLLCELPHPAFLLIPEVQLVESGGGLVQPGGSLRLSCAASGFTFSRNGMSWVRQAPGKGLEWVATVSSGGSYIYYADSVKGRFTISRDNAKNSLYLQMNSLRAEDTAVYYCARQGTTALATRFFDVWGQGTLVTVSSGGGGSGGGGSGGGGSDIQMTQSPSSLSASVGDRVTITCKASQDVGTAVAWYQQKPGKAPKLLIYSASYRSTGVPSRFSGSGSGTDFTLTISSLQPEDFATYYCQHHYSAPWTFGGGTKVEIKESK

SEQ ID NO: 35 - protein, 264 aa, Artificial Sequence
  IL13Ra2 (hu07) VlVh scFv
MLLLVTSLLLCELPHPAFLLIPDIQMTQSPSSLSASVGDRVTITCTASLSVSSTYLHWYQQKPGKAPKLLIYSTSNLASGVPSRFSGSGSGTDFTLTISSLQPEDFATYYCHQYHRSPLTFGGGTKVEIKGGGGSGGGGSGGGGSEVQLVESGGGLVQPGGSLRLSCAASGFTFTKYGVHWVRQAPGKGLEWVAVKWAGGSTDYNSALMSRFTISRDNAKNSLYLQMNSLRAEDTAVYYCARDHRDAMDYWGQGTLVTVSSESK

SEQ ID NO: 36 - protein, 264 aa, Artificial Sequence
  IL13Ra2 (hu07) VhVl scFv
MLLLVTSLLLCELPHPAFLLIPEVQLVESGGGLVQPGGSLRLSCAASGFTFTKYGVHWVRQAPGKGLEWVAVKWAGGSTDYNSALMSRFTISRDNAKNSLYLQMNSLRAEDTAVYYCARDHRDAMDYWGQGTLVTVSSGGGGSGGGGSGGGGSDIQMTQSPSSLSASVGDRVTITCTASLSVSSTYLHWYQQKPGKAPKLLIYSTSNLASGVPSRFSGSGSGTDFTLTISSLQPEDFATYYCHQYHRSPLTFGGGTKVEIKESK

SEQ ID NO: 37 - protein, 269 aa, Artificial Sequence
  oaGD2 (8B6) VlVh scFv
MLLLVTSLLLCELPHPAFLLIPDVVMTQTPLSLPVSLGDQASISCRSSQSLLKNNGNTFLHWYLQKSGQSPKLLIYKVSNRLSGVPDRFSGSGSGTYFTLKISRVEAEDLGVYFCSQSTHIPYTFGGGTKLELKRGGGGSGGGGSGGGGSEVKLVESGGGLVLPGDSLRLSCATSEFTFTDYYMTWVRQPPRKALEWLGFIRNRANGYTTEYNPSVKGRFTISRDNSQSILYLQMNTLRTEDSATYYCARVSNWAFDYWGQGTTLTVSS

SEQ ID NO: 38 - protein, 270 aa, Artificial Sequence
  ROR1 (R12) scFv
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 39 - protein, 139 aa, Artificial Sequence
  CD33 (h2H12) VhVl scFv
MLLLVTSLLLCELPHPAFLLIPQVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 40 - protein, 129 aa, Artificial Sequence
  CD33 (h2H12) VlVh scFv
MLLLVTSLLLCELPHPAFLLIPDIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIK

SEQ ID NO: 41 - protein, 150 aa, Artificial Sequence
  Mesothelin (P4) scFv
MLLLVTSLLLCELPHPAFLLIPQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGS

SEQ ID NO: 42 - protein, 448 aa, Artificial Sequence
  VAR2CSA (ID1-DBL2Xb) scFv
MLLLVTSLLLCELPHPAFLLIPSLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKY

SEQ ID NO: 43 - protein, 133 aa, Artificial Sequence
  Anti-IL13Ra2 (IL13 zetakine)
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRF

SEQ ID NO: 44 - protein, 251 aa, Artificial Sequence
  FITCE2 scFv
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVS

SEQ ID NO: 45 - protein, 251 aa, Artificial Sequence
  FITCE2 TyrH133Ala scFv
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFASYMDVWGQGTLVTVS

SEQ ID NO: 46 - protein, 250 aa, Artificial Sequence
  FITCE2 HisH131Ala scFv
VLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGAFYSYMDVWGQGTLVTVS

SEQ ID NO: 47 - protein, 254 aa, Artificial Sequence
  FL (4M5.3) scFv
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVS

SEQ ID NO: 48 - protein, 265 aa, Artificial Sequence
  FL (4D5Flu) scFv
DYKDIQMTQSPSSLSASVGDRVTITCRASQSLVHSQGNTYLRWYQQKPGKAPKVLIYKVSNRFSGVPSRFSGSGSGTDFTLTISSLQPEDFATYYCQQSTHVPWTFGQGTKVELKRAGGGGSGGGGSGGGGSSGGGSGGGGSGGGGSEVQLVESGGGLVQPGGSLRLSCAASGFTFSDYWMNWVRQAPGKGLEWVAQIRNKPYNYETYYADSVKGRFTISRDTSKNTVYLQMNSLRAEDTAVYYCTGSYYGMDYWGQGTLVTVSS

SEQ ID NO: 49 - protein, 244 aa, Artificial Sequence
  FL (4420) scFv
DVVMTQTPLSLPVSLGDQASISCRSSQSLVHSQGNTYLRWYLQKPGQSPKVLIYKVSNRFSGVPDRFSGSGSGTDFTLKISRVEAEDLGVYFCSQSTHVPWTFGGGTKLEIGGGGSGGGGSGGGGSEVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSS

SEQ ID NO: 50 - DNA, 789 bp, Artificial Sequence
  DNP scFv
cagtgtcagcagctggagcagtccggaggaggagccgaaggaggcctggtcaagcctgggggatccctggaactctgctgcaaagcctctggattctccctcagtagtagctactgcatatgttgggtccgccaggctccagggaaggggctggagtggatcggatgcatttatgctggtagtagtggtagcacttactacgcgagctgggtgaatggccgattcactctctccagagacattgaccagagcacaggttgcctacaactgaacagtctgacagccgcggacacggccatgtattactgtgcgagagccccctatagtagtggctgggtcctctactttaacttgtggggcccaggcaccctggtcattgtctcctcaggcggagggggctctggcggcggaggatctgggggagggggcagcccaggtgccacatttgcccaagtgctgacccagactccatcgcctgtgtctgcagctgtgggaggcacagtcaccatcagttgccagtccagtgagagtgtttatggtaacagccgcttagcctggtatcagcagaaaccagggcagtctcccaagctcctgatctattatgcatccactctggcatctggggtcccttcgcggttcaaaggcagtggatctgggacacagttcactctcaccattagcgacctggagtgtgacgatgctgcctcttactactgtcaaggcggttattatagtggtaatcttgatgcgcttgctttcggcggagggaccgaggtggtggtcagaggt

SEQ ID NO: 51 - protein, 263 aa, Artificial Sequence
  DNP scFv
QCQQLEQSGGGAEGGLVKPGGSLELCCKASGFSLSSSYCICWVRQAPGKGLEWIGCIYAGSSGSTYYASWVNGRFTLSRDIDQSTGCLQLNSLTAADTAMYYCARAPYSSGWVLYFNLWGPGTLVIVSSGGGGSGGGGSGGGGSPGATFAQVLTQTPSPVSAAVGGTVTISCQSSESVYGNSRLAWYQQKPGQSPKLLIYYASTLASGVPSRFKGSGSGTQFTLTISDLECDDAASYYCQGGYYSGNLDALAFGGGTEVVVRG

SEQ ID NO: 52 - DNA, 789 bp, Artificial Sequence
  DNP scFv
ccaggtgccacatttgcccaagtgctgacccagactccatcgcctgtgtctgcagctgtgggaggcacagtcaccatcagttgccagtccagtgagagtgtttatggtaacagccgcttagcctggtatcagcagaaaccagggcagtctcccaagctcctgatctattatgcatccactctggcatctggggtcccttcgcggttcaaaggcagtggatctgggacacagttcactctcaccattagcgacctggagtgtgacgatgctgcctcttactactgtcaaggcggttattatagtggtaatcttgatgcgcttgctttcggcggagggaccgaggtggtggtcagaggtggcggagggggctctggcggcggaggatctgggggagggggcagccagtgtcagcagctggagcagtccggaggaggagccgaaggaggcctggtcaagcctgggggatccctggaactctgctgcaaagcctctggattctccctcagtagtagctactgcatatgttgggtccgccaggctccagggaaggggctggagtggatcggatgcatttatgctggtagtagtggtagcacttactacgcgagctgggtgaatggccgattcactctctccagagacattgaccagagcacaggttgcctacaactgaacagtctgacagccgcggacacggccatgtattactgtgcgagagccccctatagtagtggctgggtcctctactttaacttgtggggcccaggcaccctggtcattgtctcctca

SEQ ID NO: 53 - protein, 263 aa, Artificial Sequence
  DNP scFv
PGATFAQVLTQTPSPVSAAVGGTVTISCQSSESVYGNSRLAWYQQKPGQSPKLLIYYASTLASGVPSRFKGSGSGTQFTLTISDLECDDAASYYCQGGYYSGNLDALAFGGGTEVVVRGGGGGSGGGGSGGGGSQCQQLEQSGGGAEGGLVKPGGSLELCCKASGFSLSSSYCICWVRQAPGKGLEWIGCIYAGSSGSTYYASWVNGRFTLSRDIDQSTGCLQLNSLTAADTAMYYCARAPYSSGWVLYFNLWGPGTLVIVSS

SEQ ID NO: 54 - protein, 217 aa, Artificial Sequence
  VH of DNP scFv
DVQLQESGPGLVKPSQSQSLTCTVTGYSITSDYAWNWIRQFPGNKLEWMGYMSYSGSTRYNPSLRSRISITRDTSKNQFFLQLKSVTTEDTATYFCARGWPLAYWGQGTQVSVSEAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDC

SEQ ID NO: 55 - protein, 214 aa, Artificial Sequence
  VL of DNP scFv
QIVLTQSPAIMSASPGEKVTMTCSASSSVYYMYWYQQKPGSSPRLLIYDTSNLASGVPVRFSGSGSGTSYSLTISRMEAEDAATYYCQQWSSYPPITFGVGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 56 - protein, 125 aa, Artificial Sequence
  VH of DNP scFv (Ab-2; XC)
QQLEQSGGGAEGGLVKPGGSLELCCKASGFSLSSSYCICWVRQAPGKGLEWIGCIYAGSSGSTYYASWVNGRFTLSRDIDQSTGCLQLNSLTAADTAMYYCARAPYSSGWVLYFNLWGPGTLVIV

SEQ ID NO: 57 - protein, 104 aa, Artificial Sequence
  VL of DNP scFv (Ab-2; XC)
AQVLTQTPSPVSAAVGGTVTISCQSSESVYGNSRLAWYQQKPGQSPKLLIYYASTLASGVPSRFKGSGSGTQFTLTISDLECDDAASYYCQGGYYSGNLDALAF

SEQ ID NO: 58 - protein, 125 aa, Artificial Sequence; identical to SEQ ID NO: 56
  VH of DNP scFv (Ab-3)

SEQ ID NO: 59 - protein, 119 aa, Artificial Sequence
  VL of DNP scFv (Ab-3)
PGATFAQVLTQTPSPVSAAVGGTVTISCQSSESVYGNSRLAWYQQKPGQSPKLLIYYASTLASGVPSRFKGSGSGTQFTLTISDLECDDAASYYCQGGYYSGNLDALAFGGGTEVVVRG

SEQ ID NO: 60 - protein, 263 aa, Artificial Sequence; identical to SEQ ID NO: 53
  VL-linker-VH of DNP scFv (Ab-3)

SEQ ID NO: 61 - protein, 263 aa, Artificial Sequence; identical to SEQ ID NO: 51
  VH-linker-VL of DNP scFv (Ab-3)

SEQ ID NO: 62 - protein, 446 aa, Artificial Sequence
  VH-linker-VL of DNP scFv (1BAF)
DVQLQESGPGLVKPSQSQSLTCTVTGYSITSDYAWNWIRQFPGNKLEWMGYMSYSGSTRYNPSLRSRISITRDTSKNQFFLQLKSVTTEDTATYFCARGWPLAYWGQGTQVSVSEAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGGGGSGGGGSGGGGSQIVLTQSPAIMSASPGEKVTMTCSASSSVYYMYWYQQKPGSSPRLLIYDTSNLASGVPVRFSGSGSGTSYSLTISRMEAEDAATYYCQQWSSYPPITFGVGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 63 - protein, 446 aa, Artificial Sequence
  VL-linker-VH of DNP scFv (1BAF)
QIVLTQSPAIMSASPGEKVTMTCSASSSVYYMYWYQQKPGSSPRLLIYDTSNLASGVPVRFSGSGSGTSYSLTISRMEAEDAATYYCQQWSSYPPITFGVGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSDVQLQESGPGLVKPSQSQSLTCTVTGYSITSDYAWNWIRQFPGNKLEWMGYMSYSGSTRYNPSLRSRISITRDTSKNQFFLQLKSVTTEDTATYFCARGWPLAYWGQGTQVSVSEAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDC

SEQ ID NO: 64 - protein, 246 aa, Artificial Sequence
  VH-linker-VL of DNP scFv (XC)
QQLEQSGGGAEGGLVKPGGSLELCCKASGFSLSSSYCICWVRQAPGKGLEWIGCIYAGSSGSTYYASWVNGRFTLSRDIDQSTGCLQLNSLTAADTAMYYCARAPYSSGWVLYFNLWGPGTLVIVSSGGGGSGGGGSGGGGSAQVLTQTPSPVSAAVGGTVTISCQSSESVYGNSRLAWYQQKPGQSPKLLIYYASTLASGVPSRFKGSGSGTQFTLTISDLECDDAASYYCQGGYYSGNLDALAF

SEQ ID NO: 65 - protein, 254 aa, Artificial Sequence
  VL-linker-VH of DNP scFv (XC)
AQVLTQTPSPVSAAVGGTVTISCQSSESVYGNSRLAWYQQKPGQSPKLLIYYASTLASGVPSRFKGSGSGTQFTLTISDLECDDAASYYCQGGYYSGNLDALAFGGGTEVVVRGGGGGSGGGGSGGGGSQQLEQSGGGAEGGLVKPGGSLELCCKASGFSLSSSYCICWVRQAPGKGLEWIGCIYAGSSGSTYYASWVNGRFTLSRDIDQSTGCLQLNSLTAADTAMYYCARAPYSSGWVLYFNLWGPGTLVIV

SEQ ID NO: 66 - DNA, 1338 bp, Artificial Sequence
  VH-linker-VL of DNP scFv (1BAF)
gacgtacaactgcaagaatcaggtccgggactggtcaaacctagccagtctcagtccttgacgtgcaccgtgacgggctatagtataacgagtgattacgcttggaattggattcggcagtttccaggcaataagcttgagtggatgggctatatgtcttactccggctcaactaggtataatccgagccttcggtctcggatttctatcacaagagatacgagtaagaatcagttttttcttcaactcaaaagcgtcaccacggaggataccgccacatatttctgtgctaggggatggcctcttgcttattgggggcaagggacacaagtgtctgtttccgaagccaaaacaacgcccccttcagtctatccgctggcaccgggaagcgcggcacaaaccaatagtatggtaacgctcggatgtctcgtcaaggggtattttcccgagcctgtgacagtgacatggaattctgggagtcttagcagcggagtacatacttttccggcagtacttcaatccgatttgtacacgctctcctctagtgttacagttccaagctctccacgacctagtgagaccgttacatgtaacgtcgcgcatccggcctcttccactaaagtggataaaaagattgtgcccagggactgcggcggagggggctctggcggcggaggatctgggggagggggcagccaaattgtgttgacccagtccccggccataatgtccgcttctcctggcgagaaggttactatgacttgctcagcctcctccagtgtgtattatatgtactggtatcaacaaaagccgggctcttccccccggctccttatatacgacacgagtaatctggcaagtggcgtgcctgttagattttctgggtccggctctggaacttcatactccctgacaattagccgaatggaagccgaggacgcggcgacatactactgccagcaatggtcatcctatccgcctatcacttttggagtagggaccaaattggagttgaagcgggctgatgcggctcccacagttagtattttccctccgtccagtgaacaacttacctccgggggagcctccgttgtttgctttctgaacaacttttacccgaaagatataaatgtcaagtggaagatcgacggctcagagcgccaaaacggggtactcaactcatggacagatcaggatagtaaagattcaacttacagtatgtctagtaccctgacactgacgaaagatgaatacgaaagacataatagttatacctgtgaagctacacataagacttcaacctctcctattgtaaaatcattcaaccgaaacgaatgt

SEQ ID NO: 67 - DNA, 1338 bp, Artificial Sequence
  VL-linker-VH of DNP scFv (1BAF)
caaattgtgttgacccagtccccggccataatgtccgcttctcctggcgagaaggttactatgacttgctcagcctcctccagtgtgtattatatgtactggtatcaacaaaagccgggctcttccccccggctccttatatacgacacgagtaatctggcaagtggcgtgcctgttagattttctgggtccggctctggaacttcatactccctgacaattagccgaatggaagccgaggacgcggcgacatactactgccagcaatggtcatcctatccgcctatcacttttggagtagggaccaaattggagttgaagcgggctgatgcggctcccacagttagtattttccctccgtccagtgaacaacttacctccgggggagcctccgttgtttgctttctgaacaacttttacccgaaagatataaatgtcaagtggaagatcgacggctcagagcgccaaaacggggtactcaactcatggacagatcaggatagtaaagattcaacttacagtatgtctagtaccctgacactgacgaaagatgaatacgaaagacataatagttatacctgtgaagctacacataagacttcaacctctcctattgtaaaatcattcaaccgaaacgaatgtggcggagggggctctggcggcggaggatctgggggagggggcagcgacgtacaactgcaagaatcaggtccgggactggtcaaacctagccagtctcagtccttgacgtgcaccgtgacgggctatagtataacgagtgattacgcttggaattggattcggcagtttccaggcaataagcttgagtggatgggctatatgtcttactccggctcaactaggtataatccgagccttcggtctcggatttctatcacaagagatacgagtaagaatcagttttttcttcaactcaaaagcgtcaccacggaggataccgccacatatttctgtgctaggggatggcctcttgcttattgggggcaagggacacaagtgtctgtttccgaagccaaaacaacgcccccttcagtctatccgctggcaccgggaagcgcggcacaaaccaatagtatggtaacgctcggatgtctcgtcaaggggtattttcccgagcctgtgacagtgacatggaattctgggagtcttagcagcggagtacatacttttccggcagtacttcaatccgatttgtacacgctctcctctagtgttacagttccaagctctccacgacctagtgagaccgttacatgtaacgtcgcgcatccggcctcttccactaaagtggataaaaagattgtgcccagggactgc

SEQ ID NO: 68 - DNA, 738 bp, Artificial Sequence
  VH-linker-VL of DNP scFv (XC)
cagcagctggagcagtccggaggaggagccgaaggaggcctggtcaagcctgggggatccctggaactctgctgcaaagcctctggattctccctcagtagtagctactgcatatgttgggtccgccaggctccagggaaggggctggagtggatcggatgcatttatgctggtagtagtggtagcacttactacgcgagctgggtgaatggccgattcactctctccagagacattgaccagagcacaggttgcctacaactgaacagtctgacagccgcggacacggccatgtattactgtgcgagagccccctatagtagtggctgggtcctctactttaacttgtggggcccaggcaccctggtcattgtctcctcaggcggagggggctctggcggcggaggatctgggggagggggcagcgcccaagtgctgacccagactccatcgcctgtgtctgcagctgtgggaggcacagtcaccatcagttgccagtccagtgagagtgtttatggtaacagccgcttagcctggtatcagcagaaaccagggcagtctcccaagctcctgatctattatgcatccactctggcatctggggtcccttcgcggttcaaaggcagtggatctgggacacagttcactctcaccattagcgacctggagtgtgacgatgctgcctcttactactgtcaaggcggttattatagtggtaatcttgatgcgcttgctttc

SEQ ID NO: 69 - DNA, 762 bp, Artificial Sequence
  VL-linker-VH of DNP scFv (XC)
gcccaagtgctgacccagactccatcgcctgtgtctgcagctgtgggaggcacagtcaccatcagttgccagtccagtgagagtgtttatggtaacagccgcttagcctggtatcagcagaaaccagggcagtctcccaagctcctgatctattatgcatccactctggcatctggggtcccttcgcggttcaaaggcagtggatctgggacacagttcactctcaccattagcgacctggagtgtgacgatgctgcctcttactactgtcaaggcggttattatagtggtaatcttgatgcgcttgctttcggcggagggaccgaggtggtggtcagaggtggcggagggggctctggcggcggaggatctgggggagggggcagccagcagctggagcagtccggaggaggagccgaaggaggcctggtcaagcctgggggatccctggaactctgctgcaaagcctctggattctccctcagtagtagctactgcatatgttgggtccgccaggctccagggaaggggctggagtggatcggatgcatttatgctggtagtagtggtagcacttactacgcgagctgggtgaatggccgattcactctctccagagacattgaccagagcacaggttgcctacaactgaacagtctgacagccgcggacacggccatgtattactgtgcgagagccccctatagtagtggctgggtcctctactttaacttgtggggcccaggcaccctggtcattgtc

SEQ ID NO: 70 - DNA, 789 bp, Artificial Sequence; identical to SEQ ID NO: 52
  VH-linker-VL of DNP scFv (Ab-3)

SEQ ID NO: 71 - DNA, 789 bp, Artificial Sequence; identical to SEQ ID NO: 50
  DNP scFv VH-linker-VL of DNP scFv (Ab-3)

SEQ ID NO: 72 - protein, 15 aa, Artificial Sequence
  linker
GGGGSGGGGSGGGGS

SEQ ID NO: 73 - protein, 20 aa, Artificial Sequence
  linker
GGGGSGGGGSGGGGSGGGGS

SEQ ID NO: 74 - protein, 10 aa, Artificial Sequence
  linker
GGGGSGGGGS

SEQ ID NO: 75 - protein, 5 aa, Artificial Sequence
  linker
GGGGS

SEQ ID NO: 76 - DNA, 45 bp, Artificial Sequence
  linker
ggcggaggcggctccggaggaggagggtctggaggcgggggaagt

SEQ ID NO: 77 - DNA, 60 bp, Artificial Sequence
  linker
ggcgggggaggctccgggggaggcggctccggcggcgggggaagtggcgggggaggatcc

SEQ ID NO: 78 - protein, 12 aa, Artificial Sequence
  short spacer
ESKYGPPCPPCP

SEQ ID NO: 79 - DNA, 36 bp, Artificial Sequence
  short spacer
gagtctaagtacggtccaccttgccccccctgccct

SEQ ID NO: 80 - protein, 11 aa, Artificial Sequence
  short spacer
ESKYGPPCPPC

SEQ ID NO: 81 - protein, 119 aa, Artificial Sequence
  medium spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 82 - protein, 229 aa, Artificial Sequence
  Long spacer with single (L235D) mutation
ESKYGPPCPPCPAPEFDGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - protein, 229 aa, Artificial Sequence
  Long spacer with double (L235D, N297Q) mutations
ESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - skipped

SEQ ID NO: 85 - protein, 22 aa, Artificial Sequence
  P2A
GSGATNFSLLKQAGDVEENPGP

SEQ ID NO: 86 - DNA, 66 bp, Artificial Sequence
  P2A
ggaagtggtgcaaccaacttctctcttttgaaacaggctggagatgtcgaggagaaccctgggcct

SEQ ID NO: 87 - protein, 21 aa, Artificial Sequence
  T2A
GGGEGRGSLLTCGDVEENPGP

SEQ ID NO: 88 - DNA, 63 bp, Artificial Sequence
  T2A
ggcggcggcgaagggcgaggaagtttgcttacctgcggggatgtggaggaaaaccccggtcct

SEQ ID NO: 89 - protein, 24 aa, Artificial Sequence
  E2A
GSGQCTNYALLKLAGDVESNPGPP

SEQ ID NO: 90 - protein, 25 aa, Artificial Sequence
  F2A
GSGVKQTLNFDLLKLAGDVESNPGP

SEQ ID NO: 91 - protein, 187 aa, Artificial Sequence
  DHFRdm
MVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 92 - DNA, 561 bp, Artificial Sequence
  DHFRdm
atggtaggttccctcaattgtatagtggctgtcagtcaaaacatgggtataggcaaaaatggcgacttcccgtggccgccgctcaggaacgaatcaagatacttccaacgcatgacgacaactagctctgtcgaagggaagcagaatctcgtcatcatgggtaagaagacgtggttctctataccagagaaaaatcgcccgttgaaagggaggataaacctcgtgctttcacgcgaattgaaggaacctccgcaaggggcccactttctgagtcgctcactcgacgatgcgttgaaactgactgaacagcctgagttggcaaacaaagtcgacatggtgtggatcgtgggaggaagcagtgtttataaagaagcaatgaaccaccccggacacctgaagcttttcgttacccgaataatgcaggactttgaatcagatacattcttccctgaaattgaccttgaaaaatataaacttctccctgaataccctggcgtgctgtccgacgtccaagaggaaaaaggtattaagtacaagtttgaggtatatgaaaaaaatgat

SEQ ID NO: 93 - protein, 323 aa, Artificial Sequence
  CD19t
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 94 - DNA, 969 bp, Artificial Sequence
  CD19t
atgcccccacctcgcctgctcttttttctcttgttcctcacgcccatggaagtccggcccgaggagccacttgtagtcaaggtagaagagggagacaacgcagtgctgcagtgccttaaaggcacaagtgatgggcccacccaacaactgacatggagtcgcgagtcacctcttaaacccttcctcaaattgagtctcggcttgccgggcttgggcattcatatgcgacctctggctatatggctgttcatttttaatgtttcccagcagatggggggtttttatttgtgccagccaggcccgccaagcgaaaaagcctggcagccaggatggacagtcaatgtagaaggatctggtgagctttttaggtggaacgtatctgatttggggggacttggatgcggtttgaagaaccgatcttccgagggtccgagctctcctagtgggaaactgatgtctcctaagctgtacgtttgggcgaaagaccggcccgagatttgggagggcgagccgccctgtgtgccgcctcgagactcccttaatcagtccctttcccaggacctgacgatggcacctgggagtacgctgtggcttagttgtggggtgccgcctgatagtgtctcccggggtcccctttcctggacacacgtgcatccaaaaggcccgaagtctcttttgtctttggagcttaaagacgatcgccctgctcgcgatatgtgggtgatggaaacgggattgcttctgccacgcgccaccgcccaggatgcggggaagtattactgccatcgagggaacctgacaatgagctttcatctggaaatcactgccagaccagtactgtggcactggcttttgcggacgggcggctggaaagtgagcgcagtgacactggcgtatctcattttctgcctttgtagtctcgtaggtattttgcacttgcaacgcgcgcttgtgcttcgcagaaagcgg

SEQ ID NO: 95 - protein, 135 aa, Artificial Sequence
  truncated Her2 (Her2)
MLLLVTSLLLCELPHPAFLLIPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILI

SEQ ID NO: 96 - protein, 120 aa, Artificial Sequence
  Her2tG
CHPECQPQNGSVTCFGPEADQCVACAHYKDPPFCVARCPSGVKPDLSYMPIWKFPDEEGACQPCPINCTHSCVDLDDKGCPAEQRASPLTGGGSGGGSIISAVVGILLVVVLGVVFGILI

SEQ ID NO: 97 - protein, 335 aa, Artificial Sequence
  EGFRt
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 98 - protein, 22 aa, Homo sapiens
MLLLVTSLLLCELPHPAFLLIP

SEQ ID NO: 99 - protein, 20 aa, Homo sapiens
MALPVTALLLPLALLLHAAR

SEQ ID NO: 100 - protein, 6 aa, Artificial Sequence
  His tag
HHHHHH

SEQ ID NO: 101 - protein, 7 aa, Artificial Sequence
  Flag tag
DYKDDDD

SEQ ID NO: 102 - protein, 8 aa, Artificial Sequence
  Flag tag
DYKDDDDK

SEQ ID NO: 103 - protein, 7 aa, Artificial Sequence
  Flag tag
DYKDDDK

SEQ ID NO: 104 - protein, 8 aa, Artificial Sequence
  Xpress tag
DLYDDDDK

SEQ ID NO: 105 - protein, 15 aa, Artificial Sequence
  Avi tag
GLNDIFEAQKIEWHE

SEQ ID NO: 106 - protein, 26 aa, Artificial Sequence
  Calmoduling binding peptide tag
KRRWKKNFIAVSAANRFKKISSSGAL

SEQ ID NO: 107 - protein, 6 aa, Artificial Sequence
  Polyglutamate tag
EEEEEE

SEQ ID NO: 108 - protein, 9 aa, Artificial Sequence
  HA tag
YPYDVPDYA

SEQ ID NO: 109 - protein, 9 aa, Artificial Sequence
  HA tag
YAYDVPDYA

SEQ ID NO: 110 - protein, 9 aa, Artificial Sequence
  HA tag
YDVPDYASL

SEQ ID NO: 111 - protein, 10 aa, Artificial Sequence
  Myc tag
EQKLISEEDL

SEQ ID NO: 112 - protein, 8 aa, Artificial Sequence
  Strep tag
WRHPQFGG

SEQ ID NO: 113 - protein, 8 aa, Artificial Sequence
  Strep tag II
WSHPQFEK

SEQ ID NO: 114 - protein, 13 aa, Artificial Sequence
  Softag 1
SLAELLNAGLGGS

SEQ ID NO: 115 - protein, 8 aa, Artificial Sequence
  Softag 3
TQDPSRVG

SEQ ID NO: 116 - protein, 14 aa, Artificial Sequence
  V5tag
GKPIPNPLLGLDST

SEQ ID NO: 117 - DNA, 399 bp, Artificial Sequence
  MND promoter
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 118 - protein, 775 aa, Artificial Sequence
  CAR cleaved from Selection Markers
MLLLVTSLLLCELPHPAFLLIPDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGSTSGSGKPGSGEGSTKGDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 119 - protein, 28 aa, Homo sapiens
MFWVLVVVGGVLACYSLLVTVAFIIFWV

SEQ ID NO: 120 - protein, 42 aa, Homo sapiens
KRGRKKLLYIFKQPFMRPVQTTQEEDGCSCRFPEEEEGGCEL

SEQ ID NO: 121 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - DNA, 84 bp, Homo sapiens
atgttctgggtattggtagtcgtgggcggagttctggcctgttactcattgctcgttacagtcgctttcatcatattttgggtc

SEQ ID NO: 123 - DNA, 126 bp, Homo sapiens
aaaaggggacgcaaaaaactcctttacattttcaaacagccctttatgcgacctgtacaaacaactcaggaagaggatggctgcagctgtaggtttcccgaggaagaagagggtggctgcgagctc

SEQ ID NO: 124 - DNA, 336 bp, Homo sapiens
agagtgaagttcagcagatccgccgacgctcctgcctatcagcagggacagaaccagctgtacaacgagctgaacctggggagaagagaagagtacgacgtgctggataagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctcgt

SEQ ID NO: 125 - DNA, 3979 bp, Artificial Sequence
  EGFR806CAR-ERT2-P2A-DHFRdm-T2A-CD19t
atgctgctgctggtgaccagcctgctgctgtgcgagctgccccaccccgcctttctgctgatcccccaggtgcagctgcagcagcctggcgccgagctggtgaagccaggcgccagcgtgaagctgtcctgcaaggccagcggctacaccttcaccggctactggatgcactgggtgaagcagagacccggccacggcctggaatggatcggcgagatcaaccccagcaacggccggaccaactacaacgagcggttcaagagcaaggccaccctgaccgtggacaagagcagcaccaccgccttcatgcagctgtccggcctgaccagcgaggacagcgccgtgtacttctgcgccagggactactacggcaccagctacaacttcgactactggggccagggcaccacactgaccgtgagcagcggcggagggggctctggcggcggaggatctgggggagggggcagcgacatccagatgacccagagcagcagcagcttcagcgtgagcctgggcgaccgggtgaccatcacctgtaaggccaacgaggacatcaacaaccggctggcctggtatcagcagacccccggcaacagccccaggctgctgatcagcggcgccaccaacctggtgaccggcgtgcccagccggtttagcggcagcggctccggcaaggactacaccctgaccatcacaagcctgcaggccgaggacttcgccacctactactgccagcagtactggtccacccccttcaccttcggcagcggcaccgagctggaaagagtctaagtacggtccaccttgccccccctgccctatgttctgggtattggtagtcgtgggcggagttctggcctgttactcattgctcgttacagtcgctttcatcatattttgggtcaaaaggggacgcaaaaaactcctttacattttcaaacagccctttatgcgacctgtacaaacaactcaggaagaggatggctgcagctgtaggtttcccgaggaagaagagggtggctgcgagctcagagtgaagttcagcagatccgccgacgctcctgcctatcagcagggacagaaccagctgtacaacgagctgaacctggggagaagagaagagtacgacgtgctggataagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctcgtggaggcggttcagcgggtgacatgcgagctgccaatctctggccgtcacctttgatgattaaacgatcaaagaaaaatagcctggctctgagcttgacggctgatcaaatggtaagtgcactgctcgacgccgaaccgccgatcctgtattccgagtatgaccccacacggccattctccgaagccagtatgatgggacttcttaccaaccttgcagatcgcgaactcgtacatatgataaattgggcgaagcgcgttccagggttcgtcgacctcacgttgcatgatcaggtccatctgctggaatgcgcgtggttggaaatcttgatgattggtttggtctggcgatctatggagcatccggtgaaacttcttttcgcgcccaatcttcttttggatcgcaatcaagggaaatgcgtggaaggaatggtcgaaatttttgatatgcttctcgcgaccagctcccgattcaggatgatgaatttgcagggtgaagagtttgtatgtcttaagtctattatattgctcaactccggcgtttatacattcctttctagcacacttaaatcactcgaggaaaaggaccacatccaccgggttttggataaaatcactgatacactgattcatttgatggccaaggccggactcacactccaacaacagcaccaacggcttgcacagctcctcctcatactttctcatataaggcacatgtcaaataagggaatggagcacctgtactccatgaagtgtaaaaatgtagtccctctgtatgatctgctccttgaggctgcagacgcgcatagactgcacgccccaacgtcacgagggggggcctcagtggaagagacagatcagagccatctcgcaaccgcaggatctacatccagccactctcttcagaaatactatataactggcgaagccgaagggttccctgccaccgttggaagtggtgcaaccaacttctctcttttgaaacaggctggagatgtcgaggagaaccctgggcctatggtaggttccctcaattgtatagtggctgtcagtcaaaacatgggtataggcaaaaatggcgacttcccgtggccgccgctcaggaacgaatcaagatacttccaacgcatgacgacaactagctctgtcgaagggaagcagaatctcgtcatcatgggtaagaagacgtggttctctataccagagaaaaatcgcccgttgaaagggaggataaacctcgtgctttcacgcgaattgaaggaacctccgcaaggggcccactttctgagtcgctcactcgacgatgcgttgaaactgactgaacagcctgagttggcaaacaaagtcgacatggtgtggatcgtgggaggaagcagtgtttataaagaagcaatgaaccaccccggacacctgaagcttttcgttacccgaataatgcaggactttgaatcagatacattcttccctgaaattgaccttgaaaaatataaacttctccctgaataccctggcgtgctgtccgacgtccaagaggaaaaaggtattaagtacaagtttgaggtatatgaaaaaaatgatggcggcggcgaagggcgaggaagtttgcttacctgcggggatgtggaggaaaaccccggtcctatgcccccacctcgcctgctcttttttctcttgttcctcacgcccatggaagtccggcccgaggagccacttgtagtcaaggtagaagagggagacaacgcagtgctgcagtgccttaaaggcacaagtgatgggcccacccaacaactgacatggagtcgcgagtcacctcttaaacccttcctcaaattgagtctcggcttgccgggcttgggcattcatatgcgacctctggctatatggctgttcatttttaatgtttcccagcagatggggggtttttatttgtgccagccaggcccgccaagcgaaaaagcctggcagccaggatggacagtcaatgtagaaggatctggtgagctttttaggtggaacgtatctgatttggggggacttggatgcggtttgaagaaccgatcttccgagggtccgagctctcctagtgggaaactgatgtctcctaagctgtacgtttgggcgaaagaccggcccgagatttgggagggcgagccgccctgtgtgccgcctcgagactcccttaatcagtccctttcccaggacctgacgatggcacctgggagtacgctgtggcttagttgtggggtgccgcctgatagtgtctcccggggtcccctttcctggacacacgtgcatccaaaaggcccgaagtctcttttgtctttggagcttaaagacgatcgccctgctcgcgatatgtgggtgatggaaacgggattgcttctgccacgcgccaccgcccaggatgcggggaagtattactgccatcgagggaacctgacaatgagctttcatctggaaatcactgccagaccagtactgtggcactggcttttgcggacgggcggctggaaagtgagcgcagtgacactggcgtatctcattttctgcctttgtagtctcgtaggtattttgcacttgcaacgcgcgcttgtgcttcgcagaaagcgg

SEQ ID NO: 126 - protein, 69 aa, Homo sapiens
TTTPAPRPPTPAPTIASQPLSLRPEACRPAAGGAVHTRGLDFACDIYIWAPLAGTCGVLLLSLVITLYC

SEQ ID NO: 127 - protein, 75 aa, Homo sapiens
ESKYGPAKPTTTPAPRPPTPAPTIASQPLSLRPEACRPAAGGAVHTRGLDFACDIYIWAPLAGTCGVLLLSLVIT

SEQ ID NO: 128 - protein, 21 aa, Homo sapiens
IYIWAPLAGTCGVLLLSLVIT

SEQ ID NO: 129 - protein, 48 aa, Homo sapiens
AKPTTTPAPRPPTPAPTIASQPLSLRPEACRPAAGGAVHTRGLDFACD

SEQ ID NO: 130 - protein, 154 aa, Artificial Sequence
  4-1BB/CD3z intracellular signaling domain
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - DNA, 462 bp, Artificial Sequence
  4-1BB/CD3z intracellular signaling domains
aaaaggggacgcaaaaaactcctttacattttcaaacagccctttatgcgacctgtacaaacaactcaggaagaggatggctgcagctgtaggtttcccgaggaagaagagggtggctgcgagctcagagtgaagttcagcagatccgccgacgctcctgcctatcagcagggacagaaccagctgtacaacgagctgaacctggggagaagagaagagtacgacgtgctggataagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctcgt

SEQ ID NO: 132 - DNA, 462 bp, Artificial Sequence
  4-1BB/CD3z intracellular signaling domains
aaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 133 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 134 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 135 - protein, 55 aa, Homo sapiens
KNRKAKAKPVTRGAGAGGRQRGQNKERPPPVPNPDYEPIRKGQRDLYSGLNQRRI

SEQ ID NO: 136 - protein, 26 aa, Homo sapiens
VMSVATIVIVDICITGGLLLLVYYWS

SEQ ID NO: 137 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 138 - protein, 84 aa, Homo sapiens [sequence omitted]

SEQ ID NO: 139 - protein, 45 aa, Homo sapiens
GHETGRLSGAADTQALLRNDQVYQPLRDRDDAQYSHLGGNWARNK

SEQ ID NO: 140 - protein, 21 aa, Homo sapiens
GIIVTDVIATLLLALGVFCFA

SEQ ID NO: 141 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 142 - protein, 9 aa, Homo sapiens
QSFGLLDPK

SEQ ID NO: 143 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 144 - protein, 21 aa, Homo sapiens
LCYLLDGILFIYGVILTALFL

SEQ ID NO: 145 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 146 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 147 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 148 - protein, 186 aa, Homo sapiens [sequence omitted]

SEQ ID NO: 149 - protein, 42 aa, Homo sapiens
ALYLLRRDQRLPPDAHKPPGGGSFRTPIQEEQADAHSTLAKI

SEQ ID NO: 150 - protein, 21 aa, Homo sapiens
VAAILGLGLVLGLLGPLAILL

SEQ ID NO: 151 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 152 - protein, 172 aa, Homo sapiens
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIR

SEQ ID NO: 153 - protein, 48 aa, Homo sapiens
QRRKYRSNKGESPVEPAEPCHYSCPREEEGSTIPIQEDYRKPEPACSP

SEQ ID NO: 154 - protein, 21 aa, Homo sapiens
ILVIFSGMFLVFTLAGALFLH

SEQ ID NO: 155 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 156 - protein, 361 aa, Homo sapiens
FPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGK

SEQ ID NO: 157 - protein, 189 aa, Homo sapiens
CHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 158 - protein, 27 aa, Homo sapiens
PVLDAGPVLFWVILVLVVVVGSSAFLL

SEQ ID NO: 159 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 160 - protein, 173 aa, Homo sapiens
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLR

SEQ ID NO: 161 - protein, 62 aa, Homo sapiens
KKVAKKPTNKAPHPKQEPQEINFPDDLPGSNTAAPVQETLHGCQPVTQEDGKESRISVQERQ

SEQ ID NO: 162 - protein, 22 aa, Homo sapiens
ALVVIPIIFGILFAILLVLVFI

SEQ ID NO: 163 - protein, 283 aa, Homo sapiens [sequence omitted]

SEQ ID NO: 164 - protein, 164 aa, Homo sapiens [sequence omitted]

SEQ ID NO: 165 - protein, 60 aa, Homo sapiens
CVKRRKPRGDVVKVIVSVQRKRQEAEGEATVIEALQAPPDVTTVAVEETIPSFTGRSPNH

SEQ ID NO: 166 - protein, 21 aa, Homo sapiens
WWFLSGSLVIVIVCSTVGLII

SEQ ID NO: 167 - protein, 417 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 168 - protein, 175 aa, Homo sapiens
QGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQ

SEQ ID NO: 169 - protein, 197 aa, Homo sapiens
TYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 170 - protein, 21 aa, Homo sapiens
MFWVQVLLAGLVVPLLLGATL

SEQ ID NO: 171 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 172 - protein, 164 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQI

SEQ ID NO: 173 - protein, 27 aa, Homo sapiens
IISFFLALTSTALLFLLFFLTLRFSVV

SEQ ID NO: 174 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 175 - protein, 219 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 176 - protein, 13 aa, Homo sapiens
TWQRRQRKSRRTI

SEQ ID NO: 177 - protein, 19 aa, Homo sapiens
VAVAGCVFLLISVLLLSGL

SEQ ID NO: 178 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 179 - protein, 134 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 180 - protein, 41 aa, Homo sapiens
RSKRSRLLHSDYMNMTPRRPGPTRKHYQPYAPPRDFAAYRS

SEQ ID NO: 181 - protein, 41 aa, Homo sapiens
RSKRSRGGHSDYMNMTPRRPGPTRKHYQPYAPPRDFAAYRS

SEQ ID NO: 182 - DNA, 123 bp, Homo sapiens
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 183 - protein, 27 aa, Homo sapiens
FWVLVVVGGVLACYSLLVTVAFIIFWV

SEQ ID NO: 184 - protein, 226 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 185 - protein, 111 aa, Homo sapiens
LWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNR

SEQ ID NO: 186 - protein, 61 aa, Homo sapiens
RKRWQNEKLGLDAGDEYEDENLYEGLNLDDCSMYEDISRGLQGTYQDVGSLNIGDVQLEKP

SEQ ID NO: 187 - protein, 22 aa, Homo sapiens
IITAEGIILLFCAVVPGTLLLF

SEQ ID NO: 188 - protein, 229 aa, Homo sapiens
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 189 - protein, 131 aa, Homo sapiens
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKD

SEQ ID NO: 190 - protein, 49 aa, Homo sapiens
LDKDDSKAGMEEDHTYEGLDIDQTATYEDIVTLRTGEVKWSVGEHPGQE

SEQ ID NO: 191 - protein, 21 aa, Homo sapiens
GIIMIQTLLIILFIIVPIFLL

SEQ ID NO: 192 - protein, 335 aa, Homo sapiens
MDPKGLLSLTFVLFLSLAFGASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKPWAVYAGLLGGVIMILIMVVILQLRRRGKTNHYQTTVEKKSLTIYAQVQKPGPLQKKLDSFPAQDPCTTIYVAATEPVPESVQETNSITVYASVTLPES

SEQ ID NO: 193 - protein, 217 aa, Homo sapiens
ASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKP

SEQ ID NO: 194 - protein, 77 aa, Homo sapiens
QLRRRGKTNHYQTTVEKKSLTIYAQVQKPGPLQKKLDSFPAQDPCTTIYVAATEPVPESVQETNSITVYASVTLPES

SEQ ID NO: 195 - protein, 21 aa, Homo sapiens
WAVYAGLLGGVIMILIMVVIL

SEQ ID NO: 196 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 197 - protein, 120 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLK

SEQ ID NO: 198 - protein, 38 aa, Homo sapiens
CWLTKKKYSSSVHDPNGEYMFMRAVNTAKKSRLTDVTL

SEQ ID NO: 199 - protein, 21 aa, Homo sapiens
FWLPIGCAAFVVVCILGCILI

SEQ ID NO: 200 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 201 - protein, 137 aa, Homo sapiens
QRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEP

SEQ ID NO: 202 - protein, 58 aa, Homo sapiens
QLGLHIWQLRSQCMWPRETQLLLEVPPSTEDARSCQFPEEERGERSAEEKGRLGDLWV

SEQ ID NO: 203 - protein, 21 aa, Homo sapiens
LGWLTVVLLAVAACVLLLTSA

SEQ ID NO: 204 - protein, 1154 aa, Homo sapiens [sequence omitted]

SEQ ID NO: 205 - protein, 93 aa, Homo sapiens
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 206 - protein, 29 aa, Homo sapiens
TTPGERSSLPAFYPGTSGSCSGCGSLSLP

SEQ ID NO: 207 - protein, 24 aa, Homo sapiens
LCARPRRSPAQEDGKVYINMPGRG

SEQ ID NO: 208 - protein, 21 aa, Homo sapiens
LLAGLVAADAVASLLIVGAVF

SEQ ID NO: 209 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 210 - protein, 15 aa, Homo sapiens
QAQAQSDCSCSTVSP

SEQ ID NO: 211 - protein, 52 aa, Homo sapiens
YFLGRLVPRGRGAAEAATRKQRITETESPYQELQGQRSDVYSDLNTQRPYYK

SEQ ID NO: 212 - protein, 21 aa, Homo sapiens
GVLAGIVMGDLVLTVLIALAV

SEQ ID NO: 213 - protein, 257 aa, Homo sapiens
MAPAMESPTLLCVALLFFAPDGVLAVPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQFFIPLLVVILFAVDTGLFISTQQQVTFLLKIKRTRKGFRLLNPHPKPNPKNN

SEQ ID NO: 214 - protein, 244 aa, Homo sapiens
MDTESNRRANLALPQEPSSVPAFEVLEISPQEVSSGRLLKSASSPPLHTWLTVLKKEQEFLGVTQILTAMICLCFGTVVCSVLDISHIEGDIFSSFKAGYPFWGAIFFSISGMLSIISERRNATYLVRGSLGANTASSIAGGTGITILIINLKKSLAYIHIHSCQKFFETKCFMASFSTEIVVMMLFLTILGLGSAVSLTICGAGEELKGNKVPEDRVYEELNIYSATYSELEDPGEMSPPIDL

SEQ ID NO: 215 - protein, 86 aa, Homo sapiens
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 216 - protein, 537 aa, Homo sapiens
MGCVQCKDKEATKLTEERDGSLNQSSGYRYGTDPTPQHYPSFGVTSIPNYNNFHAAGGQGLTVFGGVNSSSHTGTLRTRGGTGVTLFVALYDYEARTEDDLSFHKGEKFQILNSSEGDWWEARSLTTGETGYIPSNYVAPVDSIQAEEWYFGKLGRKDAERQLLSFGNPRGTFLIRESETTKGAYSLSIRDWDDMKGDHVKHYKIRKLDNGGYYITTRAQFETLQQLVQHYSERAAGLCCRLVVPCHKGMPRLTDLSVKTKDVWEIPRESLQLIKRLGNGQFGEVWMGTWNGNTKVAIKTLKPGTMSPESFLEEAQIMKKLKHDKLVQLYAVVSEEPIYIVTEYMNKGSLLDFLKDGEGRALKLPNLVDMAAQVAAGMAYIERMNYIHRDLRSANILVGNGLICKIADFGLARLIEDNEYTARQGAKFPIKWTAPEAALYGRFTIKSDVWSFGILLTELVTKGRVPYPGMNNREVLEQVERGYRMPCPQDCPISLHELMIHCWKKDPEERPTFEYLQSFLEDYFTATEPQYQPGENL

SEQ ID NO: 217 - protein, 509 aa, Homo sapiens
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 218 - protein, 262 aa, Homo sapiens
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 219 - protein, 893 aa, Homo sapiens
MATEEFIIRIPPYHYIHVLDQNSNVSRVEVGPKTYIRQDNERVLFAPMRMVTVPPRHYCTVANPVSRDAQGLVLFDVTGQVRLRHADLEIRLAQDPFPLYPGEVLEKDITPLQVVLPNTALHLKALLDFEDKDGDKVVAGDEWLFEGPGTYIPRKEVEVVEIIQATIIRQNQALRLRARKECWDRDGKERVTGEEWLVTTVGAYLPAVFEEVLDLVDAVILTEKTALHLRARRNFRDFRGVSRRTGEEWLVTVQDTEAHVPDVHEEVLGVVPITTLGPHNYCVILDPVGPDGKNQLGQKRVVKGEKSFFLQPGEQLEQGIQDVYVLSEQQGLLLRALQPLEEGEDEEKVSHQAGDHWLIRGPLEYVPSAKVEVVEERQAIPLDENEGIYVQDVKTGKVRAVIGSTYMLTQDEVLWEKELPPGVEELLNKGQDPLADRGEKDTAKSLQPLAPRNKTRVVSYRVPHNAAVQVYDYREKRARVVFGPELVSLGPEEQFTVLSLSAGRPKRPHARRALCLLLGPDFFTDVITIETADHARLQLQLAYNWHFEVNDRKDPQETAKLFSVPDFVGDACKAIASRVRGAVASVTFDDFHKNSARIIRTAVFGFETSEAKGPDGMALPRPRDQAVFPQNGLVVSSVDVQSVEPVDQRTRDALQRSVQLAIEITTNSQEAAAKHEAQRLEQEARGRLERQKILDQSEAEKARKELLELEALSMAVESTGTAKAEAESRAEAARIEGEGSVLQAKLKAQALAIETEAELQRVQKVRELELVYARAQLELEVSKAQQLAEVEVKKFKQMTEAIGPSTIRDLAVAGPEMQVKLLQSLGLKSTLITDGSTPINLFNTAFGLLGMGPEGQPLGRRVASGPSPGEGISPQSAQAPQAPGDNHVVPVLR

SEQ ID NO: 220 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 221 - protein, 141 aa, Homo sapiens
MSNMRCVDCGTCLGHTRRHPTLFWGKTLPGLTPVAAPAPQPAQCPPGSEEDAPATQPGPQLAGPDPPWAPVFCRRLARAGHIELCNAVGCVLWSCPRSPGRGHAVGQGLEGGGGCPWATPPSLGGADFCNTKYSLWQKKCL

SEQ ID NO: 222 - protein, 2471 aa, Homo sapiens
MPALRPALLWALLALWLCCATPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCLQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDHMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 223 - protein, 2321 aa, Homo sapiens
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPVAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 224 - protein, 1999 aa, Homo sapiens
MQPPSLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPGCEVNPDNCVSHQCQNGGTCQDGLDTYTCLCPETWTGWDCSEDVDECEAQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 225 - protein, 943 aa, Homo sapiens
MARGSALPRRPLLCIPAVWAAAALLLSVSRTSGEVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGILYILVPSIAIPLVIACLFFLVCMCRNKQKASASTPQRRQLMASPSQDMEMPLINQHKQAKLKEISLSAVRFMEELGEDRFGKVYKGHLFGPAPGEQTQAVAIKTLKDKAEGPLREEFRHEAMLRARLQHPNVVCLLGVVTKDQPLSMIFSYCSHGDLHEFLVMRSPHSDVGSTDDDRTVKSALEPPDFVHLVAQIAAGMEYLSSHHVVHKDLATRNVLVYDKLNVKISDLGLFREVYAADYYKLLGNSLLPIRWMAPEAIMYGKFSIDSDIWSYGVVLWEVFSYGLQPYCGYSNQDVVEMIRNRQVLPCPDDCPAWVYALMIECWNEFPSRRPRFKDIHSRLRAWGNLSNYNSSAQTSGASNTTQTSSLSTSPVSNVSNARYVGPKQKAPPFPQPQFIPMKGQIRPMVPPPQLYVPVNGYQPVPAYGAYLPNFYPVQIPMQMAPQQVPPQMVPKPSSHHSGSGSTSTGYVTTAPSNTSMADRAALLSEGADDTQNAPEDGAQSTVQEAEEEEEGSVPETELLGDCDTLQVDEAQVQLEA

SEQ ID NO: 226 - protein, 607 aa, Homo sapiens
MRGAARLGRPGRSCLPGARGLRAPPPPPLLLLLALLPLLPAPGAAAAPAPRPPELQSASAGPSVSLYLSEDEVRRLIGLDAELYYVRNDLISHYALSFSLLVPSETNFLHFTWHAKSKVEYKLGFQVDNVLAMDMPQVNISVQGEVPRTLSVFRVELSCTGKVDSEVMILMQLNLTVNSSKNFTVLNFKRRKMCYKKLEEVKTSALDKNTSRTIYDPVHAAPTTSTRVFYISVGVCCAVIFLVAIILAVLHLHSMKRIELDDSISASSSSQGLSQPSTQTTQYLRADTPNNATPITSYPTLRIEKNDLRSVTLLEAKGKVKDIAISRERITLKDVLQEGTFGRIFHGILIDEKDPNKEKQAFVKTVKDQASEIQVTMMLTESCKLRGLHHRNLLPITHVCIEEGEKPMVILPYMNWGNLKLFLRQCKLVEANNPQAISQQDLVHMAIQIACGMSYLARREVIHKDLAARNCVIDDTLQVKITDNALSRDLFPMDYHCLGDNENRPVRWMALESLVNNEFSSASDVWAFGVTLWELMTLGQTPYVDIDPFEMAAYLKDGYRIAQPINCPDELFAVMACCWALDPEERPKFQQLVQCLTEFHAALGAYV

SEQ ID NO: 227 - protein, 533 aa, Homo sapiens
MALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYP

SEQ ID NO: 228 - protein, 281 aa, Homo sapiens
MAGTWLLLLLALGCPALPTGVGGTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLTCSCLCDPAGPLPSPATTTRLRALGSHRLHPATETGGREATSSPRPQPRDRRWGDTPPGRKPGSPVWGEGSYLSSYPTCPAQAWCSRSRLRAPSSSLGAFFRGDLPPPLQAGAA

SEQ ID NO: 229 - protein, 121 aa, Homo sapiens [sequence omitted]

SEQ ID NO: 230 - protein, 124 aa, Homo sapiens [sequence omitted]

SEQ ID NO: 231 - protein, 186 aa, Homo sapiens
MSGISGCPFFLWGLLALLGLALVISLIFNISHYVEKQRQDKMYSYSSDHTRVDEYYIEDTPIYGNLDDMISEPMDENCYEQMKARPEKSVNKMQEATPSAQATNETQMCYASLDHSVKGKRRKPRKQNTHFSDKDGDEQLHAIDASVSKTTLVDSFSPESQAVEENIHDDPIRLFGLIRAKREPIN

SEQ ID NO: 232 - protein, 493 aa, Homo sapiens
MRLGPRWKGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 233 - protein, 1203 aa, Homo sapiens [sequence omitted]

SEQ ID NO: 234 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 235 - protein, 147 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 236 - protein, 97 aa, Homo sapiens
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 237 - protein, 21 aa, Homo sapiens
VGVVGGLLGSLVLLVWVLAVI

SEQ ID NO: 238 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 239 - protein, 220 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 240 - protein, 31 aa, Homo sapiens
RLRKGRMMDVKKCGIQDTNSKKQSDTHLEET

SEQ ID NO: 241 - protein, 21 aa, Homo sapiens
THLVILGAILLCLGVALTFIF

SEQ ID NO: 242 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 243 - protein, 201 aa, Homo sapiens
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPT

SEQ ID NO: 244 - protein, 32 aa, Homo sapiens
ALRKQLCQKLYSSKDTTKRPVTTTKREVNSAI

SEQ ID NO: 245 - protein, 21 aa, Homo sapiens
WLLHIFIPFCIIAFIFIATVI

SEQ ID NO: 246 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 247 - protein, 126 aa, Homo sapiens [sequence omitted]

SEQ ID NO: 248 - protein, 41 aa, Homo sapiens
AVSLSKMLKKRSPLTTGVYVKMPPTEPECEKQFQPYFIPIN

SEQ ID NO: 249 - protein, 21 aa, Homo sapiens
FLLWILAAVSSGLFFYSFLLT

SEQ ID NO: 250 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 251 - protein, 526 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 252 - protein, 394 aa, Homo sapiens
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPG

SEQ ID NO: 253 - protein, 74 aa, Homo sapiens
HFGKTGRASDQRDLTEHKPSVSNHTQDHSNDPPNKMNEVTYSTLNFEAQQPTQPTSASPSLTATEIIYSEVKKQ

SEQ ID NO: 254 - protein, 24 aa, Homo sapiens
AIAGIVIGVVALVALIAVALACFL

SEQ ID NO: 255 - protein, 252 aa, Homo sapiens
MGPPSASPHRECIPWQGLLLTASLLNFWNPPTTAKLTIESMPLSVAEGKEVLLLVHNLPQHLFGYSWYKGERVDGNSLIVGYVIGTQQATPGAAYSGRETIYTNASLLIQNVTQNDIGFYTLQVIKSDLVNEEATGQFHVYQENAPGLPVGAVAGIVTGVLVGVALVAALVCFLLLAKTGRTSIQRDLKEQQPQALAPGRGPSHSSAFSMSPLSTAQAPLPNPRTAASIYEELLKHDTNIYCRMDHKAEVAS

SEQ ID NO: 256 - protein, 121 aa, Homo sapiens
KLTIESMPLSVAEGKEVLLLVHNLPQHLFGYSWYKGERVDGNSLIVGYVIGTQQATPGAAYSGRETIYTNASLLIQNVTQNDIGFYTLQVIKSDLVNEEATGQFHVYQENAPGLPVGAVAG

SEQ ID NO: 257 - protein, 76 aa, Homo sapiens
AKTGRTSIQRDLKEQQPQALAPGRGPSHSSAFSMSPLSTAQAPLPNPRTAASIYEELLKHDTNIYCRMDHKAEVAS

SEQ ID NO: 258 - protein, 21 aa, Homo sapiens
IVTGVLVGVALVAALVCFLLL

SEQ ID NO: 259 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 260 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 261 - protein, 428 aa, Homo sapiens
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 262 - protein, 54 aa, Homo sapiens
HLWRRQWRPRRFSALEQGIHPPQAQSKIEELEQEPEPEPEPEPEPEPEPEPEQL

SEQ ID NO: 263 - protein, 21 aa, Homo sapiens
LLFLILGVLSLLLLVTGAFGF

SEQ ID NO: 264 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 265 - protein, 162 aa, Homo sapiens
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAA

SEQ ID NO: 266 - protein, 96 aa, Homo sapiens
YKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 267 - protein, 21 aa, Homo sapiens
ALATGACIVGILCLPLILLLV

SEQ ID NO: 268 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 269 - protein, 127 aa, Homo sapiens
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYR

SEQ ID NO: 270 - protein, 111 aa, Homo sapiens
RRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 271 - protein, 21 aa, Homo sapiens
LLPLGGLPLLITTCFCLFCCL

SEQ ID NO: 272 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 273 - protein, 120 aa, Homo sapiens
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 274 - protein, 82 aa, Homo sapiens
LTRKKKALRIHSVEGDLRRKSAGQEEWSPSAPSPPGSCVQAEAAPAGLCGEQRGEDCAELHDYFNVLSYRSLGNCSFFTETG

SEQ ID NO: 275 - protein, 21 aa, Homo sapiens
LLGAMAATLVVICTAVIVVVA

SEQ ID NO: 276 - protein, 287 aa, Homo sapiens
MSPHPTALLGLVLCLAQTIHTQEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 277 - protein, 144 aa, Homo sapiens
QEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLY

SEQ ID NO: 278 - protein, 101 aa, Homo sapiens
HRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 279 - protein, 21 aa, Homo sapiens
ILIGVSVVFLFCLLLLVLFCL

SEQ ID NO: 280 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 281 - protein, 208 aa, Homo sapiens [sequence omitted]

SEQ ID NO: 282 - protein, 25 aa, Homo sapiens
TYCFAPRCRERRRNERLRRESVRPV

SEQ ID NO: 283 - protein, 21 aa, Homo sapiens
LLPSWAITLISVNGIFVICCL

SEQ ID NO: 284 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 285 - protein, 224 aa, Homo sapiens
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 286 - protein, 61 aa, Homo sapiens
KWKKKKRPRNSYKCGTNTMEREESEQTKKREKIHIPERSDEAQRVFKSSKTSSCDKSDTCF

SEQ ID NO: 287 - protein, 21 aa, Homo sapiens
WITAVLPTVIICVMVFCLILW

SEQ ID NO: 288 - protein, 181 aa, Homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQAL

SEQ ID NO: 289 - protein, 370 aa, Homo sapiens
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 290 - protein, 208 aa, Homo sapiens
CQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWP

SEQ ID NO: 291 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 292 - protein, 21 aa, Homo sapiens
FLVIIVILSALFLGTLACFCV

SEQ ID NO: 293 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 294 - protein, 438 aa, Homo sapiens
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEA

SEQ ID NO: 295 - protein, 47 aa, Homo sapiens
CWRKIKQSCEEENAGAEDQDGEGEGSKTALQPLKHSDSKEDDGQEIA

SEQ ID NO: 296 - protein, 21 aa, Homo sapiens
LWVTVGLSVCLIALLVALAFV

SEQ ID NO: 297 - protein, 282 aa, Homo sapiens
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 298 - protein, 235 aa, Homo sapiens
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKAS

SEQ ID NO: 299 - protein, 21 aa, Homo sapiens
LCVSSFFAISWALLPLSPYLM

SEQ ID NO: 300 - protein, 341 aa, Homo sapiens
MSLMVVSMACVGFFLLQGAWPHEGVHRKPSLLAHPGPLVKSEETVILQCWSDVRFEHFLLHREGKYKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHERRFSAGPKVNGTFQADFPLGPATHGGTYRCFGSFRDSPYEWSNSSDPLLVSVTGNPSNSWPSPTEPSSKTGNPRHLHVLIGTSVVIILFILLLFFLLHRWCCNKKNAVVMDQEPAGNRTVNREDSDEQDPQEVTYAQLNHCVFTQRKITHPSQRPKTPPTDIIVYTELPNAEP

SEQ ID NO: 301 - protein, 412 aa, Homo sapiens
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLKQMESQPLPGERARSTLQKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 302 - protein, 355 aa, Homo sapiens
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 303 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 304 - protein, 366 aa, Homo sapiens [sequence omitted]

SEQ ID NO: 305 - protein, 229 aa, Homo sapiens
RDSPEDFVYQFKGMCYFTNGTERVRLVTRYIYNREEYARFDSDVGVYRAVTPQGRPVAEYWNSQKEVLERTRAELDTVCRHNYEVGYRGILQRRVEPTVTISPSRTEALNHHNLLVCSVTDFYPGQIKVQWFRNDQEETAGVVSTPLIRNGDWTFQILVMLEMTPQRGDVYTCHVEHPSLQSPITVEWRAQSESAQSKMLSGVGGFVLGLIFLGLGLIIRQRSQKGLLH

SEQ ID NO: 306 - protein, 1328 aa, Artificial Sequence
  EGFR806CAR-ERT2-P2A-DHFRdm-T2A-CD19t
MLLLVTSLLLCELPHPAFLLIPDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGSTSGSGKPGSGEGSTKGDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATVGSGATNFSLLKQAGDVEENPGPMVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKNDGGGEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 307 - protein, 4 aa, Artificial Sequence
  Degron sequence
RRRG

SEQ ID NO: 308 - protein, 5 aa, Artificial Sequence
  Degron sequence
RRRGN